(19) United States
(12) Patent Application Publication
(10) Pub. No.: US 2026/0265244 A1
(43) Pub. Date: Sep. 10, 2026

(54) HETEROCYCLIC COMPOUNDS AS STING ANTAGONISTS

(71) Applicant: Boehringer Ingelheim International GmbH, Ingelheim am Rhein (DE)

(72) Inventors: Matthias Hoffmann, Mittelbiberach (DE); Marta BRAMBILLA, Antwerpen (BE); Georg DAHMANN, Biberach (DE); Patrick GROSS, Biberach (DE); Sandra Ruth HANDSCHUH, Biberach an der Riss (DE); Jun LI, Danbury, CT (US); Camilla MAYER, Warthausen (DE); Herbert NAR, Ochsenhausen (DE); Thorsten OOST, Biberach an der Riss (DE)

(21) Appl. No.: 18/706,144

(22) PCT Filed: Oct. 26, 2023
(86) PCT No.: PCT/EP2023/079890

§ 371 Date: Apr. 30, 2024

Related U.S. Application Data
(60) Provisional application No. 63/381,349, filed on Oct. 28, 2022.

(30) Foreign Application Priority Data
Nov. 29, 2022 (EP) 22210157.8

(51) Int. Cl. C07D 471/04; A61K 31/4178; A61K 31/4184; A61K 31/437; A61K 31/444; A61K 31/496; A61K 31/5377; A61K 31/675; A61K 45/06; C07B 59/00; C07D 403/12; C07F 9/6561

(52) U.S. Cl. CPC C07D 471/04; A61K 31/4178; A61K 31/4184; A61K 31/437; A61K 31/444; A61K 31/496; A61K 31/5377; A61K 31/675; A61K 45/06; C07B 59/002; C07D 403/12; C07F 9/6561; C07B 2200/05

(57) ABSTRACT

This invention relates to compounds of formula 1;

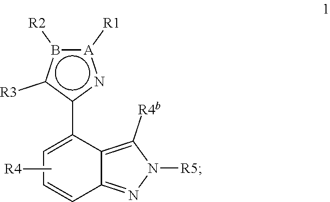

and their use as STING antagonists e.g. for the treatment of a disease selected from the group consisting of systemic lupus erythematosus (SLE), (monogenic and digenic) interferonopathies (including STING-associated vasculopathy with onset in infancy (SAVI). Aicardi-Goutieres syndrome (AGS), COPA syndrome, and familial chilblain lupus), type 1 interferonopathies with mutations in DNASE2 or ATAD3A genes, age-related macular degeneration (AMD), retinopathy, glaucoma, amyotrophic lateral sclerosis (ALS), diabetes, obesity, inflammatory bowel disease (IBD), chronic obstructive pulmonary disease (COPD), Bloom's syndrome, Sjogren's syndrome, Parkinson's disease, heart failure and cancer, systemic sclerosis (SSc), dermatomyositis, non-alcoholic fatty liver disease (NAFLD), non-alcoholic steatotic hepatitis (NASH), acute on chronic liver failure (ACLF), interstitial lung disease (ILD), idiopathic pulmonary fibrosis (IPF), aging/muscle disorders, sepsis, heart failure, rheumatoid arthritis and osteoarthritis.

### RELATED APPLICATION DISCLOSURE

This Application is a National Stage Application of International Application No. PCT/EP2023/079890 filed Oct. 26, 2023, which claims the benefit of U.S. Provisional Application No. 63/381,349 filed Oct. 28, 2022, and Foreign Priority of European Patent Application No. EP 22210157.8 filed Nov. 29, 2022, each of which is hereby incorporated by reference herein in its entirety.

### SEQUENCE DISCLOSURE

This application includes, as part of its disclosure, a “Sequence Listing XML” pursuant to 37 C.F.R. § 1.831(a) which is submitted in XML file format via the USPTO patent electronic filing system in a file named 01-3545-US-2_SL.xml, created on Apr. 6, 2026, and having a size of 4,014 bytes, which is hereby incorporated by reference herein in its entirety.

This invention relates to compounds of formula 1;

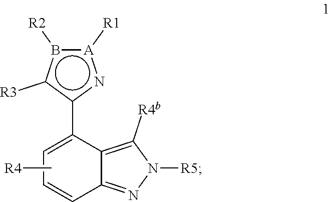

and their use as STING antagonists e.g. for the treatment of a disease selected from the group consisting of systemic lupus erythematosus (SLE), (monogenic and digenic) interferonopathies (including STING-associated vasculopathy with onset in infancy (SAVI), Aicardi-Goutieres syndrome (AGS), COPA syndrome, and familial chilblain lupus), type 1 interferonopathies with mutations in DNASE2 or ATAD3A genes, age-related macular degeneration (AMD), retinopathy, glaucoma, amyotrophic lateral sclerosis (ALS), diabetes, obesity, inflammatory bowel disease (IBD), chronic obstructive pulmonary disease (COPD), Bloom's syndrome, Sjogren's syndrome, Parkinson's disease, heart failure and cancer, systemic sclerosis (SSc), dermatomyositis, non-alcoholic fatty liver disease (NAFLD), non-alcoholic steatotic hepatitis (NASH), acute on chronic liver failure (ACLF), interstitial lung disease (ILD), idiopathic pulmonary fibrosis (IPF), aging/muscle disorders, sepsis, heart failure, rheumatoid arthritis and osteoarthritis.

### BACKGROUND OF THE INVENTION

Innate immunity is considered a first line cellular stress response defending the host cell against invading pathogens and initiating signaling to the adaptive immune system. These processes are triggered by conserved pathogen-associated molecular patterns (PAMPs) through sensing by diverse pattern recognition receptors (PRRs) and subsequent activation of cytokine and type I interferon gene expression. The major antigen-presenting cells, such as monocytes, macrophages, and dendritic cells produce type I interferons and are critical for eliciting adaptive T- and B-cell immune system responses. The major PRRs detect aberrant, i.e. mislocalized, immature or unmodified nucleic acids on either the cell surface, the inside of lysosomal membranes or within other cellular compartments (Barbalat et al., Annu. Rev. Immunol. 29, 185-214 (2011)).

“Cyclic GMP-AMP Synthase” (cGAS) is the predominant sensor for aberrant double-stranded DNA (dsDNA) originating from pathogens or mislocalization or misprocessing of nuclear or mitochondrial cellular dsDNA (Sun et al., Science 339, 786-791 (2013); Wu et al., Science 339, 826-830 (2013); Ablasser et al., Nature 498, 380-384 (2013)). Binding of dsDNA to cGAS activates the reaction of GTP and ATP to form the cyclic dinucleotide GMP-AMP (referred to as cGAMP). cGAMP then binds to and activates the endoplasmatic reticulum membrane-anchored adaptor protein, “Stimulator of Interferon Genes” (STING, UniProtKB—Q86WV6). Activated STING recruits and activates TANK-binding kinase 1 (TBK1) which in turn phosporylates the transcription factor family of interferon regulatory factors (IRFs) inducing cytokine and type I interferon mRNA expression. STING activation by cGAMP also leads to activation of NF-kB signaling pathway and downstream production of proinflammatory cytokines (Sun et al., Science 339, 786-791 (2013). Human GoF STING mutants lead to an autoinflammatory syndrome, cutaneous vasculopathy and lung fibrosis (STING-associated vasculopathy with onset in infancy, SAVI). SAVI patients have a highly activated PBMCs and dermal fibroblasts, exhibiting an upregulated type-1 IFN signature and expression of NFκB-mediated profibrotic and proinflammatory genes (e.g. TNFα, IL-6) (Liu et al., 2014).

The critical role of STING in dsDNA sensing has been established in different pathogenic bacteria and viruses. Additionally, STING is essential in various other biological processes such as cellular senescence (Yang et al., PNAS 114, E4612 (2017), Glück et al., Nat. Cell Biol. 19, 1061-1070 (2017)), autophagy and recognition of ruptured micronuclei in the surveillance of potential cancer cells (Mackenzie et al., Nature 548, 461-465 (2017); Harding et al., Nature 548, 466-470 (2017)).

While the cGAS/STING pathway is important for host defense against invading pathogens, cellular stress and genetic factors may also cause production of aberrant cellular dsDNA, e.g. by nuclear or mitochondrial leakage, and thereby trigger autoinflammatory responses. Aicardi-Goutieres syndrome (AGS; Crow et al., Nat. Genet. 38, 917-920 (2006))—a lupus-like severe autoinflammatory immune-mediated disorder—arises from genetic mutations such as loss-of-function mutations in TREX1, a primary DNA exonuclease responsible for degrading aberrant DNA in cytosol. Knock-out of STING in TREX1-deficient mice prevented otherwise lethal autoimmune responses, supporting STING as driver of interferonopathies (Gall et al., Immunity 36(1), 120-131 (2012); Gao et al., PNAS 112, E5699-E5705 (2015)). Likewise, embryonic lethality caused by deficiency of DNAse2, an endonuclease responsible for degradation of excessive DNA in lysosomes during endocytosis, was completely rescued by additional knock-out of STING (Ahn et al., PNAS 109, 19386-19391 (2012)). A STING inhibitor may provide a therapeutic strategy for preventing (monogenic and digenic) interferonopathy diseases such as SAVI, AGS, familial chilblain lupus and COPA. A STING inhibitor will block inflammation and aberrant tissue remodeling in a cluster of autoimmune and inflammatory diseases including systemic lupus erythematosus (SLE), systemic sclerosis, dermatomyositis, inflammatory bowel disease, sepsis, Sjogren's syndrome, atopic dermatitis, as well as a cluster fibrosis diseases including NASH, IPF, chronic kidney fibrosis. A STING inhibitor also has applications to additional diseases such as cancer, heart failure, AMD, retinopathy, glaucoma, aging, muscle disorders, rheumatoid arthritis, osteoarthritis, ALS, Parkinson's disease, COVID-19 (Decout et al, Nat Rev Immunol. 2021 21:548-569).

### PRIOR ART

Due to the observation that inhibition of the STING pathway may provide a therapeutic strategy for preventing autoinflammation and for treating e.g. autoimmune diseases efforts to develop STING inhibitors or inhibition of the STING signaling pathway have been undertaken.
In WO2019122202 for example, compounds C-178 or C-176 are described interfering with STING signaling pathway in HEK293T cells or bone marrow derived macrophages (BMDMs) stimulated with a cyclic dinucleotide such as e.g. cGAMP which are irreversible inhibitors blocking the palmitoylation of STING at an allosteric site of STING.
In ACS Med Chem Lett. (2019, 10, 1, pp 92-97), Siu et all described novel cGAMP competitive ligands. It is believed that inhibiting the orthosteric site of cGAMP mediated STING activation leads to a suppression of all STING mediated activation in contrast to palmitoylation inhibitors. Compound 13 or 15 in this publication inhibits the HAQ STING variant (displacement assay) with a moderate IC50 of 84 or 41 nM and shows low cellular inhibitory activity of about 11 uM based on a cGAMP stimulated INFb production in THP1 cells.
In International patent application WO2019069270, claims modulators of STING which either activate or inhibit STING accordingly.

Surprisingly it has now been found that the compounds described in the patent application are competitive inhibitors of STING.

### DETAILED DESCRIPTION OF THE INVENTION

The present invention is directed to a compound of formula 1;

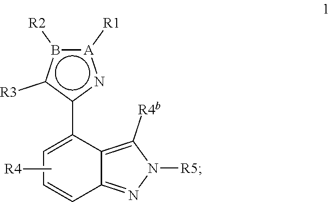

wherein
B-A is ═C—N— or —N—C═; this means A is C or N; B is C or N; but A and B are not N at the same time;
R1 is selected from

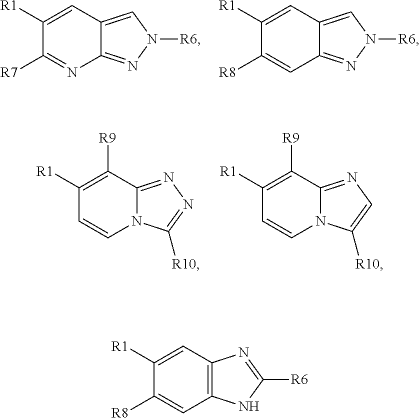

and R1 is the attaching point to the structure of formula 1;
R2 for B-A is —N—C═ has the meaning of C₁₋₆-alkyl-, C₃₋₆-cycloalkyl-, C₁₋₆-haloalkyl-;
R2 for B-A is =C—N— has the meaning of C₁₋₆-alkyl-, C₃₋₆-cycloalkyl-, C₁₋₆-haloalkyl-, C₁₋₆-alkyl-O—, HO—, H₂N—, C₁₋₆-alkyl-HN—, (C₁₋₆-alkyl)₂N—;
R3 H— or C₁₋₆-alkyl, C₃₋₇-cycloalkyl, C₂₋₆-alkenyl, C₃₋₇-heterocycloalkyl, each optionally substituted with a group selected from F—, HO—, Me-, EtO—, NH₂(O)C—;
R4 is H—, F— or HO—;
R4^{b} is H—, F—, Cl—, Br—, NC— or HO—;
R5 is selected from

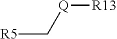

and R5 is the attaching point to the structure of formula 1;
Q is —C(R11)(R12)-, —S(O)— or —S(O)₂—;
R6 is C₂₋₆-alkenyl,
   or
   C₁₋₆-alkyl, optionally substituted independently of one another by one or two substituents selected from the group consisting of C₃₋₆-cycloalkyl-, halogen, HO—, C₁₋₆-alkyl-O—, C₁₋₆-alkyl-HN—, (C₁₋₆-alkyl)₂N—, NC—, (C₁₋₆-alkyl)₂(O)P—, (4-methoxyphenyl)methyl-, or
   a heterocycle selected from tetrahydrofuran-, 1, 4-dioxane-, pyrrolidine-, piperazine-, morpholine-, pyridine-, pyrazole-, triazole-, each optionally substituted independently of one another by one or two substituents selected from the group consisting of C₁₋₆-alkyl-, halogen, O═;
R7, R8, R9 are C₃₋₆-cycloalkyl-, optionally substituted with C₁₋₆-alkyl- or one or two halogen, or
   cyclopropylmethyl-, C₁₋₆-haloalkyl-, C₁₋₆-alkyl-O—, C₁₋₆-alkyl-HN—, (C₁₋₆-alkyl)₂N—, C₁₋₆-alkyl-S—,
   or
   C₁₋₆-alkyl, straight or branched, optionally substituted with HO—, C₁₋₆-alkyl-O—, C₁₋₆-alkyl-HN—, (C₁₋₆-alkyl)₂N— or morpholine-,
   or
   a heterocycle selected from pyrrolidine-, tetrahydrofuran-, tetrahydropyran-;
   or
   C₁₋₆-haloalkyl-O—;
R10 is C₁₋₆-alkyl- or C₁₋₆-haloalkyl-;
R11 is H, HO—, halogen-, or R14-O—, R14-NH—;
R12 is H— or F;
R13 is carbocyclyl, heterocyclyl, aryl, heteroaryl; preferably C₆₋₁₀-aryl, C₅₋₁₀-heteroaryl; each optionally substituted in with one or two substituents selected from the group consisting of C₁₋₆-alkyl, C₁₋₆-haloalkyl, HO—, NC—, halogen, R15-(CH₂)ₙ—O—, R15-(CH₂)ₙ—NH—, (R15-(CH₂)ₙ)₂—N—, R15-(CH₂)ₙ—S(O)—, R15-(CH₂)ₙ—S(O)₂—;
R14 is C₁₋₆-haloalkyl-, or C₁₋₆-alkyl, optionally substituted with C₃₋₆-cycloalkyl, C₂₋₆-alkenyl-, HO—, C₁₋₆-alkyl-O—, H₂N—C(O)—, C₁₋₆-alkyl-HN—C(O)—, (C₁₋₆-alkyl)₂N—C(O)—;
R15 is C₁₋₆-alkyl, C₁₋₆-haloalkyl, NC—, C₁₋₆-alkyl-HN—, (C₁₋₆-alkyl)₂N—, (C₁₋₆-alkyl)₂(HO)C—, aryl or heterocyclyl;
n is 0, 1, 2 or 3.

Preferred is a compound of formula 1, wherein
B-A is =C—N— or —N—C═; this means A is C or N; B is C or N; but A and B are not N at the same time;
R1 is selected from

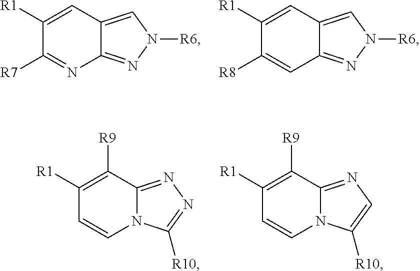

and R1 is the attaching point to the structure of formula 1;
R2 for B-A is —N—C═ has the meaning of C₁₋₆-alkyl-, C₃₋₆-cycloalkyl-, C₁₋₆-haloalkyl-;
R2 for B-A is =C—N— has the meaning of C₁₋₆-alkyl-, C₃₋₆-cycloalkyl-, C₁₋₆-haloalkyl-, C₁₋₆-alkyl-O—, HO—, H₂N—, C₁₋₆-alkyl-HN—, (C₁₋₆-alkyl)₂N—;
R3 H— or C₁₋₆-alkyl, C₃₋₇-cycloalkyl, C₂₋₆-alkenyl, C₃₋₇-heterocycloalkyl, each optionally substituted with a group selected from F—, HO—, Me-, EtO—, NH₂(O)C—;
R4 is H—, F— or HO—;
R4b is H—, F—, Cl—, Br—, NC— or HO—;
R5 is selected from

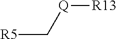

and R5 is the attaching point to the structure of formula 1;
Q is —C(R11)(R12)-, —S(O)— or —S(O)₂—;
R6 is C₂₋₆-alkenyl,
   or
   C₁₋₆-alkyl, optionally substituted independently of one another by one or two substituents selected from the group consisting of C₃₋₆-cycloalkyl-, halogen, HO—, C₁₋₆-alkyl-O—, C₁₋₆-alkyl-HN—, (C₁₋₆-alkyl)₂N—, NC—, (C₁₋₆-alkyl)₂(O)P—, (4-methoxyphenyl)methyl-,
   or
   a heterocycle selected from tetrahydrofuran-, 1, 4-dioxane-, pyrrolidine-, piperazine-, morpholine-, pyridine-, pyrazole-, triazole-, each optionally substituted independently of one another by one or two substituents selected from the group consisting of C₁₋₆-alkyl-, halogen, O═;
R7, R8, R9 are C₃₋₆-cycloalkyl-, optionally substituted with C₁₋₆-alkyl- or one or two halogen,
   or
   cyclopropylmethyl-, C₁₋₆-haloalkyl-, C₁₋₆-alkyl-O—, C₁₋₆-alkyl-HN—, (C₁₋₆-alkyl)₂N—, C₁₋₆-alkyl-S—,
   or
   C₁₋₆-alkyl, straight or branched, optionally substituted with HO—, C₁₋₆-alkyl-O—, C₁₋₆-alkyl-HN—, (C₁₋₆-alkyl)₂N— or morpholine-,
   or
   a heterocycle selected from pyrrolidine-, tetrahydrofuran-, tetrahydropyran-; or
   C₁₋₆-haloalkyl-O—;
R10 is C₁₋₆-alkyl- or C₁₋₆-haloalkyl-;
R11 is H, HO—, halogen-, or R14-O—, R14-NH—;
R12 is H— or F;
R13 is carbocyclyl, heterocyclyl, aryl, heteroaryl; preferably C₆₋₁₀-aryl, C₅₋₁₀-heteroaryl; each optionally substituted in with one or two substituents selected from the group consisting of C₁₋₆-alkyl, C₁₋₆-haloalkyl, HO—, NC—, halogen, R15-(CH₂)ₙ—O—, R15-(CH₂)ₙ—NH—, (R15-(CH₂)ₙ)₂—N—, R15-(CH₂)ₙ—S(O)—, R15-(CH₂)ₙ—S(O)₂—;
R14 is C₁₋₆-haloalkyl-, or C₁₋₆-alkyl, optionally substituted with C₃₋₆-cycloalkyl, C₂₋₆-alkenyl-, HO—, C₁₋₆-alkyl-O—, H₂N—C(O)—, C₁₋₆-alkyl-HN—C(O)—, (C₁₋₆-alkyl)₂N—C(O)—;
R15 is C₁₋₄-alkyl, C₁₋₆-haloalkyl, NC—, C₁₋₆-alkyl-HN—, (C₁₋₆-alkyl)₂N—, (C₁₋₆-alkyl)₂(HO)C—, aryl or heterocyclyl;
n is 0, 1, 2 or 3.

Preferred is a compound of formula 1, wherein
B-A is =C—N— or —N—C═; this means A is C or N; B is C or N; but A and B are not N at the same time;
R1 is selected from

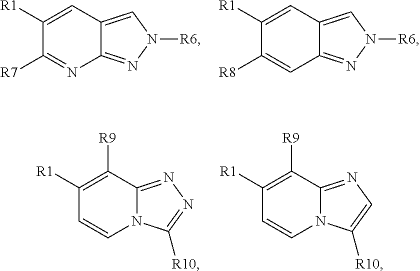

and R1 is the attaching point to the structure of formula 1;
R2 for B-A is —N—C═ has the meaning of C₁₋₆-alkyl-, C₃₋₆-cycloalkyl;
R2 for B-A is =C—N— has the meaning of C₁₋₆-alkyl-, C₃₋₆-cycloalkyl-, C₁₋₆-alkyl-O—, HO—, H₂N—, C₁₋₆-alkyl-HN—, (C₁₋₆-alkyl)₂N—;
R3 H— or C₁₋₆-alkyl, C₃₋₇-cycloalkyl, C₂₋₅-alkenyl, C₃₋₇-heterocycloalkyl, each optionally substituted with a group selected from F—, HO—, Me-, EtO—, NH₂(O)C—;
R4 is H—, F— or HO—;
R4^{b} is H—, F—, Cl—, Br—, NC— or HO—;
R5 is selected from

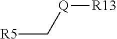

and R5 is the attaching point to the structure of formula 1;
Q is —C(R11)(R12)-, —S(O)— or —S(O)₂—;
R6 is C₂₋₄-alkenyl,
   or
   C₁₋₄-alkyl, optionally substituted independently of one another by one or two substituents selected from the group consisting of C₃₋₄-cycloalkyl-, halogen, HO—, C₁₋₄-alkyl-O—, C₁₋₄-alkyl-HN—(C₁₋₄-alkyl)₂N—, NC—, (C₁₋₄-alkyl)₂(O)P—, (4-Methoxyphenyl)methyl-, or
   a heterocycle selected from tetrahydrofuran-, 1, 4-dioxane-, pyrrolidine-, piperazine-, morpholine-, pyridine-, pyrazole-, triazole-, each optionally substituted independently of one another by one or two substituents selected from the group consisting of C₁₋₄-alkyl-, halogen, O═;
R7, R8, R9 are C₃₋₄-cycloalkyl-, optionally substituted with C₁₋₄-alkyl- or one or two halogen,
   or
   cyclopropylmethyl-, C₁₋₄-haloalkyl-, C₁₋₄-alkyl-O—, C₁₋₄-alkyl-HN—, (C₁₋₄-alkyl)₂N—, C₁₋₄-alkyl-S—,
   or
   C₁₋₄-alkyl, straight or branched, optionally substituted with HO—, C₁₋₄-alkyl-O—, C₁₋₄-alkyl-HN—, (C₁₋₄-alkyl)₂N— or morpholine-,
   or
   a heterocycle selected from pyrrolidine-, tetrahydrofuran-, tetrahydropyran-,
   or
   C₁₋₄-haloalkyl-O—;
R10 is C₁₋₄-alkyl- or C₁₋₄-haloalkyl-;
R11 is H, HO—, halogen-, or R14-O—, R14-NH—;
R12 is H— or F;
R13 is carbocyclyl, heterocyclyl, aryl, heteroaryl; preferably C₆₋₁₀-aryl, C₅₋₁₀-heteroaryl; each optionally substituted in with one or two substituents selected from the group consisting of C₁₋₄-alkyl, C₁₋₄-haloalkyl, HO—, NC—, halogen, R15-(CH₂)ₙ—O—, R15-(CH₂)ₙ—NH—, (R15-(CH₂)ₙ)₂—N—, R15-(CH₂)ₙ—S(O)—, R15-(CH₂)ₙ—S(O)₂—;
R14 is C₁₋₄-haloalkyl-, or C₁₋₅-alkyl, optionally substituted with C₃₋₄-cycloalkyl, C₂₋₄-alkenyl-, HO—, C₁₋₄-alkyl-O—, H₂N—C(O)—, C₁₋₄-alkyl-HN—C(O)—, (C₁₋₄-alkyl)₂N—C(O)—;
R15 is C₁₋₄-alkyl, C₁₋₄-haloalkyl, NC—, C₁₋₆-alkyl-HN—, (C₁₋₄-alkyl)₂N—, (C₁₋₄-alkyl)₂(HO)C—, aryl or heterocyclyl;
n is 0, 1, 2 or 3.

Preferred is a compound of formula 1, wherein
B-A is ═C—N— or —N—C═; this means A is C or N; B is C or N; but A and B are not N at the same time;
R1 is selected from

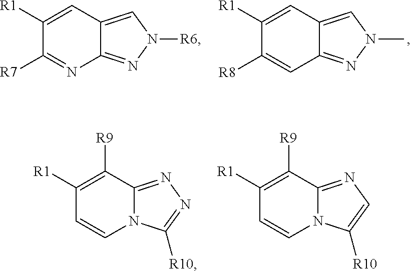

and R1 is the attaching point to the structure of formula 1;
R2 is C₁₋₄-alkyl-;
R3 is C₁₋₄-alkyl- optionally substituted with HO—,
   or
   C₃₋₄-cycloalkyl-, optionally substituted with methyl-;
R4 is H— or F—;
R5 is selected from

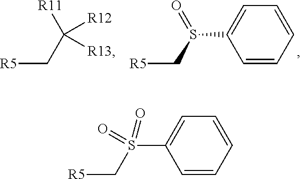

and R5 is the attaching point to the structure of formula 1;
R6 is C₂₋₄-alkenyl,
   or
   C₁₋₄-alkyl, optionally substituted independently of one another by one or two substituents selected from the group consisting of C₃₋₄-cycloalkyl-, halogen, HO—, C₁₋₄-alkyl-O—, (C₁₋₄-alkyl)₂N—, NC—, (C₁₋₄-alkyl)₂(O)P—, (4-Methoxyphenyl)methyl-,
   or
   a heterocycle selected from tetrahydrofuran-, 1, 4-dioxane-, pyrrolidine-, piperazine-, morpholine-, pyridine-, pyrazole-, triazole-, each optionally substituted independently of one another by one or two substituents selected from the group consisting of C₁₋₄-alkyl-, halogen, O═;
R7 is C₃₋₄-cycloalkyl-, optionally substituted with C₁₋₄-alkyl- or one or two halogen,
   or
   cyclopropylmethyl-, C₁₋₄-haloalkyl-, C₁₋₄-alkyl-O—, C₁₋₄-alkyl-HN—, (C₁₋₄-alkyl)₂N—, C₁₋₄-alkyl-S—,
   or
   C₁₋₄-alkyl, straight or branched, optionally substituted with HO—, C₁₋₄-alkyl-O—, C₁₋₄-alkyl-HN—, (C₁₋₄-alkyl)₂N— or morpholine-,
   or
   a heterocycle selected from pyrrolidine-, tetrahydrofuran-, tetrahydropyran-;
R8 is C₁₋₄-haloalkyl-O—;
R9 is C₁₋₄-alkyl- or C₃₋₄-cycloalkyl-;
R10 is C₁₋₄-alkyl- or C₁₋₄-haloalkyl-;
R11 is H, HO—, halogen-, or R14-O—;
R12 is H— or F;
R13 is cyclohexyl-, 3, 4-diflourphenyl-, 3-methyl-4N-pyridinyl-, or phenyl-, optionally substituted in 4-position with methyl, HO—, F—, Cl—, Br—, R15-(CH₂)ₙ—O—;
R14 is C₁₋₄-haloalkyl-, or C₁₋₅-alkyl, optionally substituted with C₃₋₄-cycloalkyl, C₂₋₄-alkenyl-, HO—, C₁₋₄-alkyl-O—, H₂N—C(O)—, (C₁₋₄-alkyl)NH—C(O)—, (C₁₋₄-alkyl)₂N—C(O)—; R15 is NC—, (C₁₋₄-alkyl)₂N—, (C₁₋₄-alkyl)₂(HO)C—, phenyl, or a heterocycle selected from oxetane-, tetrahydropyran-, morpholine-;
n is 0, 1 or 2.

Preferred is a compound of formula 1, wherein
B-A is ═C—N— or —N—C═; this means A is C or N; B is C or N; but A and B are not N at the same time;
R1 is selected from

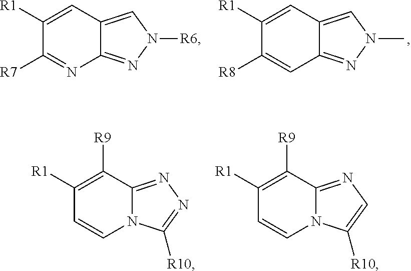

preferably

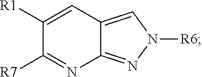

and R1 is the attaching point to the structure of formula 1;
R2 is methyl- or ethyl-, preferably methyl-;
R3 is ethyl-, iso-propyl-, cyclopropyl-, 1-methyl-cyclopropyl-, 1-hydroxy-iso-propyl-;
R4 is H— or F—;
R5 is selected from

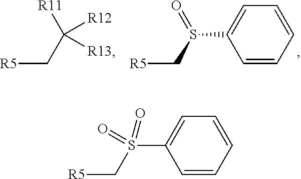

and R5 is the attaching point to the structure of formula 1;
R6 is C₂₋₄-alkenyl,
   or
   C₁₋₄-alkyl, optionally substituted independently of one another by one or two substituents selected from the group consisting of cyclopropyl-, F—, HO—, H₃C—O—, (H₃C)₂N—, NC—, (H₃C)₂(O)P—, (4-Methoxyphenyl)methyl-,
   or
   a heterocycle selected from tetrahydrofuran-, 1, 4-dioxane-, pyrrolidine-, piperazine-, morpholine-, pyridine-, pyrazole-, triazole-, each optionally substituted independently of one another by one or two substituents selected from the group consisting of H₃C—, F—, O═;
   or R6 is preferably methyl-, ethyl-, iso-propyl-, cyclopropyl-;
R7 is cyclopropyl-, 1-flourocyclopropyl-, 2, 2-diflourocyclopropyl-, 1-methyl-cyclopropyl-, cyclobutanyl-, cyclopropylmethyl-, F₂HC—, F₃C—, (iPr)—O—, (H₃C)NH—, (H₃C)₂N—, H₃C—S—,
   or
   C₁₋₄-alkyl, straight or branched, optionally substituted with HO—, H₃C—O—, H₃C—CH₂—O—, (H₃C)NH—, (H₃C)₂N— or morpholine-,
   or
   a heterocycle selected from pyrrolidine-, tetrahydrofuran-, tetrahydropyran-;
R8 is F₂HC—O—, F₃C—O—;
R9 is methyl or cyclopropyl;
R10 is methyl or F₂C—;
R11 is H, HO—, F—, or R14-O—;
R12 is H— or F; preferably H—;
R13 is cyclohexyl-, 3, 4-diflourphenyl-, 3-methyl-4N-pyridinyl-, or phenyl-, optionally substituted in 4-position with methyl, HO—, F—, Cl—, Br—, R15-(CH₂)ₙ—O—;
R14 is FH₂C—, FH₂C—CH₂—, or C₁₋₅-alkyl, optionally substituted with cyclopropyl, H₂C═CH—, HO—, H₃C—O—, H₂N—C(O)—, (H₃C)NH—C(O)—;
R15 is NC—, (H₃C)₂N—, (H₃C)₂(HO)C—, phenyl, or a heterocycle selected from oxetane-, tetrahydropyran-, morpholine-;
n is 0, 1 or 2.

Preferred is a compound of formula 1, wherein
B-A is ═C—N— or —N—C═; this means A is C or N; B is C or N; but A and B are not N at the same time;
R1 is selected from

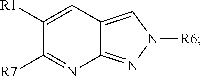

and R1 is the attaching point to the structure of formula 1;
R2 is methyl-;
R3 is ethyl-, iso-propyl-, cyclopropyl-, 1-methyl-cyclopropyl-, 1-hydroxy-iso-propyl-;
R4 is H— or F—;
R5 is selected from

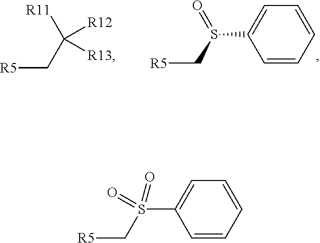

and R5 is the attaching point to the structure of formula 1;
R6 is methyl-, ethyl-, iso-propyl-, cyclopropyl-;
R7 is cyclopropyl-, 1-fluorocyclopropyl-, 2, 2-difluorocyclopropyl-, 1-methyl-cyclopropyl-, cyclobutanyl-, cyclopropylmethyl-, F₂HC—, F₃C—, (iPr)—O—, (H₃C)NH—, (H₃C)₂N—, H₃C—S—,
   or
   C₁₋₄-alkyl, straight or branched, optionally substituted with HO—, H₃C—O—, H₃C—CH₂—O—, (H₃C)NH—, (H₃C)₂N— or morpholine-,
   or
   a heterocycle selected from pyrrolidine-, tetrahydrofuran-, tetrahydropyran-;
R11 is H, HO—, F—, or R14-O—;
R12 is H—;
R13 is cyclohexyl-, 3, 4-difluorphenyl-, 3-methyl-4N-pyridinyl-, or phenyl-, optionally substituted in 4-position with methyl, HO—, F—, Cl—, Br—, R15-(CH₂)ₙ—O—;
R14 is FH₂C—, FH₂C—CH₂—, or C₁₋₅-alkyl, optionally substituted with cyclopropyl, H₂C═CH—, HO—, H₃C—O—, H₂N—C(O)—, (H₃C)NH—C(O)—;
R15 is NC—, (H₃C)₂N—, (H₃C)₂(HO)C—, phenyl, or a heterocycle selected from oxetane-, tetrahydropyran-, morpholine-;
n is 0, 1 or 2.

Preferred is a compound of formula 1, wherein
B-A is ═C—N— or —N—C═; this means A is C or N; B is C or N; but A and B are not N at the same time;
R1 is selected from

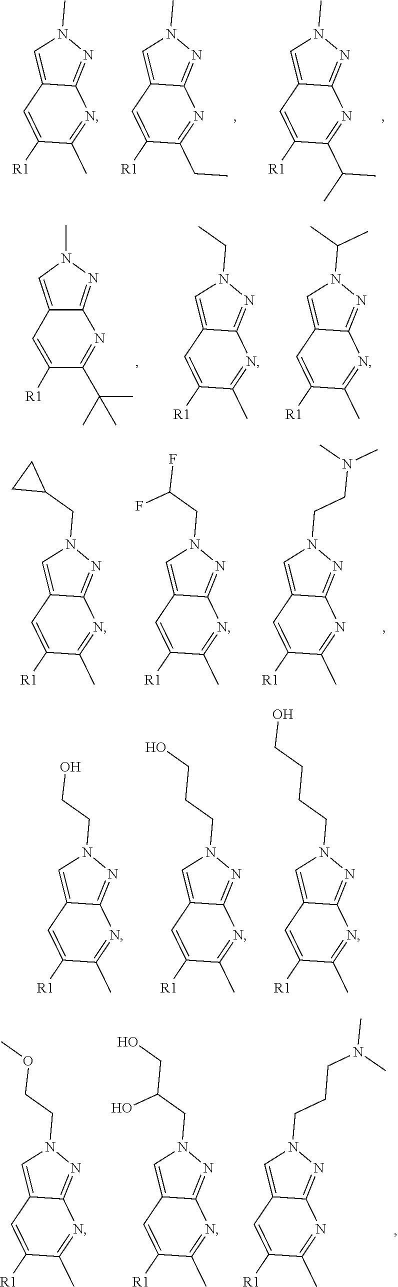

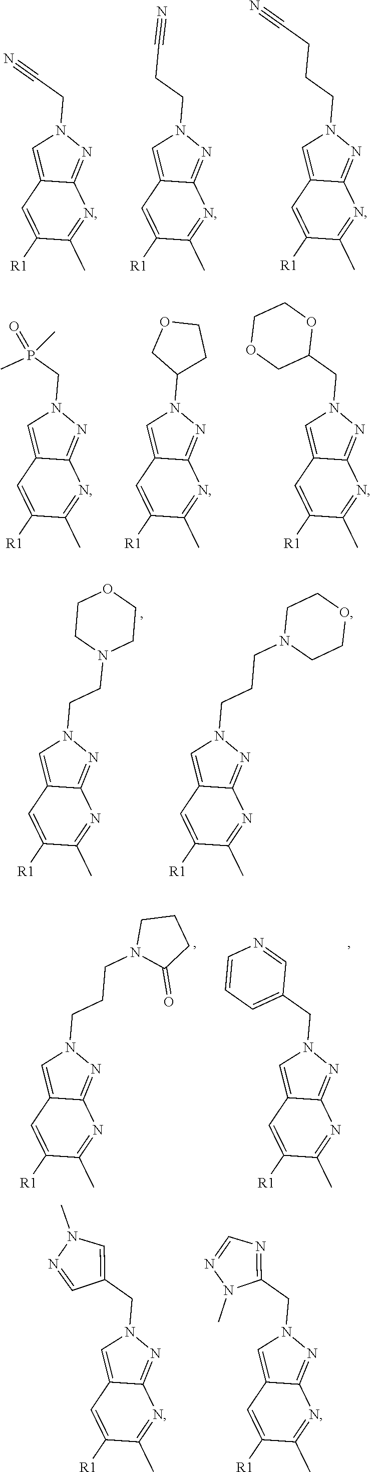

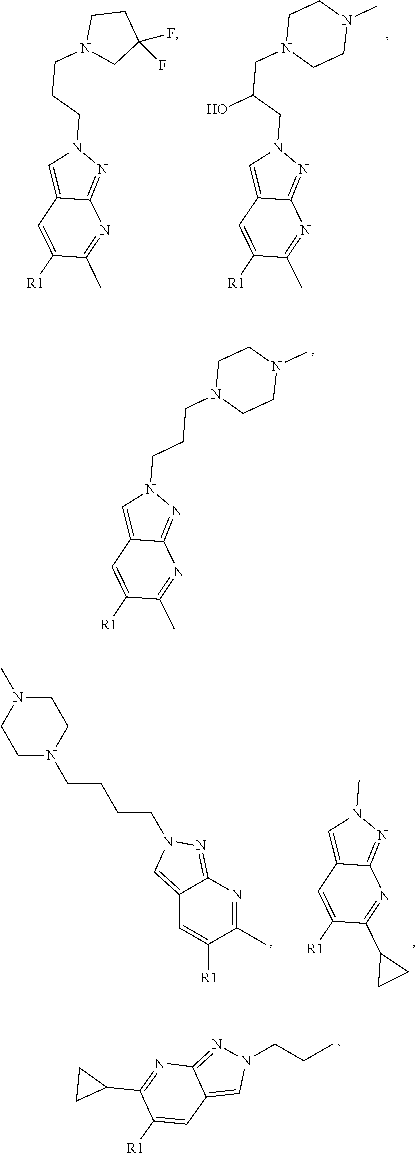

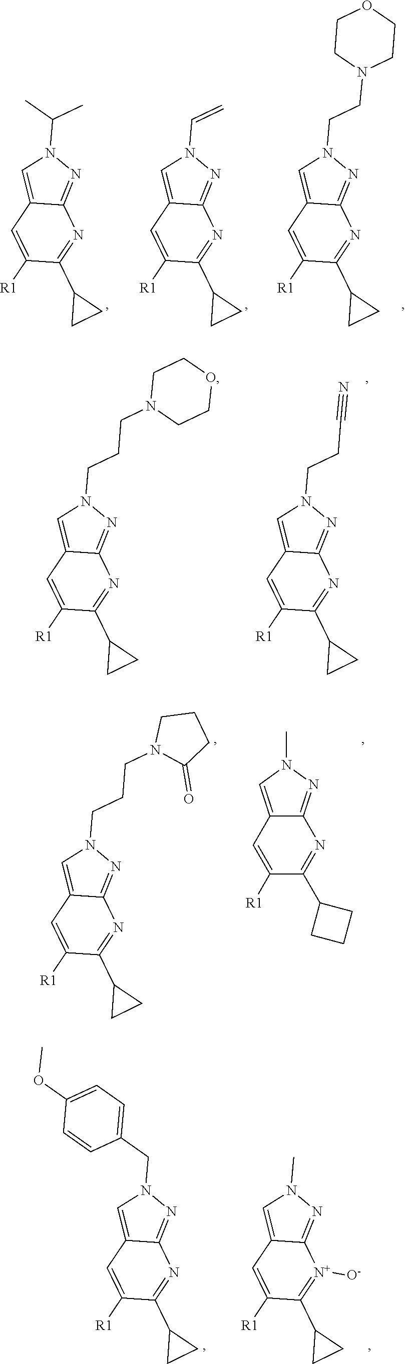

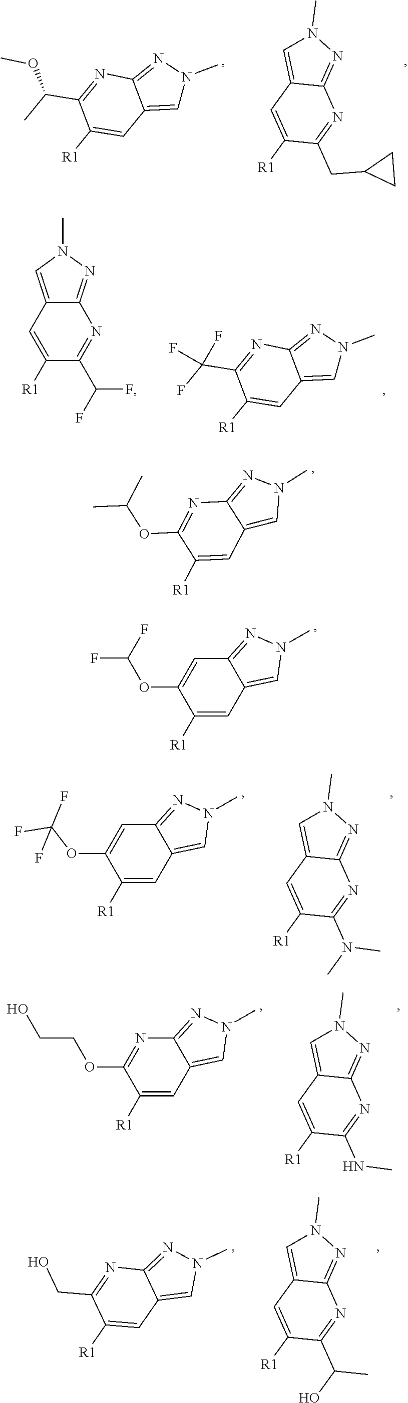

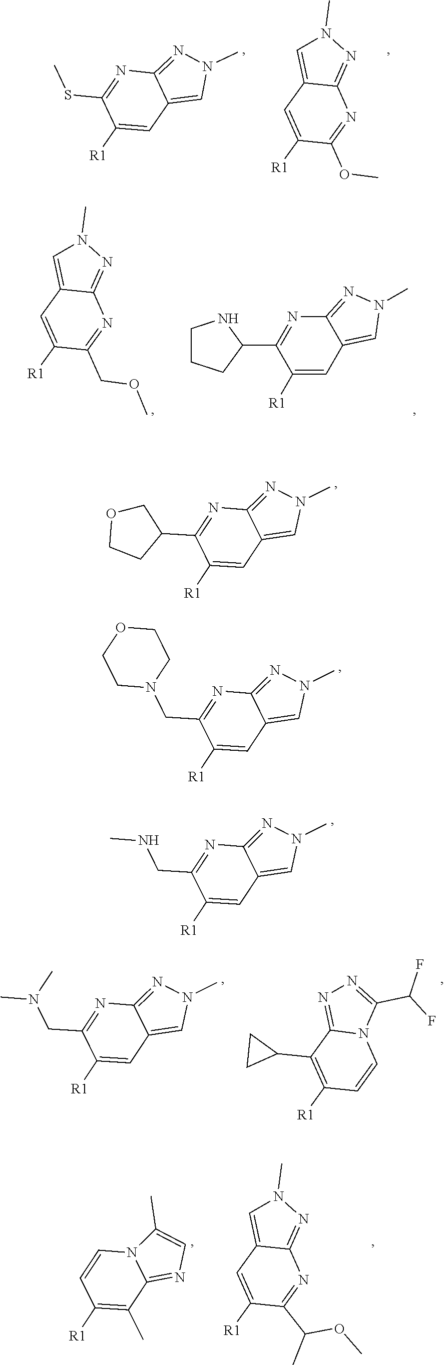

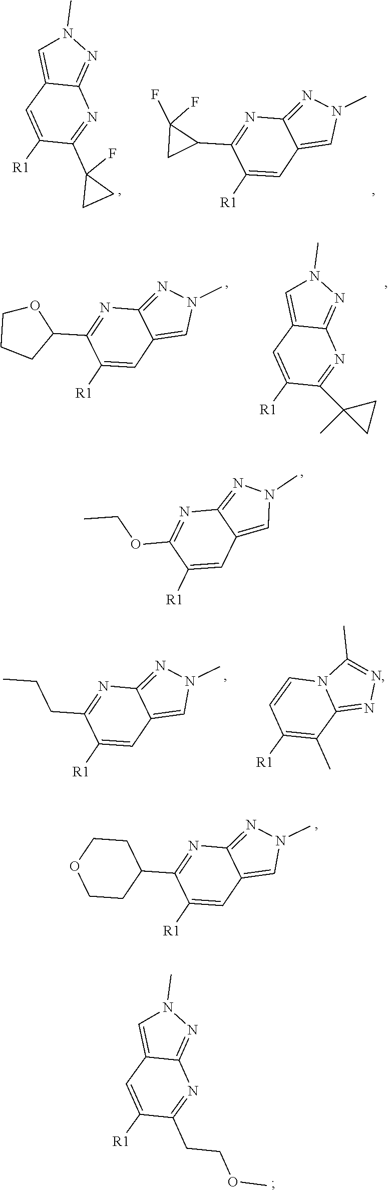

and R1 is the attaching point to the structure of formula 1;
R2 is methyl- or ethyl-, preferably methyl-;
R3 is ethyl-, iso-propyl-, cyclopropyl-, 1-methyl-cyclopropyl-, ((HO)H₂C)(H₃C)HC—;
R4 is H— or F—;
R5 is selected from

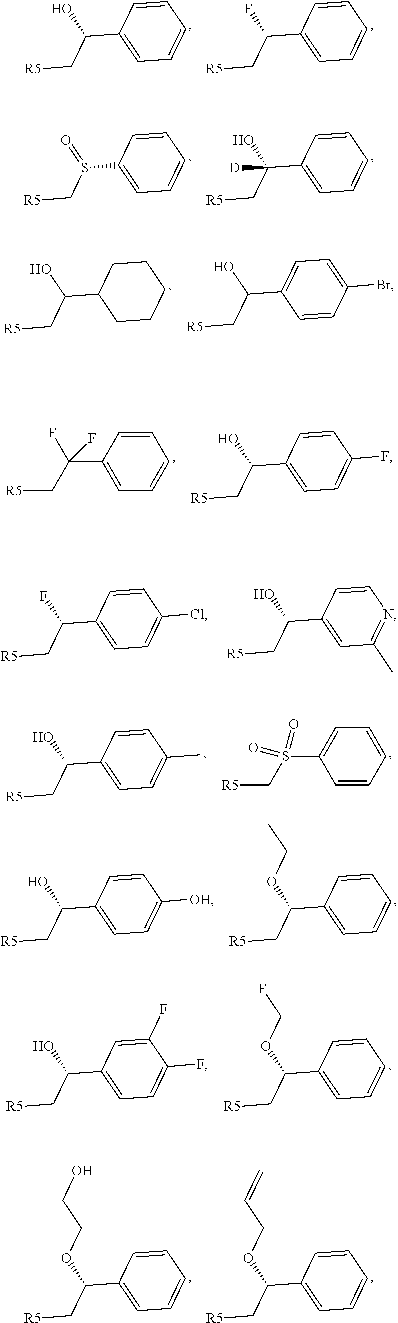

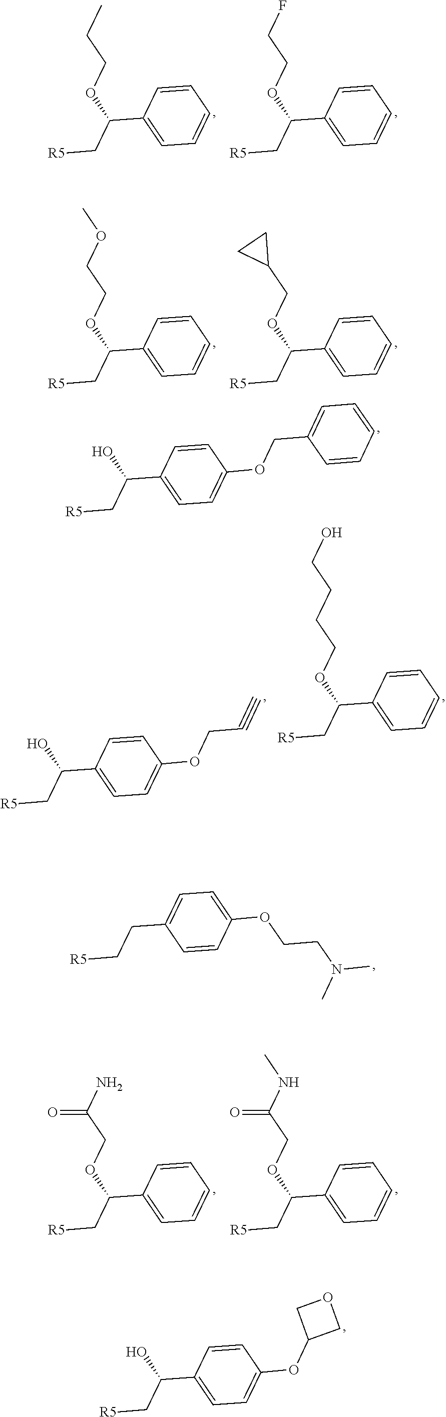

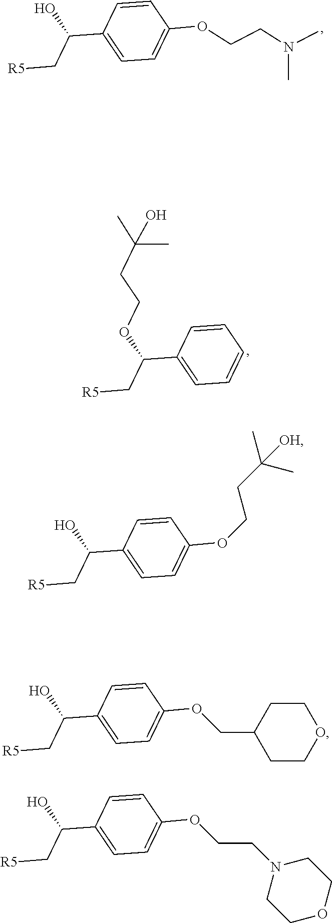

and R5 is the attaching point to the structure of formula 1. to Preferred is a compound of formula 1, wherein
B-A is ═C—N— or —N—C═; this means A is C or N; B is C or N; but A and B are not N at the same time;
R1 is selected from

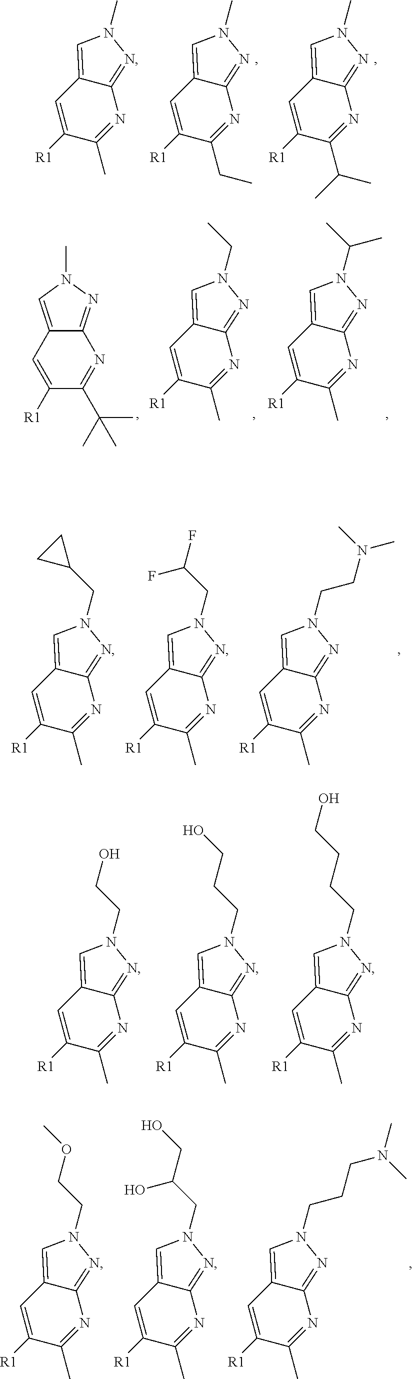

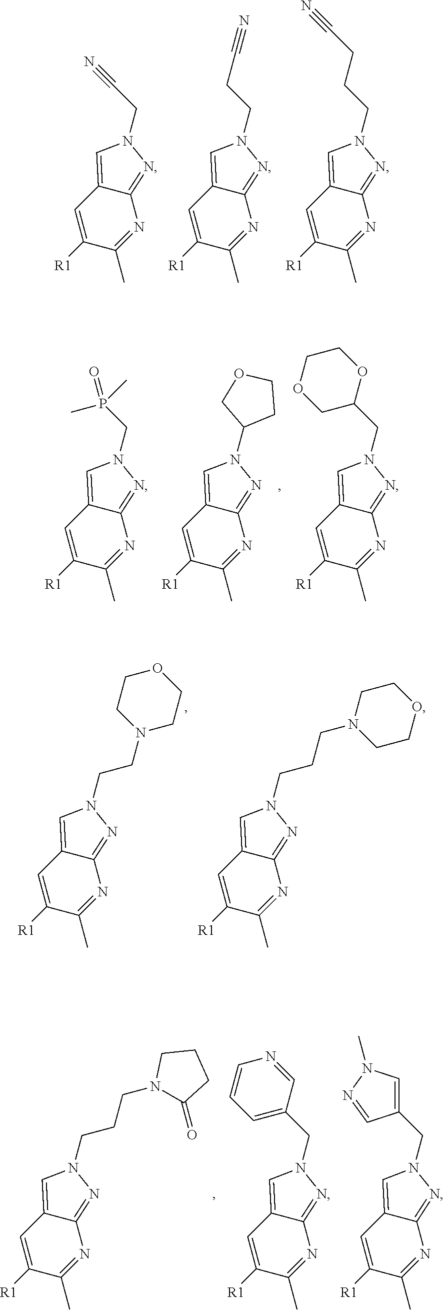

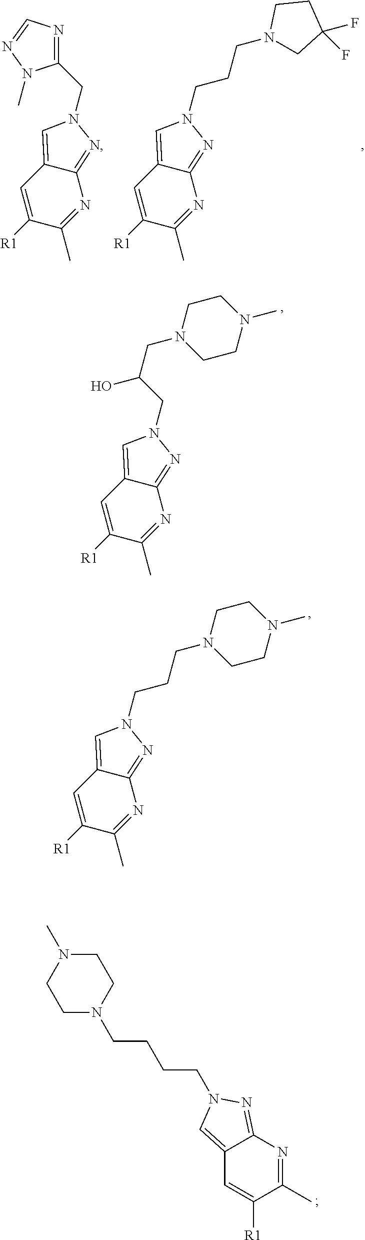

and R1 is the attaching point to the structure of formula 1;
R2 is methyl- or ethyl-, preferably methyl-;
R3 is ethyl-, iso-propyl-, cyclopropyl-, 1-methyl-cyclopropyl-, ((HO)H₂C)(H₃C)HC—;
R4 is H— or F—;
R5 is selected from

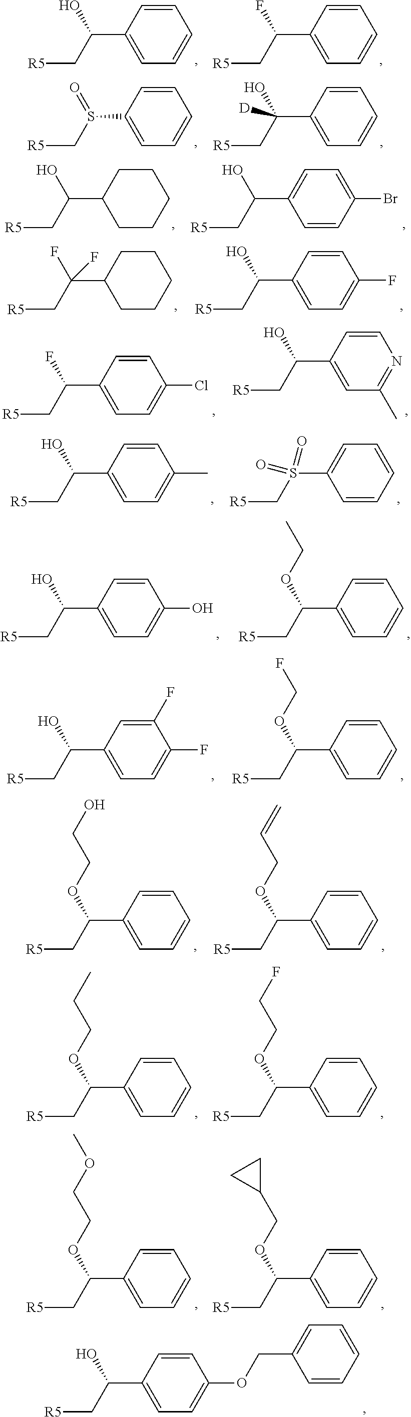

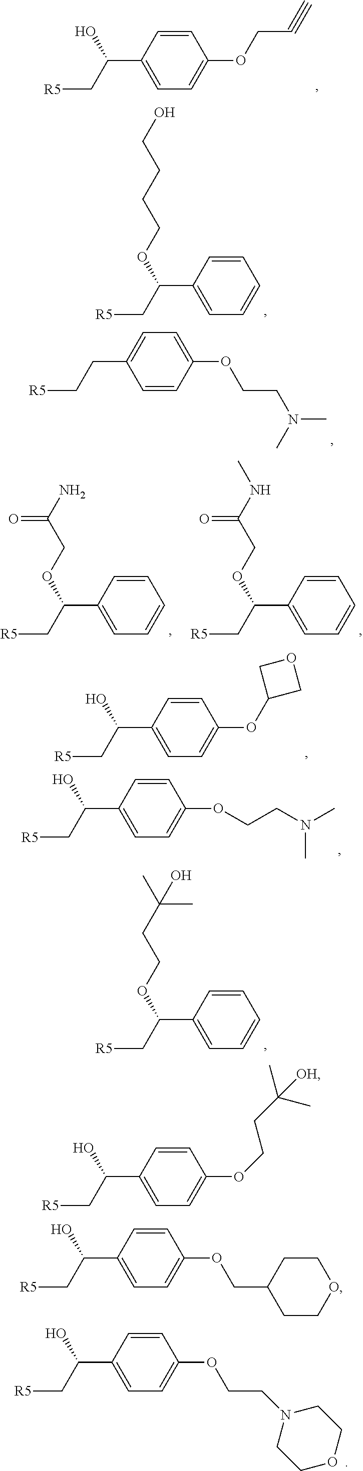

and R5 is the attaching point to the structure of formula 1.

Preferred is a compound of formula 1, wherein
B-A is ═C—N— or —N—C═; this means A is C or N; B is C or N; but A and B are not N at the same time;
R1 is selected from

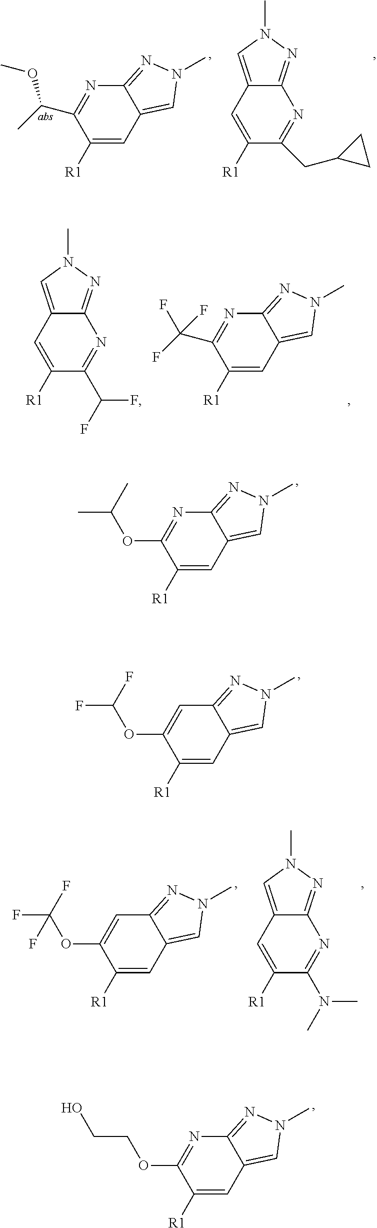

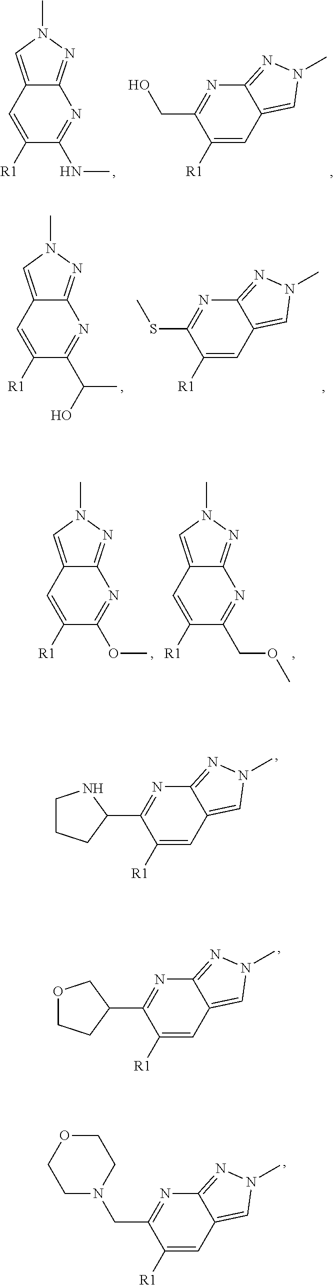

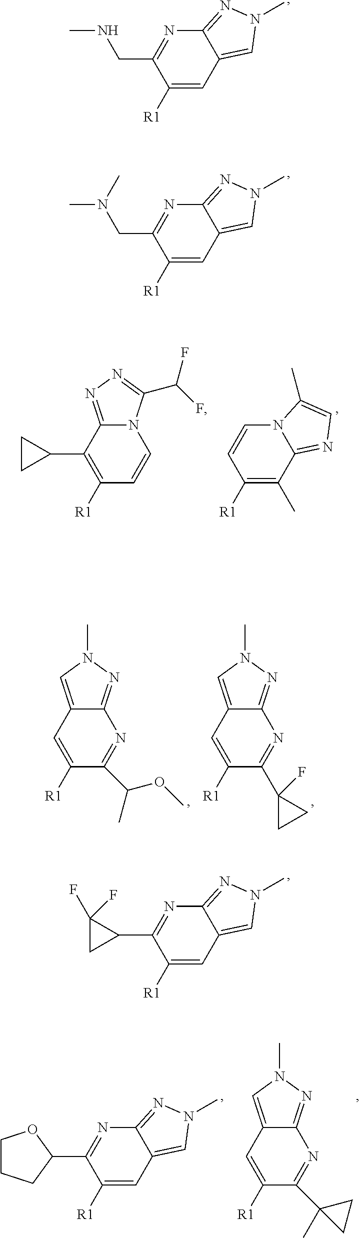

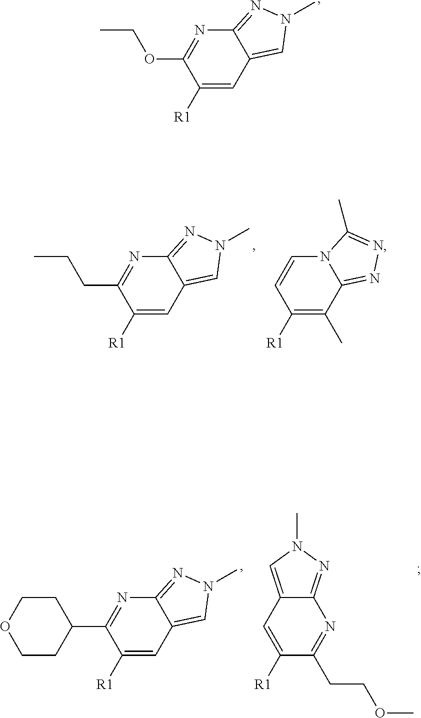

and R1 is the attaching point to the structure of formula 1;
R2 is methyl- or ethyl-, preferably methyl-;
R3 is ethyl-, iso-propyl-, cyclopropyl-, 1-methyl-cyclopropyl-, ((HO)H₂C)(H₃C)HC—;
R4 is H— or F—;
R5 is selected from

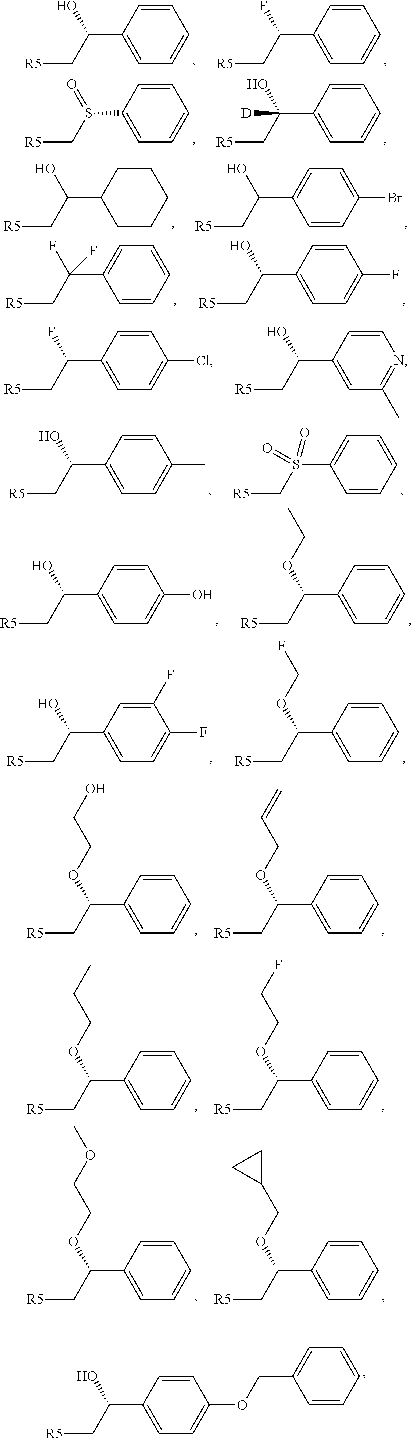

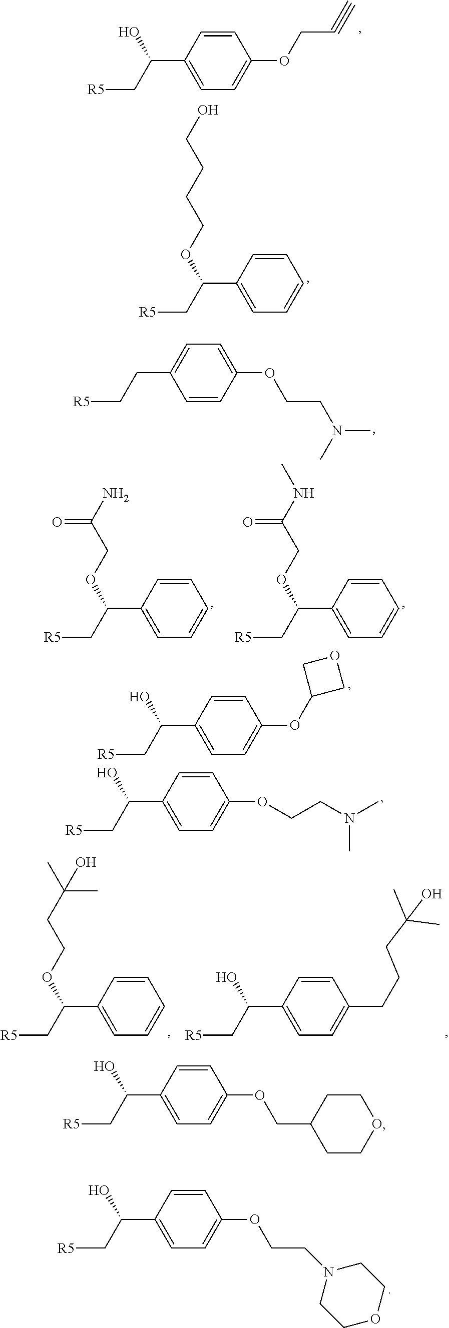

and R5 is the attaching point to the structure of formula 1.

Preferred is a compound of formula 1, wherein
B-A is ═C—N— or —N—C═; this means A is C or N; B is C or N; but A and B are not N at the same time;
R1 is selected from

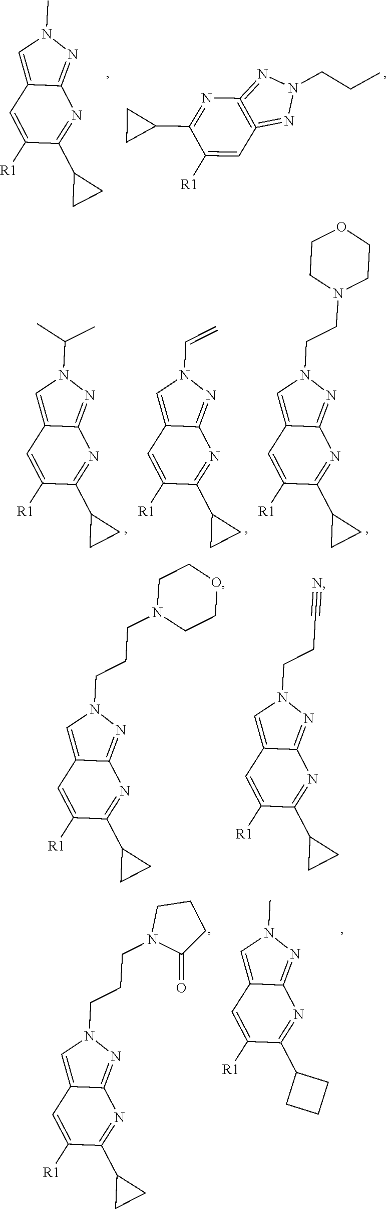

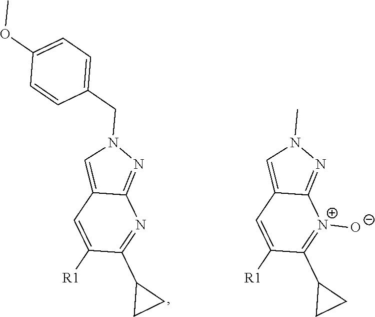

and R1 is the attaching point to the structure of formula 1;
R2 is methyl- or ethyl-, preferably methyl-;
R3 is ethyl-, iso-propyl-, cyclopropyl-, 1-methyl-cyclopropyl-, ((HO)H₂C)(H₃C)HC—;
R4 is H— or F—;
R5 is selected from

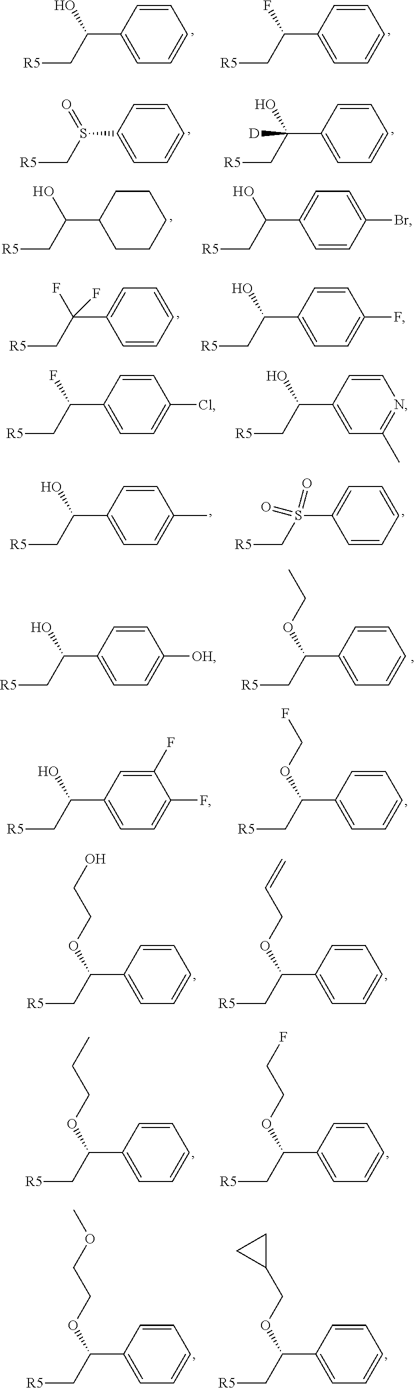

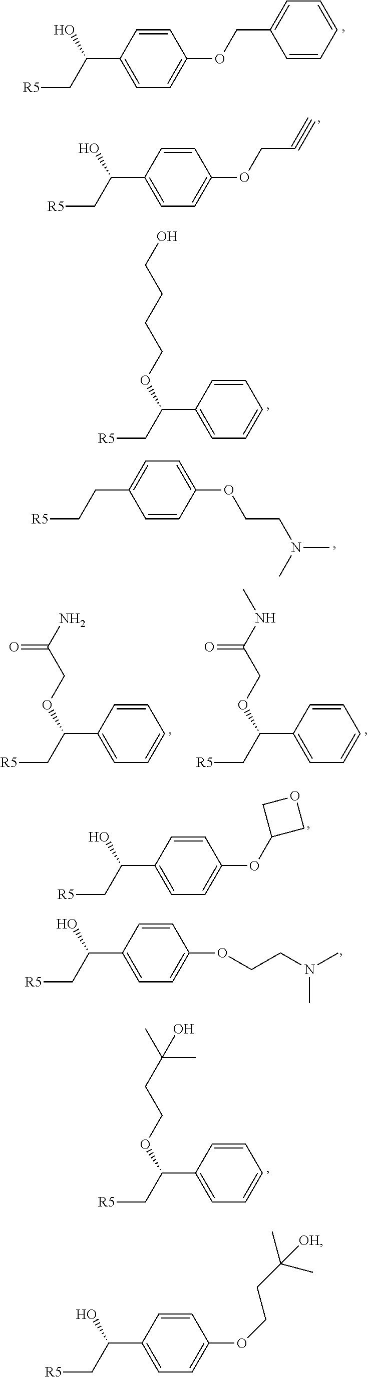

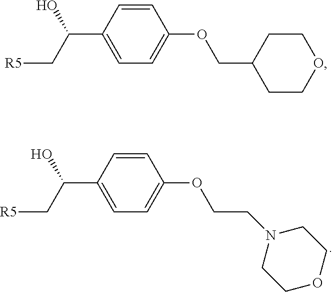

and R5 is the attaching point to the structure of formula 1.

Preferred is a compound of formula 1 wherein R1 is

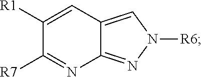

and R6 and R7 are defined as above and R1 is the attaching point to the structure of formula 1.

Preferred is a compound of formula 1 wherein R1 is

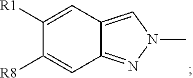

and R8 is defined as above and R1 is the attaching point to the structure of formula 1.

Preferred is a compound of formula 1 wherein R1 is

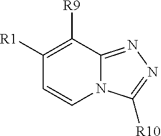

and R9 and R10 are defined as above and R1 is the attaching point to the structure of formula 1.

Preferred is a compound of formula 1 wherein R1 is

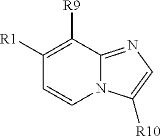

and R9 and R10 are defined as above and R1 is the attaching point to the structure of formula 1.

Preferred is a compound of formula 1 wherein R1 is selected from

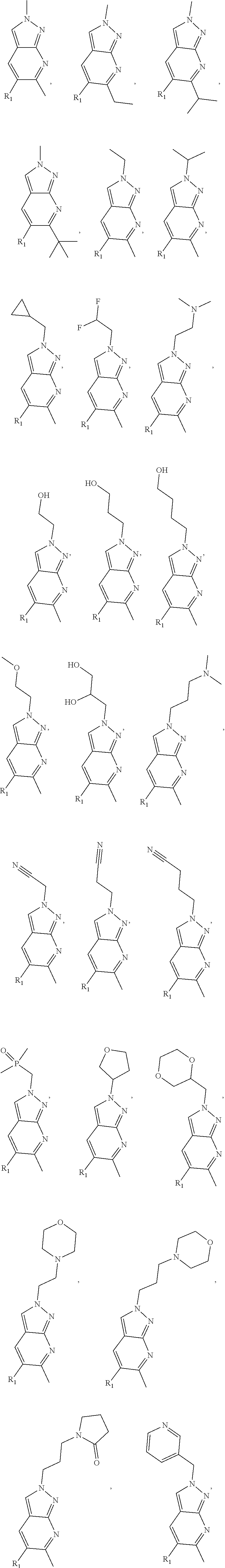

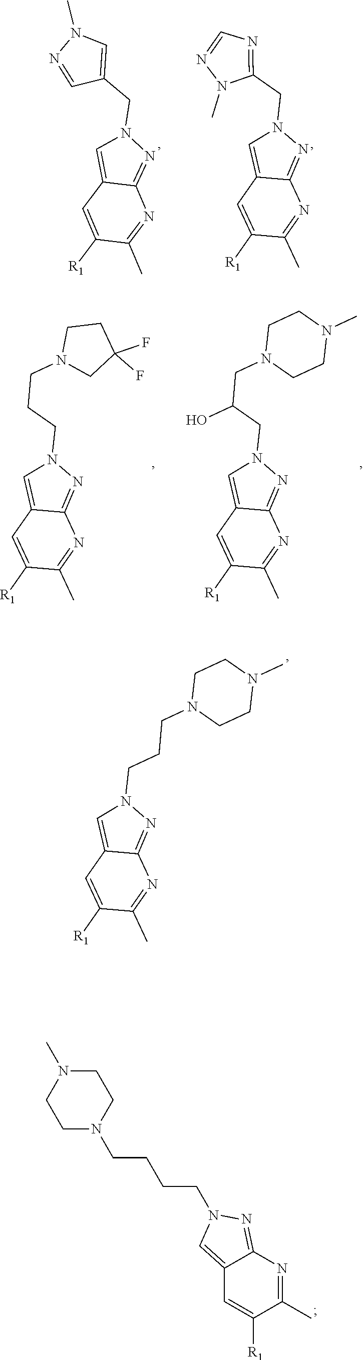

and R1 is the attaching point to the structure of formula 1.

Preferred is a compound of formula 1 wherein R1 is selected from

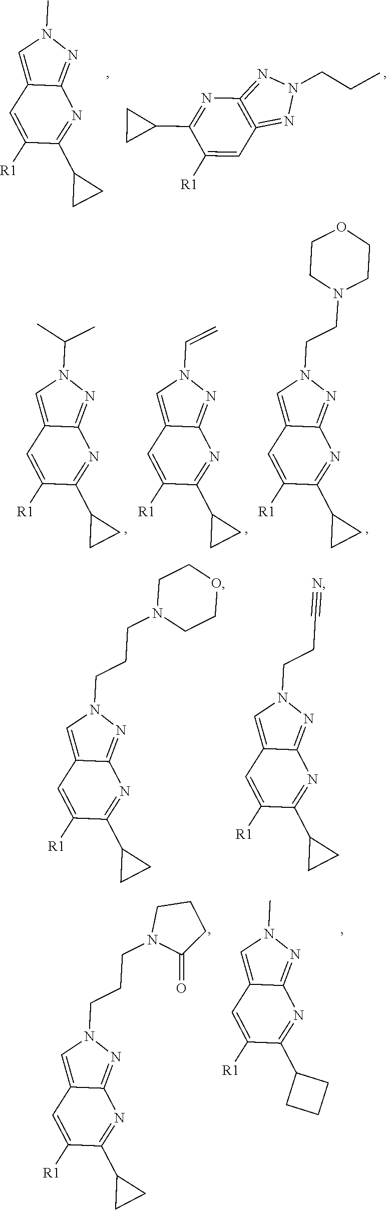

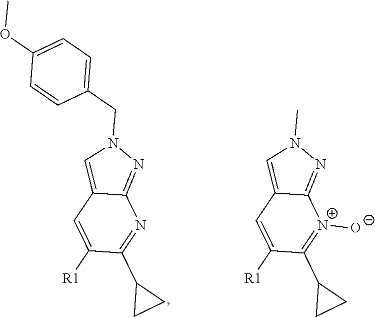

and R1 is the attaching point to the structure of formula 1.

Preferred is a compound of formula 1 wherein R1 is selected from

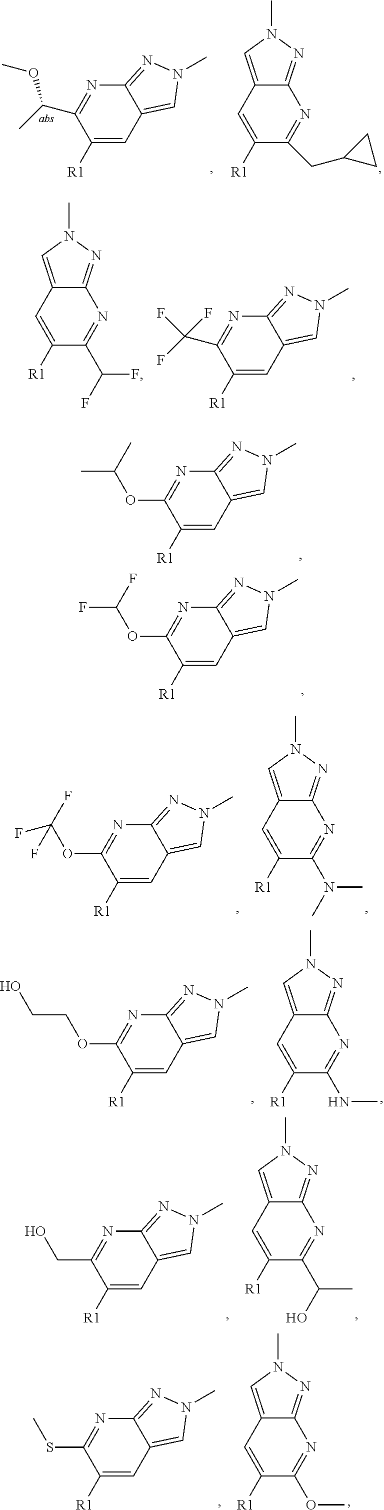

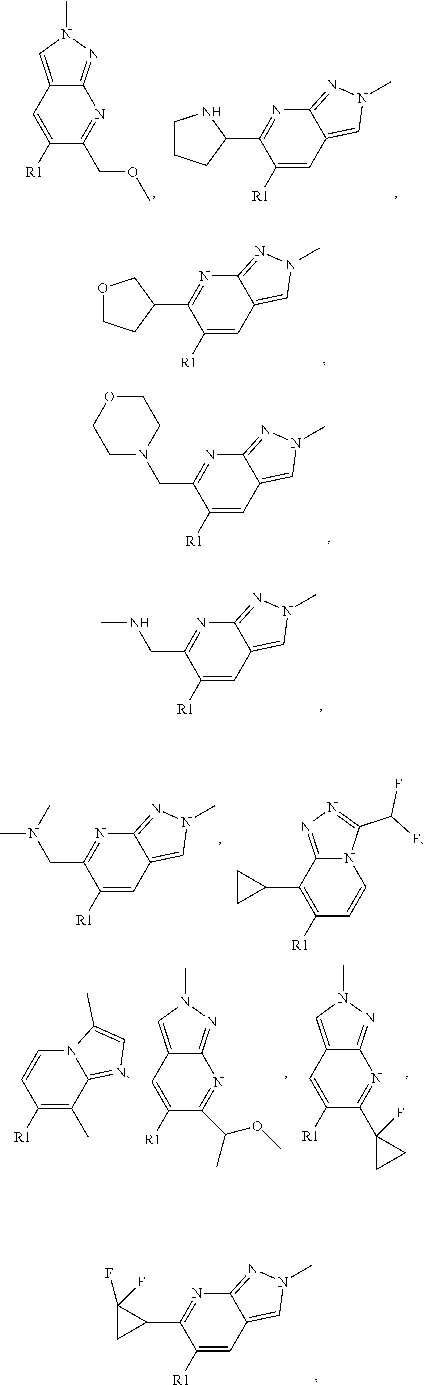

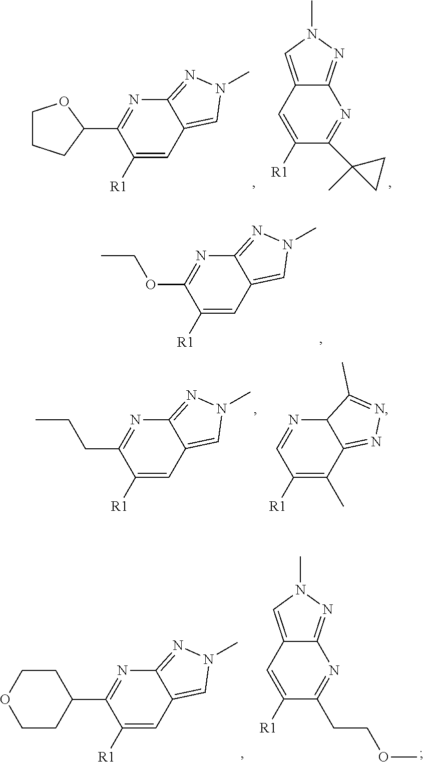

and R1 is the attaching point to the structure of formula 1.

Preferred is a compound of formula 1 wherein R5 is

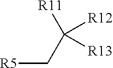

and R11, R12 and R13 are defined as above and R1 is the attaching point to the structure of formula 1.

Preferred is a compound of formula 1 wherein R5 is

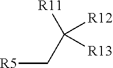

and
R11 is HO—, F—, or R14-O—;
R12 is H—;
R13 is cyclohexyl-, 3, 4-difluorphenyl-, 3-methyl-4N-pyridinyl-, or phenyl-, optionally substituted in 4-position with methyl, HO—, F—, Cl—, Br—, R15-(CH₂)ₙ—O—;
R14 is FH₂C—, FH₂C—CH₂—, or C₁₋₅-alkyl, optionally substituted with cyclopropyl, H₂C═CH—, HO—H₃C—O—, H₂N—C(O)—, (H₃C)NH—C(O)—;
R15 is NC—, (H₃C)₂N—, (H₃C)₂(HO)C—, phenyl, or a heterocycle selected from oxetane-, tetrahydropyran-, morpholine-;
n is 0, 1 or 2,
and R1 is the attaching point to the structure of formula 1.

Preferred is a compound of formula 1 wherein R5 is

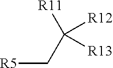

and
R11 is HO—;
R12 is H—;
R13 is phenyl-, optionally substituted in 4-position with methyl, HO—, F—, Cl—, Br—, R15-(CH₂)ₙ—O—;
R15 is NC—, (H₃C)₂N—, (H₃C)₂(HO)C—, phenyl, or a heterocycle selected from oxetane-, tetrahydropyran-, morpholine-;
n is 0, 1 or 2,
and R1 is the attaching point to the structure of formula 1.

Preferred is a compound of formula 1 wherein R5 is

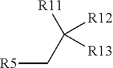

and
R11 is HO—;
R12 is H—;
R13 is phenyl-, optionally substituted in 4-position with methyl, HO—, F—, Cl—, Br—;
and R1 is the attaching point to the structure of formula 1.

Preferred is a compound of formula 1 wherein R5 is

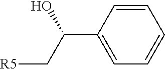

Preferred is a compound of formula 1 wherein R5 is

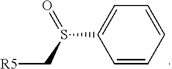

Preferred is a compound of formula 1 wherein R5 is

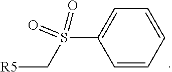

Preferred is also a compound of formula 1a.

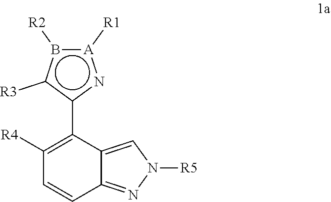

Preferred is also a compound of formula 1b.

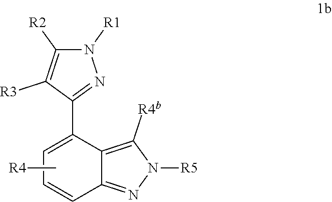

Preferred is also a compound of formula 1b1.

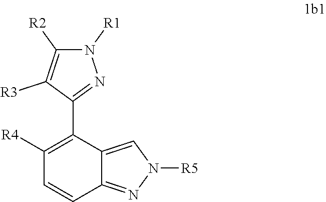

Preferred is also a compound of formula 1c.

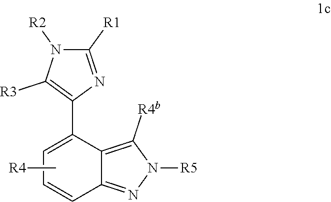

Preferred is also a compound of formula 1c1.

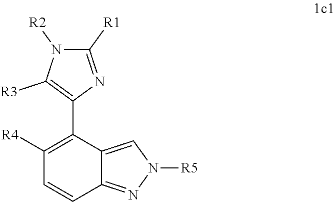

Preferred is a compound of formula 1 wherein B-A is ═C—N— and R3 is iso-propyl.

Preferred is a compound of formula 1 wherein B-A is —N—C═ and R3 is cyclopropyl.

Preferred is also a salt of a compound of formula 1, 1a, 1b, 1b1, 1c or 1c1. Furthermore, preferred is a pharmaceutically acceptable salt of a compound of formula 1, 1a, 1b, 1b1, 1c or 1c1.

In a preferred embodiment the invention refers to the above-mentioned compounds of formula 1, 1a, 1b, 1b1, 1c or 1c1 and their use as STING antagonists e.g. for the treatment of a disease selected from the group consisting of systemic lupus erythematosus (SLE), (monogenic and digenic) interferonopathies (including STING-associated vasculopathy with onset in infancy (SAVI), Aicardi-Goutieres syndrome (AGS), COPA syndrome, and familial chilblain lupus), type 1 interferonopathies with mutations in DNASE2 or ATAD3A genes, age-related macular degeneration (AMD), retinopathy, glaucoma, amyotrophic lateral sclerosis (ALS), diabetes, obesity, inflammatory bowel disease (IBD), chronic obstructive pulmonary disease (COPD), Bloom's syndrome, Sjogren's syndrome, Parkinson's disease, heart failure and cancer, systemic sclerosis (SSc), dermatomyositis, non-alcoholic fatty liver disease (NAFLD), non-alcoholic steatotic hepatitis (NASH), acute on chronic liver failure (ACLF), interstitial lung disease (ILD), idiopathic pulmonary fibrosis (IPF), aging/muscle disorders, sepsis, heart failure, rheumatoid arthritis and osteoarthritis.

In a more preferred embodiment, the invention relates to the aforementioned compounds of formula 1, 1a, 1b, 1b1, 1c or 1c1 for use in the treatment of a disease selected from the group consisting of systemic lupus erythematosus (SLE), (monogenic and digenic) interferonopathies, Aicardi-Goutieres syndrome, type 1 interferonopathies with mutations in DNASE2 or ATAD3A genes, age-related macular degeneration (AMD), amyotrophic lateral sclerosis (ALS), inflammatory bowel disease (IBD), chronic obstructive pulmonary disease (COPD), Bloom's syndrome, Sjogren's syndrome and Parkinson's disease.

In another more preferred embodiment, the invention relates to the above-mentioned compounds of formula 1, 1a, 1b, 1b1, 1c or 1c1 for use in the treatment of a fibrosing disease selected from the group consisting of systemic sclerosis (SSc), non-alcoholic steatohepatitis (NASH), acute on chronic liver failure (ACLF), (monogenic and digenic) interferonopathies, interstitial lung disease (ILD).

In another more preferred embodiment, the invention relates to the above-mentioned compounds of formula 1, 1a, 1b, 1b1, 1c or 1c1 for use in the treatment of a disease selected from the group consisting of age-related macular degeneration (AMD), retinopathy, glaucoma, aging, muscle disorders, heart failure, COVID-19/SARS-CoV-2 infection, renal inflammation, renal fibrosis, dysmetabolism, vascular diseases, cardiovascular diseases, diabetes, obesity, and cancer.

In another embodiment the invention relates to a pharmaceutical composition comprising at least one of the above-mentioned compounds and optionally one or more pharmaceutically acceptable carriers and/or excipients.

In another preferred embodiment the invention relates to a combination of a compound of formula 1, 1a, 1b, 1b1, 1c or 1c1 and one or more active agents selected from the group consisting of anti-inflammatory agents, anti-fibrotic agents, anti-allergic agents/anti-histamines, bronchodilators, beta 2 agonists/betamimetics, adrenergic agonists, anticholinergic agents, methotrexate, mycophenolate mofetil, leukotriene modulators, JAK inhibitors, anti-interleukin antibodies, non-specific immunotherapeutics such as interferons or other cytokines/chemokines, cytokine/chemokine receptor modulators, toll-like receptor agonists, immune checkpoint regulators, an anti-TNF antibody, an anti-BAFF antibody.

### EXAMPLES

The following examples are for the purpose of illustration of the invention only and are not intended in any way to limit the scope of the present invention. The absolute configuration was defined by x-ray for representative examples in a complex with the STING protein.

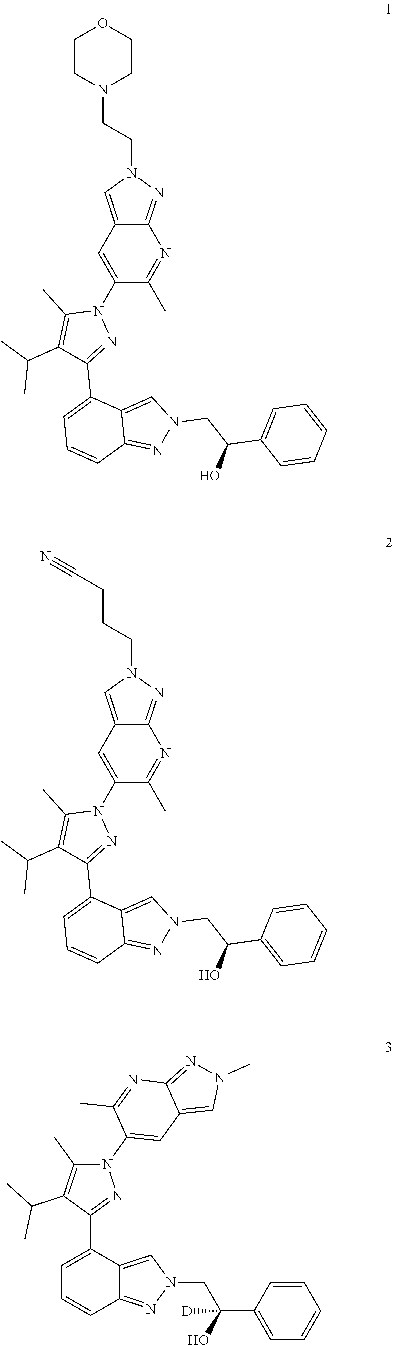 [SMILES: Cc1nc2nn(CCN3CCOCC3)cc2cc1-n1nc(-c2cccc3nn(C[C@H](O)c4ccccc4)cc23)c(C(C)C)c1C ; Cc1nc2nn(CCCC#N)cc2cc1-n1nc(-c2cccc3nn(C[C@H](O)c4ccccc4)cc23)c(C(C)C)c1C ; [2H][C@](O)(Cn1cc2c(-c3nn(-c4cc5cn(C)nc5nc4C)c(C)c3C(C)C)cccc2n1)c1ccccc1]

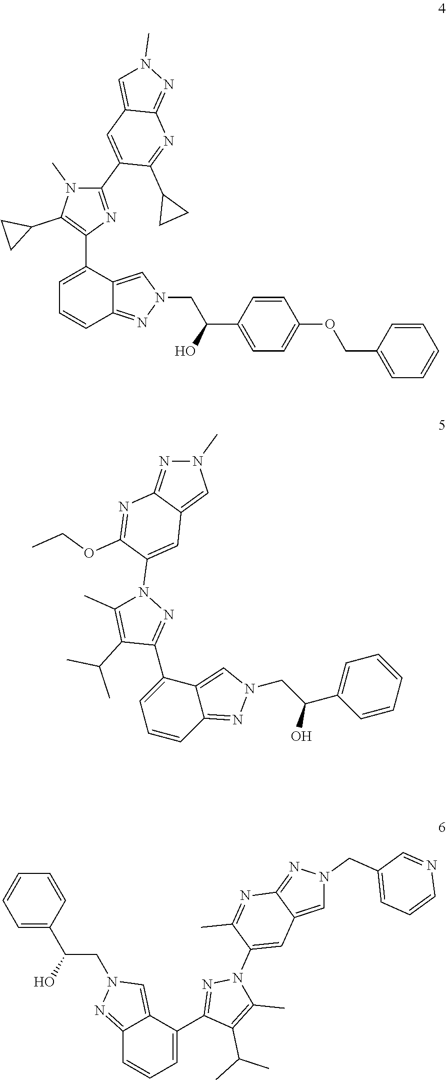 [SMILES: Cn1cc2cc(-c3nc(-c4cccc5nn(C[C@H](O)c6ccc(OCc7ccccc7)cc6)cc45)c(C4CC4)n3C)c(C3CC3)nc2n1 ; CCOc1nc2nn(C)cc2cc1-n1nc(-c2cccc3nn(C[C@H](O)c4ccccc4)cc23)c(C(C)C)c1C ; Cc1nc2nn(Cc3cccnc3)cc2cc1-n1nc(-c2cccc3nn(C[C@H](O)c4ccccc4)cc23)c(C(C)C)c1C]

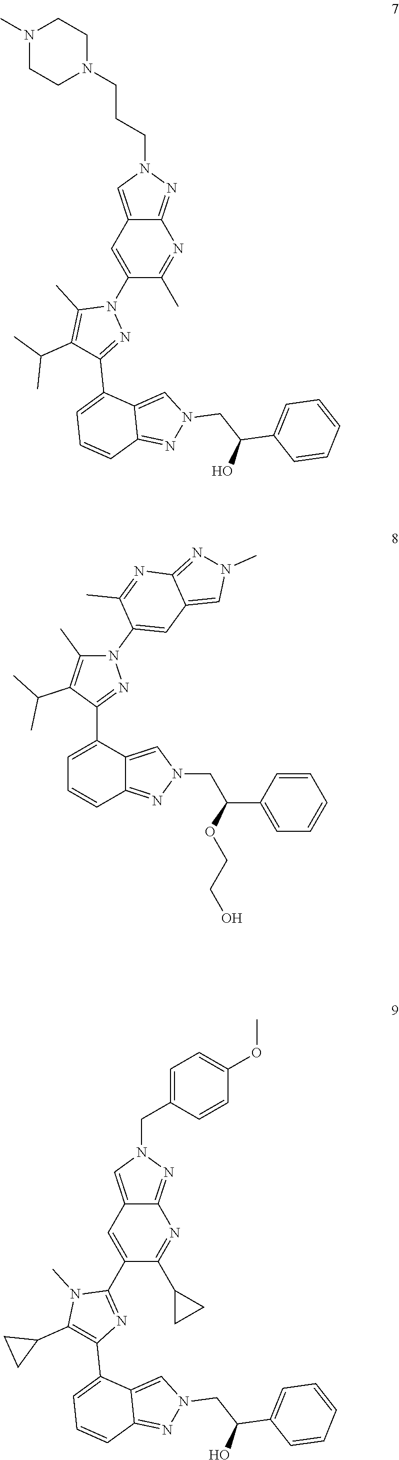 [SMILES: Cc1nc2nn(CCCN3CCN(C)CC3)cc2cc1-n1nc(-c2cccc3nn(C[C@H](O)c4ccccc4)cc23)c(C(C)C)c1C ; Cc1nc2nn(C)cc2cc1-n1nc(-c2cccc3nn(C[C@H](OCCO)c4ccccc4)cc23)c(C(C)C)c1C ; COc1ccc(Cn2cc3cc(-c4nc(-c5cccc6nn(C[C@H](O)c7ccccc7)cc56)c(C5CC5)n4C)c(C4CC4)nc3n2)cc1]

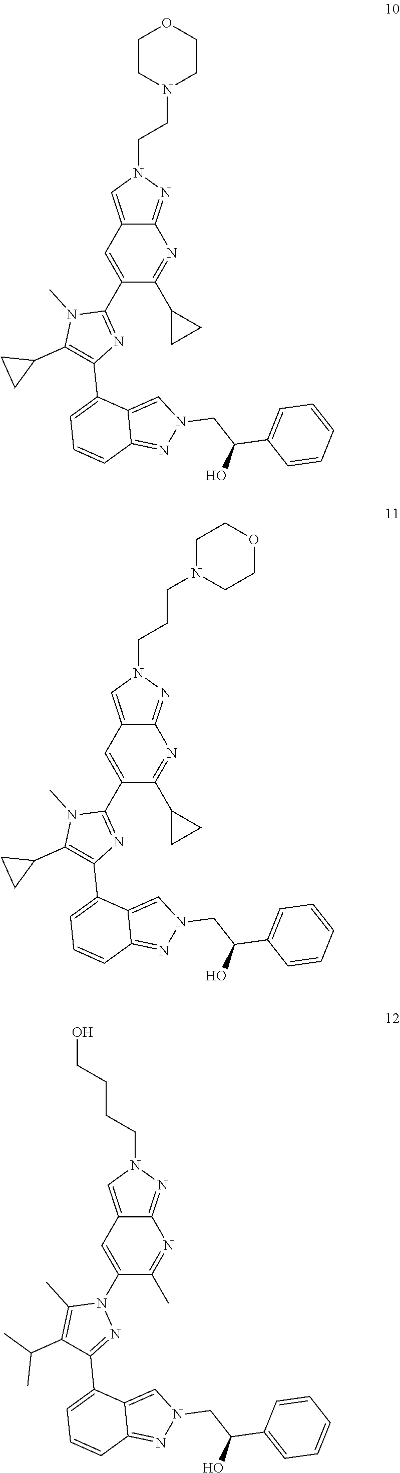 [SMILES: Cn1c(-c2cc3cn(CCN4CCOCC4)nc3nc2C2CC2)nc(-c2cccc3nn(C[C@H](O)c4ccccc4)cc23)c1C1CC1 ; Cn1c(-c2cc3cn(CCCN4CCOCC4)nc3nc2C2CC2)nc(-c2cccc3nn(C[C@H](O)c4ccccc4)cc23)c1C1CC1 ; Cc1nc2nn(CCCCO)cc2cc1-n1nc(-c2cccc3nn(C[C@H](O)c4ccccc4)cc23)c(C(C)C)c1C]

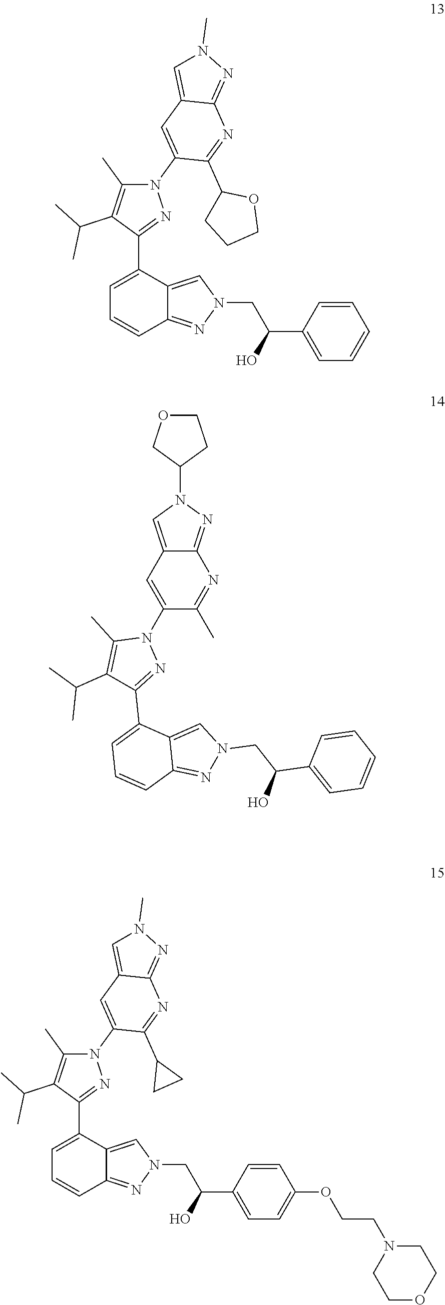 [SMILES: Cc1c(C(C)C)c(-c2cccc3nn(C[C@H](O)c4ccccc4)cc23)nn1-c1cc2cn(C)nc2nc1C1CCCO1 ; Cc1nc2nn(C3CCOC3)cc2cc1-n1nc(-c2cccc3nn(C[C@H](O)c4ccccc4)cc23)c(C(C)C)c1C ; Cc1c(C(C)C)c(-c2cccc3nn(C[C@H](O)c4ccc(OCCN5CCOCC5)cc4)cc23)nn1-c1cc2cn(C)nc2nc1C1CC1]

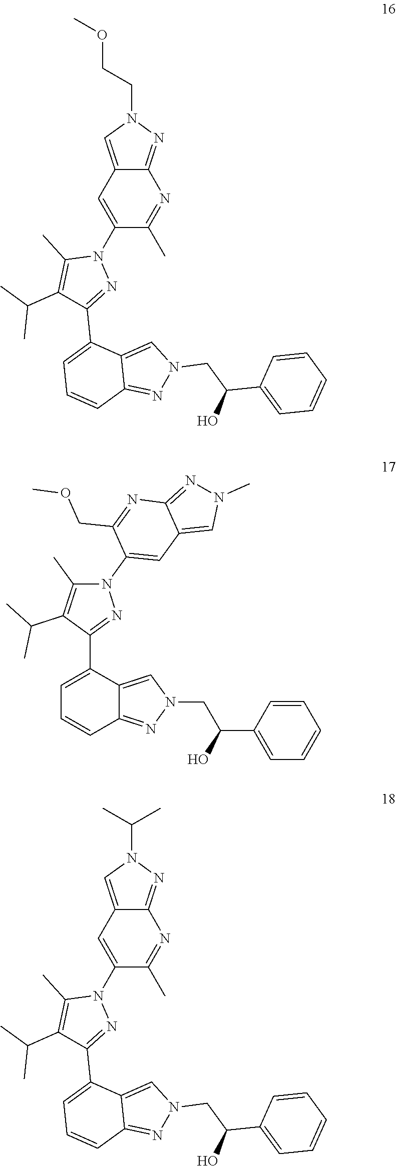 [SMILES: COCCn1cc2cc(-n3nc(-c4cccc5nn(C[C@H](O)c6ccccc6)cc45)c(C(C)C)c3C)c(C)nc2n1 ; COCc1nc2nn(C)cc2cc1-n1nc(-c2cccc3nn(C[C@H](O)c4ccccc4)cc23)c(C(C)C)c1C ; Cc1nc2nn(C(C)C)cc2cc1-n1nc(-c2cccc3nn(C[C@H](O)c4ccccc4)cc23)c(C(C)C)c1C]

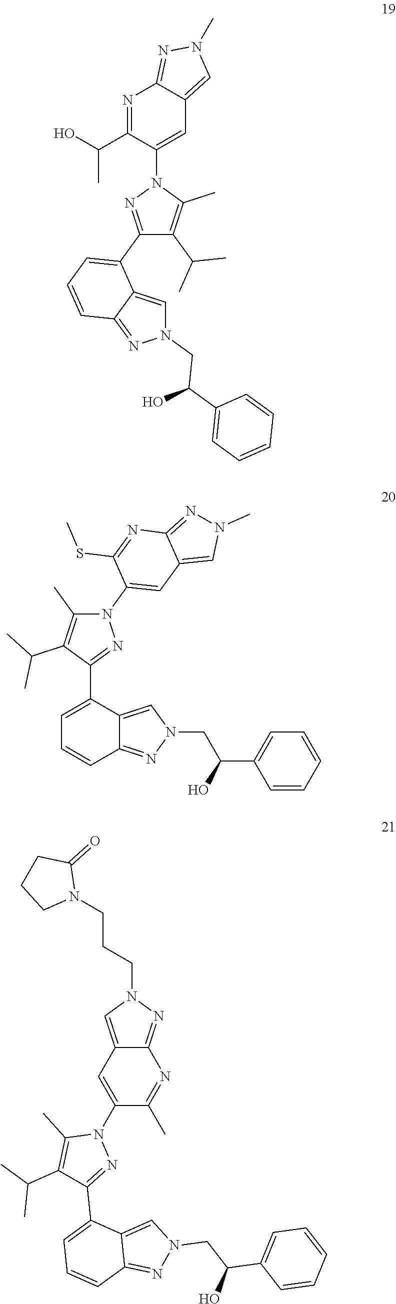 [SMILES: Cc1c(C(C)C)c(-c2cccc3nn(C[C@H](O)c4ccccc4)cc23)nn1-c1cc2cn(C)nc2nc1C(C)O ; CSc1nc2nn(C)cc2cc1-n1nc(-c2cccc3nn(C[C@H](O)c4ccccc4)cc23)c(C(C)C)c1C ; Cc1nc2nn(CCCN3CCCC3=O)cc2cc1-n1nc(-c2cccc3nn(C[C@H](O)c4ccccc4)cc23)c(C(C)C)c1C]

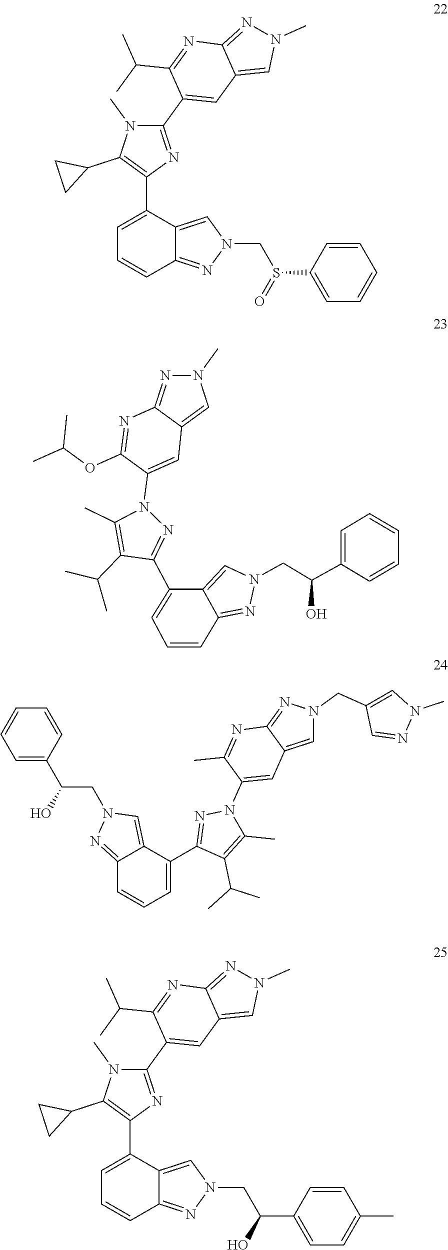 [SMILES: CC(C)c1nc2nn(C)cc2cc1-c1nc(-c2cccc3nn(C[S@@](=O)c4ccccc4)cc23)c(C2CC2)n1C ; Cc1c(C(C)C)c(-c2cccc3nn(C[C@H](O)c4ccccc4)cc23)nn1-c1cc2cn(C)nc2nc1OC(C)C ; Cc1nc2nn(Cc3cnn(C)c3)cc2cc1-n1nc(-c2cccc3nn(C[C@H](O)c4ccccc4)cc23)c(C(C)C)c1C ; Cc1ccc([C@@H](O)Cn2cc3c(-c4nc(-c5cc6cn(C)nc6nc5C(C)C)n(C)c4C4CC4)cccc3n2)cc1]

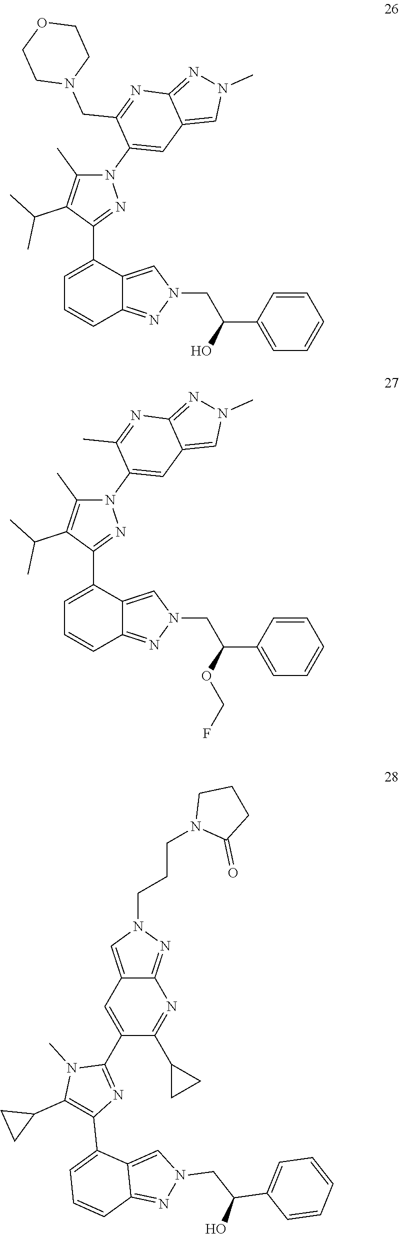 [SMILES: Cc1c(C(C)C)c(-c2cccc3nn(C[C@H](O)c4ccccc4)cc23)nn1-c1cc2cn(C)nc2nc1CN1CCOCC1 ; Cc1nc2nn(C)cc2cc1-n1nc(-c2cccc3nn(C[C@H](OCF)c4ccccc4)cc23)c(C(C)C)c1C ; Cn1c(-c2cc3cn(CCCN4CCCC4=O)nc3nc2C2CC2)nc(-c2cccc3nn(C[C@H](O)c4ccccc4)cc23)c1C1CC1]

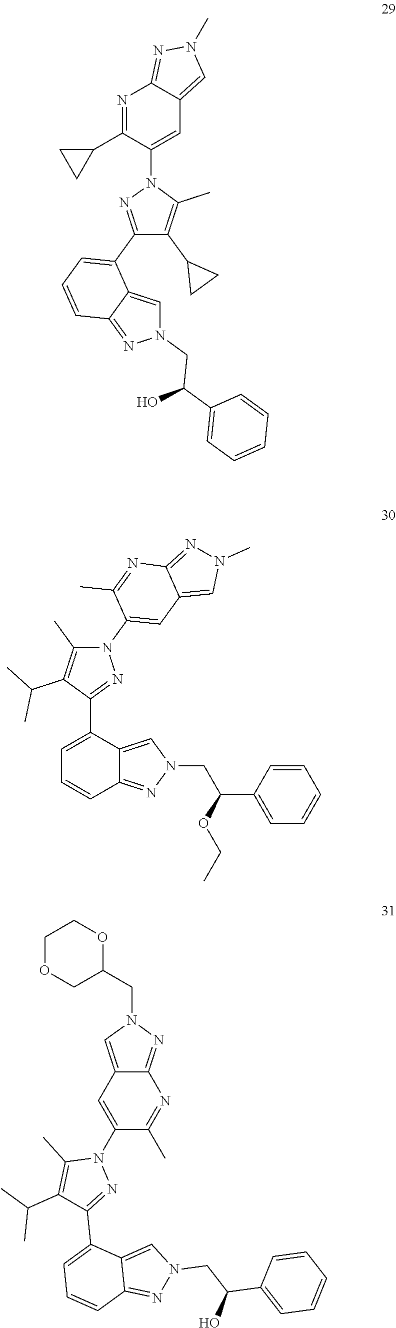 [SMILES: Cc1c(C2CC2)c(-c2cccc3nn(C[C@H](O)c4ccccc4)cc23)nn1-c1cc2cn(C)nc2nc1C1CC1 ; CCO[C@@H](Cn1cc2c(-c3nn(-c4cc5cn(C)nc5nc4C)c(C)c3C(C)C)cccc2n1)c1ccccc1 ; Cc1nc2nn(CC3COCCO3)cc2cc1-n1nc(-c2cccc3nn(C[C@H](O)c4ccccc4)cc23)c(C(C)C)c1C]

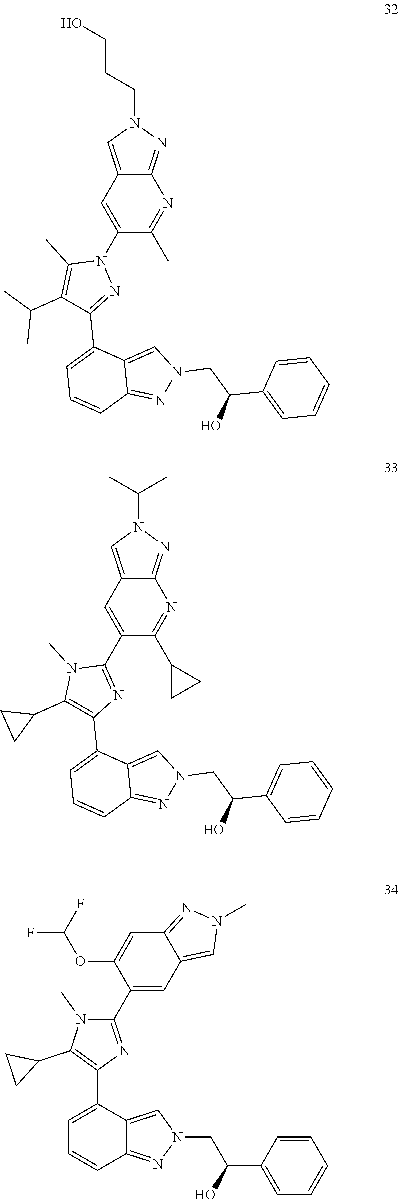 [SMILES: Cc1nc2nn(CCCO)cc2cc1-n1nc(-c2cccc3nn(C[C@H](O)c4ccccc4)cc23)c(C(C)C)c1C ; CC(C)n1cc2cc(-c3nc(-c4cccc5nn(C[C@H](O)c6ccccc6)cc45)c(C4CC4)n3C)c(C3CC3)nc2n1 ; Cn1cc2cc(-c3nc(-c4cccc5nn(C[C@H](O)c6ccccc6)cc45)c(C4CC4)n3C)c(OC(F)F)cc2n1]

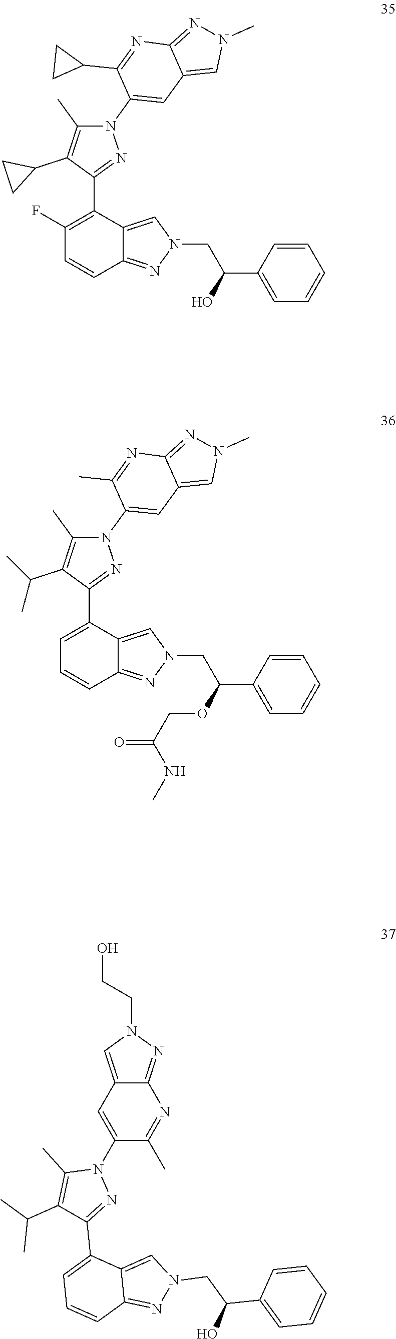 [SMILES: Cc1c(C2CC2)c(-c2c(F)ccc3nn(C[C@H](O)c4ccccc4)cc23)nn1-c1cc2cn(C)nc2nc1C1CC1 ; CNC(=O)CO[C@@H](Cn1cc2c(-c3nn(-c4cc5cn(C)nc5nc4C)c(C)c3C(C)C)cccc2n1)c1ccccc1 ; Cc1nc2nn(CCO)cc2cc1-n1nc(-c2cccc3nn(C[C@H](O)c4ccccc4)cc23)c(C(C)C)c1C]

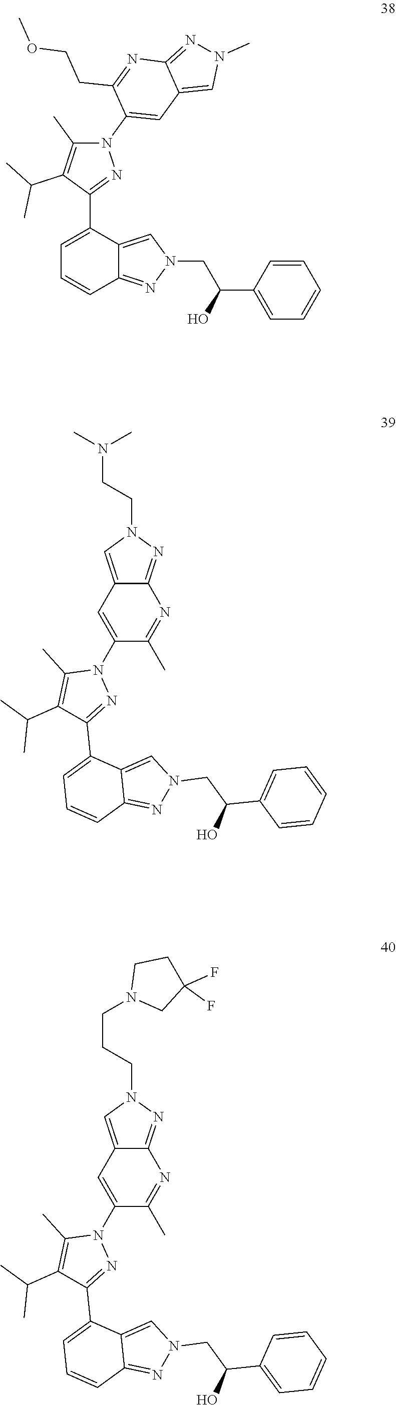 [SMILES: COCCc1nc2nn(C)cc2cc1-n1nc(-c2cccc3nn(C[C@H](O)c4ccccc4)cc23)c(C(C)C)c1C ; Cc1nc2nn(CCN(C)C)cc2cc1-n1nc(-c2cccc3nn(C[C@H](O)c4ccccc4)cc23)c(C(C)C)c1C ; Cc1nc2nn(CCCN3CCC(F)(F)C3)cc2cc1-n1nc(-c2cccc3nn(C[C@H](O)c4ccccc4)cc23)c(C(C)C)c1C]

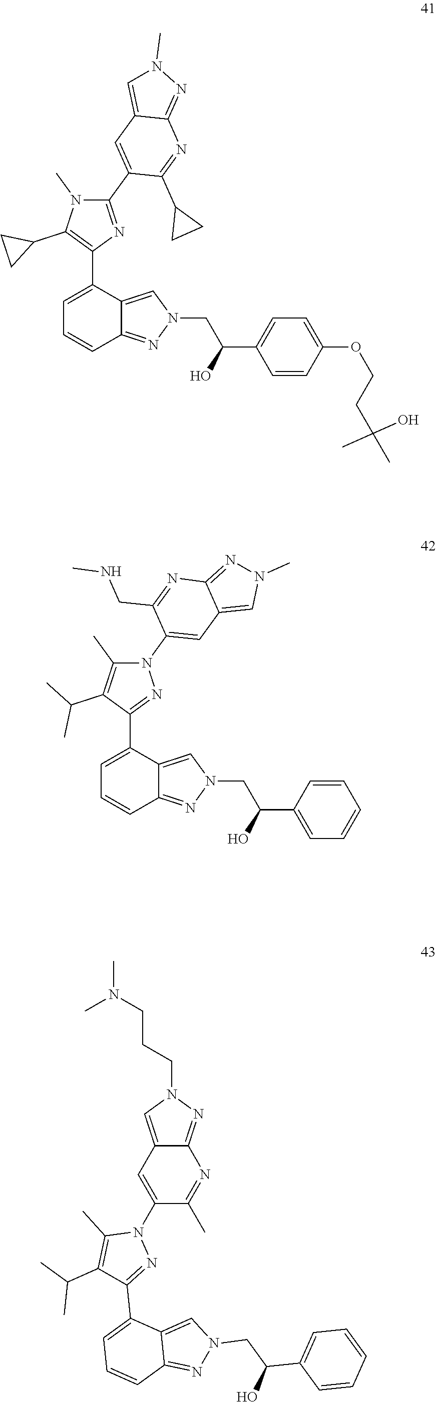 [SMILES: Cn1cc2cc(-c3nc(-c4cccc5nn(C[C@H](O)c6ccc(OCCC(C)(C)O)cc6)cc45)c(C4CC4)n3C)c(C3CC3)nc2n1 ; CNCc1nc2nn(C)cc2cc1-n1nc(-c2cccc3nn(C[C@H](O)c4ccccc4)cc23)c(C(C)C)c1C ; Cc1nc2nn(CCCN(C)C)cc2cc1-n1nc(-c2cccc3nn(C[C@H](O)c4ccccc4)cc23)c(C(C)C)c1C]

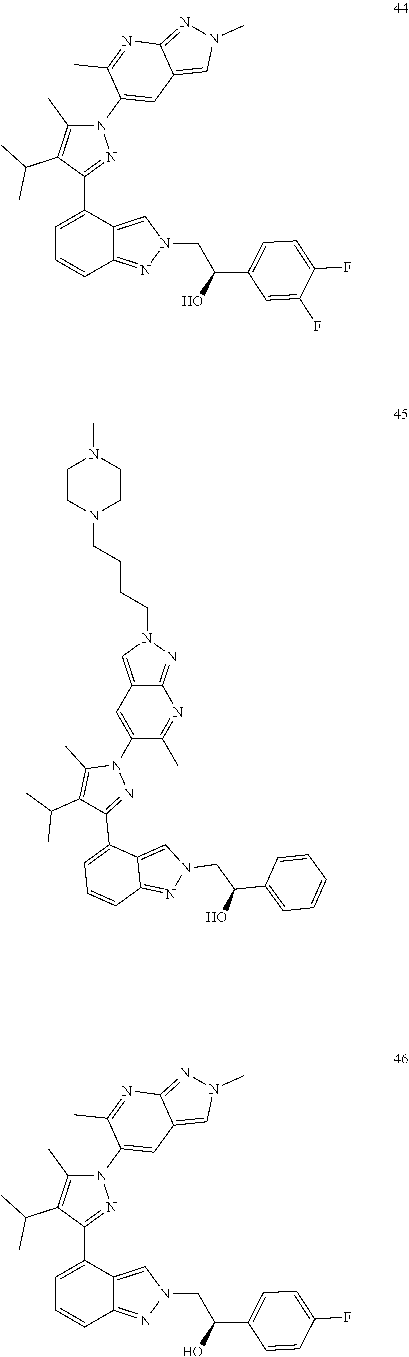 [SMILES: Cc1nc2nn(C)cc2cc1-n1nc(-c2cccc3nn(C[C@H](O)c4ccc(F)c(F)c4)cc23)c(C(C)C)c1C ; Cc1nc2nn(CCCCN3CCN(C)CC3)cc2cc1-n1nc(-c2cccc3nn(C[C@H](O)c4ccccc4)cc23)c(C(C)C)c1C ; Cc1nc2nn(C)cc2cc1-n1nc(-c2cccc3nn(C[C@H](O)c4ccc(F)cc4)cc23)c(C(C)C)c1C]

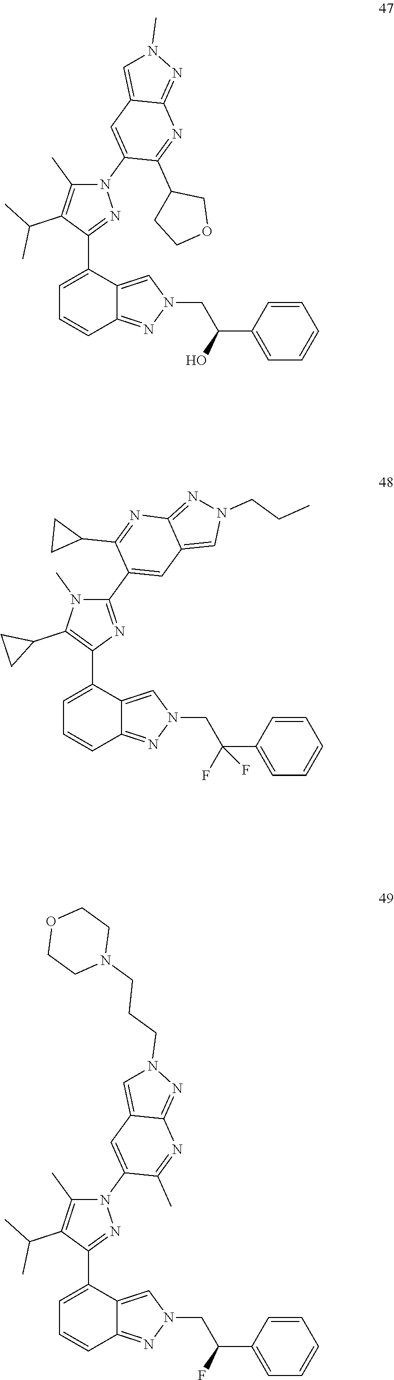 [SMILES: Cc1c(C(C)C)c(-c2cccc3nn(C[C@H](O)c4ccccc4)cc23)nn1-c1cc2cn(C)nc2nc1C1CCOC1 ; CCCn1cc2cc(-c3nc(-c4cccc5nn(CC(F)(F)c6ccccc6)cc45)c(C4CC4)n3C)c(C3CC3)nc2n1 ; Cc1nc2nn(CCCN3CCOCC3)cc2cc1-n1nc(-c2cccc3nn(C[C@H](F)c4ccccc4)cc23)c(C(C)C)c1C]

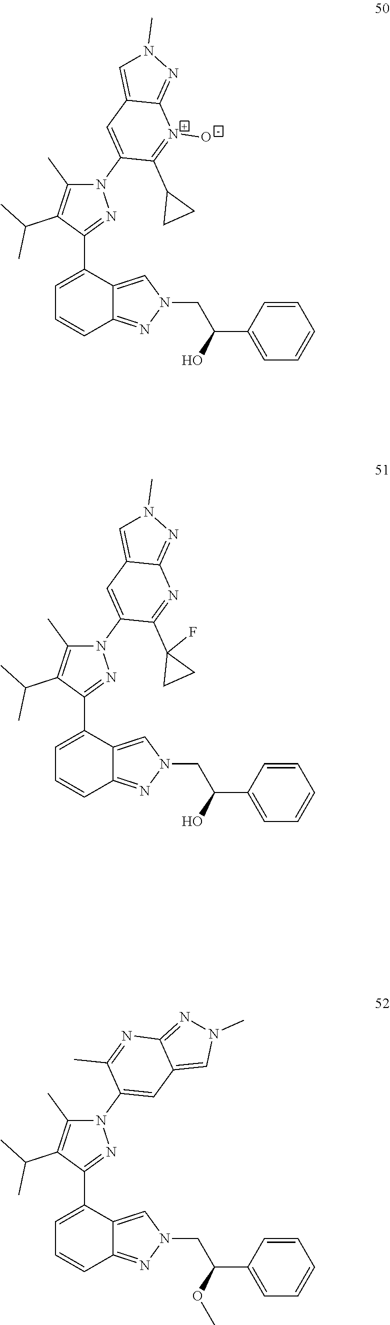

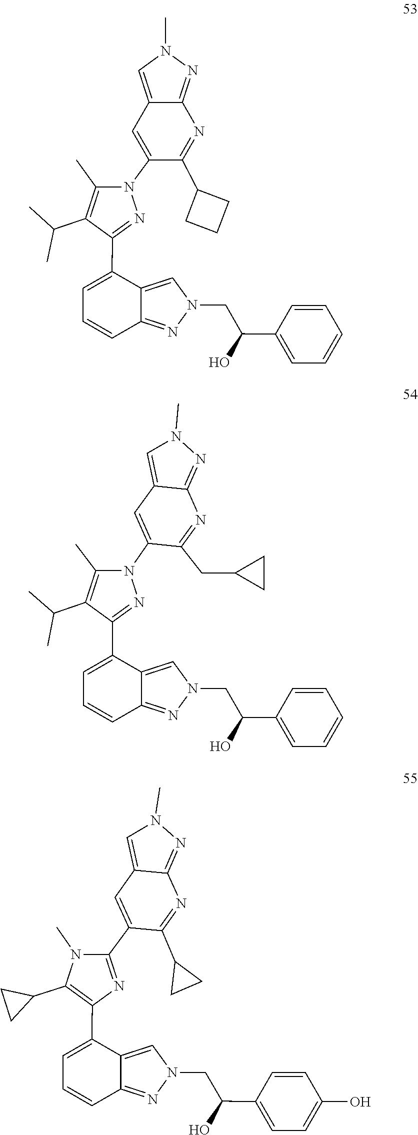 [SMILES: Cc1c(C(C)C)c(-c2cccc3nn(C[C@H](O)c4ccccc4)cc23)nn1-c1cc2cn(C)nc2nc1C1CCC1 ; Cc1c(C(C)C)c(-c2cccc3nn(C[C@H](O)c4ccccc4)cc23)nn1-c1cc2cn(C)nc2nc1CC1CC1 ; Cn1cc2cc(-c3nc(-c4cccc5nn(C[C@H](O)c6ccc(O)cc6)cc45)c(C4CC4)n3C)c(C3CC3)nc2n1]

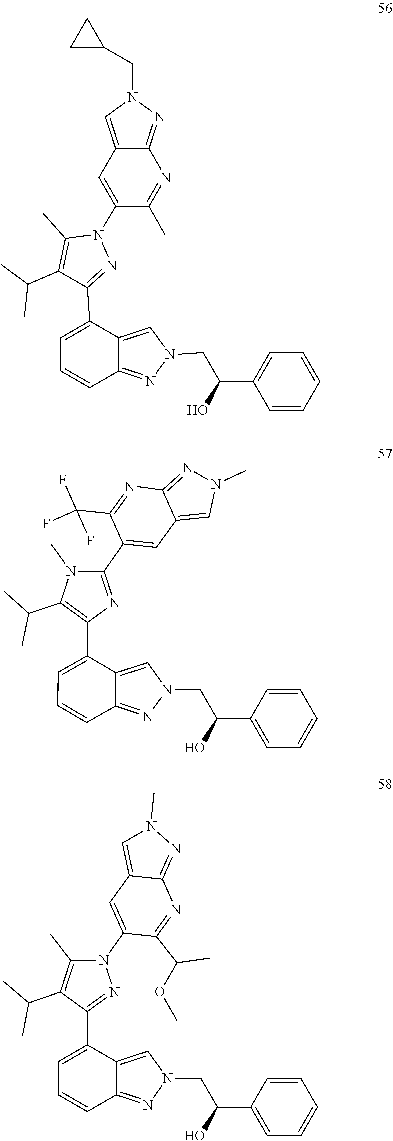 [SMILES: Cc1nc2nn(CC3CC3)cc2cc1-n1nc(-c2cccc3nn(C[C@H](O)c4ccccc4)cc23)c(C(C)C)c1C ; CC(C)c1c(-c2cccc3nn(C[C@H](O)c4ccccc4)cc23)nc(-c2cc3cn(C)nc3nc2C(F)(F)F)n1C ; COC(C)c1nc2nn(C)cc2cc1-n1nc(-c2cccc3nn(C[C@H](O)c4ccccc4)cc23)c(C(C)C)c1C]

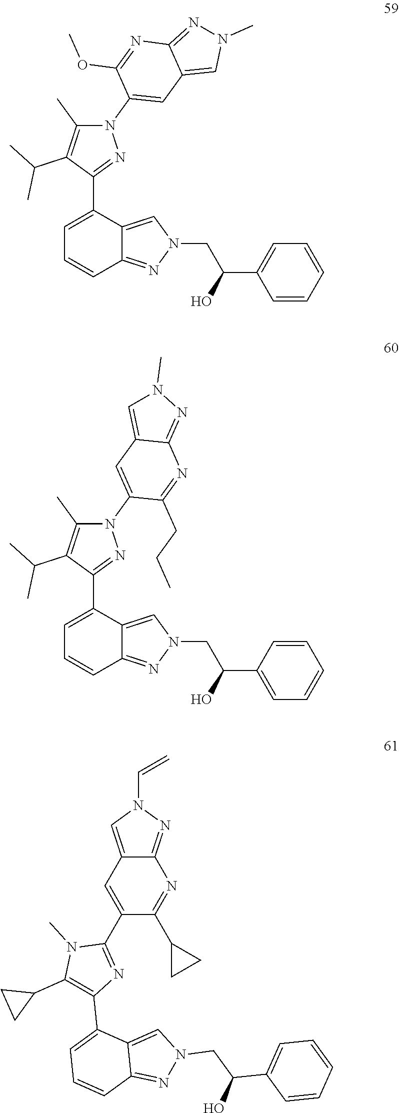 [SMILES: COc1nc2nn(C)cc2cc1-n1nc(-c2cccc3nn(C[C@H](O)c4ccccc4)cc23)c(C(C)C)c1C ; CCCc1nc2nn(C)cc2cc1-n1nc(-c2cccc3nn(C[C@H](O)c4ccccc4)cc23)c(C(C)C)c1C ; C=Cn1cc2cc(-c3nc(-c4cccc5nn(C[C@H](O)c6ccccc6)cc45)c(C4CC4)n3C)c(C3CC3)nc2n1]

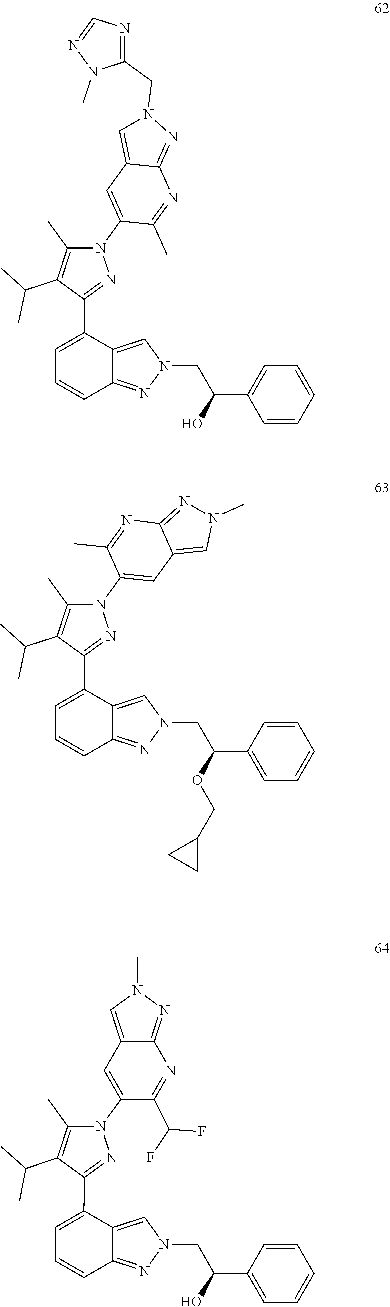 [SMILES: Cc1nc2nn(Cc3ncnn3C)cc2cc1-n1nc(-c2cccc3nn(C[C@H](O)c4ccccc4)cc23)c(C(C)C)c1C ; Cc1nc2nn(C)cc2cc1-n1nc(-c2cccc3nn(C[C@H](OCC4CC4)c4ccccc4)cc23)c(C(C)C)c1C ; Cc1c(C(C)C)c(-c2cccc3nn(C[C@H](O)c4ccccc4)cc23)nn1-c1cc2cn(C)nc2nc1C(F)F]

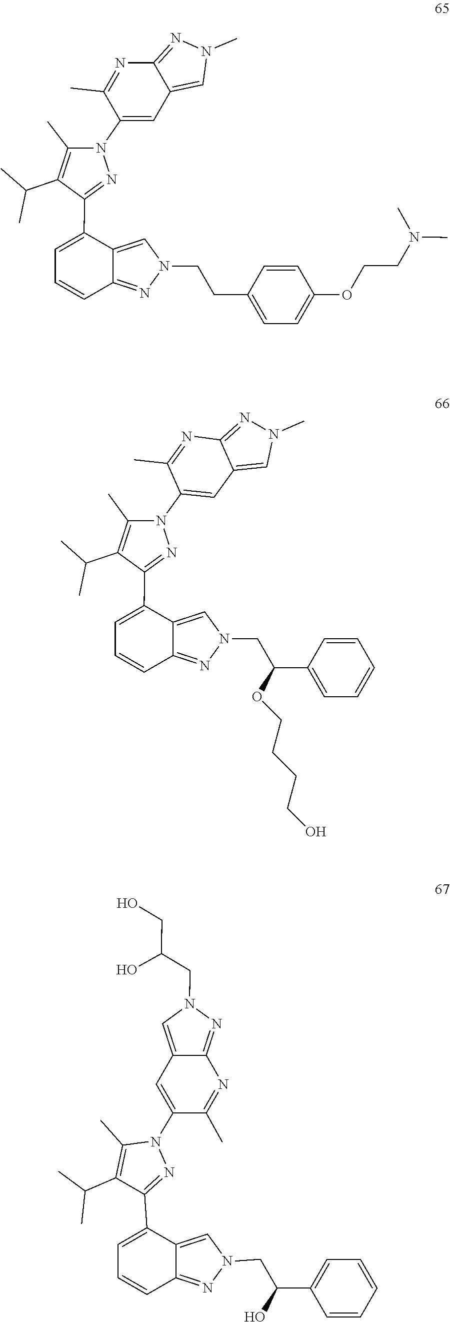

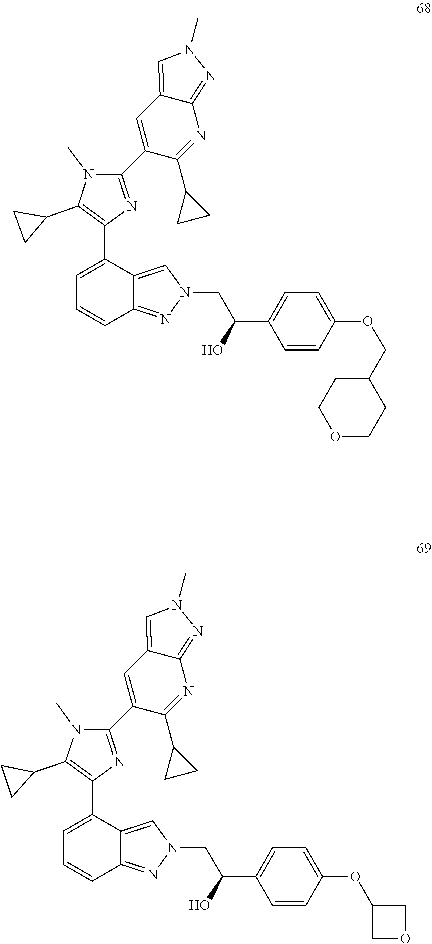 [SMILES: Cn1cc2cc(-c3nc(-c4cccc5nn(C[C@H](O)c6ccc(OCC7CCOCC7)cc6)cc45)c(C4CC4)n3C)c(C3CC3)nc2n1 ; Cn1cc2cc(-c3nc(-c4cccc5nn(C[C@H](O)c6ccc(OC7COC7)cc6)cc45)c(C4CC4)n3C)c(C3CC3)nc2n1]

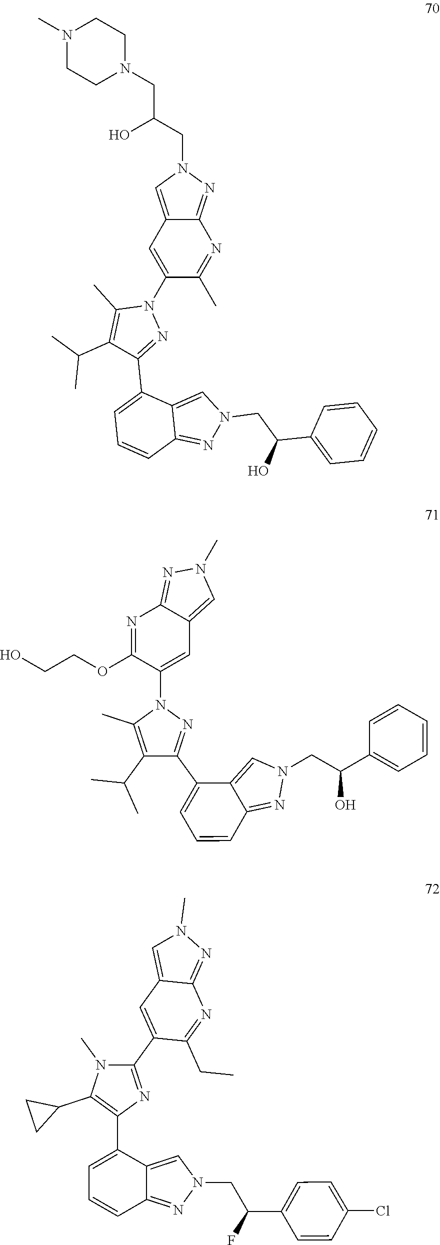 [SMILES: Cc1nc2nn(CC(O)CN3CCN(C)CC3)cc2cc1-n1nc(-c2cccc3nn(C[C@H](O)c4ccccc4)cc23)c(C(C)C)c1C ; Cc1c(C(C)C)c(-c2cccc3nn(C[C@H](O)c4ccccc4)cc23)nn1-c1cc2cn(C)nc2nc1OCCO ; CCc1nc2nn(C)cc2cc1-c1nc(-c2cccc3nn(C[C@H](F)c4ccc(Cl)cc4)cc23)c(C2CC2)n1C]

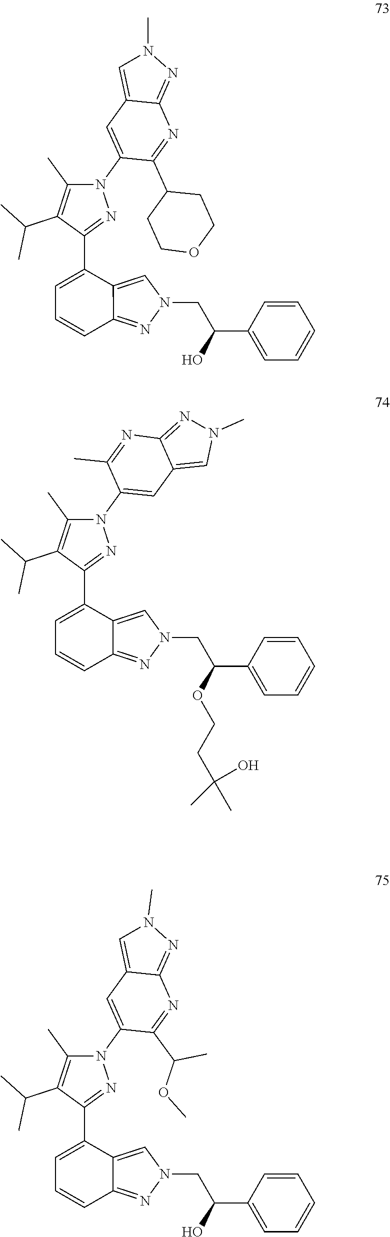 [SMILES: Cc1c(C(C)C)c(-c2cccc3nn(C[C@H](O)c4ccccc4)cc23)nn1-c1cc2cn(C)nc2nc1C1CCOCC1 ; Cc1nc2nn(C)cc2cc1-n1nc(-c2cccc3nn(C[C@H](OCCC(C)(C)O)c4ccccc4)cc23)c(C(C)C)c1C ; COC(C)c1nc2nn(C)cc2cc1-n1nc(-c2cccc3nn(C[C@H](O)c4ccccc4)cc23)c(C(C)C)c1C]

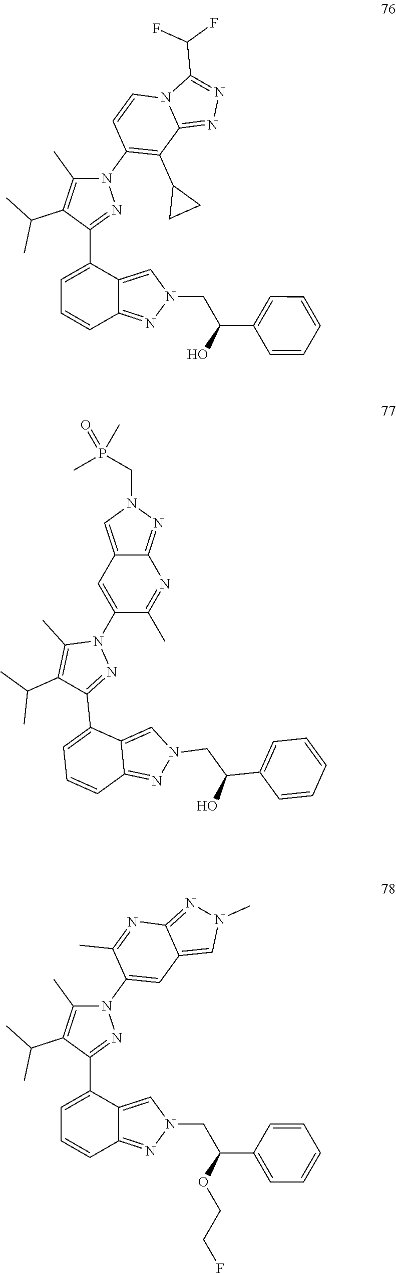 [SMILES: Cc1c(C(C)C)c(-c2cccc3nn(C[C@H](O)c4ccccc4)cc23)nn1-c1ccn2c(C(F)F)nnc2c1C1CC1 ; Cc1nc2nn(CP(C)(C)=O)cc2cc1-n1nc(-c2cccc3nn(C[C@H](O)c4ccccc4)cc23)c(C(C)C)c1C ; Cc1nc2nn(C)cc2cc1-n1nc(-c2cccc3nn(C[C@H](OCCF)c4ccccc4)cc23)c(C(C)C)c1C]

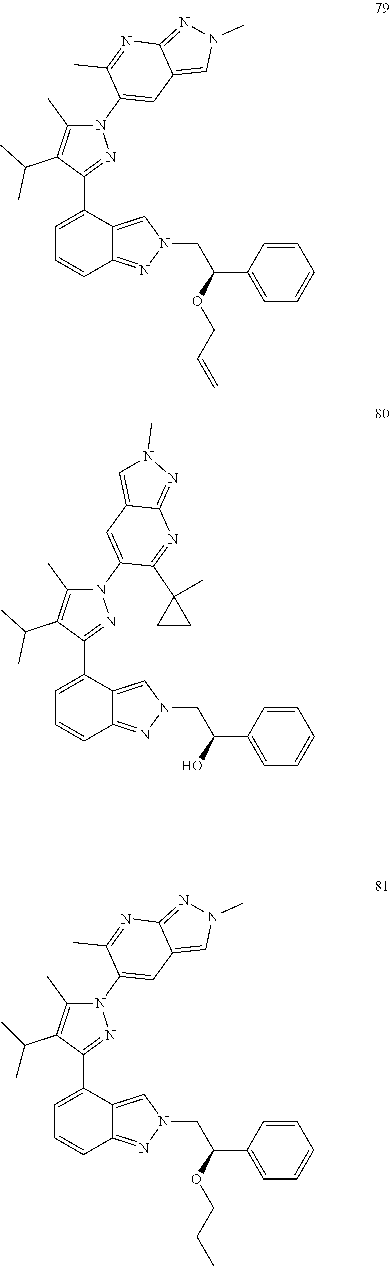 [SMILES: C=CCO[C@@H](Cn1cc2c(-c3nn(-c4cc5cn(C)nc5nc4C)c(C)c3C(C)C)cccc2n1)c1ccccc1 ; Cc1c(C(C)C)c(-c2cccc3nn(C[C@H](O)c4ccccc4)cc23)nn1-c1cc2cn(C)nc2nc1C1(C)CC1 ; CCCO[C@@H](Cn1cc2c(-c3nn(-c4cc5cn(C)nc5nc4C)c(C)c3C(C)C)cccc2n1)c1ccccc1]

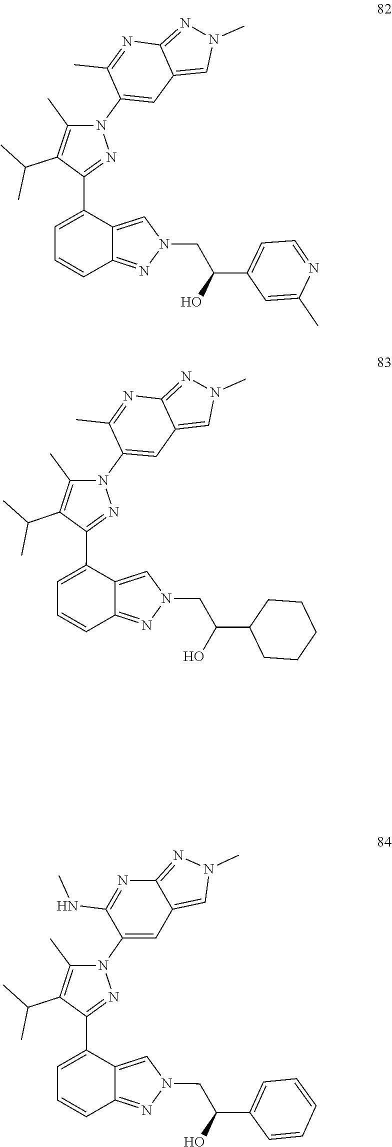

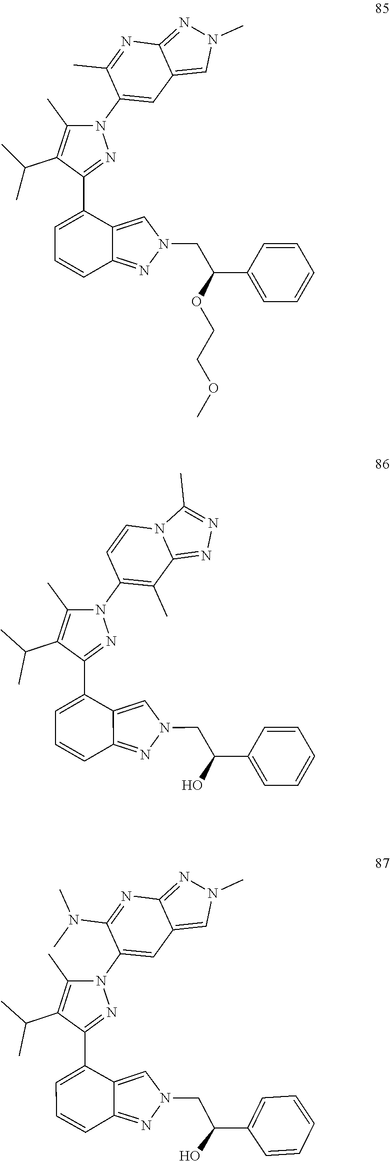 [SMILES: COCCO[C@@H](Cn1cc2c(-c3nn(-c4cc5cn(C)nc5nc4C)c(C)c3C(C)C)cccc2n1)c1ccccc1 ; Cc1c(-n2nc(-c3cccc4nn(C[C@H](O)c5ccccc5)cc34)c(C(C)C)c2C)ccn2c(C)nnc12 ; Cc1c(C(C)C)c(-c2cccc3nn(C[C@H](O)c4ccccc4)cc23)nn1-c1cc2cn(C)nc2nc1N(C)C]

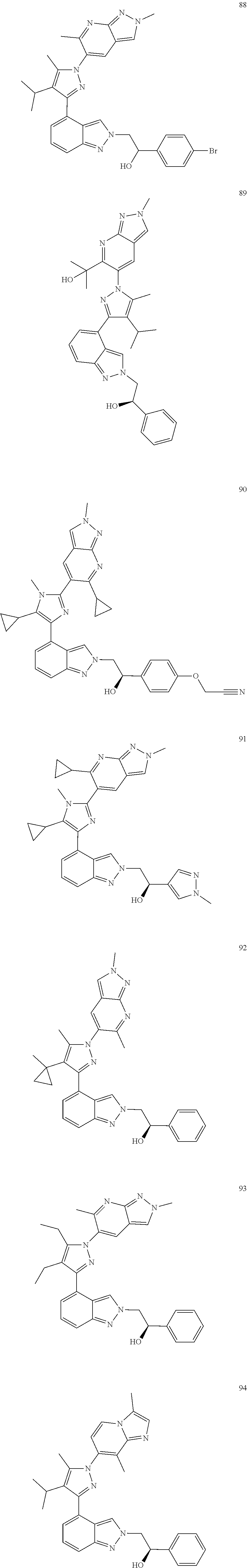

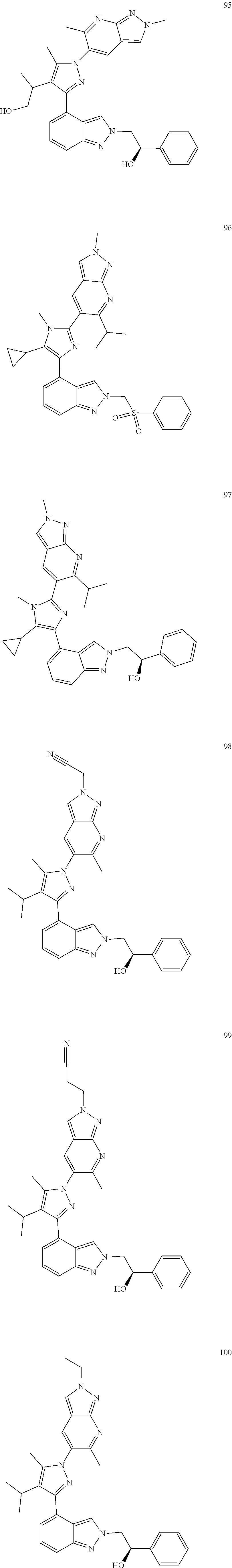 [SMILES: Cc1nc2nn(C)cc2cc1-n1nc(-c2cccc3nn(C[C@H](O)c4ccccc4)cc23)c(C(C)CO)c1C ; CC(C)c1nc2nn(C)cc2cc1-c1nc(-c2cccc3nn(CS(=O)(=O)c4ccccc4)cc23)c(C2CC2)n1C ; CC(C)c1nc2nn(C)cc2cc1-c1nc(-c2cccc3nn(C[C@H](O)c4ccccc4)cc23)c(C2CC2)n1C ; Cc1nc2nn(CC#N)cc2cc1-n1nc(-c2cccc3nn(C[C@H](O)c4ccccc4)cc23)c(C(C)C)c1C ; Cc1nc2nn(CCC#N)cc2cc1-n1nc(-c2cccc3nn(C[C@H](O)c4ccccc4)cc23)c(C(C)C)c1C ; CCn1cc2cc(-n3nc(-c4cccc5nn(C[C@H](O)c6ccccc6)cc45)c(C(C)C)c3C)c(C)nc2n1]

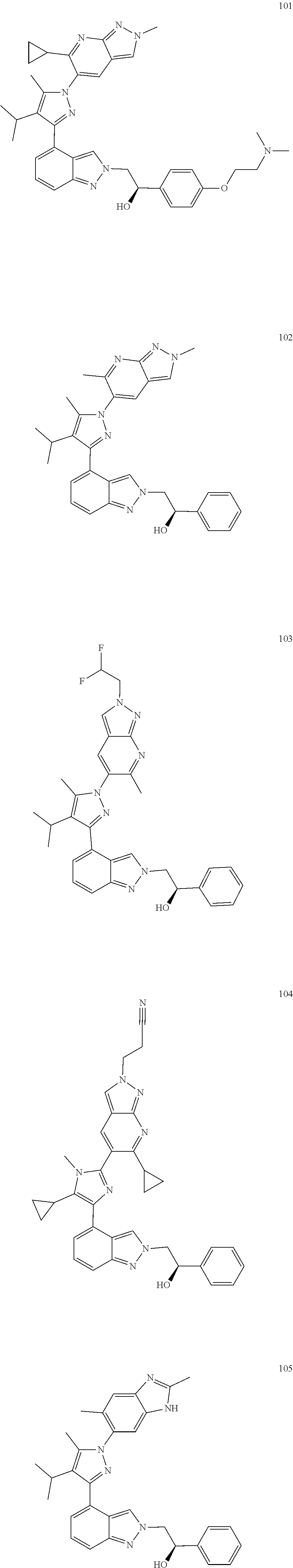

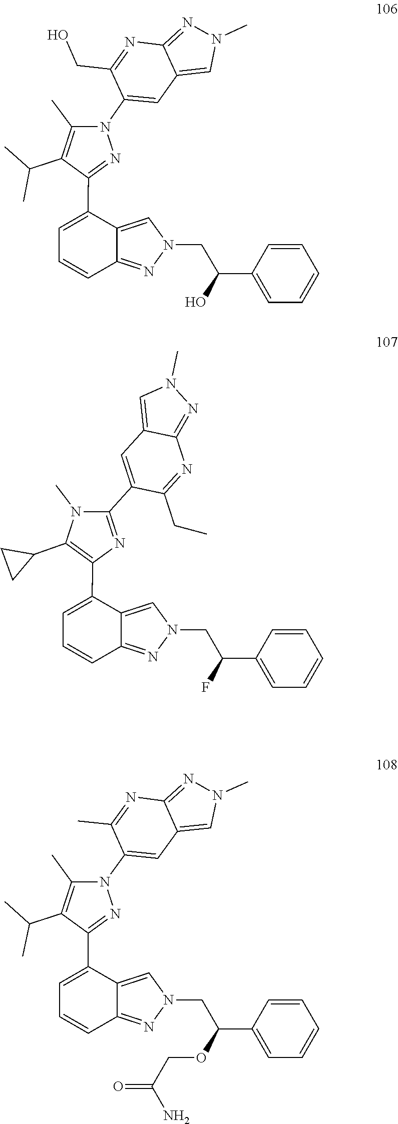 [SMILES: Cc1c(C(C)C)c(-c2cccc3nn(C[C@H](O)c4ccccc4)cc23)nn1-c1cc2cn(C)nc2nc1CO ; CCc1nc2nn(C)cc2cc1-c1nc(-c2cccc3nn(C[C@H](F)c4ccccc4)cc23)c(C2CC2)n1C ; Cc1nc2nn(C)cc2cc1-n1nc(-c2cccc3nn(C[C@H](OCC(N)=O)c4ccccc4)cc23)c(C(C)C)c1C]

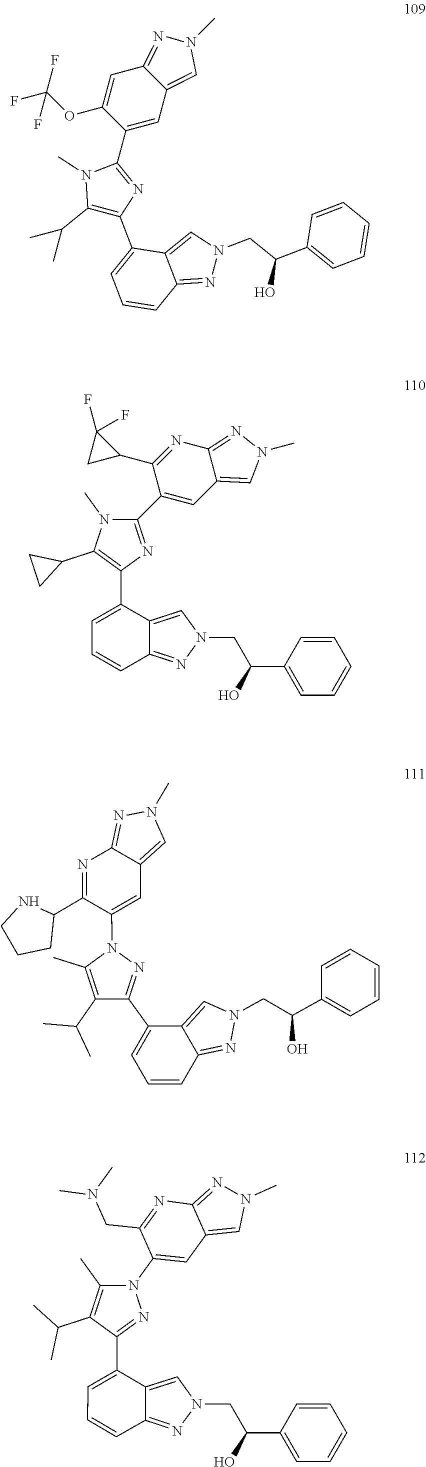 [SMILES: CC(C)c1c(-c2cccc3nn(C[C@H](O)c4ccccc4)cc23)nc(-c2cc3cn(C)nc3cc2OC(F)(F)F)n1C ; Cn1cc2cc(-c3nc(-c4cccc5nn(C[C@H](O)c6ccccc6)cc45)c(C4CC4)n3C)c(C3CC3(F)F)nc2n1 ; Cc1c(C(C)C)c(-c2cccc3nn(C[C@H](O)c4ccccc4)cc23)nn1-c1cc2cn(C)nc2nc1C1CCCN1 ; Cc1c(C(C)C)c(-c2cccc3nn(C[C@H](O)c4ccccc4)cc23)nn1-c1cc2cn(C)nc2nc1CN(C)C]

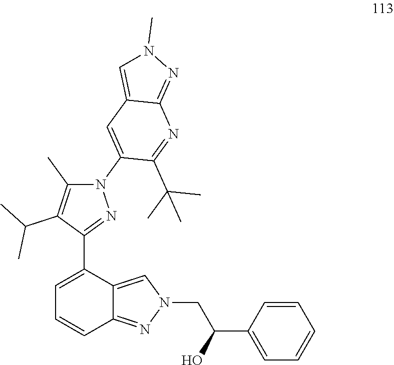 [SMILES: Cc1c(C(C)C)c(-c2cccc3nn(C[C@H](O)c4ccccc4)cc23)nn1-c1cc2cn(C)nc2nc1C(C)(C)C]

### General Terms and Definitions

Terms not specifically defined herein should be given the meanings that would be given to them by one of skill in the art in light of the disclosure and the context. As used in the specification, however, unless specified to the contrary, the following terms have the meaning indicated and the following conventions are adhered to.

In the groups, radicals, or moieties defined below, the number of carbon atoms is often specified preceding the group, for example, C₁₋₆-alkyl means an alkyl group or radical having 1 to 6 carbon atoms. In general in groups like HO, H₂N, (O)S, (O)₂S, NC (cyano), HOOC, F₃C or the like, the skilled artisan can see the radical attachment point(s) to the molecule from the free valences of the group itself. For combined groups comprising two or more subgroups, the last named subgroup is the radical attachment point, for example, the substituent “aryl-C₁₋₃-alkylene” means an aryl group which is bound to a C₁₋₃-alkyl-group, the latter of which is bound to the core or to the group to which the substituent is attached.

In case a compound of the present invention is depicted in the form of a chemical name and as a formula, in case of any discrepancy the formula shall prevail.

The numeration of the atoms of a substituent starts with the atom which is closest to the core or to the group to which the substituent is attached. For example, the term “3-carboxypropyl-group” represents the following substituent:

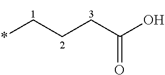 [SMILES: *CCCC(=O)O]

wherein the carboxy group is attached to the third carbon atom of the propyl group. The terms “1-methylpropyl-”, “2, 2-dimethylpropyl-” or “cyclopropylmethyl-” group represent the following groups:

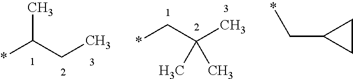

The asterisk may be used in sub-formulas to indicate the bond which is connected to the core molecule as defined.

When it is referred in the claims or the description, that a residue R # in an exemplified structure is the attaching point to the structure of formula 1, this can be read in this context also as an attaching point to the structure of formula 1a, 1b or 1c.

The term “substituted” as used herein, means that one or more hydrogens on the designated atom are replaced by a group selected from a defined group of substituents, provided that the designated atom's normal valence is not exceeded, and that the substitution results in a stable compound. Likewise, the term “substituted” may be used in connection with a chemical moiety instead of a single atom, e.g. “substituted alkyl”, “substituted aryl” or the like.

Unless specifically indicated, throughout the specification and the appended claims, a given chemical formula or name shall encompass tautomer's and all stereo, optical and geometrical isomers (e.g. enantiomers, diastereomers, E/Z isomers etc.,) and racemates thereof as well as mixtures in different proportions of the separate enantiomers, mixtures of diastereomers, or mixtures of any of the foregoing forms where such isomers and enantiomers exist, as well as solvates thereof such as for instance hydrates.

Unless specifically indicated, also “pharmaceutically acceptable salts” as defined in more detail below shall encompass solvates thereof such as for instance hydrates.

In general, substantially pure stereoisomers can be obtained according to synthetic principles known to a person skilled in the field, e.g. by separation of corresponding mixtures, by using stereochemically pure starting materials and/or by stereoselective synthesis. It is known in the art how to prepare optically active forms, such as by resolution of racemic forms or by synthesis, e.g. starting from optically active starting materials and/or by using chiral reagents.

Enantiomerically pure compounds of this invention or intermediates may be prepared via asymmetric synthesis, for example by preparation and subsequent separation of appropriate diastereomeric compounds or intermediates which can be separated by known methods (e.g. by chromatographic separation or crystallization) and/or by using chiral reagents, such as chiral starting materials, chiral catalysts, or chiral auxiliaries.

Further, it is known to the person skilled in the art how to prepare enantiomerically pure compounds from the corresponding racemic mixtures, such as by chromatographic separation of the corresponding racemic mixtures on chiral stationary phases; or by resolution of a racemic mixture using an appropriate resolving agent, e.g. by means of diastereomeric salt formation of the racemic compound with optically active acids or bases, subsequent resolution of the salts and release of the desired compound from the salt; or by derivatization of the corresponding racemic compounds with optically active chiral auxiliary reagents, subsequent diastereomer separation and removal of the chiral auxiliary group; or by kinetic resolution of a racemate (e.g. by enzymatic resolution); by enantioselective crystallization from a conglomerate of enantiomorphous crystals under suitable conditions; or by (fractional) crystallization from a suitable solvent in the presence of an optically active chiral auxiliary.

The phrase “pharmaceutically acceptable” is employed herein to refer to those compounds, materials, compositions, and/or dosage forms which are, within the scope of sound medical judgment, suitable for use in contact with the tissues of human beings without excessive toxicity, irritation, allergic response, or other problem or complication, and commensurate with a reasonable benefit/risk ratio.

As used herein, “pharmaceutically acceptable salt” refers to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like.

For example, such salts include salts from benzenesulfonic acid, benzoic acid, citric acid, ethanesulfonic acid, fumaric acid, gentisic acid, hydrobromic acid, hydrochloric acid, maleic acid, malic acid, malonic acid, mandelic acid, methanesulfonic acid, 4-methyl-benzenesulfonic acid, phosphoric acid, salicylic acid, succinic acid, sulfuric acid and tartaric acid. Further pharmaceutically acceptable salts can be formed with cations from ammonia, L-arginine, calcium, 2, 2′-iminobisethanol, L-lysine, magnesium, N-methyl-D-glucamine, potassium, sodium and tris(hydroxymethyl)-aminomethane.

The pharmaceutically acceptable salts of the present invention can be synthesized from the parent compound which contains a basic or acidic moiety by conventional chemical methods. Generally, such salts can be prepared by reacting the free acid or base forms of these compounds with a sufficient amount of the appropriate base or acid in water or in an organic diluent such as ether, ethyl acetate, ethanol, isopropanol, or acetonitrile, or a mixture thereof.

Salts of other acids than those mentioned above which for example are useful for purifying or isolating the compounds of the present invention (e.g. trifluoro acetate salts,) also comprise a part of the invention.

The term halogen denotes fluorine, chlorine, bromine and iodine. The term “C₁₋ₙ-alkyl”, wherein n is an integer selected from 2, 3, 4, 5 or 6, preferably 4, 5, or 6, either alone or in combination with another radical, denotes an acyclic, saturated, branched or linear hydrocarbon radical with 1 to n C atoms. For example the term C₁₋₅-alkyl embraces the radicals H₃C—, H₃C—CH₂—, H₃C—CH₂—CH₂—, H₃C—CH(CH₃)—, H₃C—CH₂—CH₂—CH₂—, H₃C—CH₂—CH(CH₃)—, H₃C—CH(CH₃)—CH₂—, H₃C—C(CH₃)₂—, H₃C—CH₂—CH₂—CH₂—CH₂—, H₃C—CH₂—CH₂—CH(CH₃)—, H₃C—CH₂—CH(CH₃)—CH₂—, H₃C—CH(CH₃)—CH₂—CH₂—, H₃C—CH₂—C(CH₃)₂—, H₃C—C(CH₃)₂—CH₂—, H₃C—CH(CH₃)—CH(CH₃)— and H₃C—CH₂—CH(CH₂CH₃)—.

The term “C₂-m-alkenyl” is used for a group “C₂-m-alkyl” wherein m is an integer selected from 3, 4, 5 or 6, preferably 4, 5 or 6, if at least two carbon atoms of said group are bonded to each other by a double bond.

The term “C₃₋ₖ-cycloalkyl”, wherein k is an integer selected from 3, 4, 5, 7 or 8, preferably 4, 5 or 6, either alone or in combination with another radical, denotes a cyclic, saturated, unbranched hydrocarbon radical with 3 to k C atoms. For example the term C₃₋₇-cycloalkyl includes cyclopropyl, cyclobutyl, cyclopentyl, cyclohexyl and cycloheptyl.

The term “halo” added to an “alkyl”, “alkylene” or “cycloalkyl” group (saturated or unsaturated) defines an alkyl, alkylene or cycloalkyl group wherein one or more hydrogen atoms are replaced by a halogen atom selected from among fluorine, chlorine or bromine, preferably fluorine and chlorine, particularly preferred is fluorine. Examples include: H₂FC—, HF₂C—, F₃C—.

The term “carbocyclyl”, either alone or in combination with another radical, means a mono-, bi- or tricyclic ring structure consisting of 3 to 14 carbon atoms. The term “carbocyclyl” refers to fully saturated, partially saturated and aromatic ring systems. The term “carbocyclyl” encompasses fused, bridged and spirocyclic systems.

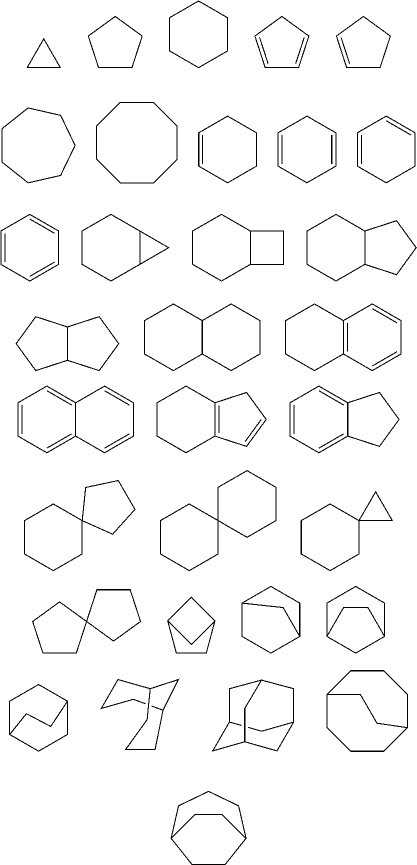

The term “aryl” as used herein, either alone or in combination with another radical, denotes a carbocyclic aromatic monocyclic group containing 6 carbon atoms which is optionally further fused to a second five- or six-membered, carbocyclic group which is aromatic, saturated or unsaturated. Aryl includes, but is not limited to, phenyl, indanyl, indenyl, naphthyl, anthracenyl, phenanthrenyl, tetrahydronaphthyl and dihydronaphthyl.

The term “heterocyclyl” means a saturated or unsaturated mono- or polycyclic ring system optionally comprising aromatic rings, containing one or more heteroatoms selected from N, O, S, SO or SO₂ consisting of 3 to 14 ring atoms wherein none of the heteroatoms is part of the aromatic ring. The term “heterocyclyl” is intended to include all the possible isomeric forms.

Thus, the term “heterocyclyl” includes the following exemplary structures (not depicted as radicals as each form is optionally attached through a covalent bond to any atom so long as appropriate valences are maintained):

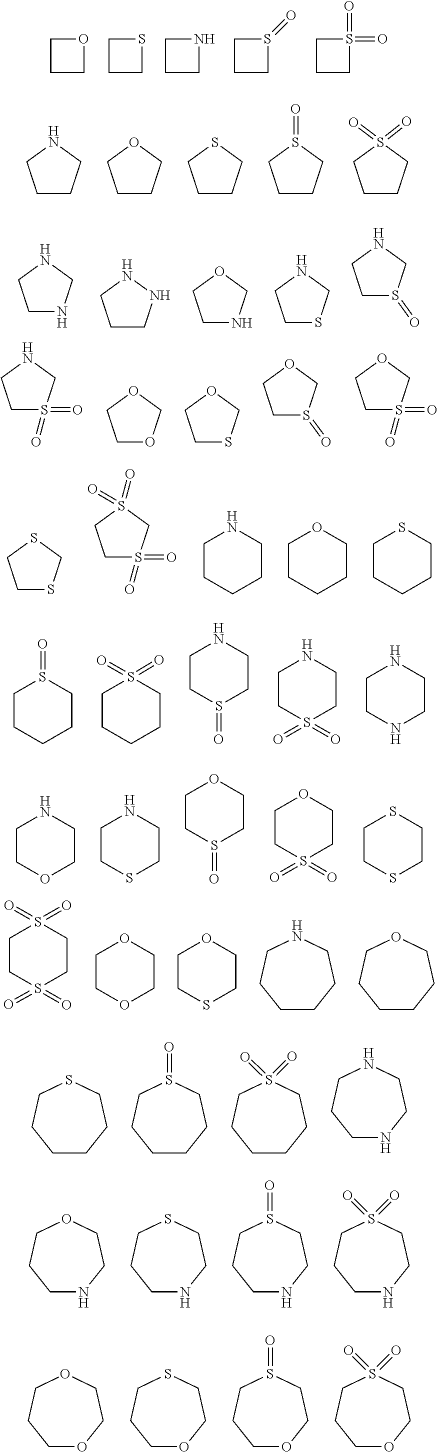

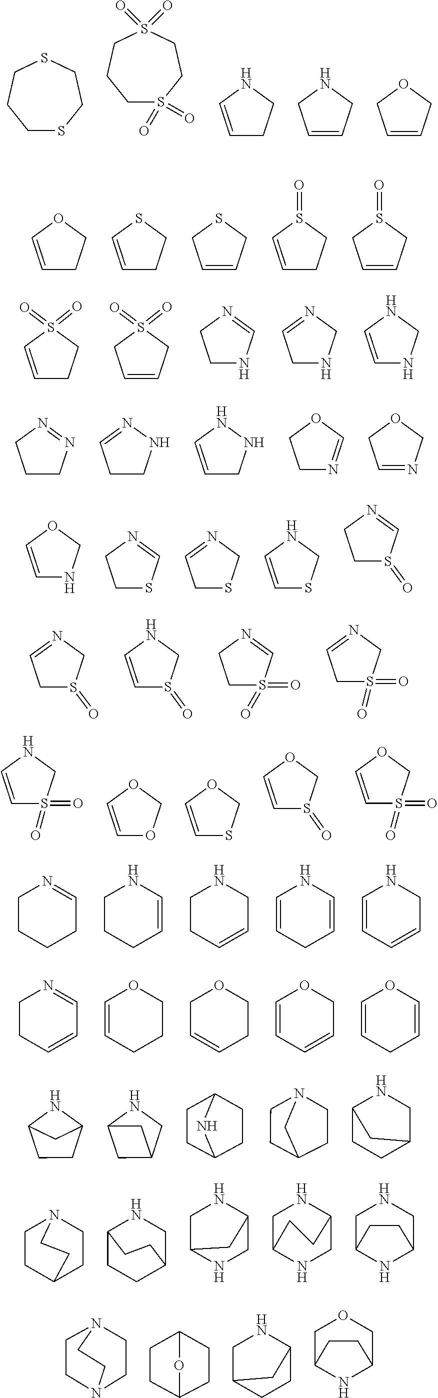

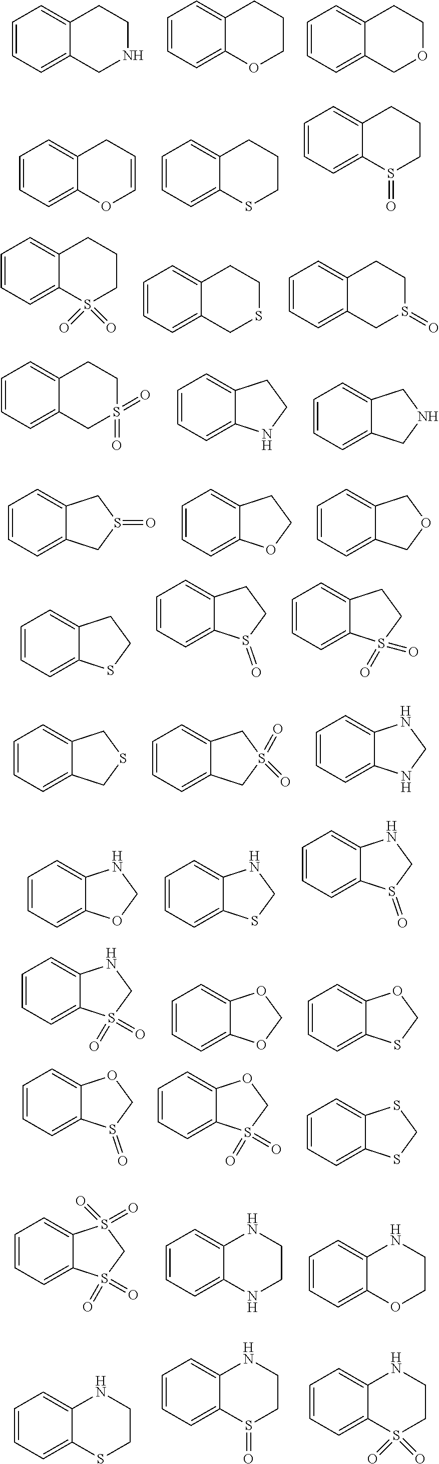

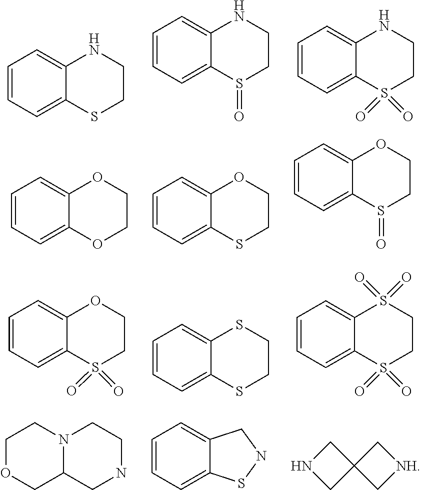

The term “heteroaryl” means a mono- or polycyclic ring system, comprising at least one aromatic ring, containing one or more heteroatoms selected from N, O, S, SO or SO₂, consisting of 5 to 14 ring atoms wherein at least one of the heteroatoms is part of an aromatic ring. The term “heteroaryl” is intended to include all the possible isomeric forms.

Thus, the term “heteroaryl” includes the following exemplary structures (not depicted as radicals as each form is optionally attached through a covalent bond to any atom so long as appropriate valences are maintained):

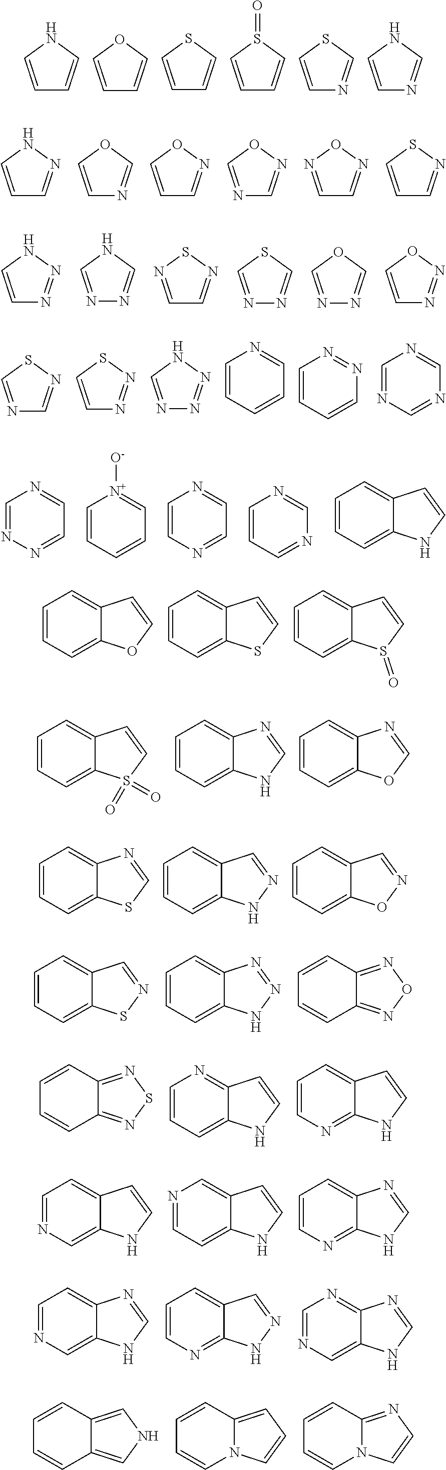

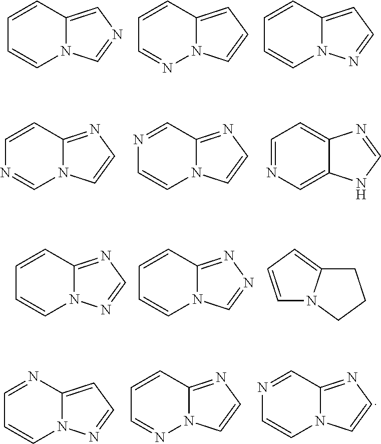

Many of the terms given above may be used repeatedly in the definition of a formula or group and in each case have one of the meanings given above, independently of one another.

The term “bicyclic ring systems” means groups consisting of joined cyclic substructures including spirocyclic, fused, and bridged ring systems.

### METHODS OF PREPARATION

The following examples are for the purpose of illustration of the invention only and are not intended in any way to limit the scope of the present invention. The term “room temperature” designate a temperature of about 20° C., e.g., 15 to 25° C. As a rule, ¹H-NMR and/or mass spectra have been obtained for the compounds prepared. Flash chromatography or MPLC is performed with commercial silica gel and is equivalent to silica gel chromatography. Absolute configuration of representative examples is either defined via the chemical starting material, single crystal x-ray structure determination or protein-ligand X-ray determinations. Unless otherwise specified, compounds containing chiral centers have the stereochemistry depicted. The assignment of stereochemistry has been made either by use of a chiral starting material of known stereochemistry, by stereoselective synthesis of known stereochemistry, or by biological activity.

### Scheme 1: General Synthesis Scheme for Patent Examples

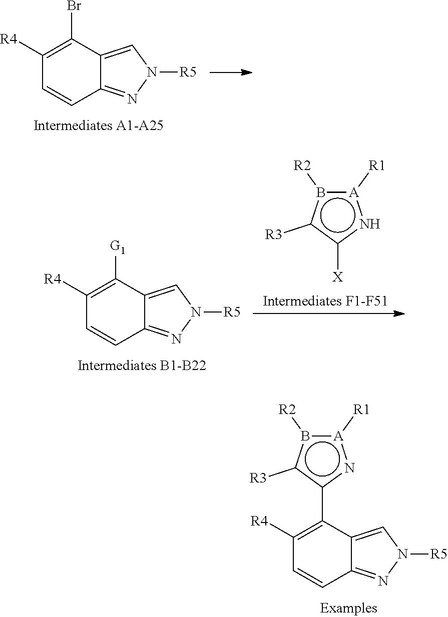

R4 is H, F;
B-A is ═C—N— or —N—C═; this means A is C or N; B is C or N; but A and B are not N at the same time.

### Scheme 2a: General Synthesis of Intermediates A: e.g. A1, A13, A14, A24

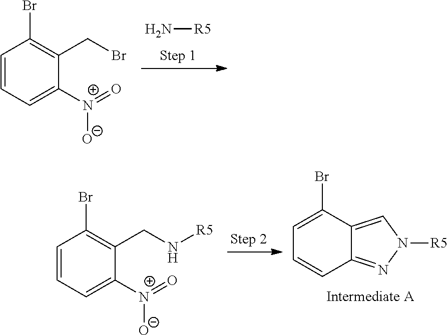

### Scheme 2b: Alternative Synthesis of Intermediates A: E.g. A2, A5, A19-A23, A25

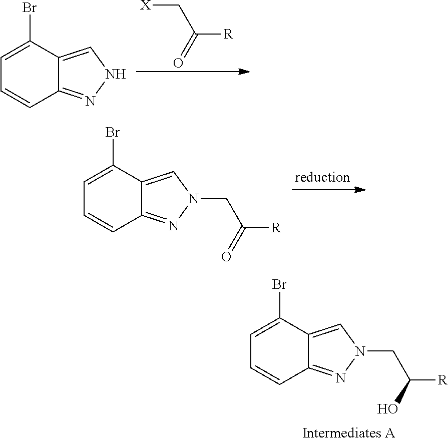

### Scheme 2c: Alternative Synthesis of Intermediates A: E.g. A4, A7

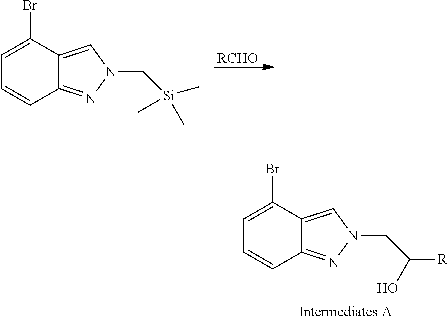

### Scheme 3: Synthesis of Intermediates F1-F20, F23-F39, F49

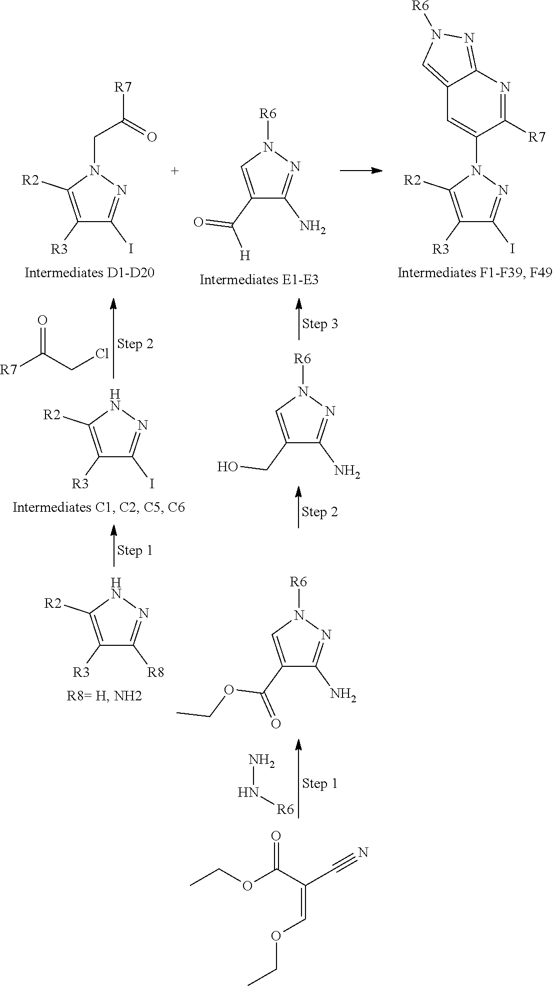

### Scheme 4: Synthesis of Intermediates F21, F22

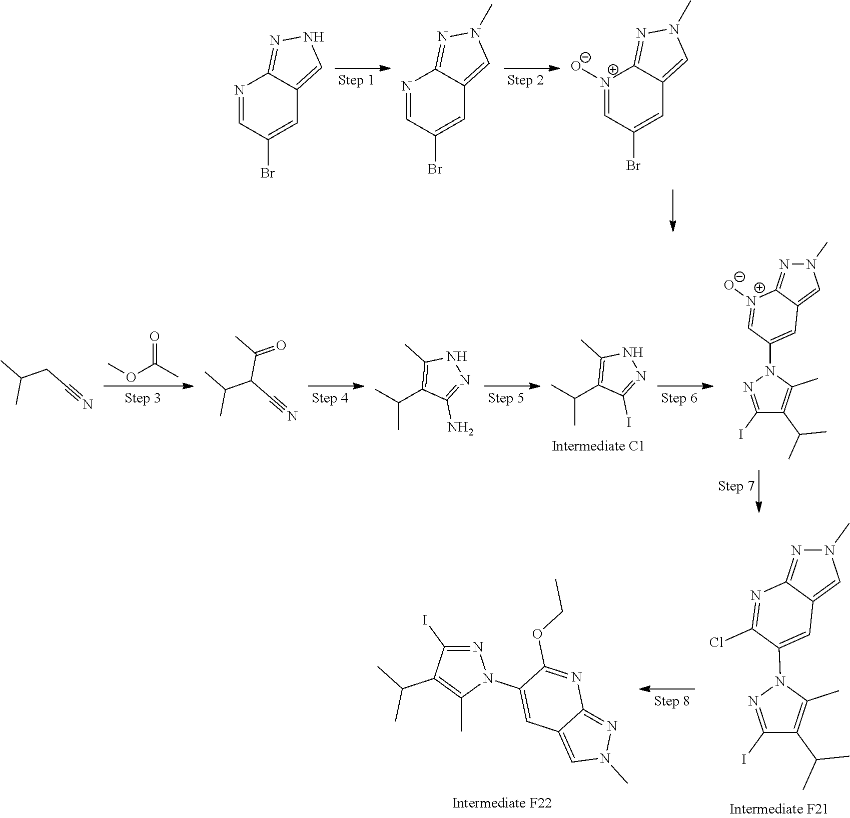

### Scheme 5: Synthesis of Imidazole Intermediates F40-F47

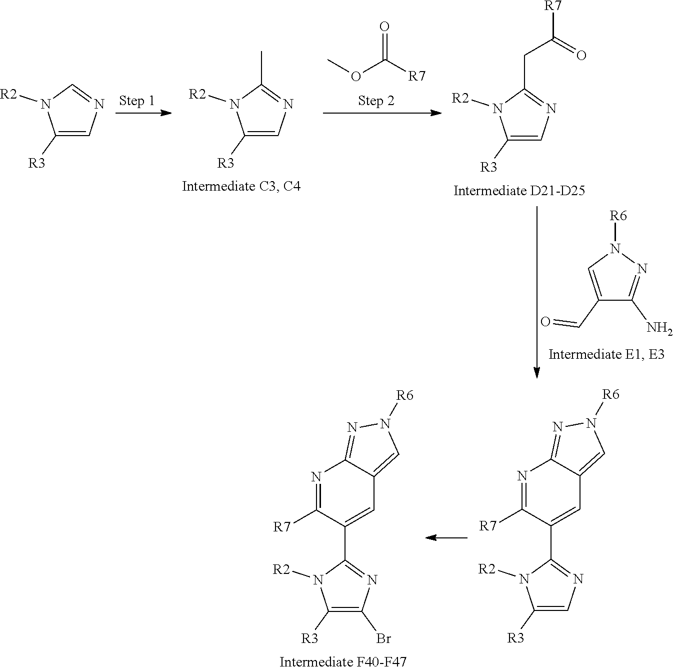

All starting materials not described are either commercially available or described in literature.

### Synthesis of Intermediates A1-A25

### Synthesis of Intermediate A1

### Step 1: Synthesis of (1R)-2-{[(2-bromo-6-nitrophenyl) methyl]amino}-1-phenylethan-1-ol

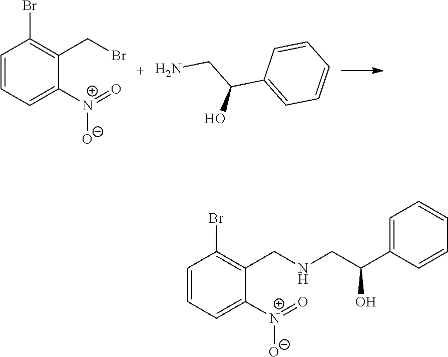 [SMILES: O=[N+]([O-])c1cccc(Br)c1CBr ; [NH3+]C[C@H](O)c1ccccc1 ; O=[N+]([O-])c1cccc(Br)c1CNC[C@H](O)c1ccccc1]

(1R)-2-Amino-1-phenylethan-1-ol (5.00 g, 17.0 mmol) is dissolved in ACN (20 mL) and DIPEA (8.75 mL, 50.9 mmol). 1-Bromo-2-(bromomethyl)-3-nitrobenzene (6.98 g, 50.9 mmol) is slowly added. The reaction mixture is stirred at RT for 2 h. The reaction mixture is concentrated and purified by flash chromatography (CycH/EtOAc 100/0 to CycH/EtOAc 10/90) to afford the desired compound.

Analysis (method A): Rₜ: 0.36 min, [M+H]⁺: 351/353 (Br)

### Step 2: Synthesis of (1R)-2-(4-bromo-2H-indazol-2-yl)-1-phenylethan-1-ol

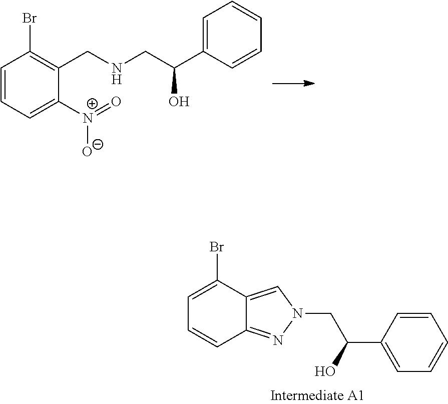 [SMILES: O=[N+]([O-])c1cccc(Br)c1CNC[C@H](O)c1ccccc1 ; O[C@@H](Cn1cc2c(Br)cccc2n1)c1ccccc1]

(1R)-2-{[(2-Bromo-6-nitrophenyl) methyl]amino}-1-phenylethan-1-ol (5.44 g, 15.5 mmol) is suspended in MeOH (25 mL). Zinc (5.06 g, 77.45 mmol) is added, and then ammonium formate (977 mg, 15.5 mmol) in MeOH (5 mL) is added dropwise over 5 min. The reaction mixture is stirred at RT overnight. The reaction mixture is filtered, washed with MeOH and the filtrate is concentrated. The residue is triturated with water. The precipitate is filtered and re-crystallized from ACN to afford the title compound.

Analysis (method A): Rₜ: 0.58 min, [M+H]⁺: 317/319 (Br)

### Synthesis of Intermediate A2 and A23

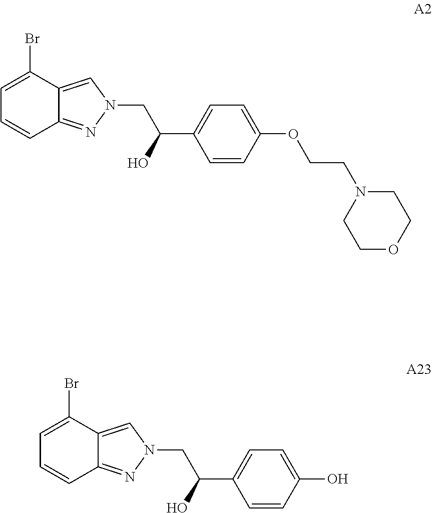 [SMILES: O[C@@H](Cn1cc2c(Br)cccc2n1)c1ccc(OCCN2CCOCC2)cc1 ; Oc1ccc([C@@H](O)Cn2cc3c(Br)cccc3n2)cc1]

### Step 1: Synthesis of 2-(4-bromo-2H-indazol-2-yl)-1-(4-hydroxyphenyl) ethan-1-one

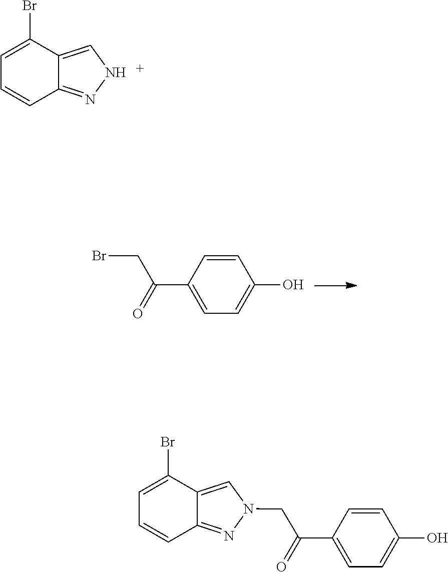 [SMILES: Brc1cccc2n[nH]cc12 ; O=C(CBr)c1ccc(O)cc1 ; O=C(Cn1cc2c(Br)cccc2n1)c1ccc(O)cc1]

2-Bromo-1-(4-hydroxyphenyl) ethan-1-one (5.00 g, 25.4 mmol), 4-bromo-2H-indazole (5.46 g, 25.4 mmol), and aluminum powder (1.37 g, 50.8 mmol, −100+325 mesh) are added to DMF/water 3/1 (48 mL). The reaction mixture is stirred at RT for 5 d. The reaction mixture is filtered, and the precipitate is washed with water. The precipitate is suspended in MeOH/DCM/DMF (100 mL/50 mL/100 mL) and the aluminum is filtered off. The filtrate is concentrated, and the residue is triturated with n-heptane (100 mL) to afford the desired product.

Analysis (method D): Rₜ: 0.31 min, [M+H]⁺: 331/333 (Br)

### Step 2: Synthesis of 4-[(1R)-2-(4-bromo-2H-indazol-2-yl)-1-hydroxyethyl]phenol

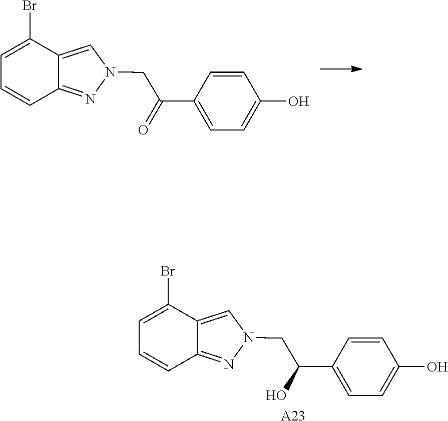

Under an argon atmosphere, 2-(4-bromo-2H-indazol-2-yl)-1-(4-hydroxyphenyl) ethan-1-one (2.00 g, 5.13 mmol, 85% purity) is dissolved in THE (40 mL). At 0° C. formic acid triethylamine complex 5:2 (10.73 mL, 25.67 mmol) is added dropwise. After stirring 5 min at 0° C. chloro([(1S, 2S)-(−)-2-amino-1, 2-diphenylethyl](4-toluenesulfonyl) amido) (mesitylene) ruthenium (II) (0.16 g, 0.26 mmol) is added and the reaction mixture is stirred at RT overnight. The reaction mixture is diluted with acetone and water and is concentrated. The residue is purified by flash chromatography (DCM/MeOH 100/0 to DCM/MeOH 95/5) to afford the desired product A23.

Analysis (method F): Rₜ: 0.74 min, [M+H]⁺: 333/335 (Br)

Chiral Analysis (method T): Rₜ: 3.80 min, >98% ee

### Step 3: Synthesis of (1R)-2-(4-bromo-2H-indazol-2-yl)-1-{4-[2-(morpholin-4-yl) ethoxy]phenyl}ethan-1-ol

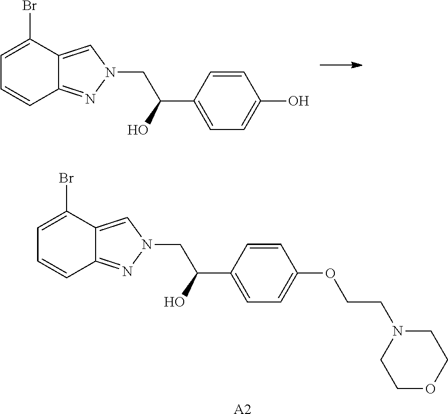 [SMILES: Oc1ccc([C@@H](O)Cn2cc3c(Br)cccc3n2)cc1 ; O[C@@H](Cn1cc2c(Br)cccc2n1)c1ccc(OCCN2CCOCC2)cc1 ; "A2"]

A mixture of 4-[(1R)-2-(4-bromo-2H-indazol-2-yl)-1-hydroxyethyl]phenol (250 mg, 0.75 mmol), 4-(2-chloroethyl) morpholine hydrochloride (279 mg, 1.50 mmol), DIPEA (260 μL, 1.50 mmol) and DMF (8 mL) are stirred at 75° C. for 4 h, then K₂CO₃ (104 mg, 0.75 mmol) and acetone (8 mL) are added, and the reaction mixture is stirred at 70° C. overnight. K₂CO₃ is filtered off and the filtrate is concentrated. The residue is purified by reversed phase chromatography (HPLC; ACN/water including NH₃) to afford compound A2.

Analysis (method H): Rₜ: 0.97 min, [M+H]⁺: 446/448 (Br)

### Synthesis of Intermediate A3

### Step 1: Synthesis of 2-[(benzenesulfinyl)methyl]-4-bromo-2H-indazole

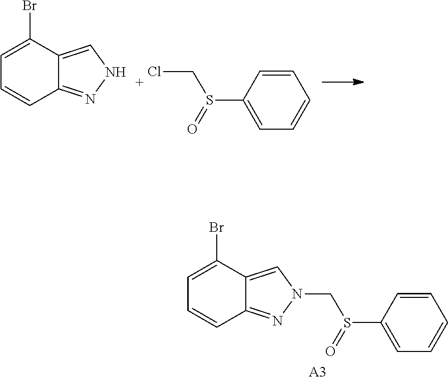

4-Bromo-1H-indazole (400 mg, 1.99 mmol) is dissolved in DMF (5 mL). K₂CO₃ (1.10 g, 7.96 mmol) and chloromethyl phenyl sulfoxide (716 mg, 3.98 mmol) are added and the reaction mixture is stirred at 50° C. overnight. K₂CO₃ (1.10 g, 7.96 mmol) is added, and the reaction mixture is stirred at 70° C. overnight. The reaction mixture is filtered, and the filtrate is purified by reversed phase chromatography (HPLC; ACN/water including TFA) to afford compound A3.

Analysis (method G): Rₜ: 0.90 min, [M+H]⁺: 335/337 (Br)

### Synthesis of Intermediate A4 and A14

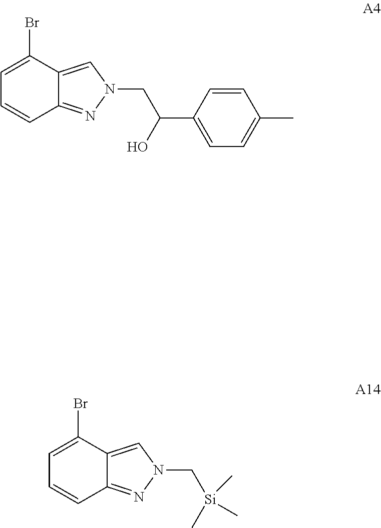 [SMILES: Cc1ccc(C(O)Cn2cc3c(Br)cccc3n2)cc1 ; C[Si](C)(C)Cn1cc2c(Br)cccc2n1]

### Step 1: Synthesis of 1-bromo-2-(bromomethyl)-3-nitrobenzene

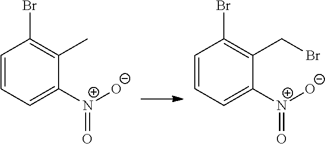

2-Bromo-6-nitrotoluene (50 g, 231 mmol) is dissolved in DCE (300 mL). A suspension of NBS (61.8 g, 347 mmol) in DCE (400 mL) is added at RT and the reaction mixture is stirred at reflux. Then AIBN (2.66 g, 16.2 mmol) in DCM (35 mL) is slowly added (syringe pump, pump rate approximately 1 drop per 6 seconds), and the reaction mixture is stirred at reflux overnight. The reaction mixture is concentrated, the residue is dissolved in DCM (500 mL) and washed 3× with water. The organic layer is dried over MgSO4 and filtered through a short plug of silica, and the silica is washed with DCM (50 ml). The filtrate is concentrated and dried in high vacuum to afford the desired product.

TLC: silica gel, CycH/EtOAc 5/1: Rf: 0.4

### Step 2: Synthesis of [(2-bromo-6-nitrophenyl) methyl][(trimethylsilyl)methyl]amine

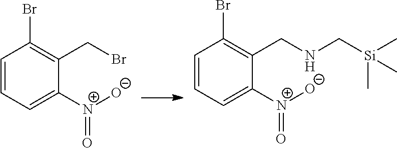 [SMILES: O=[N+]([O-])c1cccc(Br)c1CBr ; C[Si](C)(C)CNCc1c(Br)cccc1[N+](=O)[O-]]

(Trimethylsilyl)methylamine (13 mL, 97.1 mmol) and DIPEA (34.00 mL, 196.6 mmol) are dissolved in ACN (200 mL). A solution of 1-bromo-2-(bromomethyl)-3-nitrobenzene (20.00 g, 67.14 mmol) in ACN (200 mL) is added dropwise and the reaction mixture is stirred at RT for 1 h. The reaction mixture is concentrated, the residue is dissolved in DCM and washed 3× with water. The organic layer is filtered through silica, the silica is washed with DCM and the filtrate is concentrated to afford the desired product.

Analysis (method G): Rₜ: 0.75 min, [M+H]⁺: 317/319 (Br)

### Step 3: Synthesis of 4-bromo-2-[(trimethylsilyl)methyl]-2H-indazole

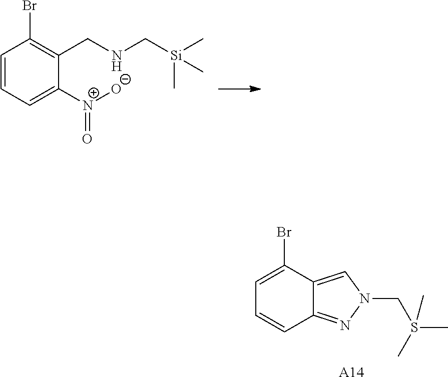 [SMILES: C[Si](C)(C)CNCc1c(Br)cccc1[N+](=O)[O-] ; CS(C)(C)Cn1cc2c(Br)cccc2n1 ; "A14"]

[(2-Bromo-6-nitrophenyl) methyl][(trimethylsilyl)methyl]amine (18.64 g, 58.75 mmol) is dissolved in MeOH (1.6 L). Zinc (19.2 g, 294 mmol) is added and ammonium formate (5.55 g, 88.12 mmol) in MeOH (38 mL) is slowly added dropwise at 50° C. over 7 h. The reaction mixture is filtered over Celite, washed with MeOH and the filtrate is concentrated. The residue is purified by flash chromatography (CycH/EtOAc 88/12 to CycH/EtOAc 0/100) to afford compound A14.

Analysis (method G): Rₜ: 1.18 min, [M+H]⁺: 283/285 (Br)

### Step 4: Synthesis of 2-(4-bromo-2H-indazol-2-yl)-1-(4-methylphenyl) ethan-1-ol

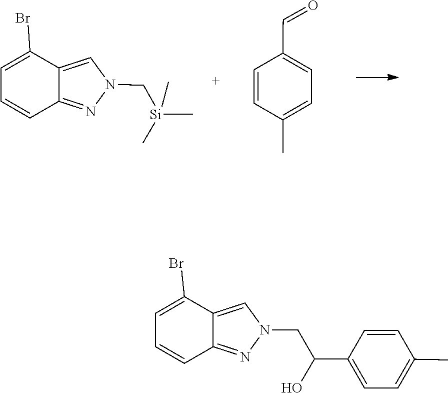

p-Tolualdehyde (103 μL, 0.85 mmol) and CsF (107 mg, 0.71 mmol) are suspended in DMF (1.5 mL). A solution of 4-bromo-2-[(trimethylsilyl)methyl]-2H-indazole (200 mg, 0.71 mmol) in DMF (2.5 mL) is added dropwise at RT to the reaction mixture which is stirred at RT for 2 h. The reaction mixture is filtered, the filtrate is concentrated in vacuo, diluted with ACN and purified by reversed phase chromatography (HPLC; ACN/water/NH₄OH) to afford the desired product.

Analysis (method H): Rₜ: 0.94 min, [M+H]⁺: 331/333 (Br)

### Synthesis of Intermediate A5:(1R)-2-[4-(5, 5-dimethyl-1, 3, 2-dioxaborinan-2-yl)-5-fluoro-2H-indazol-2-yl]-1-phenylethan-1-ol

### (1R)-2-(4-bromo-5-fluoro-2H-indazol-2-yl)-1-phenylethan-1-ol

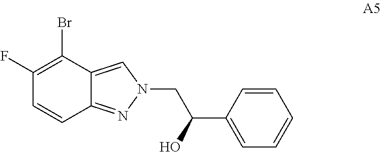 [SMILES: O[C@@H](Cn1cc2c(Br)c(F)ccc2n1)c1ccccc1]

### Step 1: Synthesis of 2-(4-bromo-5-fluoro-2H-indazol-2-yl)-1-phenylethan-1-one

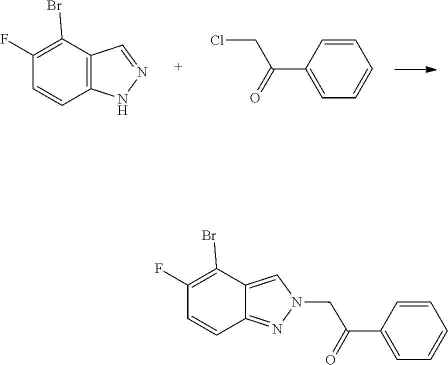 [SMILES: Fc1ccc2[nH]ncc2c1Br ; O=C(CCl)c1ccccc1 ; O=C(Cn1cc2c(Br)c(F)ccc2n1)c1ccccc1]

4-Bromo-5-fluoro-1H-indazole (800 mg, 3.72 mmol) and 2-chloro-1-phenylethan-1-one (1.15 g, 7.44 mmol) are combined in a microwave tube and the reaction mixture is stirred in the microwave at 130° C. for 45 min. The reaction mixture is purified by reversed phase chromatography (HPLC; ACN/water/TFA) to afford the desired compound.

Analysis (method A): Rₜ: 0.62 min, [M+H]⁺: 333/335 (Br)

### Step 2: Synthesis of (1R)-2-(4-bromo-5-fluoro-2H-indazol-2-yl)-1-phenylethan-1-ol

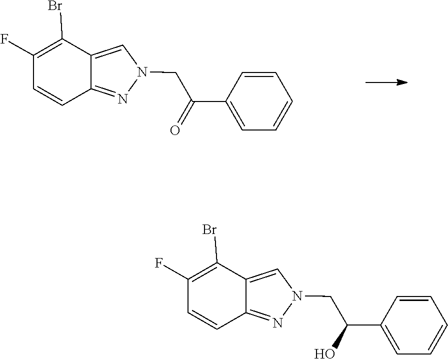 [SMILES: O=C(Cn1cc2c(Br)c(F)ccc2n1)c1ccccc1 ; O[C@@H](Cn1cc2c(Br)c(F)ccc2n1)c1ccccc1]

2-(4-Bromo-5-fluoro-2H-indazol-2-yl)-1-phenylethan-1-one (660 mg, 1.98 mmol) is dissolved in THE (10 mL). At 0° C. formic acid triethylamine complex 5:2 (4.14 mL, 9.91 mmol) is added dropwise Chloro([(1S, 2S)-(−)-2-amino-1, 2-diphenylethyl](4-toluenesulfonyl) amido) (mesitylene)ruthenium (II) (61.6 mg, 0.10 mmol) is added and the reaction mixture is stirred at RT for 2 h. The reaction mixture is quenched with water, the THF is evaporated, and the residue is purified by reversed phase chromatography (HPLC; ACN/water including TFA). The lyophilized fractions are re-crystallized from ACN/MeOH to afford the desired compound A5.

Analysis (method A): Rₜ: 0.62 min, [M+H]⁺: 335/337 (Br)

Chiral analysis (method ZD): Rₜ: 2.57 min, >98% ee

### Synthesis of Intermediate A6

### Synthesis of ethyl 2-[(1R)-2-(4-bromo-2H-indazol-2-yl)-1-phenylethoxy]acetate

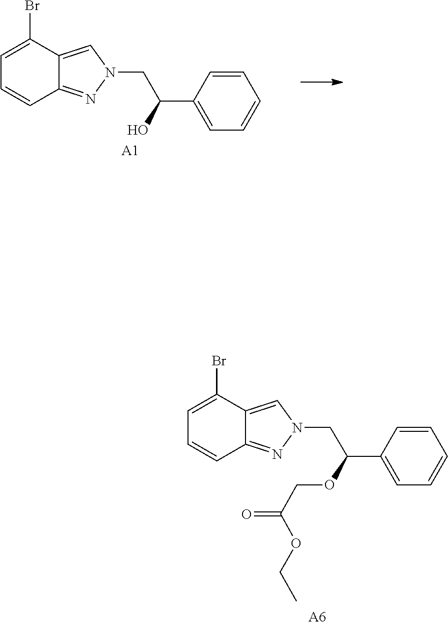

Under an argon atmosphere, (1R)-2-(4-bromo-2H-indazol-2-yl)-1-phenylethan-1-ol (5 g, 15.8 mmol) is dissolved in DCM (140 mL). Rhodium (II) acetate dimer (125 mg, 0.28 mmol) is added and ethyl diazoacetate (16.6 mL, 158 mmol) in DCM (8 mL) is added dropwise over 24 h at RT. The reaction mixture is filtered, and the filtrate is purified by reversed phase chromatography (HPLC; ACN/water including—TFA to afford the desired compound A6.

Analysis (method F): Rₜ: 1.05 min, [M+H]⁺: 403/405 (Br)

### Synthesis of Intermediate A7

### Step 1: Synthesis of 2-(4-bromo-2H-indazol-2-yl)-1-(3, 4-difluorophenyl) ethan-1-ol

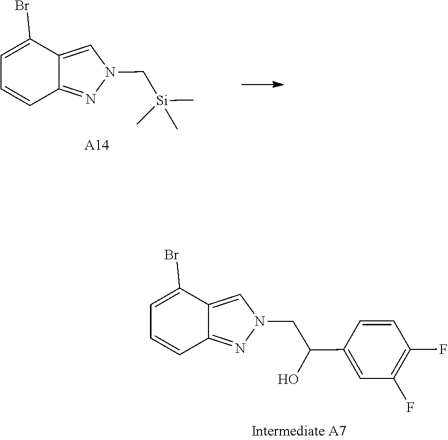

3, 4-Difluorbenzaldehyde (351 mg, 2.47 mmol), CsF (268 mg, 1.8 mmol) and 4-bromo-2-[(trimethylsilyl)methyl]-2H-indazole (A14, 500 mg, 1.76 mmol) are suspended in DMF (6 mL) and stirred at RT for 2 h. The reaction mixture is diluted with HOAc and DMF and purified by reversed phase chromatography (HPLC; ACN/water including NH3) to afford the desired compound A7.

Analysis (method G): Rₜ: 0.91 min, [M+H]⁺: 353/355 (Br)

### Synthesis of Intermediate A8

### Step 1: Synthesis of 2-(4-bromo-2H-indazol-2-yl)-1-phenylethan-1-one

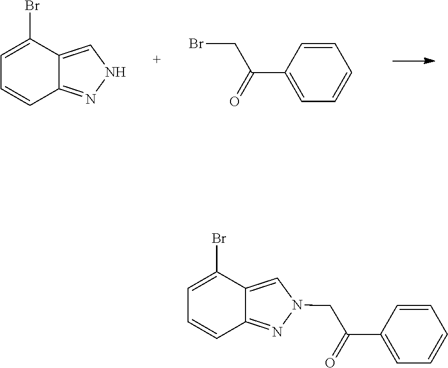 [SMILES: Brc1cccc2n[nH]cc12 ; O=C(CBr)c1ccccc1 ; O=C(Cn1cc2c(Br)cccc2n1)c1ccccc1]

4-Bromo-2H-indazole (2 g, 10.2 mmol), 2-bromo-1-phenylethan-1-one (4.04 g, 20.3 mmol) and aluminum powder (548 mg, 20.3 mmol; −100+325 mesh) are dissolved in DMF/water 3/1 (20 mL). The reaction mixture is stirred at 80° C. overnight. The aluminum is filtered off and washed with DMF. The filtrate is diluted with water and extracted with EtOAc. The organic layer is dried (Na₂SO₄), filtered, and concentrated. The residue is first triturated with water and then with MeOH. The precipitate is filtered to afford the desired compound.

Analysis (method A): Rₜ: 0.60 min, [M+H]⁺: 315/317 (Br)

### Step 2: Synthesis of 4-bromo-2-(2, 2-difluoro-2-phenylethyl)-2H-indazole

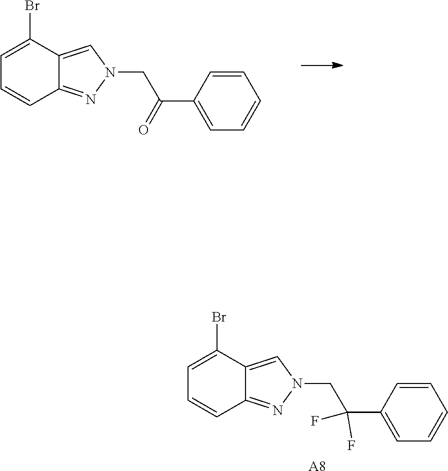

2-(4-Bromo-2H-indazol-2-yl)-1-phenylethan-1-one (1.08 g, 3.41 mmol) is dissolved in toluene (6 mL) and DCM (6 mL). At 0° C. DAST (3.93 mL, 30 mmol) is added dropwise, and the reaction mixture is stirred at RT for 4 d. The reaction mixture is quenched with a saturated NaHCO₃ solution and extracted with DCM. The organic layer is dried (Na₂SO₄), filtered, and concentrated. The residue is purified by flash chromatography (CycH/EtOAc 70/30) to afford the desired compound A8.

Analysis (method A): Rₜ: 0.68 min, [M+H]⁺: 337/339 (Br)

### Synthesis of Intermediates A9 to A12

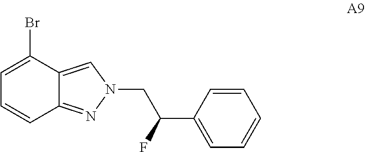 [SMILES: F[C@@H](Cn1cc2c(Br)cccc2n1)c1ccccc1]

Step 1-2: are synthesized by following a procedure analogous to that described for Intermediate A1 with racemic 2-amino-1-phenylethan-1-ol as starting material.

### Step 3: Synthesis of 4-bromo-2-(2-fluoro-2-phenylethyl)-2H-indazole

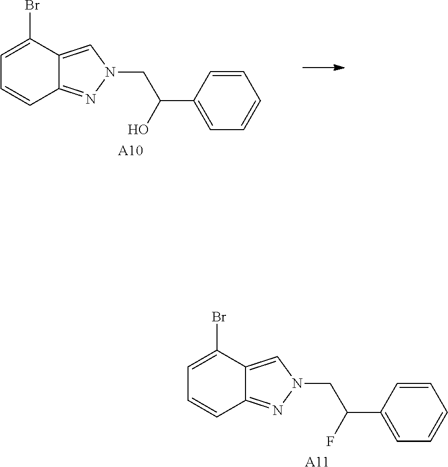

Racemic 2-(4-bromo-2H-indazol-2-yl)-1-phenylethan-1-ol (A10, 6.10 g, 17.3 mmol) is dissolved in DCM (45 mL) and [bis(2-methoxyethyl) amino]sulfur trifluoride (9.70 mL, 26.3 mmol, 50% in toluene) is added dropwise. The reaction mixture is stirred at RT for 4 d. The reaction mixture is quenched with a saturated NaHCO₃ solution and extracted with DCM. The organic layer is dried (Na₂SO₄), filtered, and concentrated. The residue is purified by reversed phase chromatography (HPLC; ACN/water/formic acid) to afford the desired product A11.

Analysis (method G): Rₜ: 1.12 min, [M+H]⁺: 319/321 (Br)

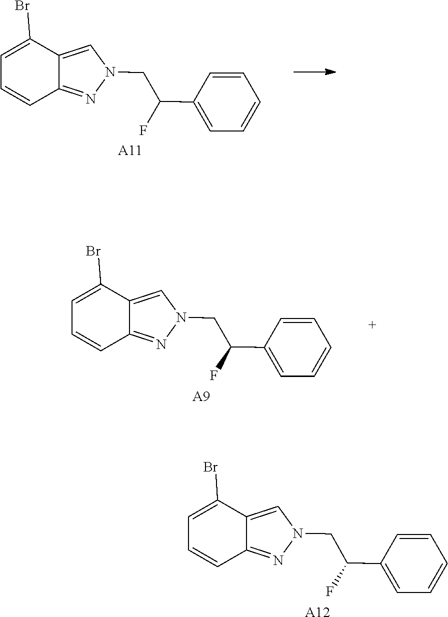

4-Bromo-2-(2-fluoro-2-phenylethyl)-2H-indazole (455 mg) is separated by chiral purification method ZC to afford the compounds 4-bromo-2-[(2R)-2-fluoro-2-phenylethyl]-2H-indazole A9 (--. (Method ZC): Rₜ: 4.52 min) and 4-bromo-2-[(2S)-2-fluoro-2-phenylethyl]-2H-indazole A12—(Analysis: (method ZC): Rₜ: 3.14 min).

### Synthesis of Intermediate A13

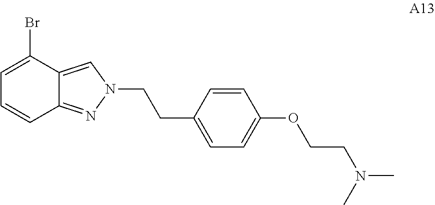 [SMILES: CN(C)CCOc1ccc(CCn2cc3c(Br)cccc3n2)cc1]

### Step 1: Synthesis of tert-butyl N-(2-{4-[2-(dimethyl amino)ethoxy]phenyl}ethyl)carbamate

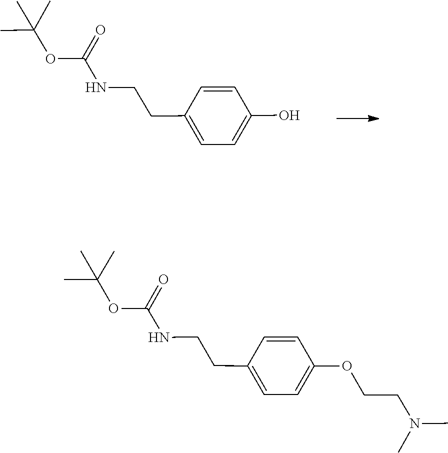 [SMILES: CC(C)(C)OC(=O)NCCc1ccc(O)cc1 ; CN(C)CCOc1ccc(CCNC(=O)OC(C)(C)C)cc1]

Tert-butyl N-[2-(4-hydroxyphenyl) ethyl]carbamate (7.10 g, 30 mmol) is dissolved in acetone (70 mL). (2-Chloroethyl) dimethylamine hydrochloride (5.50 g, 38.2 mmol) and Cs₂CO₃ (20.00 g, 61.4 mmol) are added, and the reaction mixture is stirred at 75° C. overnight. The reaction mixture is filtered, and the filtrate is concentrated. The residue is purified by flash chromatography (DCM/MeOH 100/0 to DCM/MeOH 85/15) to afford the desired product.

Analysis (method H): Rₜ: 1.01 min, [M+H]⁺: 309

### Step 2: Synthesis of 2-{4-[2-(dimethylamino) ethoxy]phenyl}ethan-1-amine hydrochloride

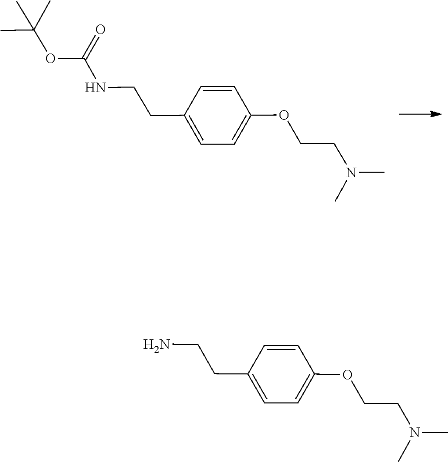 [SMILES: CN(C)CCOc1ccc(CCNC(=O)OC(C)(C)C)cc1 ; CN(C)CCOc1ccc(CCN)cc1]

Tert-butyl N-(2-{4-[2-(dimethylamine) ethoxy]phenyl}ethyl) carbamate (6.30 g, 20.4 mmol) is dissolved in dioxane (5 mL). 4 M HCl in dioxane (24 mL, 96 mmol) is added and the reaction mixture is stirred at RT for 2 h. The reaction mixture is concentrated to afford the desired product.

Analysis (method G): Rₜ: 0.76 min, [M+H]⁺: 209

### Step 3: Synthesis of [(2-bromo-6-nitrophenyl) methyl](2-{4-[2-(dimethylamino)ethoxy]phenyl}ethyl) amine

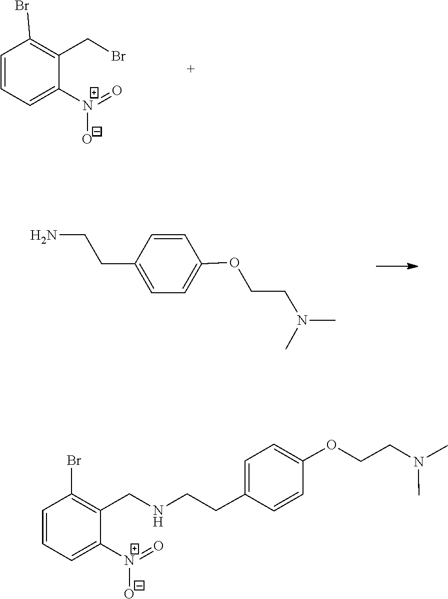

1-Bromo-2-(bromomethyl)-3-nitrobenzene (900 mg, 3.05 mmol) and 2-{4-[2-(dimethylamino) ethoxy]phenyl}ethan-1-amine hydrochloride (800 mg, 3.27 mmol) are dissolved in ACN (10 mL). DIPEA (3.50 mL, 20.3 mmol) is added, and the reaction mixture is stirred at RT overnight. The reaction mixture is concentrated and purified by flash chromatography (DCM/MeOH 100/0 to DCM/MeOH 65/35) to afford the desired product.

Analysis (method G): Rₜ: 0.63 min, [M+H]⁺: 422/424 (Br)

### Step 4: Synthesis of (2-{4-[2-(4-bromo-2H-indazol-2-yl) ethyl]phenoxy}ethyl) dimethylamine

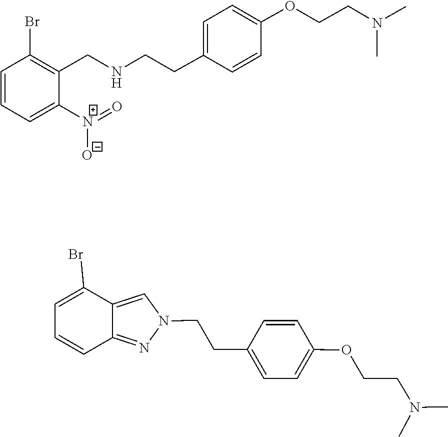

[(2-Bromo-6-nitrophenyl) methyl](2-{4-[2-(dimethylamine) ethoxy]phenyl}ethyl) amine (1.20 g, 2.56 mmol) is suspended in MeOH. Zinc (836 mg, 12.8 mmol) and ammonium formate (110 mg, 1.74 mmol) are added, and the reaction mixture is stirred at RT overnight. The reaction mixture is filtered, washed with MeOH and the filtrate is concentrated. The residue is purified by flash chromatography (DCM/MeOH 100/0 to DCM/MeOH 72/28) to afford intermediate A13.

Analysis (method G): Rₜ: 0.83 min, [M+H]⁺: 388/390 (Br)

### Synthesis of Intermediate A15 to A18

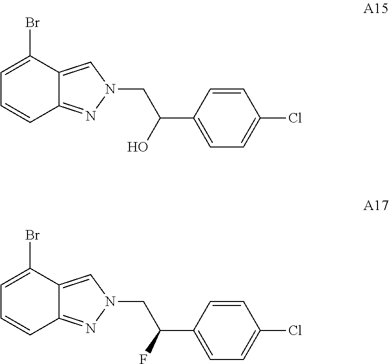 [SMILES: OC(Cn1cc2c(Br)cccc2n1)c1ccc(Cl)cc1 ; F[C@@H](Cn1cc2c(Br)cccc2n1)c1ccc(Cl)cc1]

A15 is synthesized by following a procedure analogous to that described for Intermediate A4 (step 4) using A13 and 4-chlorobenzaldehyde as starting material.

### Step 1: Synthesis of 4-bromo-2-[2-(4-chlorophenyl)-2-fluoroethyl]-2H-indazole

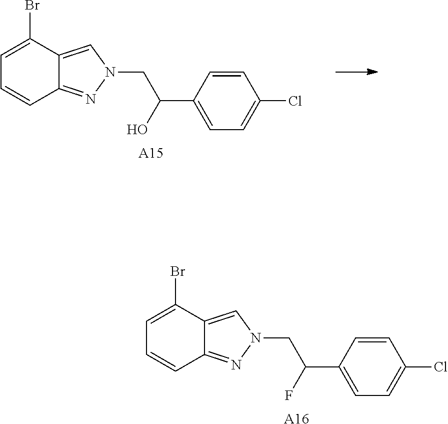

2-(4-Bromo-2H-indazol-2-yl)-1-(4-chlorophenyl) ethan-1-ol (1.23 g, 3.50 mmol) is dissolved in DCM (20 mL). At 0° C. [bis(2-methoxyethyl) amino]sulfur trifluoride (2.28 mL, 5.25 mmol, 50% in THF) is added dropwise and the reaction mixture is stirred at RT for 2.5 h. The reaction mixture is quenched and basified with a 2 M solution of Na₂CO₃ and extracted with DCM. The organic layer is washed with a saturated NaHCO₃ solution, dried (Na₂SO₄), filtered, and concentrated. The residue is purified by reversed phase chromatography (HPLC; ACN/water including TFA) to afford the desired product.

Analysis (method F): Rₜ: 1.10 min, [M+H]⁺: 353/355 (Br)

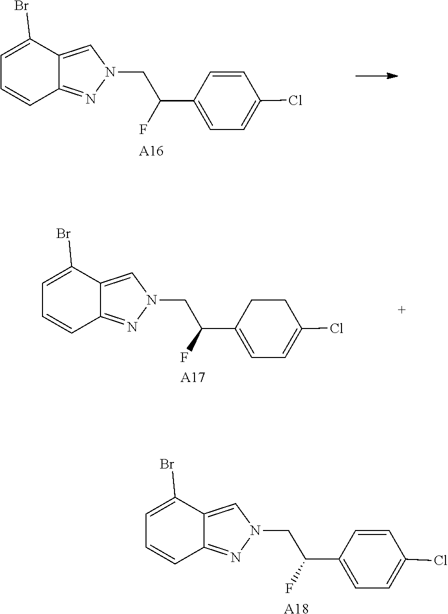

### Step 2: Synthesis of 4-bromo-2-[(2R)-2-(4-chlorophenyl)-2-fluoroethyl]-2H-indazole

4-Bromo-2-[2-(4-chlorophenyl)-2-fluoroethyl]-2H-indazole (750 mg, 2.12 mmol) is separated by chiral purification method ZB to afford the compounds 4-bromo-2-[(2R)-2-(4-chlorophenyl)-2-fluoroethyl]-2H-indazole A17 ((Analysis method ZB): Rₜ: 0.95 min) and 4-bromo-2-[(2S)-2-(4-chlorophenyl)-2-fluoroethyl]-2H-indazole A18 ((Analysis: (method ZB): Rₜ: 0.79 min).

### Synthesis of Intermediate A19

### Step 1: Synthesis of 2-(4-bromo-2H-indazol-2-yl)-1-cyclohexylethan-1-one

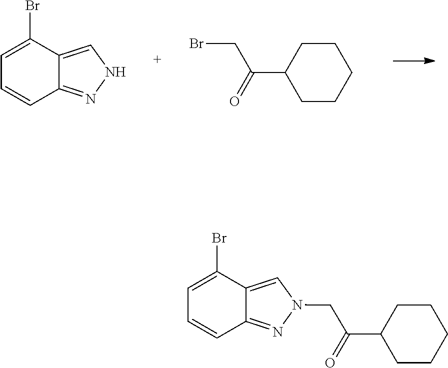 [SMILES: Brc1cccc2n[nH]cc12 ; O=C(CBr)C1CCCCC1 ; O=C(Cn1cc2c(Br)cccc2n1)C1CCCCC1]

4-Bromo-1H-indazole (500 mg, 2.54 mmol) is dissolved in ACN (8 mL). K₂CO₃ (877 mg, 6.34 mmol) and 2-bromo-1-cyclohexyletan-1-one (521 mg, 2.54 mmol) are added, and the reaction mixture is stirred at 50° C. overnight. The reaction mixture is purified by reversed phase chromatography (HPLC; ACN/water including NH₃) to afford the desired product

Analysis (method G): Rₜ: 1.13 min, [M+H]⁺: 321/323 (Br)

### Step 2: Synthesis of 2-(4-bromo-2H-indazol-2-yl)-1-cyclohexylethan-1-ol

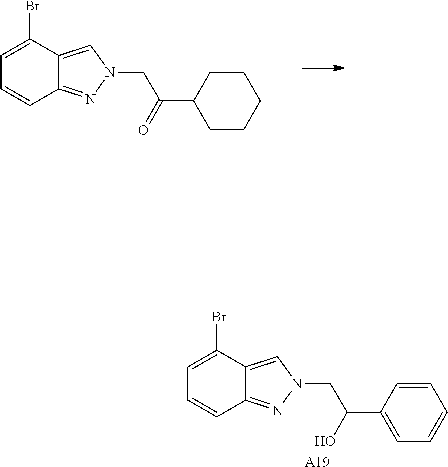

2-(4-Bromo-2H-indazol-2-yl)-1-cyclohexylethan-1-one (200 mg, 0.62 mmol) is dissolved in THE (3 mL) and MeOH (3 mL). At 0° C. sodium borohydride (23.6 mg, 0.62 mmol) is added, and the reaction mixture is stirred at 0° C. for 2 h. The reaction mixture is quenched with 1 M HCl and stirred at RT for 15 min. Then the reaction mixture is basified with a saturated NaHCO₃ solution and extracted with EtOAc. The organic layer is dried (Na₂SO₄), filtered and concentrated to afford the desired product A19.

Analysis (method G): Rₜ: 1.13 min, [M+H]⁺: 323/325 (Br)

### Synthesis of Intermediate A20

### Step 1: Synthesis of 1-[4-(benzyloxy) phenyl]-2-(4-bromo-2H-indazol-2-yl) ethan-1-one

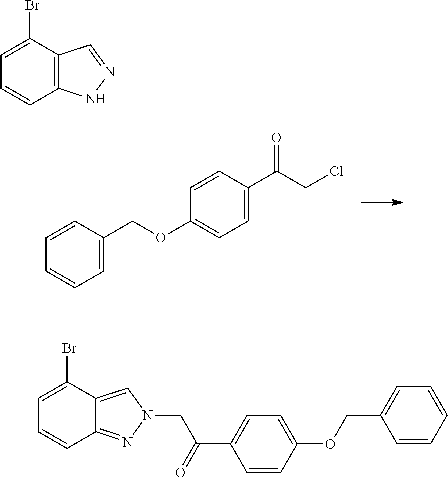

4-Bromo-1H-indazole (3.5 g, 17.8 mmol) and 1-[4-(benzyloxy) phenyl]-2-chloroethan-1-one (5.7 g, 19.7 mmol) are heated to 135° C. and the melted mixture stirred for 2 h. After cooling the mixture is triturated with ACN and the formed solid is collected and extracted between DCM and aqueous Na₂CO₃ solution. The organic phase was concentrated and triturated in a mixture of ACN/EtOAc 2:1 to provide the desired product.

Analysis (method A): Rₜ: 0.78 min, [M+H]⁺: 421/435 (Br)

### Step 2: Synthesis of (1R)-1-[4-(benzyloxy) phenyl]-2-(4-bromo-2H-indazol-2-yl)ethan-1-ol

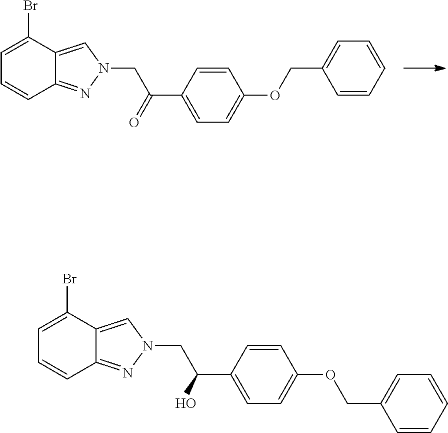 [SMILES: O=C(Cn1cc2c(Br)cccc2n1)c1ccc(OCc2ccccc2)cc1 ; O[C@@H](Cn1cc2c(Br)cccc2n1)c1ccc(OCc2ccccc2)cc1]

1-[4-(Benzyloxy) phenyl]-2-(4-bromo-2H-indazol-2-yl) ethan-1-one (2.05 g, 4.86 mmol) is dissolved in THE (100 mL), formic acid triethylamine complex 5:2 (10, 15 mL) and chloro([(1S, 2S)-(−)-2-amino-1, 2-diphenylethyl](4-toluenesulfonyl) amido) (mesitylene) ruthenium(II) (181 mg) is added and the mixture stirred at RT overnight. The mixture is concentrated and extracted with DCM/aqueous Na₂CO₃ solution. The organic phase was concentrated to provide the desired product A20.

Analysis (method A): Rₜ: 0.75 min, [M+H]⁺: 423/425 (Br)

Chiral HPLC (method V): >98% ee

### Synthesis of Intermediate A21

### Synthesis of 2-(4-bromo-2H-indazol-2-yl)-1-(2-methylpyridin-4-yl) ethan-1-ol

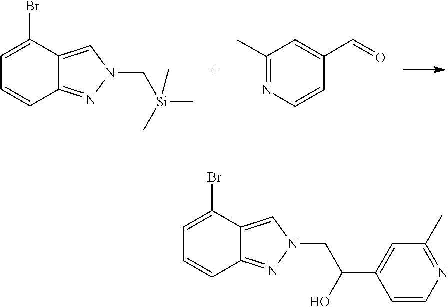

4-Bromo-2-[(trimethylsilyl)methyl]-2H-indazole (500 mg, 1.76 mmol) and 1-methyl-1H-pyrazole-4-carbaldehyde (231 mg, 2.1 mmol) are dissolved in 4 mL DMF, CsF (227 mg, 1.5 mmol) is added, and the mixture stirred for 1.5 h at RT. Then the mixture was diluted with HOAc and DMF and purified by preparative reversed phase HPLC Sunfire C18, ACN/water including formic acid) to provide the desired product A21.

Analysis (method G): Rₜ: 0.84 min, [M+H]⁺: 321/323 (Br)

### Synthesis of Intermediate A22

### Synthesis of 2-(4-bromo-2H-indazol-2-yl)-1-(1-methyl-1H-pyrazol-4-yl) ethan-1-ol

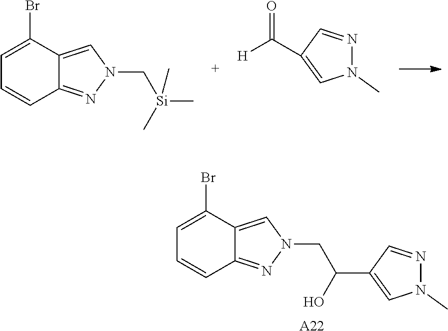

4-Bromo-2-[(trimethylsilyl)methyl]-2H-indazole (500 mg, 1.76 mmol) and 2-methylpyridine-4-carbaldehyde (290 mg, 2.39 mmol) are dissolved in 1.8 mL DMF, and CsF (286 mg, 1.765 mmol) is added, and the mixture stirred for 3 h at RT. Then the mixture was diluted with HOAc and DMF and purified by preparative reversed phase HPLC (Sunfire C18, ACN/water including formic acid) to provide the desired product A22.

Analysis (method G): Rₜ: 0.72 min, [M+H]⁺: 332/334 (Br)

### Synthesis of Intermediate A24

### Step 1: (1R)-2-{[(2-bromo-6-nitrophenyl) methyl]amino}-1-(4-fluorophenyl) ethan-1-ol

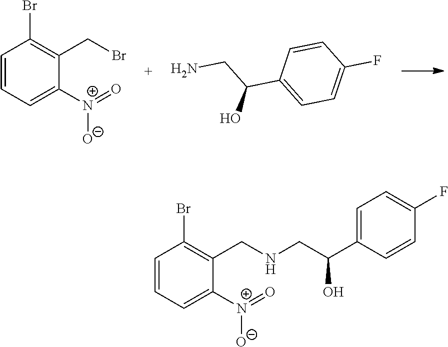 [SMILES: O=[N+]([O-])c1cccc(Br)c1CBr ; NC[C@H](O)c1ccc(F)cc1 ; O=[N+]([O-])c1cccc(Br)c1CNC[C@H](O)c1ccc(F)cc1]

(1R)-2-Amino-1-(4-fluorophenyl) ethan-1-ol hydrochloride (292 mg, 1.08 mmol) is dissolved in ACN (5 mL) and DIPEA (0.525 mL, 3.05 mmol). 1-Bromo-2-(bromomethyl)-3-nitrobenzene (300 mg, 1.02 mmol) is added dropwise. The reaction mixture is stirred at RT for 2 h. The reaction mixture is concentrated and purified by reversed phase chromatography to afford the desired product.

Analysis (method G): Rₜ: 0.73 min, [M+H]⁺: 369/371 (Br)

### Step 2: Synthesis of (1R)-2-(4-bromo-2H-indazol-2-yl)-1-(4-fluorophenyl) ethan-1-ol

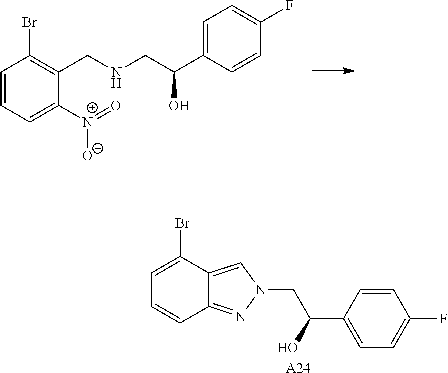

(1R)-2-{[(2-Bromo-6-nitrophenyl) methyl]amino}-1-(4-fluorophenyl) ethan-1-ol (338 mg, 0.96 mmol) is suspended in MeOH (5 mL). Zinc (300 mg, 4.6 mmol) is added and then ammonium formate (57.7 mg, 0.92 mmol) in MeOH (3 mL) is added dropwise. The reaction mixture is stirred at RT for 2 days. The reaction mixture is filtered through Celite, washed with MeOH and the filtrate is concentrated. The residue is dissolved in DMF and purified by preparative reversed phase HPLC to afford the desired product A24.

Analysis (method G): Rₜ: 1.022 min, [M+H]⁺: 335/337 (Br)

### Synthesis of Intermediate A25

### Step 1: Synthesis of 2-(4-bromo-2H-indazol-2-yl)-1-{4-[2-(dimethylamino) ethoxy]phenyl}ethan-1-one

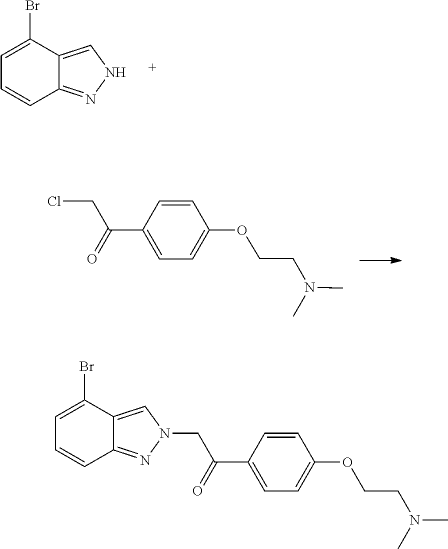 [SMILES: Brc1cccc2n[nH]cc12 ; CN(C)CCOc1ccc(C(=O)CCl)cc1 ; CN(C)CCOc1ccc(C(=O)Cn2cc3c(Br)cccc3n2)cc1]

2-Chloro-1-{4-[2-(dimethylamino) ethoxy]phenyl}ethan-1-one triflate (1.5 g, 3.75 mmol) and 4-bromo-1H-indazole (0.739 g, 3.75 mmol) are heated at 140° C. for 90 min. After cooling the mixture was purified by preparative reversed phase HPLC (Sunfire C18, ACN/water including TFA) to provide the desired product.

Analysis (method B): Rₜ: 0.60 min, [M+H]⁺: 402/404 (Br)

### Step 2: Synthesis of (1R)-2-(4-bromo-2H-indazol-2-yl)-1-{4-[2-(dimethylamino) ethoxy]phenyl}ethan-1-ol

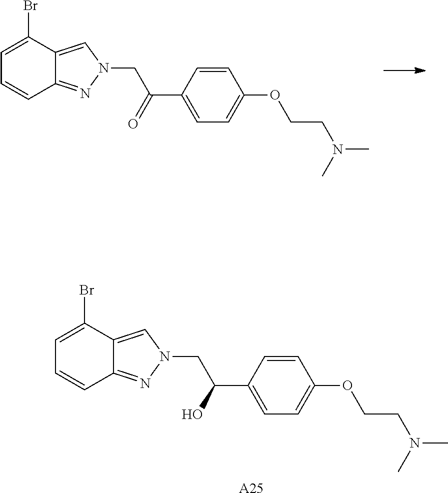 [SMILES: CN(C)CCOc1ccc(C(=O)Cn2cc3c(Br)cccc3n2)cc1 ; CN(C)CCOc1ccc([C@@H](O)Cn2cc3c(Br)cccc3n2)cc1 ; "A25"]

2-(4-Bromo-2H-indazol-2-yl)-1-{4-[2-(dimethylamino) ethoxy]phenyl}ethan-1-one (0.85 g, 1.65 mmol as triflate) is dissolved in THE (20 mL, and cooled water/ice bath. Within 5 min formic acid triethylamine complex 5:2 (3.44 mL, 8.32 mmol) is added. Then after 5 min chloro([(1S, 2S)-(−)-2-amino-1, 2-diphenylethyl](4-toluenesulfonyl) amido) (mesitylene) ruthenium (II) (51 mg, 0.082 mmol) is added and the mixture stirred at RT for 3.5 h. Then 60 mL of MeTHF and 5 mL of 2M aq. Na₂CO₃ solution is added, the organic phase is separated and dried over Na₂SO₄ to yield the desired product A25.

Analysis (method C): Rₜ: 0.61 min, [M+H]⁺: 404/406 (Br)

### Synthesis of Intermediates B1-B20

### Synthesis of (1R)-1-phenyl-2-[4-(4, 4, 5, 5-tetramethyl-1, 3, 2-dioxaborolan-2-yl)-2H-indazol-2-yl]ethan-1-ol (B1)

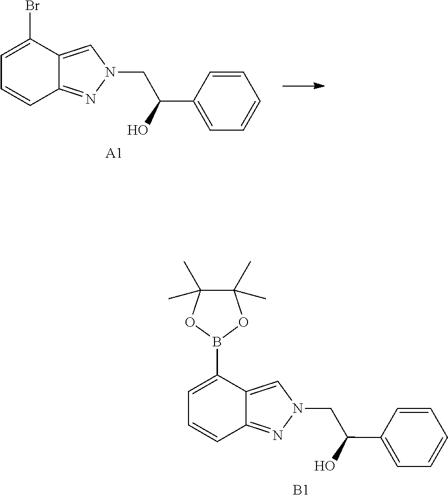

(1R)-2-(4-Bromo-2H-indazol-2-yl)-1-phenylethan-1-ol (A1, 10 g, 31.5 mmol) is dissolved in dioxane (200 mL). Bis(pinacolato)diboron (9.61 g, 37.8 mmol), potassium acetate (8.03 g, 82 mmol) and Pd(dppf)Cl₂×DCM (2.00 g, 2.45 mmol) are added at RT, and the reaction mixture is stirred at 80° C. overnight. After the reaction mixture is cooled to RT the formed precipitate is filtered and washed with 3×20 mL dioxane. The filtrate is concentrated, and the residue is suspended and triturated in CycH overnight. The precipitate is filtered, washed with 2×20 mL CycH and dried in an oven at 50° C. overnight to afford the desired product B1.

Analysis (method G): Rₜ: 1.12 min, [M+H]⁺: 365

### Synthesis of (1R)-2-[4-(5, 5-dimethyl-1, 3, 2-dioxaborinan-2-yl)-2H-indazol-2-yl]-1-phenylethan-1-ol (B2) and {2-[(2R)-2-hydroxy-2-phenylethyl]-2H-indazol-4-yl}boronic acid (B3)

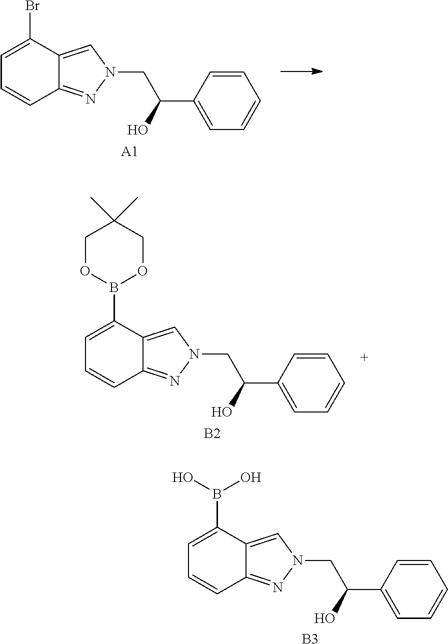

(1R)-2-(4-Bromo-2H-indazol-2-yl)-1-phenylethan-1-ol (7.50 g, 23.7 mmol) is dissolved in dioxane (60 mL). Bis (neopentyl glycolato) diboron (8.01 g, 35.5 mmol), potassium acetate (7.00 g, 71.3 mmol) are added, and the mixture is purged with nitrogen. Pd(dppf)Cl₂×DCM (0.71 g, 0.87 mmol) is added, and the reaction mixture is stirred at 85° C. for 4.5 h. After the reaction mixture is cooled to RT, it is filtered through Celite and Thiol-Resin, and the filtrate is concentrated. The residue is diluted with DCM, and the organic phase is washed 2× with water and with brine. The organic layer is dried (Na₂SO₄), filtered and evaporated. The residue is triturated with diethyl ether and the formed precipitate is filtered and dried in an oven at 50° C. overnight to afford compound B2.

Analysis (method G): Rₜ: 0.77 min, [M+H]⁺: 351

The filtrate is concentrated, and the residue is purified by reversed phase chromatography (HPLC; Sunfire, ACN/water including TFA) to afford compound B3.

Analysis (method G): Rₜ: 0.66 min, [M+H]⁺: 283

### Synthesis of ({2-[(2R)-2-(4-fluorophenyl)-2-hydroxyethyl]-2H-indazol-4-yl}boronic acid (B4)

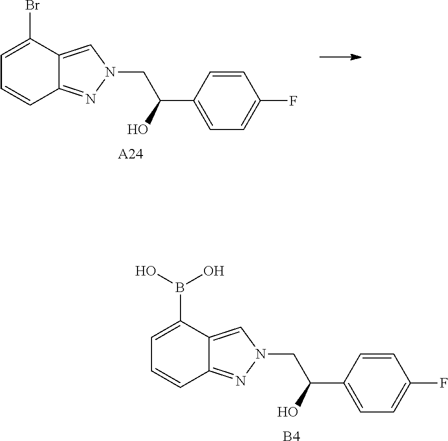

(1R)-2-(4-Bromo-2H-indazol-2-yl)-1-(4-fluorophenyl) ethan-1-ol (50 mg, 0.149 mmol), KOAc (38 mg), [1, 1′-bis(diphenylphosphino)ferrocene]dichloropalladium(II), complex with dichloromethane (1:1) (12.2 mg) and 2-(5, 5-dimethyl-1, 3, 2-dioxaborinan-2-yl)-5, 5-dimethyl-1, 3, 2-dioxaborinane (37 mg) is suspended in 1 mL of dioxane and heated for 2 h at 100° C. The mixture is filtered and then purified by reversed phase preparative HPLC to yield the desired product B4.

Analysis (method G): Rₜ: 0.78 min, [M+H]⁺: 301

### Synthesis of 4-(4, 4, 5, 5-tetramethyl-1, 3, 2-dioxaborolan-2-yl)-2-[(trimethylsilyl)methyl]-2H-indazole (B5)

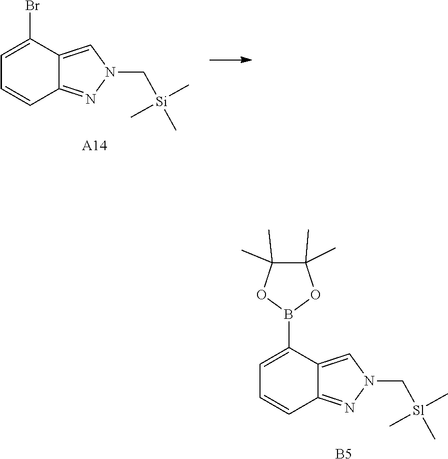

(1R)-2-(4-Bromo-2H-indazol-2-yl)-1-(4-fluorophenyl) ethan-1-ol (1.5 g, 5.3 mmol), KOAc (1.6 g), [1, 1′-bis(diphenylphosphino)ferrocene]dichloropalladium(II), complex with dichloromethane (1:1) (460 mg) and 2-(5, 5-dimethyl-1, 3, 2-dioxaborinan-2-yl)-5, 5-dimethyl-1, 3, 2-dioxaborinane (2.9 g) is suspended in 12 mL dioxane and heated overnight at 90° C. The mixture is filtered through Celite and charcoal and washed with dioxane. The organic phase is concentrated and purified by silica chromatography CycH/EtOAc to yield the desired product B5.

Analysis (method G): Rₜ: 1.23 min, [M+H]⁺: 331

### Synthesis of (1R)-1-{4-[2-(morpholin-4-yl) ethoxy]phenyl}-2-[4-(4, 4, 5, 5-tetramethyl-1, 3, 2-dioxaborolan-2-yl)-2H-indazol-2-yl]ethan-1-ol (B6)

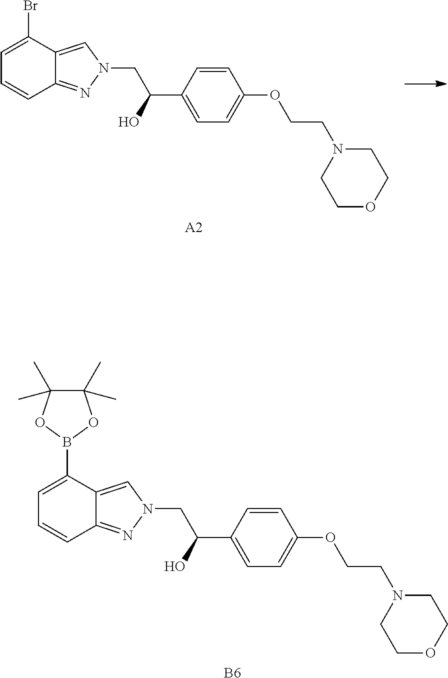 [SMILES: O[C@@H](Cn1cc2c(Br)cccc2n1)c1ccc(OCCN2CCOCC2)cc1 ; "A2" ; CC1(C)OB(c2cccc3nn(C[C@H](O)c4ccc(OCCN5CCOCC5)cc4)cc23)OC1(C)C ; "B6"]

Under an argon atmosphere, (1R)-2-(4-bromo-2H-indazol-2-yl)-1-{4-[2-(morpholin-4-yl) ethoxy]phenyl}ethan-1-ol (89.3 mg, 0.20 mmol) is dissolved in dioxane (4 mL). Bis(pinacolato)diboron (50.8 mg, 0.20 mmol), potassium acetate (51 mg, 0.52 mmol) and Pd(dppf)Cl₂×DCM (16.3 mg, 0.02 mmol) are added, and the reaction mixture is stirred at 95° C. for 2 h. The formed precipitate is filtered off, washed with dioxane, and the filtrate is concentrated to afford the desired product B6.

Analysis (method G): Rₜ: 0.87 min, [M+H]⁺: 494

### Synthesis of (1R)-1-[4-(benzyloxy)phenyl]-2-[4-(4, 4, 5, 5-tetramethyl-1, 3, 2-dioxaborolan-2-yl)-2H-indazol-2-yl]ethan-1-ol (B7)

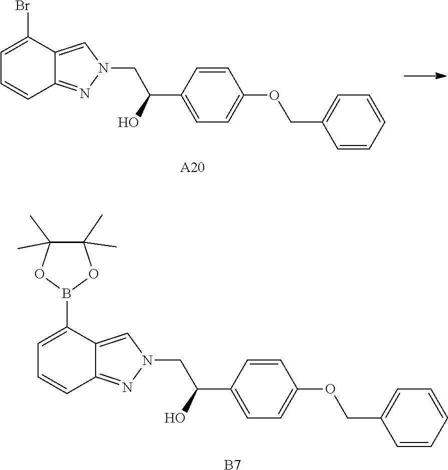

Under an argon atmosphere, (1R)-1-[4-(benzyloxy) phenyl]-2-(4-bromo-2H-indazol-2-yl)ethan-1-ol (400 mg, 0.95 mmol) is dissolved in dioxane (10 mL). Bis(pinacolato)diboron (480 mg, 1.89 mmol), potassium acetate (278 mg, 2.8 mmol) and Pd(dppf)Cl₂×DCM (77 mg) are added, and the reaction mixture is stirred at 90° C. for 17 h. The mixture is diluted with methanol and filtered over cellulose. The concentrated filtrate is purified by MPLC using CycH/EtOAc as eluent to yield the desired product B7.

Analysis (method A): Rₜ: 0.79 min, [M+H]⁺: 471

### Synthesis of 2-[(benzenesulfinyl)methyl]-4-(5, 5-dimethyl-1, 3, 2-dioxaborinan-2-yl)-2H-indazole (B8)

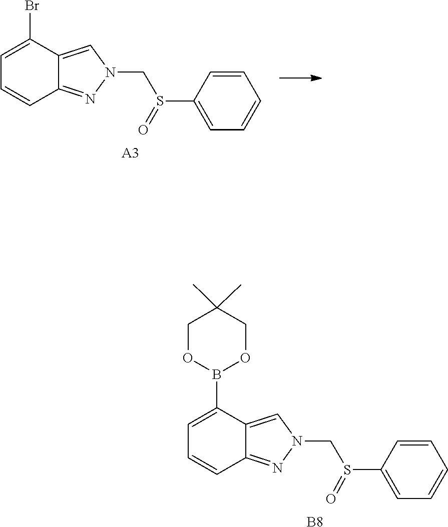

Under an argon atmosphere, 2-[(benzenesulfinyl)methyl]-4-bromo-2H-indazole (150 mg, 0.45 mmol) is dissolved in dioxane (2.5 mL). Bis (neopentyl glycolato)diboron (152 mg, 0.67 mmol), potassium acetate (131.8 mg, 1.34 mmol) and Pd(dppf)Cl₂×DCM (97 mg, 0.12 mmol) are added and the reaction mixture is stirred at 100° C. for 2 h. The reaction mixture is filtered, and the filtrate is concentrated to yield the desired product B8 which is used in the next step without purification.

Analysis (method G): Rₜ: 0.68 min, [M+H]⁺: 301 (mass of corresponding boronic acid)

### Synthesis of 2-[4-(5, 5-dimethyl-1, 3, 2-dioxaborinan-2-yl)-2H-indazol-2-yl]-1-(4-methylphenyl)ethan-1-ol (B9)

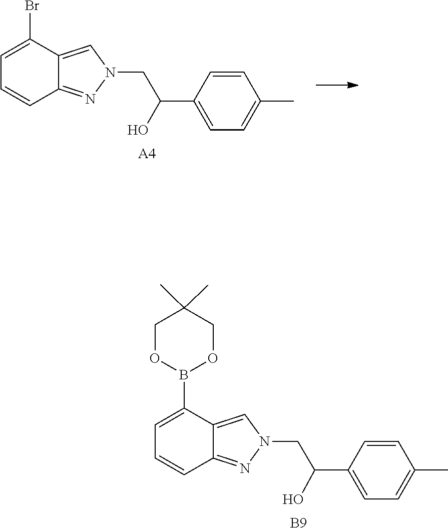

Under an argon atmosphere, 2-(4-bromo-2H-indazol-2-yl)-1-(4-methylphenyl) ethan-1-ol (120 mg, 0.36 mmol) is dissolved in dioxane (2.5 mL). Bis (neopentyl glycolato)diboron (123 mg, 0.54 mmol), potassium acetate (107 mg, 1.09 mmol) and Pd(dppf)Cl₂×DCM (78.4 mg, 0.096 mmol) are added and the reaction mixture is stirred at 100° C. for 2 h. The reaction mixture is filtered, and the filtrate is concentrated to yield the desired product which is used in the next step without purification.

Analysis (method G): Rₜ: 0.75 min, [M+H]⁺: 297 (mass of corresponding boronic acid)

### Synthesis of {2-[2-hydroxy-2-(2-methylpyridin-4-yl)ethyl]-2H-indazol-4-yl}boronic acid (B10)

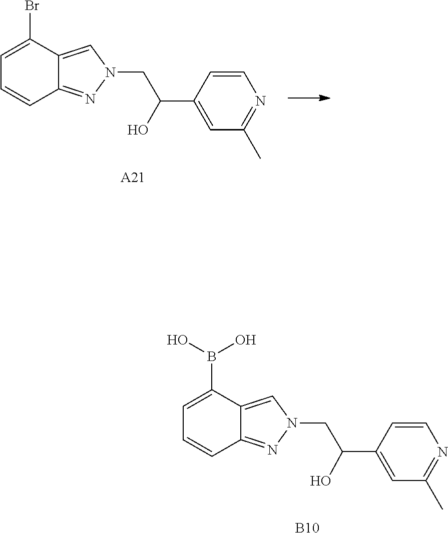

Under an argon atmosphere, 2-(4-bromo-2H-indazol-2-yl)-1-(2-methylpyridin-4-yl) ethan-1-ol (A21, 221 mg, 0.665 mmol) is dissolved in dioxane (3 mL). Bis (neopentyl glycolato)diboron (180 mg, 0.796 mmol), Potassium acetate (196 mg, 2 mmol) and Pd(dppf)Cl₂×DCM (54 mg) are added and the reaction mixture is stirred at 90° C. for 3 h. The reaction mixture is filtered, diluted with HOAc and purified by reversed phase preparative HPLC to yield the desired product B10.

Analysis (method G): Rₜ: 0.75 min, [M+H]⁺: 298

### Synthesis of 1-(1-methyl-1H-pyrazol-4-yl)-2-[4-(4, 4, 5, 5-tetramethyl-1, 3, 2-dioxaborolan-2-yl)-2H-indazol-2-yl]ethan-1-ol (B11)

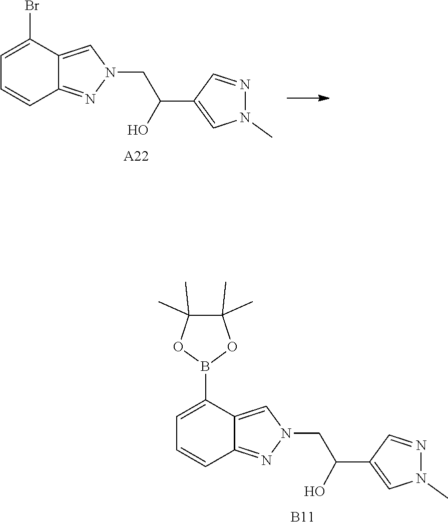

Under an argon atmosphere, 2-(4-bromo-2H-indazol-2-yl)-1-(1-methyl-1H-pyrazol-4-yl) ethan-1-ol (A1, 118 mg, 0.367 mmol) is dissolved in dioxane (2.5 mL). Bis(pinacolato)diboron (373 mg, 1.47 mmol), potassium acetate (108 mg, 1 mmol) and Pd(dppf)Cl₂×DCM (30 mg) are added and the reaction mixture is stirred at 90° C. for 4 h. The reaction mixture is filtered, then concentrated and purified by MPLC (Silica, DCM/MeOH) to yield the desired product B11.

Analysis (method G): Rₜ: 0.94 min, [M+H]⁺: 369

### Synthesis of (1R)-2-[4-(5, 5-dimethyl-1, 3, 2-dioxaborinan-2-yl)-5-fluoro-2H-indazol-2-yl]-1-phenylethan-1-ol (B12)

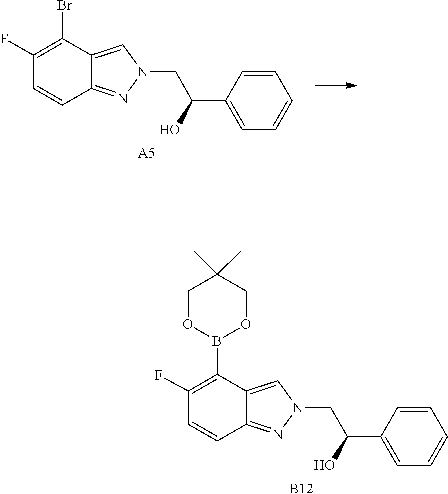

Under an argon atmosphere, (1R)-2-(4-bromo-5-fluoro-2H-indazol-2-yl)-1-phenylethan-1-ol (A5, 608 mg, 1.81 mmol) is dissolved in dioxane (8 mL). Bis (neopentyl glycolato) diboron (1.23 g, 5.44 mmol), potassium acetate (890 mg, 9.07 mmol) and Pd(dppf)Cl₂×DCM (74 mg, 0.09 mmol) are added, and the reaction mixture is stirred at 80° C. for 3 h and RT overnight. After the reaction mixture is cooled to RT, it is filtered through a Thiol-Resin and the filtrate is concentrated. The residue is diluted with water and is extracted with EtOAc. The organic layer is dried (Na₂SO₄), filtered, and concentrated. The residue is crystallized with cold MeOH to afford the product B12.

Analysis (method A): Rₜ: 0.39 min, [M+H]⁺: 301 (mass of the corresponding boronic acid)

### Synthesis of {2-[(2R)-2-(2-ethoxy-2-oxoethoxy)-2-phenylethyl]-2H-indazol-4-yl}boronic acid (B13)

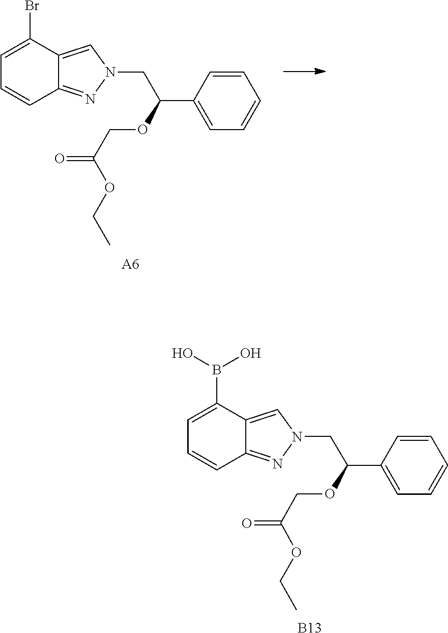

Under an argon atmosphere, ethyl 2-[(1R)-2-(4-bromo-2H-indazol-2-yl)-1-phenylethoxy]acetate (A6, 1.93 g, 4.79 mmol) is dissolved in dioxane (25 mL). Bis (neopentyl glycolato)diboron (1.19 g, 5.26 mmol), potassium acetate (1.64 g, 16.8 mmol) and Pd(dppf)Cl₂×DCM (0.30 g, 0.37 mmol) are added and the reaction mixture is stirred at 90° C. for 1.5 h. The reaction mixture is diluted with DCM and filtered. The filtrate is concentrated, and the residue is purified by reversed phase chromatography (HPLC; ACN/water including TFA) to afford the desired product B13.

Analysis (method F): Rₜ: 0.72 min, [M+H]⁺: 369

### Synthesis of 4-[(1R)-1-hydroxy-2-[4-(4, 4, 5, 5-tetramethyl-1, 3, 2-dioxaborolan-2-yl)-2H-indazol-2-yl]ethyl]phenol (B14)

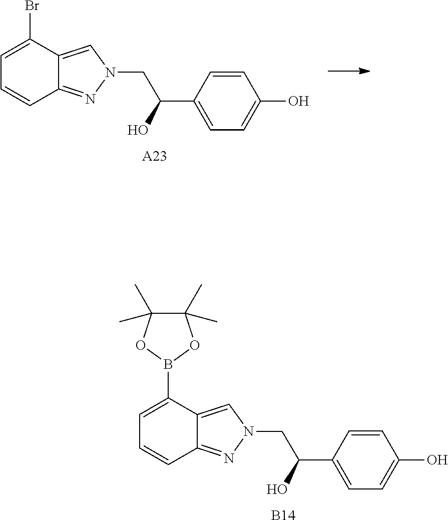

Under an argon atmosphere, 4-[(1R)-2-(4-bromo-2H-indazol-2-yl)-1-hydroxyethyl]phenol (A23, 1.45 g, 4.35 mmol) is dissolved in dioxane (40 mL). Bis(pinacolato)diborane (1.11 g, 4.35 mmol), potassium acetate (1.28 g, 13.1 mmol) and Pd(dppf)Cl₂×DCM (355 mg, 0.44 mmol) are added, and the reaction mixture is stirred at 90° C. for 2 h. The reaction mixture is diluted with EtOAc and water, MetS-Thiol scavenger resin (for removal of the catalyst) and active charcoal are added, and the reaction mixture is stirred 5 min. Then it is filtered, washed, and extracted with EtOAc. The organic layer is dried (Na₂SO₄), filtered, and concentrated. The residue is triturated with EtOAc/CycH 1/1. The precipitate is filtered, washed with EtOAc/CycH 1/1 and dried to afford 840 mg of the product. The filtrate is purified by flash chromatography (CycH/EtOAc 75/25 to CycH/EtOAc 25/75) to afford the product B14.

Analysis (method A): Rₜ: 0.56 min, [M+H]⁺: 381

### Synthesis of {2-[2-(3, 4-difluorophenyl)-2-hydroxyethyl]-2H-indazol-4-yl}boronic acid (B15)

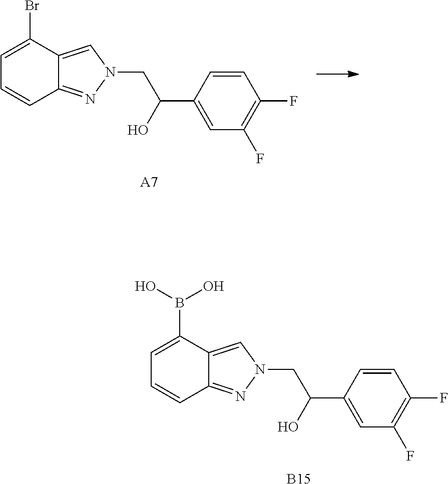

(1R)-2-(4-Bromo-2H-indazol-2-yl)-1-(3, 4-difluorophenyl) ethan-1-ol (A7, 1300 mg, 0.37 mmol), bis (neopentyl glycolato)diboron (116 mg, 0.51 mmol), potassium acetate (113 mg, 1.15 mmol) and Pd(dppf)Cl₂×DCM (40 mg, 0.05 mmol) are dissolved in dioxane (4 mL) and the reaction mixture is stirred at 85° C. overnight. The reaction mixture is filtered and purified by reversed phase chromatography (HPLC; ACN/water including TFA) to afford the desired product B15.

Analysis (method G): Rₜ: 0.81 min, [M+H]⁺: 319

### Synthesis of [2-(2, 2-difluoro-2-phenylethyl)-2H-indazol-4-yl]boronic acid (B16)

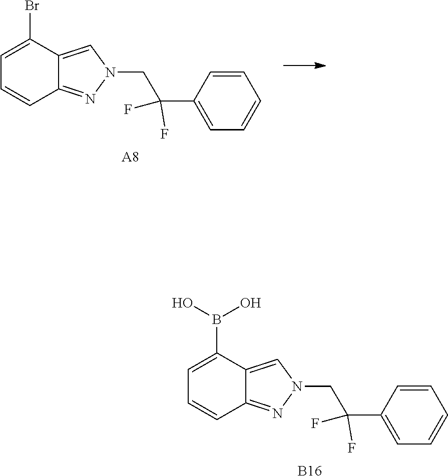

Under an argon atmosphere, 4-bromo-2-(2, 2-difluoro-2-phenylethyl)-2H-indazole (A8, 677 mg, 2.01 mmol), bis (neopentyl glycolato)diboron (1.36 g, 6.02 mmol), potassium acetate (985 mg, 10 mmol) and Pd(dppf)Cl₂×DCM (82 mg, 0.10 mmol) are dissolved in dioxane (9 mL) and the reaction mixture is stirred at 80° C. for 3 h. The reaction mixture is filtered with ACN through a thiol scavenger resin and the filtrate is evaporated. The residue is purified by reversed phase chromatography (HPLC; ACN/water including TFA) to afford the desired product B16.

Analysis (method A): Rₜ: 0.46 min, [M+H]⁺: 303

### Synthesis of 4-(5, 5-dimethyl-1, 3, 2-dioxaborinan-2-yl)-2-[(2R)-2-fluoro-2-phenylethyl]-2H-indazole (B17)

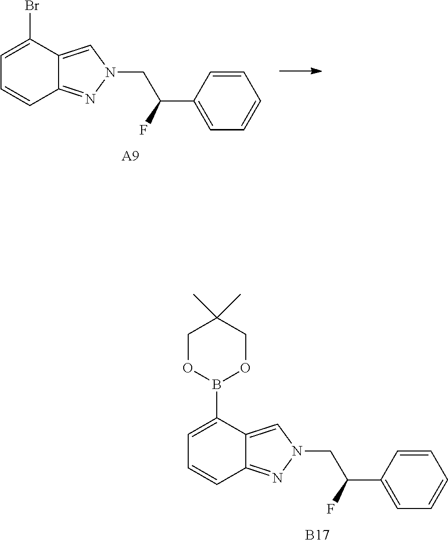

Under an argon atmosphere, 4-bromo-2-[(2R)-2-fluoro-2-phenylethyl]-2H-indazole (100 mg, 0.31 mmol), bis (neopentyl glycolato)diboron (77.9 mg, 0.345 mmol), potassium acetate (92.3 mg, 0.940 mmol) and Pd(dppf)Cl₂×DCM (29.9 mg, 0.037 mmol) are dissolved in dioxane (2 mL) and the reaction mixture is stirred at 90° C. for 3 h. The reaction mixture is diluted with DCM and brine and is extracted. The organic layer is dried (Na₂SO₄), filtered and concentrated to afford the desired product B17 which was used without further purification in the next step.

Analysis (method F): Rₜ: 0.67 min, [M+H]⁺: 285 (mass of corresponding boronic acid)

### Synthesis of [2-(2-{4-[2-(dimethylamino) ethoxy]phenyl}ethyl)-2H-indazol-4-yl]boronic acid (B18)

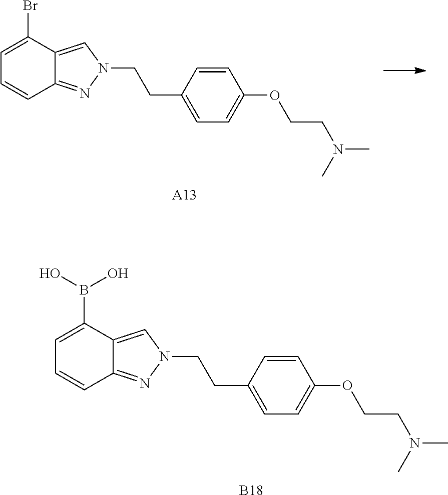

(2-{4-[2-(4-Bromo-2H-indazol-2-yl) ethyl]phenoxy}ethyl) dimethylamine (A13, 1.00 g, 2.32 mmol,) is dissolved in dioxane (7 mL). Bis (neopentyl glycolato)diboron (628 mg, 2.78 mmol), potassium acetate (683 mg, 6.95 mmol) and Pd(dppf)Cl₂×DCM (151 mg, 0.19 mmol) are added and the reaction mixture is stirred at 90° C. overnight. The reaction mixture is filtered, and the residue is purified by reversed phase chromatography (HPLC; ACN/water including TFA) to afford the title compound B18.

Analysis (method G): Rₜ: 0.67 min, [M+H]⁺: 354

### Synthesis of {2-[(2R)-2-(4-chlorophenyl)-2-fluoroethyl]-2H-indazol-4-yl}boronic acid (B19)

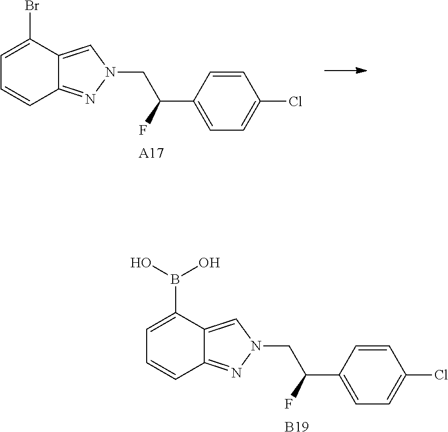

Under an argon atmosphere, 4-bromo-2-[(2R)-2-(4-chlorophenyl)-2-fluoroethyl]-2H-indazole (A17, 340 mg, 0.96 mmol) is dissolved in dioxane (10 mL). Bis (neopentyl glycolato)diboron (239 mg, 1.06 mmol), potassium acetate (245 mg, 2.5 mmol) and Pd(dppf)Cl₂×DCM (78.5 mg, 0.096 mmol) are added and the reaction mixture is stirred at 100° C. for 2 h. The reaction mixture is filtered through a thiol scavenger resin and the residue is purified by reversed phase chromatography (HPLC; ACN/water including TFA) to afford the title compound B19.

Analysis (method F): Rₜ: 0.78 min, [M+H]⁺: 319

### Synthesis of 1-cyclohexyl-2-[4-(4, 4, 5, 5-tetramethyl-1, 3, 2-dioxaborolan-2-yl)-2H-indazol-2-yl]ethan-1-ol (B20)

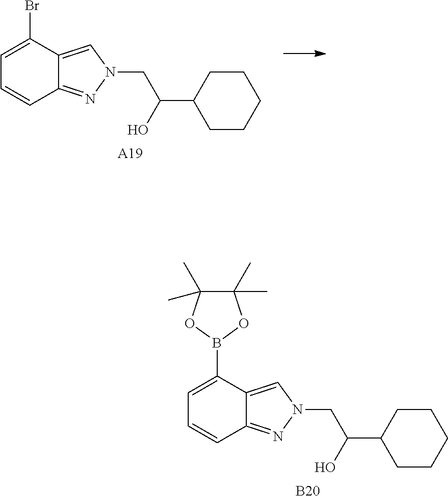

Under an argon atmosphere, 2-(4-bromo-2H-indazol-2-yl)-1-cyclohexylethan-1-ol (A19, 133 mg, 0.41 mmol) and bis (neopentyl glycolato)diboron (125.4 mg, 0.49 mmol) are dissolved in dioxane (1.5 mL). Potassium acetate (121.2, 1.23 mmol) and Pd(dppf)Cl₂×DCM (33.6 mg, 0.04 mmol) are added, and the reaction mixture is stirred at 85° C. overnight. The reaction mixture is concentrated and purified by flash chromatography (CycH/EtOAc 100/0 to CycH/EtOAc 50/50) to afford the title compound B20.

Analysis (method G): Rₜ: 1.19 min, [M+H]⁺: 371

### Synthesis of {2-[(2R)-2-(4-fluorophenyl)-2-hydroxyethyl]-2H-indazol-4-yl}boronic acid (B21)

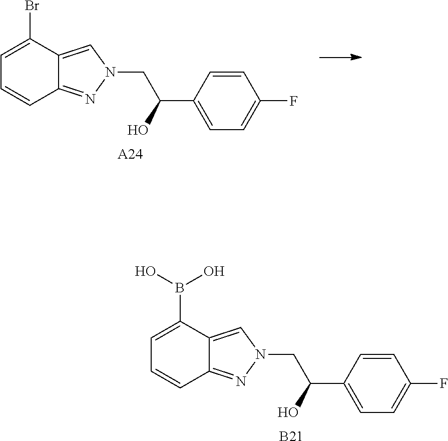

Under an argon atmosphere, (1R)-2-(4-bromo-2H-indazol-2-yl)-1-(4-fluorophenyl) ethan-1-ol (A24, 100 mg, 0.298 mmol) and bis (neopentyl glycolato)diboron (74 mg, 0.33 mmol) are dissolved in dioxane (2 mL). Potassium acetate (76 mg, 1 mmol) and Pd(dppf)Cl₂×DCM (25 mg) are added, and the reaction mixture is stirred at 95° C. overnight. The reaction mixture is filtered, diluted with HOAc and purified by preparative reversed-phase HPLC to afford the product B21.

Analysis (method G): Rₜ: 0.77 min, [M+H]⁺: 301

### Synthesis of {2-[(2R)-2-{4-[2-(dimethylamino) ethoxy]phenyl}-2-hydroxyethyl]-2H-indazol-4-yl}boronic acid (B22)

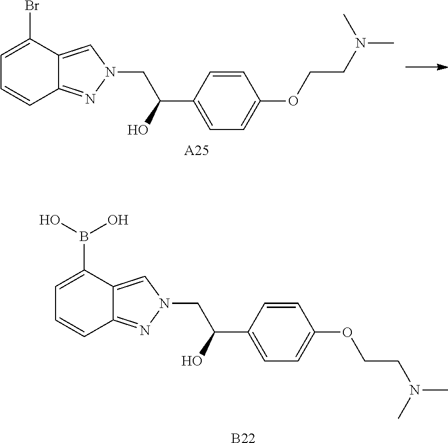

Under an argon atmosphere, (1R)-2-(4-bromo-2H-indazol-2-yl)-1-{4-[2-(dimethylamino) ethoxy]phenyl}ethan-1-ol (A25, 80 mg, 0.196 mmol) and bis (neopentyl glycolato)diboron (49 mg, 0.22 mmol) are dissolved in dioxane (1.5 mL). Potassium acetate (68 mg, 0.7 mmol) and Pd(dppf)Cl₂×DCM (16 mg) are added, and the reaction mixture is stirred at 90° C. for 2 h. The reaction mixture is diluted with DCM and filtered through Celite and then purified by preparative reversed-phase HPLC (sunfire C18, ACN/water including TFA) to afford the desired product B22.

Analysis (method A): Rₜ: 0.28 min, [M+H]⁺: 370

### Synthesis of Intermediates C

### Synthesis of Intermediate C1

### Step 1: Synthesis of 2-acetyl-3-methylbutanenitrile

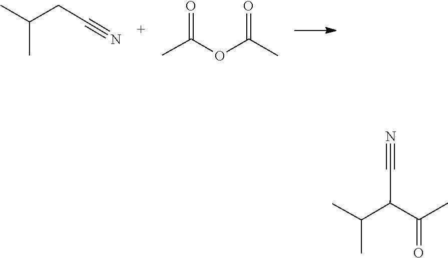

A solution of diisopropylamine (177 mL, 1.25 mol) in T (1.17 L) is cooled to −78° C. N-butyllithium 2.5M in hexane (469 mL, 1.17 mol) is added and the reaction mixture is allowed to warm to 0° C. and stirred 1 h at 0° C. The reaction mixture is cooled to −78° C. and 3-methyl-butyronitrile (81.8 mL, 0.782 mol) in 80 mL THE is added dropwise, while maintaining the temperature below −65° C. The reaction mixture is stirred at −78° C. for 1 h. After that, a solution of acetic anhydride (88.7 mL, 0.938 mol) in 80 mL THE is added dropwise over 30 min. The reaction mixture is allowed to warm to 0° C. for 1 h, then 15° C. for 0.5 h. The reaction mixture is quenched with citric acid (10%, 200 mL) and extracted with EtOAc. The organic layer is dried (Na₂SO₄), filtered and concentrated to afford the desired product, which is used without further purification in the next step.

Analysis (TLC): Rf: 0.3 (20% EtOAc/petrol ether)

### Step 2: Synthesis of 5-methyl-4-(propan-2-yl)-1H-pyrazol-3-amine

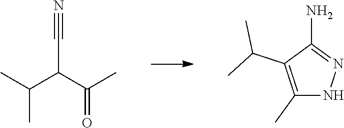

2-Acetyl-3-methylbutanenitrile (80 g, 0.64 mol) is dissolved in EtOH (352 mL). Glacial acetic acid (47.6 mL, 0.83 mol) and hydrazine monohydrate (49.6 mL, 1.02 mol) are added, and the reaction mixture is stirred at 80° C. overnight. After cooling to 0° C. the pH is carefully adjusted to 9 with a saturated NaHCO₃ solution. The reaction mixture is then diluted with water and extracted 3× with EtOAc. The organic layer is washed with brine, dried (Na₂SO₄), filtered and concentrated to afford the desired product.

Analysis (method C): Rₜ: 0.36 min, [M+H]⁺: 140

### Step 3: Synthesis of 3-iodo-5-methyl-4-(propan-2-yl)-1H-pyrazole

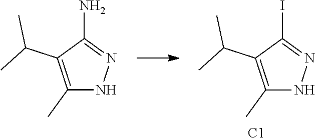

5-Methyl-4-(propan-2-yl)-1H-pyrazol-3-amine (1 g, 7.2 mmol) is dissolved in ACN (8 mL). Under cooling with ice/acetone, sodium nitrite (0.59 g, 8.6 mmol) is added and the reaction mixture is stirred at −5° C. for 30 min. Then KI (1.55 g, 9.34 mmol) is added, and the reaction mixture is stirred at 0° C. for 1.5 h. The reaction mixture is quenched with Na₂S2O₃ and is diluted with Me-THF (10 mL) and water (10 mL). After stirring for 1 h the layers are separated. The organic layer is washed with brine, dried (Na₂SO₄), and concentrated to afford 1.6 g of the desired product C1.

Analysis (method A): Rₜ: 0.52 min, [M+H]⁺: 251

### Alternative Synthesis of Intermediate C1

### Step 1: Synthesis of 5-iodo-3-methyl-4-(propan-2-yl)-1H-pyrazole

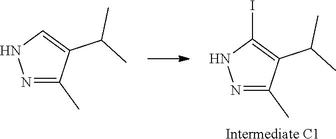

3-Methyl-4-(propan-2-yl)-1H-pyrazole (10 g, 80.5 mmol) is dissolved in ACN (150 mL). NIS (25 g, 111 mmol) is added, and the reaction mixture is stirred at 80° C. overnight. The reaction mixture is filtered, and the filtrate is evaporated. The residue is quenched with a half saturated Na₂S2O₃ solution and extracted three times with DCM. The combined organic layers are dried (Na₂SO₄), filtered and concentrated. The crude product is purified by flash chromatography (CycH/EtOAc 95/5 to CycH/EtOAc 76/24) to afford intermediate C1.

Analysis (method G): Rₜ: 0.93 min, [M+H]⁺: 251

### Synthesis of Intermediate C2

### Step 1: Synthesis of 2-cyclopropyl-3-oxobutanenitrile

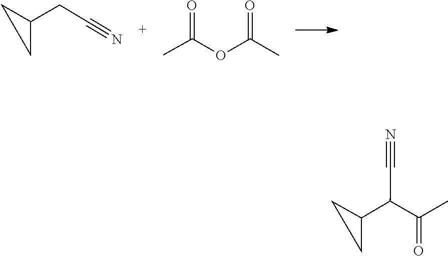

To a stirred solution of 2-cyclopropylacetonitrile (3.7 mL; 40 mmol) in THE (20 mL) LDA (2M, 6.44 g, 60 mmol) is added dropwise at −78° C. over a time period of 20 min. The reaction mixture is stirred at −78° C. for 1 h. A solution of acetic anhydride (4.5 mL) in THE is added dropwise for min and the mixture is allowed to stir at 0° C. for 1 h. The mixture is quenched with saturated citric acid and extracted with diethyl ether (3×20 mL). The organic layer is dried (Na₂SO₄), filtered and concentrated to afford the title compound that is used in the next step without further purification.

Analysis (TLC): Rf: 0.7, 10% EtOAc in hexane

### Step 2: Synthesis of 5-methyl-4-(cyclopropanyl)-1H-pyrazol-3-amine

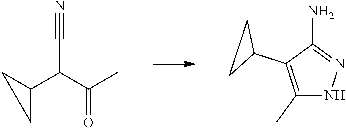

2-Cyclopropyl-3-oxobutanenitrile (4.9 g, 40 mmol) is dissolved in EtOH (18 mL). Glacial acetic acid (3 mL, 51.4 mmol) and hydrazine monohydrate (3.15 mL, 64.9 mol) are added, and the reaction mixture is stirred at 80° C. overnight. After cooling to 0° C. the pH is adjusted to 9 with a saturated NaHCO₃ solution. The reaction mixture is then diluted with water and extracted 4× with EtOAc. The organic layer is washed with brine, dried (Na₂SO₄), filtered and concentrated to afford the desired product.

Analysis (method A): Rₜ: 0.26 min, [M+H]⁺: 138

### Step 3: Synthesis of 3-iodo-5-methyl-4-(cyclopropanyl)-1H-pyrazole

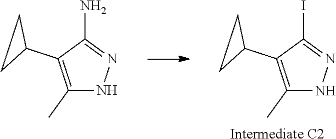

5-Methyl-4-(cyclopropanyl)-1H-pyrazol-3-amine (5.17 g, 37.6 mmol) is dissolved in ACN (108 mL) and cooled to 0° C. Then acetic acid (6.5 mL, 112 mmol) is carefully added, followed by dropwise addition of potassium iodide (11.2 mL, 93.9 mmol dissolved in 54 mL of water) is added. The mixture is stirred for 10 min at 0° C., then tert-butyl nitrite (11.2 mL, 94 mmol, dissolved in ACN) is added dropwise. The mixture is stirred for 10 min at ° C. followed by 1 h at RT. The reaction mixture is adjusted to pH 8 by addition of aqueous saturated sodium bicarbonate and extracted with EtOAc. The organic phase is washed with 0.5 M, 200 mL Na₂S2O₃ and brine and then dried over Na₂SO₄. The concentrated organic phase is then purified by flash column chromatography using EtOAC/CycH mixtures as eluent to yield the desired product C2.

Analysis (method C): Rₜ: 0.57 min, [M+H]⁺: 249

### Synthesis of Intermediate C3

### Step 1: Synthesis of 5-cyclopropyl-1-methyl-1H-imidazole

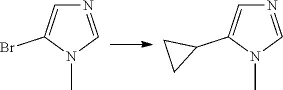

Under an argon atmosphere, 5-bromo-1-methyl-1H-imidazole (7.50 g, 46.6 mmol), cyclopropylzinc bromide (132 mL, 65.90 mmol, 0.5 M in THF) and Pd(dppf)Cl₂ (2.20 g, 3.01 mmol) are mixed together, and the reaction mixture is stirred at 70° C. for 20 h. The reaction mixture is concentrated, and the residue is purified by flash chromatography (DCM/MeOH 100/0 to DCM/MeOH 90/10) to afford the desired product.

Analysis (method C): Rₜ: 0.31 min, [M+H]⁺: 123

### Step 2: Synthesis of 5-cyclopropyl-1, 2-dimethyl-1H-imidazole

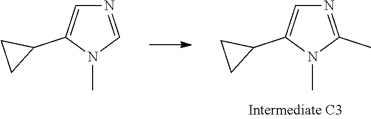

Under an argon atmosphere, 5-cyclopropyl-1-methyl-1H-imidazole (1 g, 8.12 mmol) is dissolved in THE (15.00 mL) and cooled to −78° C. n-BuLi (6.1 mL, 9.8 mmol, 1.6 M) is added dropwise and the reaction mixture is stirred at −78° C. for 30 min. Then Mel (663 μL, 10.6 mmol) is added dropwise and the reaction mixture is stirred at −78° C. for 1 h. The reaction mixture is quenched with a half saturated NH₄Cl solution and stirred for 10 min. 2 mL of aqueous NH₄OH (25%) is added and the mixture is stirred for 30 min. The layers are separated, and the aqueous layer is extracted 3× with EtOAc. The combined organic layers are dried (Na₂SO₄), filtered and evaporated to afford intermediate C3.

Analysis (method H): Rₜ: 0.72 min, [M+H]⁺: 137

### Synthesis of Intermediate C4: 1, 2-dimethyl-5-(propan-2-yl)-1H-imidazole

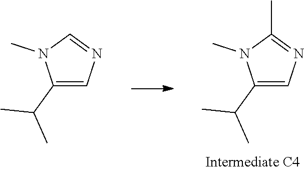

5-Isopropyl-1-methyl-1H-imidazole (1 g, 8.05 mmol) is dissolved in THE (25 mL) and cooled to −65° C. Then 5.4 mL BuLi (1.6M, 8.64 mmol) is added slowly. After 30 min Mel (0.8 mL, 12.9 mmol) is added and the mixture warmed to room temperature; water and aqueous ammonia (1 mL) is added under stirring, then aqueous NH₄Cl solution is added, and the mixture extracted with EtOAc. Purification by silica gel chromatography using DCM:MeOH 9/1 plus ammonia yielded the desired product C4.

Analysis (method H): Rₜ: 0.76 min, [M+H]⁺: 139

### Synthesis of Intermediate C5:3, 4-diethyl-5-iodo-1H-pyrazole

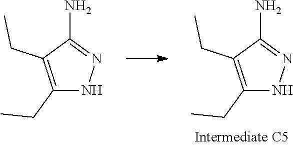

4, 5-Diethyl-1H-pyrazol-3-amine (916 mg, 6.58 mmol) is dissolved in concentrated HCl (10 mL), and sodium nitrite (1.62 g dissolved 10 mL of water) is added slowly over 30 min at 0° C. Potassium iodide (4.41 g, dissolved in 4 mL water) is added slowly and the mixture is stirred overnight at room temperature. The mixture is diluted with water and extracted with TBME, the organic phase is washed with sodium thiosulfate solution and concentrated. The crude product is dissolved in acetonitrile and the formed solid collected to yield the desired product C5.

Analysis (method A): Rₜ: 0.53 min, [M+H]⁺: 251

### Synthesis of Intermediate C6: methyl 2-(3-iodo-5-methyl-1H-pyrazol-4-yl)acetate

### Step 1: Synthesis of methyl 2-(5-methyl-1H-pyrazol-4-yl) acetate

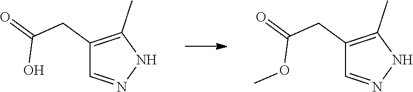

2-(5-Methyl-1H-pyrazol-4-yl) acetic acid (1 g, 7.14 mmol) is dissolved in DCM (20 mL) and MeOH (10 mL) and cooled to −8° C., trimethylsilyl diazomethane (6.07 mL in hexane, 12.3 mmol) is slowly added and the mixture is stirred for 2 h. The mixture is concentrated and used in the next step as crude product (g).

### Step 2: Synthesis of methyl 2-(3-iodo-5-methyl-1H-pyrazol-4-yl)acetate

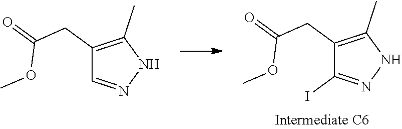

Methyl 2-(5-methyl-1H-pyrazol-4-yl) acetate (1.1 g) is dissolved in 10 mL ACN and NIS (1.284 g, 5.7 mmol) is added, and the mixture is stirred at 80° C. overnight. The mixture is then filtered, concentrated, and purified by silica gel chromatography using CycH/EtOAc gradient to yield the desired product C6.

Analysis (method H): Rₜ: 0.75 min, [M+H]⁺: 281

### Synthesis of Intermediates D

### Synthesis of Intermediate D1: 1-[3-iodo-5-methyl-4-(propan-2-yl)-1H-pyrazol-1-yl]propan-2-one

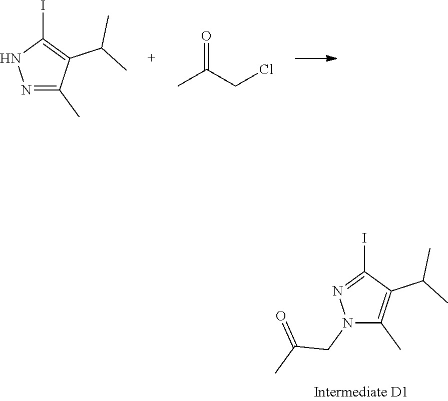

5-Iodo-3-methyl-4-(propan-2-yl)-1H-pyrazole (C1, 2.90 g, 11.6 mmol) is dissolved in ACN (60 mL), K₂CO₃(4.01 g, 29 mmol) and chloroacetone (1.85 mL, 23.2 mmol) are added. The reaction mixture is stirred at 50° C. for 2 h. Chloroacetone (0.2 mL) is added, and the reaction mixture is stirred at 50° C. for 1 h. The reaction mixture is filtered, washed with ACN and the filtrate is evaporated. The residue is purified by flash chromatography (CycH/EtOAc 90/10 to CycH/EtOAc 60/40) to afford the product D1.

Analysis (method F): Rₜ: 0.82 min, [M+H]⁺: 307

The intermediates compiled in the following table are obtained by following a procedure analogous to that described for intermediate D1.

| | | | | MS | |
|---|---|---|---|---|---|
| | | | | (ESI+): | |
| | Starting materials | | LC | m/z | t_{R} |
| Intermediate | and conditions | Structure/Name | Method | [M + H]⁺ | [min] |
| D2 | 2-bromo-1- cyclopropylethan- 1-one and C1 K₂CO₃ 50° C. 3 h RT 17 h | 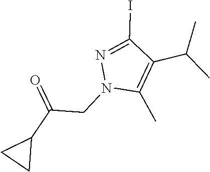 [SMILES: Cc1c(C(C)C)c(I)nn1CC(=O)C1CC1] | F | 333 | 0.91 |
| | | 1-cyclopropyl-2-[3-iodo-5- | | | |
| | | methyl-4-(propan-2-yl)-1H- | | | |
| | | pyrazol-1-yl] ethan-1-one | | | |
| D3 | 1-bromo-3- methoxypropan-2- one and C1 K₂CO₃ 50° C. 19 h | 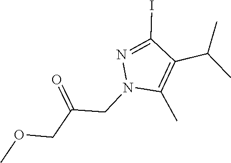 [SMILES: COCC(=O)Cn1nc(I)c(C(C)C)c1C] | G | 337 | 1.00 |
| | | 1-[3-iodo-5-methyl-4-(propan- | | | |
| | | 2-yl)-1H-pyrazol-1-yl]-3- | | | |
| | | methoxypropan-2-one | | | |
| D4 | 2-bromo-1- cyclobutylethan-1- one and C1 K₂CO₃ 50° C. 16 h | 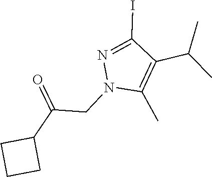 [SMILES: Cc1c(C(C)C)c(I)nn1CC(=O)C1CCC1] | G | 347 | 1.15 |
| | | 1-cyclobutyl-2-[3-iodo-5- | | | |
| | | methyl-4-(propan-2-yl)-1H- | | | |
| | | pyrazol-1-ylJethan-1-one | | | |
| D5 | 1-chloropentan-2- one and C1 NaH, DMF 80° C. 1 h | 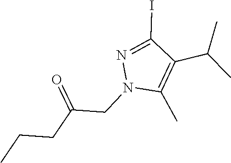 [SMILES: CCCC(=O)Cn1nc(I)c(C(C)C)c1C] | H | 335 | 1.10 |
| | | 1-[3-iodo-5-methyl-4-(propan- | | | |
| | | 2-yl)-1H-pyrazol-1-yl]pentan-2-one | | | |
| D6 | 2-bromo-1- (tetrahydro-pyran- 4-yl) ethenone and C1 K₂CO₃ 50° C. 16 h | 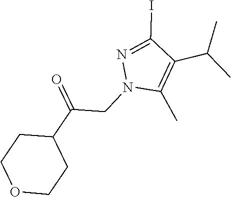 [SMILES: Cc1c(C(C)C)c(I)nn1CC(=O)C1CCOCC1] | G | 377 | 1.06 |
| | | 2-[3-iodo-5-methyl-4-(propan- | | | |
| | | 2-yl)-1H-pyrazol-1-yl]-1-(oxan- | | | |
| | | 4-yl)ethan-1-one | | | |
| D7 | 2-bromo-1- cyclopropylethan- 1-one and C2 K₂CO₃ 50° C. 3 h RT 17 h | 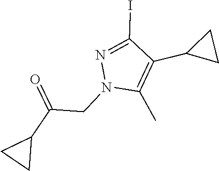 [SMILES: Cc1c(C2CC2)c(I)nn1CC(=O)C1CC1] | F | 331 | 0.87 |
| | | 1-cyclopropyl-2-(4-cyclopropyl- | | | |
| | | 3-iodo-5-methyl-1H-pyrazol-1- | | | |
| | | yl)ethan-1-one | | | |
| D8 | 1-chloro-4- methoxybutan-2- one and C1 NaH, NMP 130° C. 4 h | 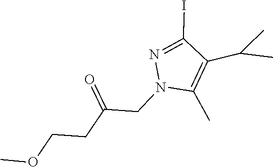 [SMILES: COCCC(=O)Cn1nc(I)c(C(C)C)c1C] | G | 351 | 0.98 |
| | | 1-(4-cyclopropyl-3-iodo-5- | | | |
| | | methyl-1H-pyrazol-1-yl)-4- | | | |
| | | methoxybutan-2-one | | | |
| D9 | Chloroacetone and C5 80° C., 2 h ACN, K₂CO₃ | 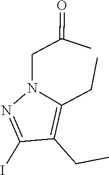 | G | 307 | 1.01 |
| | | 1-(4, 5-diethyl-3-iodo-1H- | | | |
| | | pyrazol-1-yl)propan-2-one | | | |
| D10 | Chloroacetone and C6 50° C., overnight ACN, K₂CO₃ | 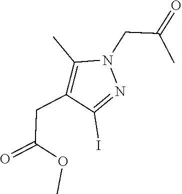 [SMILES: COC(=O)Cc1c(I)nn(CC(C)=O)c1C] | H | 337 | 0.79 |
| | | methyl 2-[3-iodo-5-methyl-1-(2- | | | |
| | | oxopropyl)-1H-pyrazol-4- | | | |
| | | yl]acetate | | | |
| D11 | 1-bromopinacolone and C1 50° C., overnight ACN, K₂CO₃ | 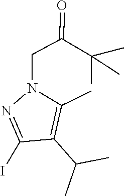 [SMILES: Cc1c(C(C)C)c(I)nn1CC(=O)C(C)(C)C] | G | 349 | 1.17 |
| | | 1-[3-iodo-5-methyl-4-(propan- | | | |
| | | 2-yl)-1H-pyrazol-1- | | | |
| | | yl]-3, 3-dimethylbutan-2-one | | | |
| D12 | tert-butyl 2-(2- bromoacetyl) pyrrolidine-1- carboxylate and C1 50° C., overnight ACN, K₂CO₃ | 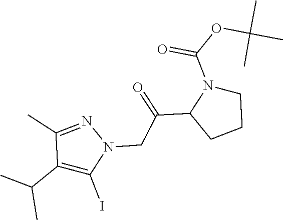 [SMILES: Cc1nn(CC(=O)C2CCCN2C(=O)OC(C)(C)C)c(I)c1C(C)C] | G | 462 | 1.06 |
| | | tert-butyl 2-{2-[5-iodo-3-methyl-4- | | | |
| | | (propan-2-yl)-1H-pyrazol-1- | | | |
| | | yl]acetyl}pyrrolidine-1-carboxylate | | | |

### Synthesis of Intermediate D13: 2-[3-iodo-5-methyl-4-(propan-2-yl)-1H-pyrazol-1-yl]-1-(oxolan-3-yl) ethan-1-one

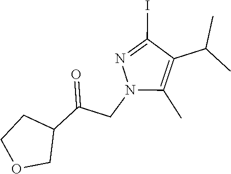 [SMILES: Cc1c(C(C)C)c(I)nn1CC(=O)C1CCOC1]

### Intermediate D13

### Step 1: Synthesis of oxolane-3-carbonyl chloride

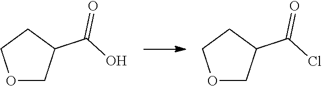 [SMILES: O=C(O)C1CCOC1 ; O=C(Cl)C1CCOC1]

Oxolane-3-carboxylic acid (2 g, 17.2 mmol) is dissolved in DCM (50 mL). DMF (24 μL, 0.295 mmol) is added, and oxalyl chloride (2.14 mL, 25 mmol) is added dropwise at 0° C. and the reaction mixture is stirred at RT for 3 h. The reaction mixture is filtered, washed with 10 mL of DCM and the filtrate is concentrated to afford the product which is employed in the next step without further purification.

### Step 2: Synthesis of 2-chloro-1-(oxolan-3-yl) ethan-1-one

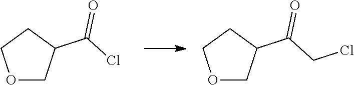 [SMILES: O=C(Cl)C1CCOC1 ; O=C(CCl)C1CCOC1]

Oxolane-3-carbonyl chloride (484 mg, 3.6 mmol) is dissolved in THE (2 mL) and ACN (2 mL). Trimethylsilyl diazomethane (3.06 g, 7.92 mmol, 2 M) is added dropwise and the reaction mixture is stirred at RT overnight. At 0° C. the reaction mixture is quenched with 4 M HCl in dioxane (2.70 mL, 10.8 mmol) and it is stirred at RT for 3 h. The reaction mixture is concentrated to afford the product which is employed in the next step without further purification.

Analysis Rt (EI): 3.58 min

### Step 3: Synthesis of 2-[3-iodo-5-methyl-4-(propan-2-yl)-1H-pyrazol-1-yl]-1-(oxolan-3-yl) ethan-1-one

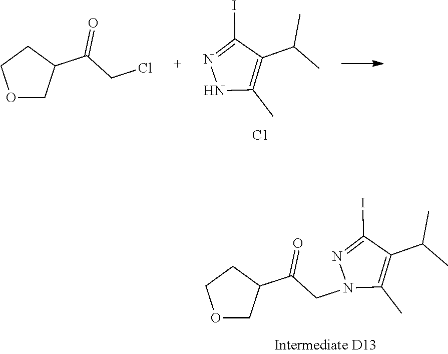

Intermediate D13 is synthesized by following a procedure analogous to that described for intermediate D1 using 2-chloro-1-(oxolan-3-yl) ethan-1-one, intermediate C1 and K₂CO₃ in DMF to react for 2 h at 80° C. to afford the intermediate D13.

Analysis (method G): Rₜ: 1.03 min, [M+H]⁺: 363

The intermediates compiled in the following table are obtained by following a reaction sequence analogous to that described for Intermediate D13.

| | | | | MS | |
|---|---|---|---|---|---|
| | | | | (ESI+): | |
| | Starting materials | | LC | m/z | t_{R} |
| Intermediate | and conditions | Structure/Name | Method | [M + H]⁺ | [min] |
| D14 | 2-chloro-1-(1- fluorocyclopropyl) ethan-1- one and C1 K₂CO₃ 80° C. 2 h | 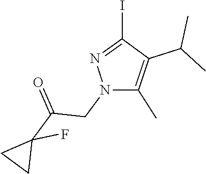 [SMILES: Cc1c(C(C)C)c(I)nn1CC(=O)C1(F)CC1] | G | 351 | 1.11 |
| | | 1-(1-fluorocyclopropyl)-2-[3-iodo-5- | | | |
| | | methyl-4-(propan-2-yl)-1H-pyrazol-1- | | | |
| | | yl]ethan-1-one | | | |
| D15 | 1-chloro-3- cyclopropylpro pan-2-one and C1 K₂CO₃ 80° C. 2 h | 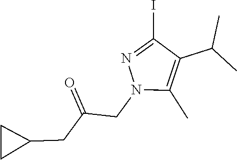 [SMILES: Cc1c(C(C)C)c(I)nn1CC(=O)CC1CC1] | E | 347 | 0.88 |
| | | 1-cyclopropyl-3-[3-iodo-5-methyl-4- | | | |
| | | (propan-2-yl)-1H-pyrazol-1- | | | |
| | | yl]propan-2-one | | | |
| D16 | 1-chloro-3- methoxybutan- 2-one and C1 K₂CO₃ 80° C. 2 h | 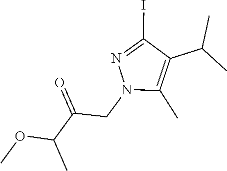 [SMILES: COC(C)C(=O)Cn1nc(I)c(C(C)C)c1C] | H | 351 | 1.05 |
| | | (3R)-1-[3-iodo-5-methyl-4-(propan-2- | | | |
| | | yl)-1H-pyrazol-1-yl]-3-methoxybutan- | | | |
| | | 2-one | | | |
| D17 | 2-chloro-1-(1- methylcyclopropyl) ethan-1-one and C1 K₂CO₃ 80° C. 2 h | 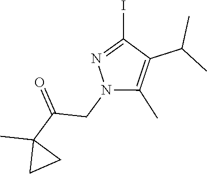 [SMILES: Cc1c(C(C)C)c(I)nn1CC(=O)C1(C)CC1] | H | 347 | 1.09 |
| | | 2-[3-iodo-5-methyl-4-(propan-2-yl)- | | | |
| | | 1H-pyrazol-1-yl]-1-(1- | | | |
| | | methylcyclopropyl)ethan-1-one | | | |
| D18 | 2-chloro-1- [oxolan-2-yl] ethan-1-one and C1 80° C. 2 h | 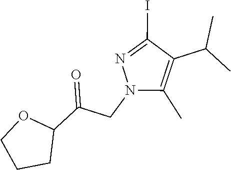 [SMILES: Cc1c(C(C)C)c(I)nn1CC(=O)C1CCCO1] | G | 363 | 1.08 |
| | | 2-[3-iodo-5-methyl-4-(propan-2-yl)- | | | |
| | | 1H-pyrazol-1-yl]-1-[(2R)-oxolan-2- | | | |
| | | yl]ethan-1-one | | | |

### Synthesis of Intermediate D19

### 3-hydroxy-1-[3-iodo-5-methyl-4-(propan-2-yl)-1H-pyrazol-1-yl]-3-methylbutan-2-one

### Step 1: Synthesis of 1-bromo-3-hydroxy-3-methylbutan-2-one

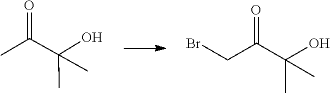

3-Hydroxy-3-methylbutan-2-one (500 mg, 4.9 mmol) is dissolved in acetic acid (2 mL). Bromine (0.25 mL, 4.9 mmol) in acetic acid (3 mL) is added dropwise and the reaction mixture is stirred at RT for 2 h. The reaction mixture is concentrated to afford the product which is employed in the next step without further purification.

### Step 2: Synthesis of 3-hydroxy-1-[3-iodo-5-methyl-4-(propan-2-yl)-1H-pyrazol-1-yl]-3-methylbutan-2-one

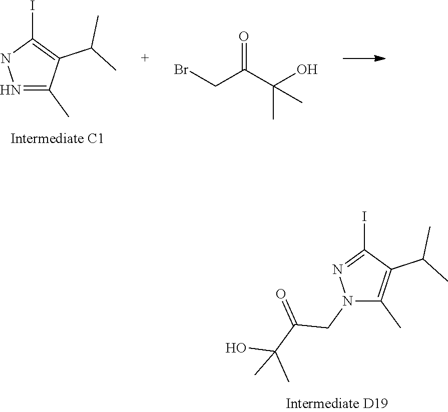

5-Iodo-3-methyl-4-(propan-2-yl)-1H-pyrazole (intermediate C1) (150 mg, 0.60 mmol) is dissolved in ACN (2 mL). K₂CO₃ (207 mg, 1.50 mmol) and 1-bromo-3-hydroxy-3-methylbutan-2-one (217 mg, 1.20 mmol) are added, and the reaction mixture is stirred at 50° C. overnight. The reaction mixture is purified by reversed phase chromatography (HPLC; ACN/water including TFA) to afford the product D19.

Analysis (method G): Rₜ: 1.02 min, [M+H]⁺: 351

### Synthesis of Intermediate D20: 1-[(tert-butyldiphenylsilyl) oxy]-3-[3-iodo-5-methyl-4-(propan-2-yl)-1H-pyrazol-1-yl]propan-2-ol

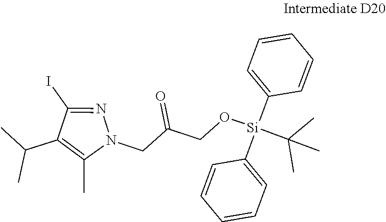 [SMILES: Cc1c(C(C)C)c(I)nn1CC(=O)CO[Si](c1ccccc1)(c1ccccc1)C(C)(C)C]

### Step 1

### Synthesis of 3-iodo-5-methyl-1-(prop-2-en-1-yl)-4-(propan-2-yl)-1H-pyrazole

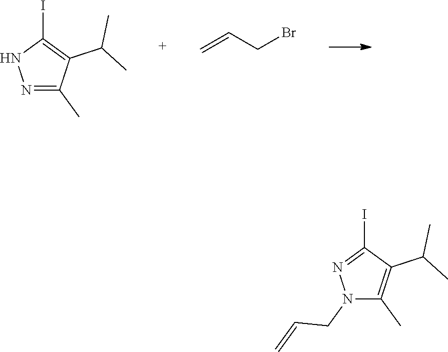

5-Iodo-3-methyl-4-(propan-2-yl)-1H-pyrazole (intermediate C1)(5.0 g, 19.9 mmol) is dissolved in ACN (50 mL), K₂CO₃ (6.9 g, 49.8 mmol) and allyl bromide (4.87 g, 40 mmol) are added. The reaction mixture is stirred at 50° C. overnight. The reaction mixture is filtered, washed with ACN and the filtrate is evaporated. The residue is purified by flash chromatography (CycH/EtOAc 90/10 to CycH/EtOAc 60/40) to afford the desired product.

Analysis (method H): Rₜ: 1.09 min, [M+H]⁺: 291

### Step 2: Synthesis of 3-[3-iodo-5-methyl-4-(propan-2-yl)-1H-pyrazol-1-yl]propane-1, 2-diol

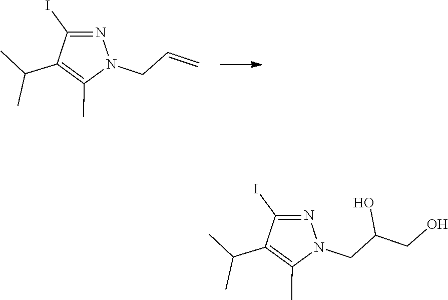

3-Iodo-5-methyl-1-(prop-2-en-1-yl)-4-(propan-2-yl)-1H-pyrazole (3.32 g, 11.4 mmol) is dissolved in THE (45 mL). At 0° C. an aqueous osmium tetroxide solution (4.2 mL, 0.68 mmol, 4% in water) is added dropwise. Then N-methyl-morpholine-N-oxide (2.01 g, 17.2 mmol) is added and the reaction mixture is stirred at RT overnight. The reaction mixture is quenched with a half saturated Na₂S₂O₃ solution (10 mL) and stirred at RT for 1 h. The THF is evaporated in vacuo. The aqueous residue is extracted several times with EtOAc. The combined organic layers are washed with brine, dried (Na₂SO₄), filtered and concentrated to afford the product.

Analysis (method G): Rₜ: 0.76 min, [M+H]⁺: 325

### Step 3: Synthesis of 1-[(tert-butyldiphenylsilyl) oxy]-3-[3-iodo-5-methyl-4-(propan-2-yl)-1H-pyrazol-1-yl]propan-2-ol

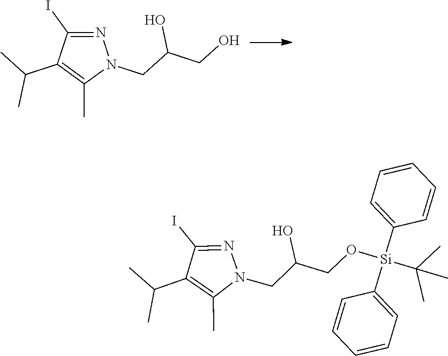 [SMILES: Cc1c(C(C)C)c(I)nn1CC(O)CO ; Cc1c(C(C)C)c(I)nn1CC(O)CO[Si](c1ccccc1)(c1ccccc1)C(C)(C)C]

3-[3-Iodo-5-methyl-4-(propan-2-yl)-1H-pyrazol-1-yl]propane-1, 2-diol (3.70 g, 11.4 mmol) and imidazole (2.49 g, 36.5 mmol) are dissolved in DCM (35 mL). tert-Butyl(chloro)diphenylsilane (3.56 mL, 13.7 mmol) is added and the reaction mixture is stirred at RT for 2 h. The reaction mixture is diluted with water and extracted with DCM. The organic layer is dried (Na₂SO₄), filtered, and concentrated. The residue is purified by flash chromatography (CycH/EtOAc 93/7 to CycH/EtOAc 40/60) to afford the desired product.

Analysis (method G): Rₜ: 1.21 min, [M+H]⁺: 563

### Step 4: Synthesis of 1-[(tert-butyldiphenylsilyl) oxy]-3-[3-iodo-5-methyl-4-(propan-2-yl)-1H-pyrazol-1-yl]propan-2-one

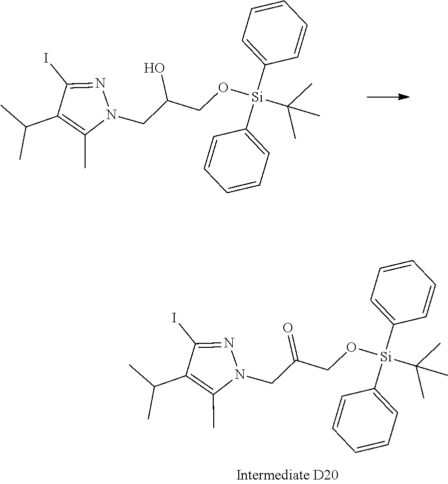

1-[(tert-Butyldiphenylsilyl) oxy]-3-[3-iodo-5-methyl-4-(propan-2-yl)-1H-pyrazol-1-yl]propan-2-ol (4.80 g, 8.53 mmol) is dissolved in DCM (60 mL). DMP (5.43 g, 12.8 mmol) is added, and the reaction mixture is stirred at RT for 2 h. The reaction mixture is concentrated, and the residue is purified by flash chromatography (CycH/EtOAc 100/0 to CycH/EtOAc 60/40) to afford intermediate D20.

Analysis (method G): Rₜ: 1.21 min, [M+H]⁺: 561

### Synthesis of the Imidazole Intermediates D21-D25

### Synthesis of 1-(5-cyclopropyl-1-methyl-1H-imidazol-2-yl)-3-methylbutan-2-one (D21)

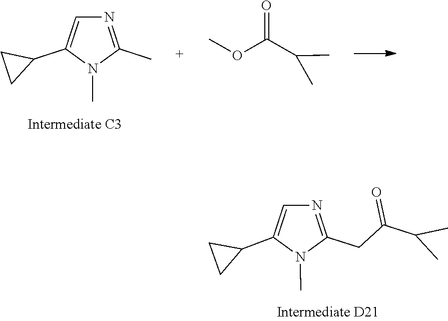

5-Cyclopropyl-1, 2-dimethyl-1H-imidazole (2.55 g, 18.7 mmol) is dissolved in THE (120 mL). At −78° C. n-BuLi (17.5 mL, 28 mmol, 1.6 M) is added dropwise and the reaction mixture is stirred 1 h at −78° C. Then methylisobutyrate (3.54 mL, 28 mmol) is added dropwise, and the reaction mixture is stirred at −78° C. for 1 h. The reaction mixture is quenched with a saturated NH₄Cl solution and extracted 3×EtOAc. The combined organic layers are dried (Na₂SO₄), filtered and concentrated and purified by silica gel chromatography (gradient: DCM/MeOH 100/0 to 94/4) to yield the desired product D21.

Analysis (method C): Rₜ: 0.45 min, [M+H]⁺: 207

### Synthesis of 1-cyclopropyl-2-(5-cyclopropyl-1-methyl-1H-imidazol-2-yl) ethan-1-one (D22)

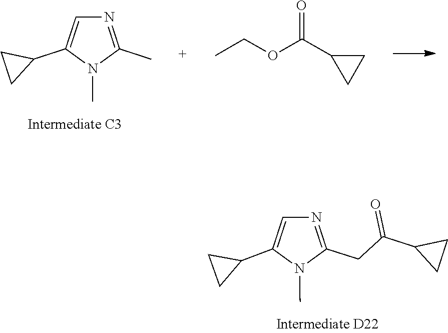

5-Cyclopropyl-1, 2-dimethyl-1H-imidazole (700 mg, 5.14 mmol) is dissolved in THE (7 mL). At −78° C. n-BuLi (6.42 mL, 10.28 mmol, 1.6 M) is added dropwise and the reaction mixture is stirred 30 min at −78° C. Then ethyl cyclopropane carboxylate (1.10 mL, 9.25 mmol) is added dropwise, and the reaction mixture is stirred at −78° C. for 30 min and then allowed to reach RT. The reaction mixture is quenched with a saturated NH₄Cl solution and extracted 3× with DCM/IPA 8/2. The combined organic layers are dried (Na₂SO₄), filtered, and concentrated to afford the product D22.

Analysis (method C): Rₜ: 0.45 min, [M+H]⁺: 205

The intermediates compiled in the following table are obtained by following a procedure analogous to that described for intermediate D22.

| | | | | MS | |
|---|---|---|---|---|---|
| | | | | (ESI+): | |
| Interme- | Starting | | LC | m/z | t_{R} |
| diate | materials | Structure/Name | Method | [M + H]⁺ | [min] |
| D23 | methyl 2, 2,2- trifluoroacetate and C4 | 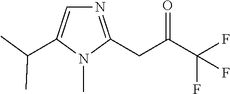 [SMILES: CC(C)c1cnc(CC(=O)C(F)(F)F)n1C] | H | 235 | 0.84 |
| | | 1,1,1-trifluoro-3-[1-methyl-5-(propan-2-yl)- | | | |
| | | 1H-imidazol-2-yl]propan-2-one | | | |

### Synthesis of Intermediate D24

### 4-bromo-5-cyclopropyl-2-{6-ethyl-2-methyl-2H-pyrazolo[3, 4-b]pyridin-5-yl}-1-methyl-1H-imidazole

### Step 1: Synthesis of 5-cyclopropyl-1-methyl-1H-imidazole-2-carbaldehyde

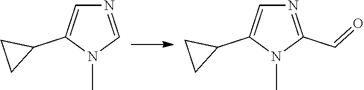

Under an argon atmosphere, 5-cyclopropyl-1-methyl-1H-imidazole (6.30 g, 51.6 mmol) is dissolved in THE (63 mL). At −78° C. n-BuLi (38.7 mL, 61.9 mmol, 1.6 M) is added dropwise and the reaction mixture is stirred at −78° C. for 1 h. Then DMF (5.03 mL, 61.9 mmol) is added dropwise, and the reaction mixture is stirred at −78° C. for 30 min. The reaction mixture is quenched with a saturated NH₄Cl solution and water. Then it is extracted 3× with diethyl ether. The combined organic layers are dried (Na₂SO₄), filtered and concentrated to afford the product which is used in the next step without further purification.

Analysis (method C): Rₜ: 0.35 min, [M+H]⁺: 151

### Step 2: Synthesis of 4-bromo-5-cyclopropyl-1-methyl-1H-imidazole-2-carbaldehyde

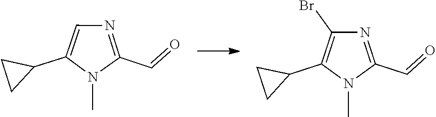

5-Cyclopropyl-1-methyl-1H-imidazole-2-carbaldehyde (17.8 g, 94.8 mmol) is dissolved in DCM (300 mL). At 0° C. NBS (16.9 g, 94.8 mmol) is added, and the reaction mixture is stirred at 0° C. for 1 h. The reaction mixture is washed with a 0.5 M solution of Na₂S₂O₃, water and brine. The organic layer is dried (Na₂SO₄), filtered, and concentrated. The residue is purified by flash chromatography (CycH/EtOAc 100/0 to CycH/EtOAc 50/50) to afford the product.

Analysis (method C): Rₜ: 0.49 min, [M+H]⁺: 229/231 (Br)

### Step 3: Synthesis of 4-bromo-5-cyclopropyl-1-methyl-2-[(1E)-2-nitrobut-1-en-1-yl]-1H-imidazole

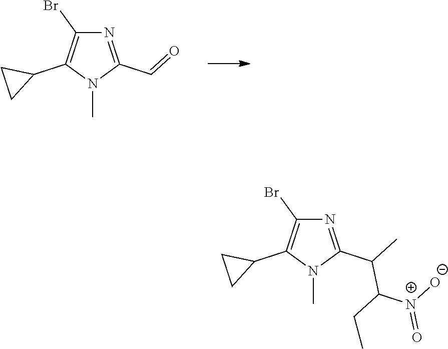 [SMILES: Cn1c(C=O)nc(Br)c1C1CC1 ; CCC(C(C)c1nc(Br)c(C2CC2)n1C)[N+](=O)[O-]]

4-Bromo-5-cyclopropyl-1-methyl-1H-imidazole-2-carbaldehyde (10.1 g, 40.7 mmol) is dissolved in nitropropane (18.1 mL, 203 mmol). Ammonium acetate (6.27 g, 81.4 mmol) is added, and the reaction mixture is stirred at 60° C. for 4 d. The reaction mixture is quenched with a half saturated NaCl solution and is extracted 3× with EtOAc. The combined organic layers are dried (Na₂SO₄), filtered and concentrated to afford the product which is used without further purification in the next step.

Analysis (method C): Rₜ: 0.76 min, [M+H]⁺: 300/302 (Br)

### Step 4: Synthesis of 1-(4-bromo-5-cyclopropyl-1-methyl-1H-imidazol-2-yl) butan-2-one

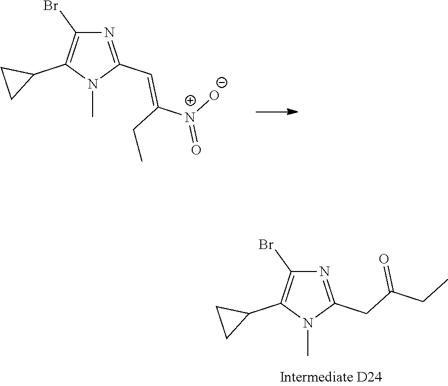 [SMILES: CC/C(=C\c1nc(Br)c(C2CC2)n1C)[N+](=O)[O-] ; CCC(=O)Cc1nc(Br)c(C2CC2)n1C]

Iron powder (11.16 g, 0.200 mol) is suspended in acetic acid (150 mL) and the mixture is heated to 60° C. 4-Bromo-5-cyclopropyl-1-methyl-2-[(1E)-2-nitrobut-1-en-1-yl]-1H-imidazole (12 g, 0.040 mol) in acetic acid (50 mL) is added slowly dropwise and the reaction mixture is stirred at 60° C. for 1.5 h and at 70° C. for 2 h. The hot reaction mixture is filtered, and the solids are washed with acetic acid. The filtrate is diluted with EtOAc and basified with a 2 M solution of Na₂CO₃. Charcoal is added and the reaction mixture is filtered through celite. The layers are separated, and the organic layer is dried (Na₂SO₄), filtered, and concentrated. The residue is purified by flash chromatography (CycH/EtOAc 20/80 to CycH/EtOAc 0/100) to afford the product D24.

Analysis (method F): Rₜ: 0.46 min, [M+H]⁺: 271/273 (Br)

### Synthesis of Intermediate D25

### Synthesis of 2-(5-cyclopropyl-1-methyl-1H-imidazol-2-yl)-1-(2, 2-difluorocyclopropyl) ethan-1-one

### Step 1

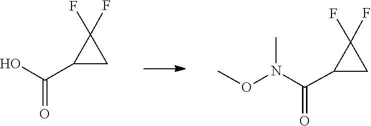

2, 2-Difluorocyclopropanecarboxylic acid (3.4 g, 27.8 mmol), N, O-dimethyl hydroxylamine hydrochloride (3.6 g, 36.9 mmol) and 1-ethyl-3-(3-dimethylaminopropyl)carbodiimide hydrochloride (8.4 g, 42.9 mmol) are dissolved in DCM (40 mL), the mixture cooled to 0° C., then DIPEA (20 mL) is slowly added and the mixture stirred overnight at RT. The mixture is then cooled again to 0° C. and 4 M HCL (35 mL) is added. The organic phase is separated, concentrated, and the product is purified by silica gel chromatography (DCM), and evaporation of the combined fractions at 35° C./90 mbar yields the desired product.

Analysis: ESI [M]⁺: 166

### Step 2

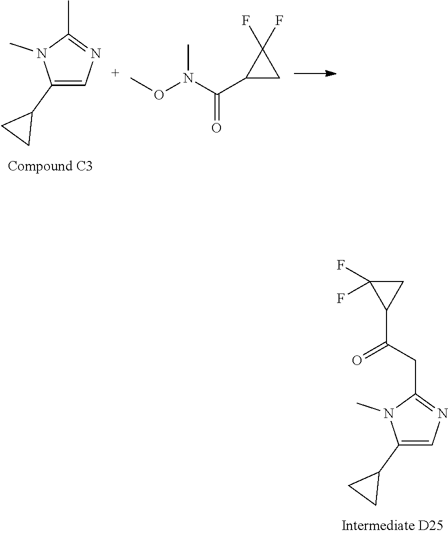

5-Cyclopropyl-1, 2-dimethyl-1H-imidazole (150 mg, 1.1 mmol) is dissolved in THE (4 mL), cooled to −78° C. and BuLi (0.76 mL, 1.6 M) is slowly added. After 15 min, 2, 2-difluoro-N-methoxy-N-methylcyclopropane-1-carboxamide (250 mg, 1.1 mmol, dissolved in 0.5 mL THF) is added and the mixture is stirred for 30 min. Then aqueous NH₄Cl solution is added, and the mixture is extracted with EtOAc. The organic phase is concentrated and purified by preparative HPLC to yield intermediate D25.

Analysis (method G): Rₜ: 0.65 min, [M+H]⁺: 241

### Synthesis of Intermediates E

### Synthesis of Intermediate E1: 3-amino-1-[(4-methoxyphenyl) methyl]-1H-pyrazole-4-carbaldehyde

### Step 1: Synthesis of ethyl 3-amino-1-[(4-methoxyphenyl) methyl]-1H-pyrazole-4-carboxylate

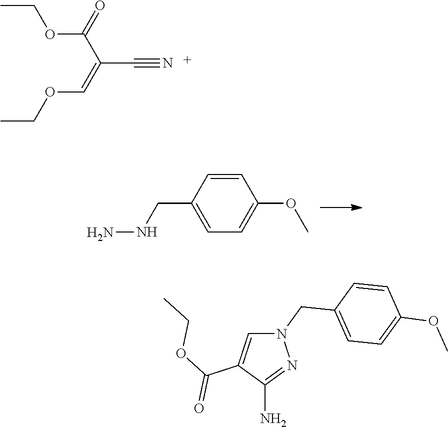 [SMILES: CCO/C=C(/C#N)C(=O)OCC ; COc1ccc(CNN)cc1 ; CCOC(=O)c1cn(Cc2ccc(OC)cc2)nc1N]

A solution of NaOEt is prepared (using 4.08 g Na (177 mmol) and 100 mL of EtOH) to which [(4-methoxyphenyl) methyl]hydrazine hydrochloride (11.2 g, 59 mmol) is added. Then a solution of ethyl (2Z)-2-cyano-3-ethoxyprop-2-enoate (10 g, 59 mmol) in THF (50 mL) is added dropwise over 45 min at 0° C. under Argon. The reaction mixture is stirred at 0° C. for 90 min. The reaction mixture is quenched with 4 M HCl in dioxane (29.6 mL, 118 mmol) and concentrated to dryness. Then the residue is dissolved in EtOAc and washed with a sat. NaHCO₃ solution. The aqueous layer is extracted with EtOAc. The combined organic layers are dried (Na₂SO₄), filtered and concentrated to afford the product.

Analysis (method Q): Rₜ: 1.55 min, [M−H]⁻: 274

### Step 2: Synthesis of {3-amino-1-[(4-methoxyphenyl) methyl]-1H-pyrazol-4-yl}methanol

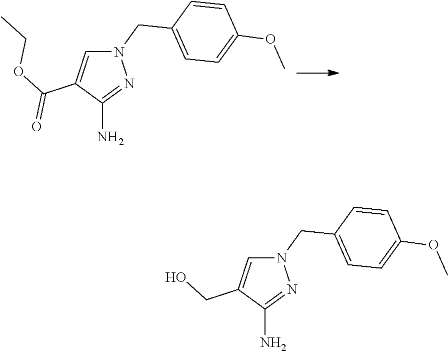 [SMILES: CCOC(=O)c1cn(Cc2ccc(OC)cc2)nc1N ; COc1ccc(Cn2cc(CO)c(N)n2)cc1]

Ethyl 3-amino-1-[(4-methoxyphenyl) methyl]-1H-pyrazole-4-carboxylate (16.3 g, 56.3 mmol, 95% purity) is dissolved in THF (81.5 mL), and at −7° C. LiAlH₄ (2 M in THF, 28.1 mL, 56.3 mmol) is added dropwise over 30 min. The reaction mixture is stirred at RT for 3 h. The reaction mixture is quenched with 2 V of THF/H₂O 8/2 and 1 V of aq. sat. Na₂SO₄ solution and stirred 30 min at RT. The reaction mixture is filtered through Celite and washed with MeOH and DCM/MeOH. The filtrate is dried (Na₂SO₄), filtered, concentrated and co-evaporated with toluene to afford the product.

Analysis (method Q): Rₜ: 1.34 min, [M+H]⁺: 234

### Step 3: Synthesis of 3-amino-1-[(4-methoxyphenyl) methyl]-1H-pyrazole-4-carbaldehyde

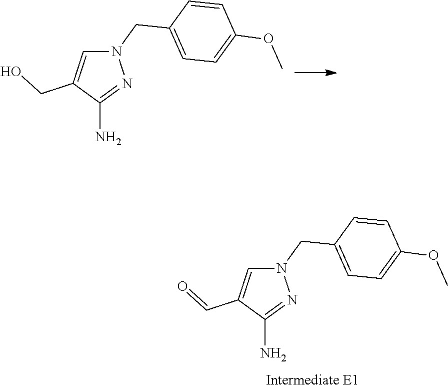 [SMILES: COc1ccc(Cn2cc(CO)c(N)n2)cc1 ; COc1ccc(Cn2cc(C=O)c(N)n2)cc1]

{3-Amino-1-[(4-methoxyphenyl) methyl]-1H-pyrazol-4-yl}methanol (13.1 g, 50.5 mmol, 90% purity) is dissolved in ACN (131 mL) and water (26.2 mL), then MnO₂ (34.2 g, 354 mmol) is added, and the reaction mixture is stirred at RT for 2 h. The reaction mixture is filtered through Celite and washed with DCM/acetone. The filtrate is concentrated to dryness and the residue is triturated with MTBE to afford the product E1.

TLC: silica gel, DCM/MeOH 95/5: Rf: 0.55

Analysis (method Q): Rₜ: 1.55 min

### Synthesis of Intermediate E2

### Step 1: Synthesis of 3-amino-1-[3-(morpholin-4-yl)propyl]-1H-pyrazole-4-carbonitrile

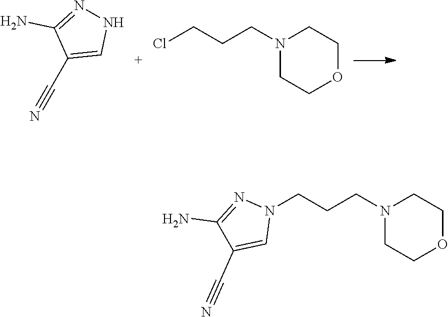 [SMILES: N#Cc1c[nH]nc1N ; ClCCCN1CCOCC1 ; N#Cc1cn(CCCN2CCOCC2)nc1N]

3-Amino-4-cyanopyrazole (2 g, 18.5 mmol) is dissolved in ACN (20 mL) and K₂CO₃ (3.2 g, 23.2 mmol) is added. At 70° C. 4-(3-chloropropyl)morpholine (3.60 g, 22 mmol) in ACN (10 mL) is added dropwise and the reaction mixture is stirred at 70° C. for 2.5 h. The reaction mixture is filtered and the filtrate is purified by reversed phase chromatography (HPLC; ACN/water including NH₃) to afford the product.

Analysis (method C): Rₜ: 0.27 min, [M+H]⁺: 236

### Step 2: Synthesis of 3-amino-1-[3-(morpholin-4-yl)propyl]-1H-pyrazole-4-carbaldehyde

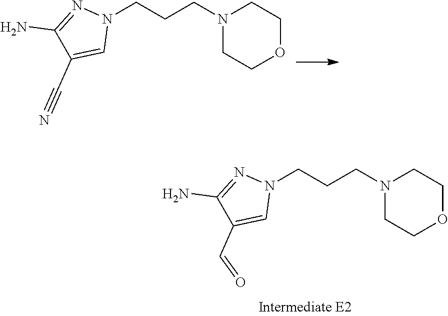 [SMILES: N#Cc1cn(CCCN2CCOCC2)nc1N ; Nc1nn(CCCN2CCOCC2)cc1C=O]

Under an argon atmosphere, 3-amino-1-[3-(morpholin-4-yl)propyl]-1H-pyrazole-4-carbonitrile (0.50 g, 2.13 mmol) is suspended in toluene (5 mL) and at −70° C. DIBALH (5.80 mL, 6.38 mmol, 1.1 M in CycH) is added dropwise. The reaction mixture is stirred at −70° C. for 20 min. Then the reaction mixture is warmed to −10° C. and quenched with an aqueous HCl (2.66 mL, 10.6 mmol, 4 M). The reaction mixture is stirred at RT for 30 min. NH₄OH (1 mL, 28%) and ACN is added and the reaction is filtered through cellulose. The filtrate is concentrated and purified by reversed phase chromatography (HPLC; ACN/water including NH₃) to afford the product E2.

Analysis (method D): Rₜ: 0.26 min, [M+H]⁺: 239

### Synthesis of Intermediate E3

### Synthesis of 3-amino-1-methyl-1H-pyrazole-4-carbaldehyde

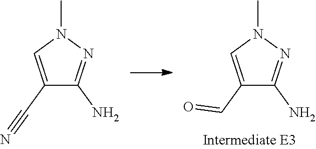

DIBALH in hexane 1M (515 mL; 0.52 mol) is added slowly to a suspension of 3-amino-1-methyl-1H-pyrazole-4-carbonitrile (21.6 g; 0.18 mol) in toluene (432 mL) at −78° C. under argon. After addition the solution is stirred for 20 min and then warmed to RT. The reaction mixture is slowly poured at 0° C. into 4M HCl aq. (177 mL; 0.71 mol) and stirred for 1 h.

The pH is adjusted with potassium carbonate to pH ~9, and the mixture is extracted with IPA/DCM 25/75 (1750 mL) and concentrated to yield intermediate E3.

¹H-NMR (DMSO-d6, 300 MHz): d=9.61 (1H, s), 8.03 (1H, s), 5.66 (2H, s, br), 3.64 (3H, s)

### Synthesis of Intermediates F

### Synthesis of Intermediate F1

### 3-iodo-1-{2-[(4-methoxyphenyl) methyl]-6-methyl-2H-pyrazolo[3, 4-b]pyridin-5-yl}-5-methyl-4-(propan-2-yl)-1H-pyrazole

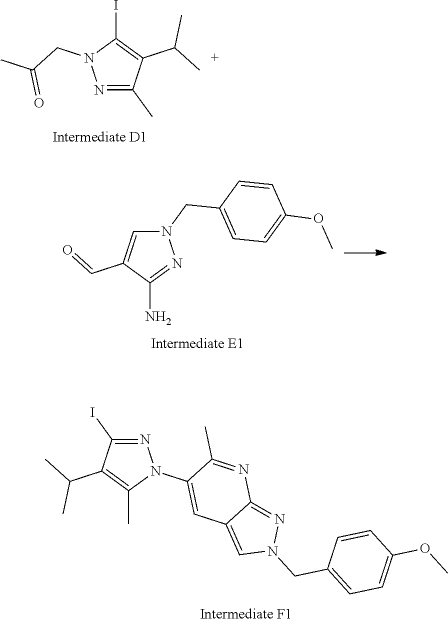 [SMILES: CC(=O)Cn1nc(C)c(C(C)C)c1I ; COc1ccc(Cn2cc(C=O)c(N)n2)cc1 ; COc1ccc(Cn2cc3cc(-n4nc(I)c(C(C)C)c4C)c(C)nc3n2)cc1]

1-[3-Iodo-5-methyl-4-(propan-2-yl)-1H-pyrazol-1-yl]propan-2-one (D1) (5 g, 16.3 mmol) is dissolved in EtOH (75 mL), piperidine (4.03 mL, 40.8 mmol) is added and the reaction mixture is heated to reflux. 3-Amino-1-[(4-methoxyphenyl) methyl]-1H-pyrazole-4-carbaldehyde (E1) (3.40 g, 14.7 mmol) is added at 80° C. and the mixture is stirred overnight at this temperature. The reaction mixture is concentrated, and the residue is purified by flash chromatography (DCM/acetone 90/10 isocratic) to afford intermediate F1.

TLC: silica gel, DCM/acetone 90/10: Rf: 0.20

Analysis (method A): Rₜ: 0.71 min, [M+H]⁺: 502

### Synthesis of Intermediate F2

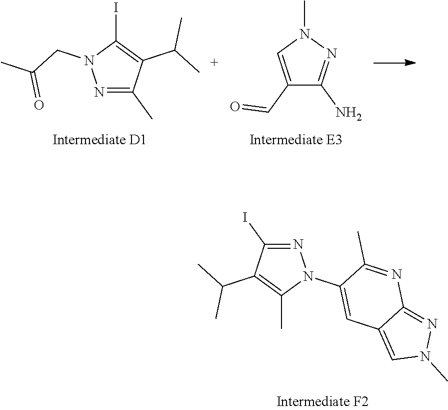

1-{2, 6-dimethyl-2H-pyrazolo[3, 4-b]pyridin-5-yl}-3-iodo-5-methyl-4-(propan-2-yl)-1H-pyrazole 1-[3-Iodo-5-methyl-4-(propan-2-yl)-1H-pyrazol-1-yl]propan-2-one (D1) (2.7 g, 8.82 mmol), 3-amino-1-methyl-1 h-pyrazole-4-carbaldehyde (E3) (1.1 g, 8.82 mmol) are dissolved in EtOH, piperidine (2.18 mL, 22.1 mmol) is added and the reaction mixture is stirred in a closed vial at 80° C. for 4 h. The reaction mixture is concentrated, and the residue is purified by flash chromatography (DCM/MeOH 100/0 to DCM/MeOH 90/10) to afford intermediate F2.

Analysis (method F): Rₜ: 0.79 min, [M+H]⁺: 396

The intermediates compiled in the following table are obtained by following a procedure analogous to that described for intermediate F2.

| | Starting | | | MS | |
|---|---|---|---|---|---|
| | materials | | | (ESI+): | |
| Interme- | and | | LC | m/z | t_{R} |
| diate | conditions | Structure/Name | Method | [M + H]⁺ | [min] |
| F3 | D18 + E3 NaOMe piperidine 90° 1 h | 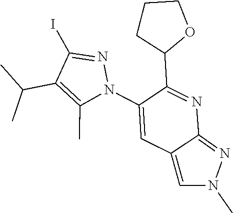 [SMILES: Cc1c(C(C)C)c(I)nn1-c1cc2cn(C)nc2nc1C1CCCO1] | H | 452 | 1.03 |
| | | 3-iodo-5-methyl-1-{2-methyl-6-[oxolan-2- | | | |
| | | yl]-2H-pyrazolo[3,4-b]pyridin-5-yl}-4- | | | |
| | | (propan-2-yl)-1H-pyrazole | | | |
| F4 | D2 + E3 piperidine 80° 24 h | 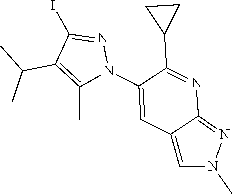 [SMILES: Cc1c(C(C)C)c(I)nn1-c1cc2cn(C)nc2nc1C1CC1] | C | 422 | 0.70 |
| | | 1-{6-cyclopropyl-2-methyl-2H-pyrazolo[3, | | | |
| | | 4-b]pyridin-5-yl}-3-iodo-5-methyl-4- | | | |
| | | (propan-2-yl)-1H-pyrazole | | | |
| F5 | D3 + E3 piperidine 100° C. 16 h | 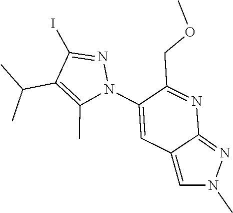 [SMILES: COCc1nc2nn(C)cc2cc1-n1nc(I)c(C(C)C)c1C] | G | 426 | 0.99 |
| | | 3-iodo-1-[6-(methoxymethyl)-2-methyl- | | | |
| | | 2H-pyrazolo[3,4-b]pyridin-5-yl]-5-methyl- | | | |
| | | 4-(propan-2-yl)-1H-pyrazole | | | |
| F6 | D7 + E3 piperidine 80° 24 h | 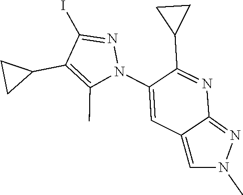 [SMILES: Cc1c(C2CC2)c(I)nn1-c1cc2cn(C)nc2nc1C1CC1] | C | 420 | 0.67 |
| | | 4-cyclopropyl-1-{6-cyclopropyl-2-methyl- | | | |
| | | 2H-pyrazolo[3,4-b]pyridin-5-yl}-3-iodo-5- | | | |
| | | methyl-1H-pyrazole | | | |
| F7 | D8 + E3 piperidine 80° 16 h | 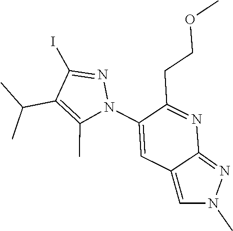 [SMILES: COCCc1nc2nn(C)cc2cc1-n1nc(I)c(C(C)C)c1C] | G | 440 | 0.99 |
| | | 3-iodo-1-[6-(2-methoxyethyl)-2-methyl- | | | |
| | | 2H-pyrazolo[3,4-b]pyridin-5-yl]-5-methyl- | | | |
| | | 4-(propan-2-yl)-1H-pyrazole | | | |
| F8 | D13 + E3 NaOMe; piperidine 90° 1 h | 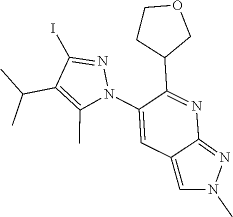 [SMILES: Cc1c(C(C)C)c(I)nn1-c1cc2cn(C)nc2nc1C1CCOC1] | G | 452 | 1.05 |
| | | 3-iodo-5-methyl-1-[2-methyl-6-(oxolan-3- | | | |
| | | yl)-2H-pyrazolo[3,4-b]pyridin-5-yl]-4- | | | |
| | | (propan-2-yl)-1H-pyrazole | | | |
| F9 | D14 + E3 NaOMe; piperidine 90° 1 h | 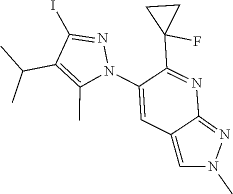 [SMILES: Cc1c(C(C)C)c(I)nn1-c1cc2cn(C)nc2nc1C1(F)CC1] | H | 440 | 1.08 |
| | | 1-[6-(1-fluorocyclopropyl)-2-methyl-2H- | | | |
| | | pyrazolo[3,4-b]pyridin-5-yl]-3-iodo-5- | | | |
| | | methyl-4-(propan-2-yl)-1H-pyrazole | | | |
| F10 | D4 + E3 NaOMe; piperidine 90° 2 h | 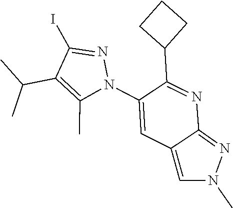 [SMILES: Cc1c(C(C)C)c(I)nn1-c1cc2cn(C)nc2nc1C1CCC1] | H | 436 | 1.16 |
| | | 1-{6-cyclobutyl-2-methyl-2H-pyrazolo[3, | | | |
| | | 4-b]pyridin-5-yl}-3-iodo-5-methyl-4- | | | |
| | | (propan-2-yl)-1H-pyrazole | | | |
| F11 | D15 + E3 NaOMe; piperidine 90° 2 h | 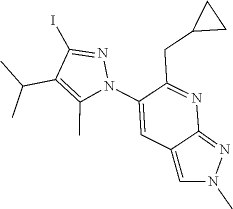 [SMILES: Cc1c(C(C)C)c(I)nn1-c1cc2cn(C)nc2nc1CC1CC1] | H | 436 | 1.09 |
| | | 1-[6-(cyclopropylmethyl)-2-methyl-2H- | | | |
| | | pyrazolo[3,4-b]pyridin-5-yl]-3-iodo-5- | | | |
| | | methyl-4-(propan-2-yl)-1H-pyrazole | | | |
| F12 | D16 + E3 NaOMe; piperidine 90° 1 h | 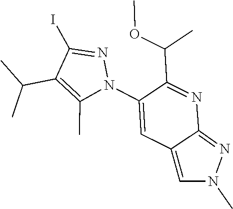 [SMILES: COC(C)c1nc2nn(C)cc2cc1-n1nc(I)c(C(C)C)c1C] | H | 440 | 1.03 |
| | | 3-iodo-1-{6-[(1R)-1-methoxyethyl]-2- | | | |
| | | methyl-2H-pyrazolo[3,4-b]pyridin-5-yl}-5- | | | |
| | | methyl-4-(propan-2-yl)-1H-pyrazole | | | |
| F13 | D5 + E3 NaOMe; piperidine 90° 1 h | 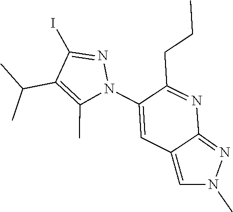 [SMILES: CCCc1nc2nn(C)cc2cc1-n1nc(I)c(C(C)C)c1C] | H | 424 | 1.10 |
| | | 3-iodo-5-methyl-1-{2-methyl-6-propyl-2H- | | | |
| | | pyrazolo[3,4-b]pyridin-5-yl}-4-(propan-2- | | | |
| | | yl)-1H-pyrazole | | | |
| F14 | D6 + E3 NaOMe; piperidine 90° 2 h | 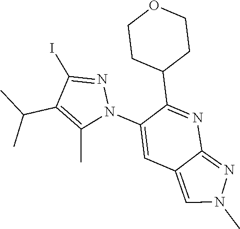 [SMILES: Cc1c(C(C)C)c(I)nn1-c1cc2cn(C)nc2nc1C1CCOCC1] | H | 466 | 1.07 |
| | | 3-iodo-5-methyl-1-[2-methyl-6-(oxan-4- | | | |
| | | yl)-2H-pyrazolo[3,4-b]pyridin-5-yl]-4- | | | |
| | | (propan-2-yl)-1H-pyrazole | | | |
| F15 | D17 + E3 NaOMe; piperidine 90° 1 h | 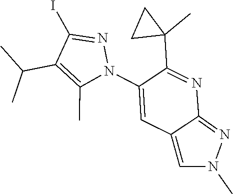 [SMILES: Cc1c(C(C)C)c(I)nn1-c1cc2cn(C)nc2nc1C1(C)CC1] | H | 436 | 1.10 |
| | | 3-iodo-5-methyl-1-[2-methyl-6-(1- | | | |
| | | methylcyclopropyl)-2H-pyrazolo[3,4- | | | |
| | | b]pyridin-5-yl]-4-(propan-2-yl)-1H- | | | |
| | | pyrazole | | | |
| F16 | D19 + E3 NaOMe; piperidine 90° 1 h | 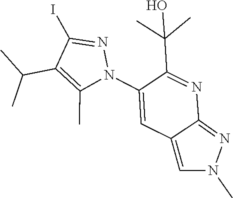 [SMILES: Cc1c(C(C)C)c(I)nn1-c1cc2cn(C)nc2nc1C(C)(C)O] | G | 440 | 1.04 |
| | | 2-{5-[3-iodo-5-methyl-4-(propan-2-yl)-1H- | | | |
| | | pyrazol-1-yl]-2-methyl-2H-pyrazolo[3,4- | | | |
| | | b]pyridin-6-yl}propan-2-ol | | | |
| F17 | D9 + E3 piperidine 80° C., 6 h | 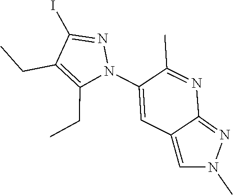 [SMILES: CCc1c(I)nn(-c2cc3cn(C)nc3nc2C)c1CC] | G | 396 | 0.99 |
| | | 1-{2,6-dimethyl-2H-pyrazolo[3,4- | | | |
| | | b]pyridin-5-yl}-4,5-diethyl-3-iodo-1H- | | | |
| | | pyrazole | | | |
| F18 | D10 + E3 Ethanol; piperidine 80° C., overnight | 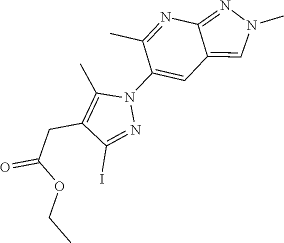 [SMILES: CCOC(=O)Cc1c(I)nn(-c2cc3cn(C)nc3nc2C)c1C] | H | 440 | 0.89 |
| | | ethyl 2-(1-{2,6-dimethyl-2H-pyrazolo[3, | | | |
| | | 4-b]pyridin-5-yl}-3-iodo-5-methyl-1H- | | | |
| | | pyrazol-4-yl)acetate | | | |
| F19 | D12 + E3 | 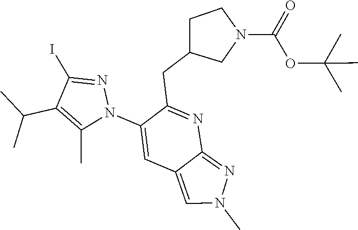 [SMILES: Cc1c(C(C)C)c(I)nn1-c1cc2cn(C)nc2nc1CC1CCN(C(=O)OC(C)(C)C)C1] | H | 551 | 1.18 |
| | | tert-butyl 2-{5-[3-iodo-5-methyl-4- | | | |
| | | (propan-2-yl)-1H-pyrazol-1-yl]-2-methyl- | | | |
| | | 2H-pyrazolo[3,4-b]pyridin-6- | | | |
| | | yl}pyrrolidine-1-carboxylate | | | |
| F20 | D11 + E3 | 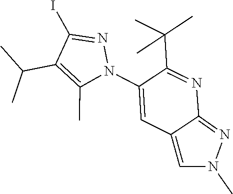 [SMILES: Cc1c(C(C)C)c(I)nn1-c1cc2cn(C)nc2nc1C(C)(C)C] | G | 438 | 1.21 |
| | | 1-{6-tert-butyl-2-methyl-2H-pyrazolo[3,4- | | | |
| | | b]pyridin-5-yl}-3-iodo-5-methyl-4-(propan- | | | |
| | | 2-yl)-1H-pyrazole | | | |

### Synthesis of Intermediate F21

### 1-{6-chloro-2-methyl-2H-pyrazolo[3, 4-b]pyridin-5-yl}-3-iodo-5-methyl-4-(propan-2-yl)-1H-pyrazole

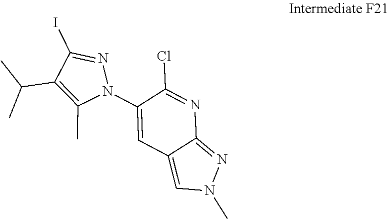 [SMILES: Cc1c(C(C)C)c(I)nn1-c1cc2cn(C)nc2nc1Cl]

### Step 1: Synthesis of 5-bromo-2-methyl-2H-pyrazolo[3, 4-b]pyridine

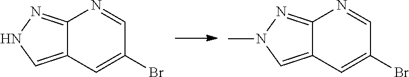

To an ice-cooled solution of 5-bromo-2H-pyrazolo[3, 4-b]pyridine (10 g, 0.050 mol) in dry THE (100 mL), sodium bis(trimethylsilyl)amide solution 1.0 M in THE (75.8 mL, 0.076 mol) and iodomethane (9.43 mL, 0.151 mol) are added. The resulting mixture is allowed to warm to RT and stirred for additional 2 h. DCM (150 mL) and 1 M NH₄Cl (150 mL) are added to the reaction mixture. The aqueous layer is extracted with DCM. The combined organic layers are dried (Na₂SO₄), filtered and concentrated. The residue is purified by flash chromatography (hexane/EtOAc 100/0 to hexane/EtOAc 0/100) to afford the product.

Analysis (method S): Rₜ: 1.01 min, [M+H]⁺: 212/214 (Br)

### Step 2: Synthesis of 5-bromo-2-methyl-2H-pyrazolo[3, 4-b]pyridin-7-ium-7-olate

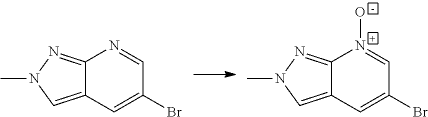

5-Bromo-2-methyl-2H-pyrazolo[3, 4-b]pyridine (5 g, 23.6 mmol) is suspended in DCM (160 mL) and MCPBA (10 g, 43.5 mmol, 75% purity) is added. The reaction mixture is stirred at RT overnight. Then the mixture is filtered, and the filtrate is evaporated. Saturated NaHCO₃ solution is added to the residue, and this mixture is extracted with DCM and a little bit of MeOH. The combined organic phases are dried (Na₂SO₄), filtered, and concentrated. The residue is triturated with MTBE and the precipitate is filtered and dried in an oven at 50° C. overnight to afford the product.

Analysis (method H): Rₜ: 0.48 min, [M+H]⁺: 228/230 (Br)

### Step 3: Synthesis of 5-[3-iodo-5-methyl-4-(propan-2-yl)-1H-pyrazol-1-yl]-2-methyl-2H-pyrazolo[3, 4-b]pyridin-7-ium-7-olate

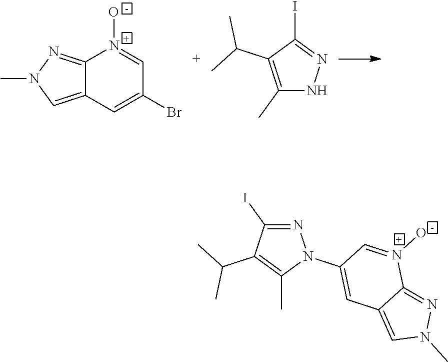

5-Bromo-2-methyl-2H-pyrazolo[3, 4-b]pyridin-7-ium-7-olate (1.50 g, 3.95 mmol, 60% purity) and 3-iodo-5-methyl-4-(propan-2-yl)-1H-pyrazole (C1) (1.04 g, 4.14 mmol) are dissolved in NMP (5 mL). Under a nitrogen atmosphere, K₂CO₃ (1.64 g, 11.8 mmol), trans-(1R, 2R)—N, N′-bismethyl-1, 2-cyclohexane diamine (0.50 mL, 3.17 mmol) and copper (1) iodide (601 mg, 3.16 mmol) are added and the reaction mixture is stirred in a closed vial at 105° C. for 13 h. The reaction mixture is filtered, washed with MeOH and the filtrate is concentrated. The residue is purified by flash chromatography (DCM/MeOH 100/0 to DCM/MeOH 86/14) to afford the desired product.

Analysis (method H): Rₜ: 0.88 min, [M+H]⁺: 398

### Step 4: Synthesis of 1-{6-chloro-2-methyl-2H-pyrazolo[3, 4-b]pyridin-5-yl}-3-iodo-5-methyl-4-(propan-2-yl)-1H-pyrazole

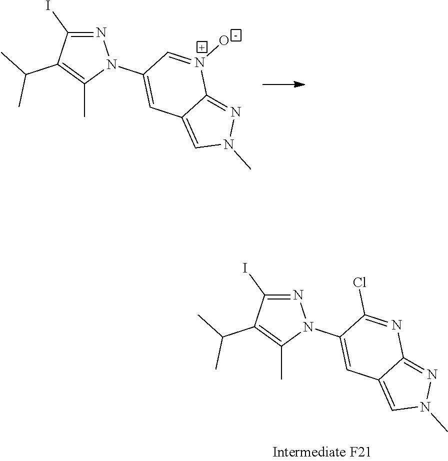

5-[3-Iodo-5-methyl-4-(propan-2-yl)-1H-pyrazol-1-yl]-2-methyl-2H-pyrazolo[3, 4-b]pyridin-7-ium-7-olate (554.0 mg, 1.12 mmol, 80% purity) is dissolved in ACN (5 mL). POCl₃ (500 μL, 5.31 mmol) is added and the reaction mixture is stirred at 50° C. for 45 min. The reaction mixture is concentrated, and the residue is purified by flash chromatography (DCM/MeOH 100/0 to DCM/MeOH 90/10) to afford the desired product F21.

Analysis (method H): Rₜ: 1.04 min, [M+H]⁺: 416

### Synthesis of Intermediate F22: 1-{6-ethoxy-2-methyl-2H-pyrazolo[3, 4-b]pyridin-5-yl}-3-iodo-5-methyl-4-(propan-2-yl)-1H-pyrazole

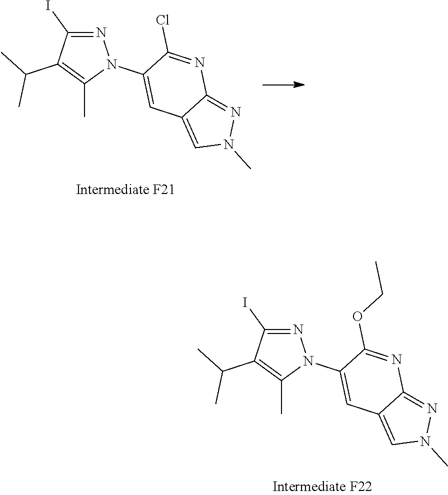 [SMILES: Cc1c(C(C)C)c(I)nn1-c1cc2cn(C)nc2nc1Cl ; CCOc1nc2nn(C)cc2cc1-n1nc(I)c(C(C)C)c1C]

1-{6-Chloro-2-methyl-2H-pyrazolo[3, 4-b]pyridin-5-yl}-3-iodo-5-methyl-4-(propan-2-yl)-1H-pyrazole (60.0 mg, 0.12 mmol, 80% purity) is dissolved in EtOH (2.0 mL). LiHMDS (400 μL, 0.40 mmol) is added and the reaction mixture is stirred at 40° C. for 3 d and at 60° C. overnight. The reaction mixture is quenched with some drops of water and is concentrated. The residue is purified by flash chromatography (DCM/MeOH 100/0 to DCM/MeOH 80/20) to afford the product F22.

Analysis (method H): Rₜ: 1.08 min, [M+H]⁺: 426

The intermediates compiled in the following table are obtained by following a procedure analogous to that described for Intermediate F22.

| | | | | MS | |
|---|---|---|---|---|---|
| | | | | (ESI+): | |
| Interme- | Starting | | LC | m/z | t_{R} |
| diate | materials | Structure/Name | Method | [M + H]⁺ | [min] |
| F23 | isopropanol and F21 | 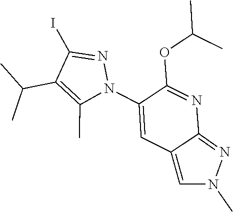 [SMILES: Cc1c(C(C)C)c(I)nn1-c1cc2cn(C)nc2nc1OC(C)C] | H | 440 | 1.11 |
| | | 3-iodo-5-methyl-1-[2-methyl-6-(propan-2- | | | |
| | | yloxy)-2H-pyrazolo[3,4-b]pyridin-5-yl]-4- | | | |
| | | (propan-2-yl)-1H-pyrazole | | | |
| F24 | MeOH and F21 | 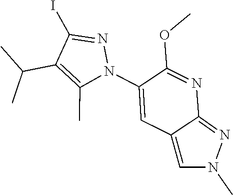 [SMILES: COc1nc2nn(C)cc2cc1-n1nc(I)c(C(C)C)c1C] | H | 412 | 1.03 |
| | | 3-iodo-1-{6-methoxy-2-methyl-2H-pyrazolo[3, | | | |
| | | 4-b]pyridin-5-yl}-5-methyl-4-(propan-2-yl)-1H- | | | |
| | | pyrazole | | | |
| F25 | ethylene glycol and F21 | 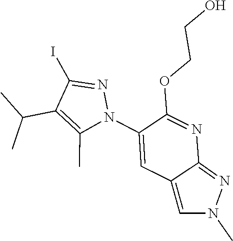 [SMILES: Cc1c(C(C)C)c(I)nn1-c1cc2cn(C)nc2nc1OCCO] | H | 442 | 0.94 |
| | | 2-({5-[3-iodo-5-methyl-4-(propan-2-yl)-1H- | | | |
| | | pyrazol-1-yl]-2-methyl-2H-pyrazolo[3,4- | | | |
| | | b]pyridin-6-yl}oxy)ethan-1-ol | | | |
| F26 | methylamine and F21 | 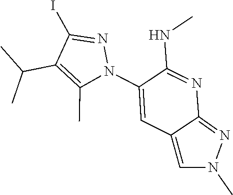 [SMILES: CNc1nc2nn(C)cc2cc1-n1nc(I)c(C(C)C)c1C] | H | 411 | 0.98 |
| | | 5-[3-iodo-5-methyl-4-(propan-2-yl)-1H- | | | |
| | | pyrazol-1-yl]-N,2-dimethyl-2H-pyrazolo[3,4- | | | |
| | | b]pyridin-6-amine | | | |
| F27 | dimethylamine and F21 | 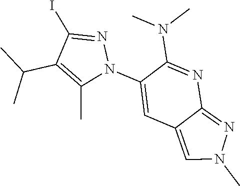 [SMILES: Cc1c(C(C)C)c(I)nn1-c1cc2cn(C)nc2nc1N(C)C] | G | 425 | 0.91 |
| | | 5-[3-iodo-5-methyl-4-(propan-2-yl)-1H- | | | |
| | | pyrazol-1-yl]-N,N,2-trimethyl-2H- | | | |
| | | pyrazolo[3,4-b]pyridin-6-amine | | | |

### Synthesis of Intermediate F28

### Step 1: Synthesis of 1-(6-{[(tert-butyldiphenylsilyl)oxy]methyl}-2-methyl-2H-pyrazolo[3, 4-b]pyridin-5-yl)-3-iodo-5-methyl-4-(propan-2-yl)-1H-pyrazole

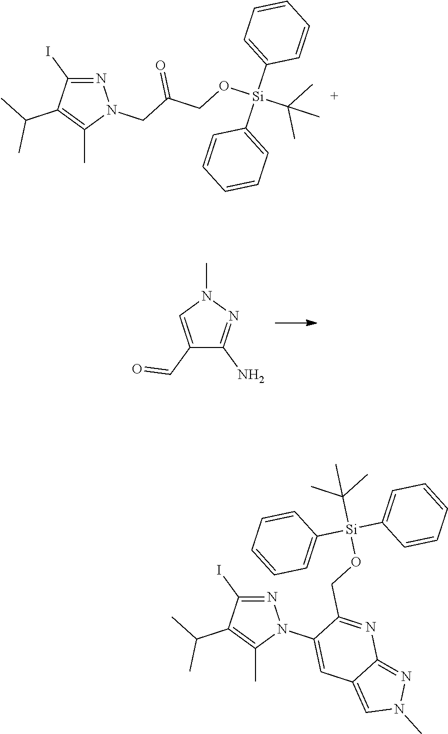

1-[(Tert-butyldiphenylsilyl)oxy]-3-[3-iodo-5-methyl-4-(propan-2-yl)-1H-pyrazol-1-yl]propan-2-one (3.98 g, 7.10 mmol) and 3-amino-1-methyl-1H-pyrazole-4-carbaldehyde (0.89 g, 7.10 mmol) are dissolved in EtOH (20 mL). Piperidine (1.76 mL, 17.8 mmol) is added and the reaction mixture is stirred at 100° C. overnight. The reaction mixture is concentrated to afford the product, which was used in the next step without further purification.

### Step 2: Synthesis of {5-[3-iodo-5-methyl-4-(propan-2-yl)-1H-pyrazol-1-yl]-2-methyl-2H-pyrazolo[3, 4-b]pyridin-6-yl}methanol

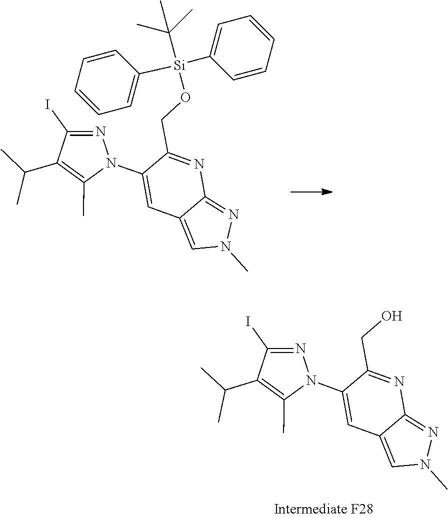 [SMILES: Cc1c(C(C)C)c(I)nn1-c1cc2cn(C)nc2nc1CO[Si](c1ccccc1)(c1ccccc1)C(C)(C)C ; Cc1c(C(C)C)c(I)nn1-c1cc2cn(C)nc2nc1CO]

1-(6-{[(Tert-butyldiphenylsilyl)oxy]methyl}-2-methyl-2H-pyrazolo[3, 4-b]pyridin-5-yl)-3-iodo-5-methyl-4-(propan-2-yl)-1H-pyrazole (4.60 g, 7.08 mmol) is dissolved in THE (40 mL). TBAF (8.5 mL, 8.5 mmol, 1 M) is added and the reaction mixture is stirred at RT for 1.5 h. The reaction mixture is concentrated and the residue purified by reversed phase chromatography (HPLC; ACN/water including TFA) to afford the product F28.

Analysis (method G): Rₜ: 0.93 min, [M+H]⁺: 412

### Synthesis of Intermediate F29

### Step 1: Synthesis of 5-[3-iodo-5-methyl-4-(propan-2-yl)-1H-pyrazol-1-yl]-2-methyl-2H-pyrazolo[3, 4-b]pyridine-6-carbaldehyde

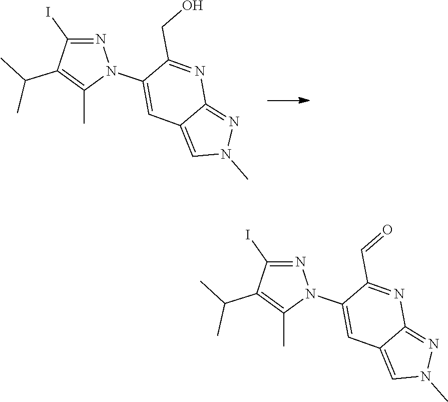 [SMILES: Cc1c(C(C)C)c(I)nn1-c1cc2cn(C)nc2nc1CO ; Cc1c(C(C)C)c(I)nn1-c1cc2cn(C)nc2nc1C=O]

{5-[3-Iodo-5-methyl-4-(propan-2-yl)-1H-pyrazol-1-yl]-2-methyl-2H-pyrazolo[3, 4-b]pyridin-6-yl}methanol (F28) (100 mg, 0.24 mmol) is dissolved in DCM (2 mL). DMP (155 mg, 0.37 mmol) is added and the reaction mixture is stirred at RT for 2 h. The reaction mixture is concentrated and the residue is purified by flash chromatography (DCM/MeOH 100/0 to DCM/MeOH 60/40) to afford the product.

Analysis (method G): Rₜ: 0.84 min, [M+H]⁺: 410

### Step 2: Synthesis of 1-{5-[3-iodo-5-methyl-4-(propan-2-yl)-1H-pyrazol-1-yl]-2-methyl-2H-pyrazolo[3, 4-b]pyridin-6-yl}ethan-1-ol

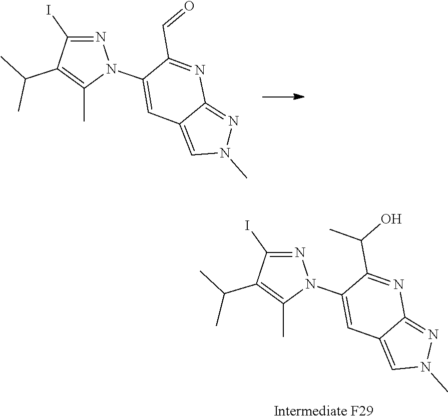 [SMILES: Cc1c(C(C)C)c(I)nn1-c1cc2cn(C)nc2nc1C=O ; Cc1c(C(C)C)c(I)nn1-c1cc2cn(C)nc2nc1C(C)O]

5-[3-Iodo-5-methyl-4-(propan-2-yl)-1H-pyrazol-1-yl]-2-methyl-2H-pyrazolo[3, 4-b]pyridine-6-carbaldehyde (125 mg, 0.21 mmol) is dissolved in THE (2 mL). At 0° C. methylmagnesium bromide (0.22 mL, 3 M in diethyl ether, 0.66 mmol) is added dropwise and the reaction mixture is stirred at 0° C. for 10 min and at RT for 2 h. The reaction mixture is quenched with a saturated NH₄Cl solution and extracted 2× with EtOAc. The combined organic layers are washed with brine, dried (Na₂SO₄), filtered and concentrated. The residue is purified by reversed phase chromatography (HPLC; ACN/water including NH3) to afford the product F29

Analysis (method H): Rₜ: 0.99 min, [M+H]⁺: 426

### Synthesis of Intermediate F30:3-iodo-5-methyl-1-[2-methyl-6-(methylsulfanyl)-2H-pyrazolo[3, 4-b]pyridin-5-yl]-4-(propan-2-yl)-1H-pyrazole

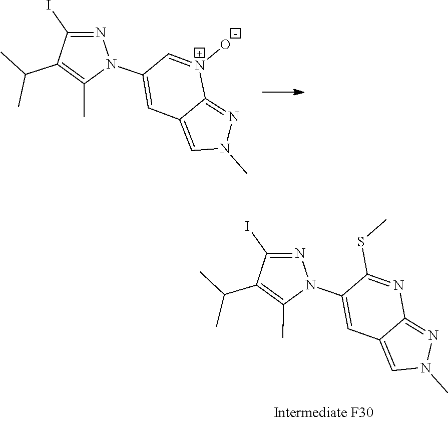

5-[3-Iodo-5-methyl-4-(propan-2-yl)-1H-pyrazol-1-yl]-2-methyl-2H-pyrazolo[3, 4-b]pyridin-7-ium-7-olate (30 mg, 0.08 mmol, cf. synthesis F21, step 4) is dissolved in ACN (2 mL). POCl₃ (36 μL, 0.378 mmol) is added and the reaction mixture is stirred at 60° C. for 45 min. The reaction mixture is concentrated, and the residue is dissolved in DMF (2 mL). NaH (18 mg, 0.41 mmol, 55%) and sodium methane thiolate (24 mg, 0.34 mmol) are added and the reaction mixture is stirred at RT overnight. The reaction mixture is quenched with water and purified by reversed phase chromatography (HPLC; ACN/water including NH3) to afford the product F30.

Analysis (method H): Rₜ: 1.07 min, [M+H]⁺: 428

### Synthesis of Intermediates F31 and F32

### 4-({5-[3-iodo-5-methyl-4-(propan-2-yl)-1H-pyrazol-1-yl]-2-methyl-2H-pyrazolo[3, 4-b]pyridin-6-yl}methyl) morpholine

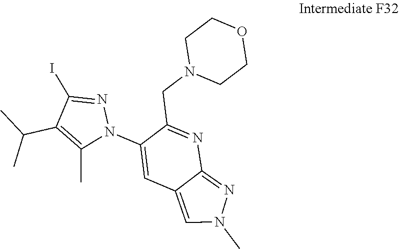 [SMILES: Cc1c(C(C)C)c(I)nn1-c1cc2cn(C)nc2nc1CN1CCOCC1]

### Step 1: Synthesis of {5-[3-iodo-5-methyl-4-(propan-2-yl)-1H-pyrazol-1-yl]-2-methyl-2H-pyrazolo[3, 4-b]pyridin-6-yl}methyl methanesulfonate

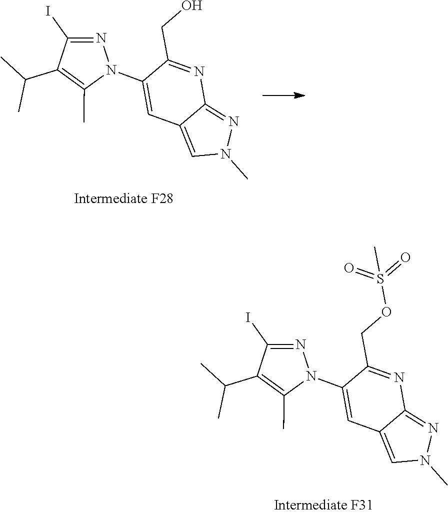 [SMILES: Cc1c(C(C)C)c(I)nn1-c1cc2cn(C)nc2nc1CO ; Cc1c(C(C)C)c(I)nn1-c1cc2cn(C)nc2nc1COS(C)(=O)=O]

{5-[3-Iodo-5-methyl-4-(propan-2-yl)-1H-pyrazol-1-yl]-2-methyl-2H-pyrazolo[3, 4-b]pyridin-6-yl}methanol (F28, 200 mg, 0.49 mmol) and TEA (149 μL, 1.07 mmol) are dissolved in DCM (2 mL). Methanesulfonyl chloride (45 μL, 0.58 mmol) is added and the reaction mixture is stirred at RT for 1 h. The reaction mixture is diluted with DCM and washed with a saturated NaHCO₃ solution. The organic layer is dried (Na₂SO₄), filtered and concentrated to afford intermediate F31.

Analysis (method G): Rₜ: 0.85 min, [M+H]⁺: 490

### Step 2: Synthesis of 4-({5-[3-iodo-5-methyl-4-(propan-2-yl)-1H-pyrazol-1-yl]-2-methyl-2H-pyrazolo[3, 4-b]pyridin-6-yl}methyl) morpholine

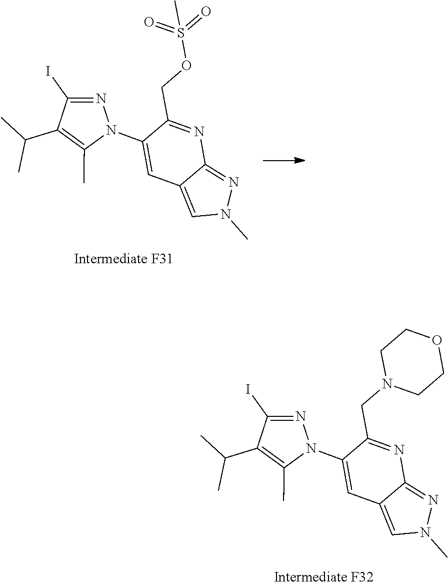 [SMILES: Cc1c(C(C)C)c(I)nn1-c1cc2cn(C)nc2nc1COS(C)(=O)=O ; Cc1c(C(C)C)c(I)nn1-c1cc2cn(C)nc2nc1CN1CCOCC1]

{5-[3-Iodo-5-methyl-4-(propan-2-yl)-1H-pyrazol-1-yl]-2-methyl-2H-pyrazolo[3, 4-b]pyridin-6-yl}methyl methanesulfonate (F31) (60 mg, 0.12 mmol) is dissolved in THE (1 mL). Morpholine (53 μL, 0.61 mmol) is added and the reaction mixture is stirred at RT for 1 h. The reaction mixture is purified by reversed phase chromatography (HPLC; ACN/water including NH₃) to afford the intermediate F32.

Analysis (method H): Rₜ: 1.02 min, [M+H]⁺: 481

The intermediates compiled in the following table are obtained by following a procedure analogous to that described for Intermediate F32.

| | | | | MS | |
|---|---|---|---|---|---|
| | | | | (ESI+): | |
| Interme- | Starting | | LC | m/z | t_{R} |
| diate | materials | Structure/Name | Method | [M + H]⁺ | [min] |
| F33 | methyl amine and F31 | 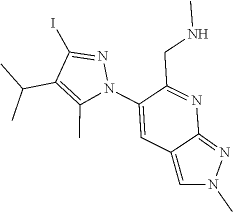 [SMILES: CNCc1nc2nn(C)cc2cc1-n1nc(I)c(C(C)C)c1C] | G | 425 | 0.79 |
| F34 | dimethylamine and F31 | 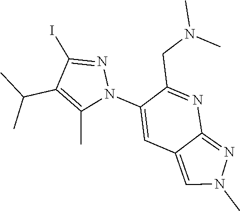 [SMILES: Cc1c(C(C)C)c(I)nn1-c1cc2cn(C)nc2nc1CN(C)C] | H | 439 | 1.07 |
| | | ({5-[3-iodo-5-methyl-4-(propan-2-yl)-1H- | | | |
| | | pyrazol-1-yl]-2-methyl-2H-pyrazolo[3,4- | | | |
| | | b]pyridin-6-yl}methyl)dimethylamine | | | |

### Synthesis of Intermediate F35

### 4-(3-{5-[3-iodo-5-methyl-4-(propan-2-yl)-1H-pyrazol-1-yl]-6-methyl-2H-pyrazolo[3, 4-b]pyridin-2-yl)propyl)morpholine

### Synthesis of 4-(3-{5-[3-iodo-5-methyl-4-(propan-2-yl)-1H-pyrazol-1-yl]-6-methyl-2H-pyrazolo[3, 4-b]pyridin-2-yl)propyl)morpholine

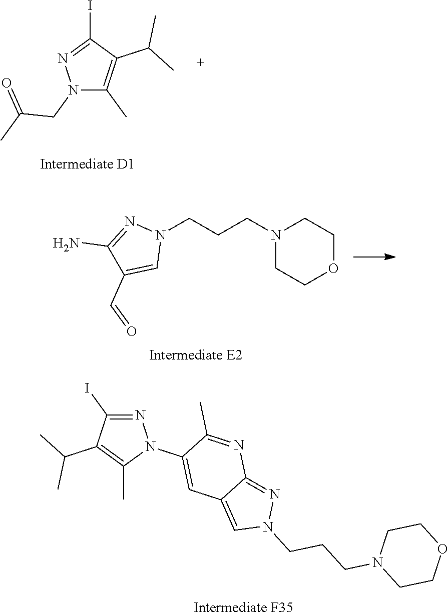 [SMILES: CC(=O)Cn1nc(I)c(C(C)C)c1C ; Nc1nn(CCCN2CCOCC2)cc1C=O ; Cc1nc2nn(CCCN3CCOCC3)cc2cc1-n1nc(I)c(C(C)C)c1C]

3-Amino-1-[3-(morpholin-4-yl)propyl]-1H-pyrazole-4-carbaldehyde (E2, 115 mg, 0.48 mmol) is dissolved in EtOH (2 mL). Piperidine (122 μL, 1.23 mmol) and 1-[3-iodo-5-methyl-4-(propan-2-yl)-1H-pyrazol-1-yl]propan-2-one (01, 150 mg, 0.49 mmol) are added and the reaction mixture is stirred at 80° C. for 6 h and at RT for 8 h. The reaction mixture is acidified with HOAc (2 mL) and TEA (0.5 mL), diluted with water and purified by reversed phase chromatography (HPLC; ACN/water including TEA) to afford the product F35.

Analysis (method F): Rₜ: 0.62 min, [M+H]⁺: 509

### Synthesis of Intermediate F36:6-cyclopropyl-5-[3-iodo-5-methyl-4-(propan-2-yl)-1H-pyrazol-1-yl]-2-methyl-2H-pyrazolo[3, 4-b]pyridin-7-ium-7-olate

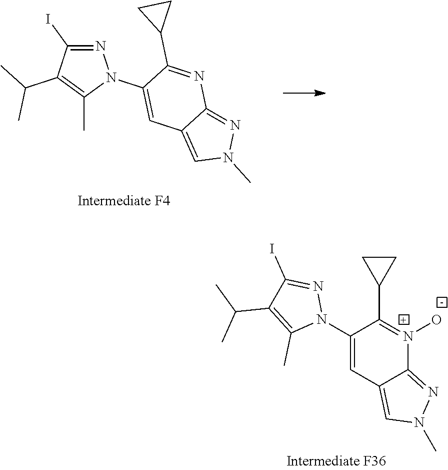

1-{6-Cyclopropyl-2-methyl-2H-pyrazolo[3, 4-b]pyridin-5-yl}-3-iodo-5-methyl-4-(propan-2-yl)-1H-pyrazole (F4) (600 mg, 1.42 mmol) is dissolved in DCM (3 mL). At 0° C. methyltrioxorhenium (VII) (71 mg, 0.28 mmol) and H₂O₂(890 μL, 15.7 mmol, 50%) are added and the reaction mixture is stirred at 0° C. for 3 h and at RT overnight. The reaction mixture is diluted with DCM and water. The organic phase is separated, dried (Na₂SO₄), filtered and concentrated. The residue is purified by flash chromatography (DCM/MeOH 100/0 to DCM/MeOH 90/10) to afford the product F36.

Analysis (method A): Rₜ: 0.57 min, [M+H]⁺: 438

### Synthesis of Intermediate F37

### 1-[6-(difluoromethyl)-2-methyl-2H-pyrazolo[3, 4-b]pyridin-5-yl]-3-iodo-5-methyl-4-(propan-2-yl)-1H-pyrazole

### Step 1: Synthesis of 5-bromo-6-(difluoromethyl)-2-methyl-2H-pyrazolo[3, 4-b]pyridine

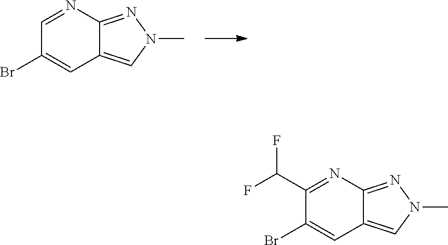

5-Bromo-2-methyl-2H-pyrazolo[3, 4-b]pyridine (1 g, 4.7 mmol) and zinc difluoromethane sulfinate (3.48 g, 11.8 mmol) are dissolved in DCM (50 mL) and water (10 mL). TFA (351 μL, 4.72 mmol) and tert-butyl hydroperoxide (3.26 mL, 24 mmol, 70% in water) are added and the reaction mixture is stirred at RT over the weekend. The reaction mixture is diluted with water and the layers are separated. The aqueous layer is extracted with DCM and the combined organic layers are dried (Na₂SO₄), filtered, and concentrated. The residue is purified by reversed phase chromatography (HPLC; ACN/water including TFA) to afford the product.

Analysis (method H): Rₜ: 0.84 min, [M+H]⁺: 262/264 (Br)

### Step 2: Synthesis of 6-(difluoromethyl)-5-(5, 5-dimethyl-1, 3, 2-dioxaborinan-2-yl)-2-methyl-2H-pyrazolo[3, 4-b]pyridine

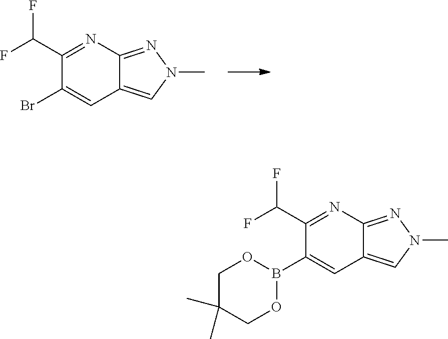

Under an argon atmosphere, 5-bromo-6-(difluoromethyl)-2-methyl-2H-pyrazolo[3, 4-b]pyridine (270 mg, 1.03 mmol) is dissolved in dioxane (10 mL). bis (neopentyl glycolato)diboron (512 mg, 2.27 mmol), potassium acetate (313 mg, 3.19 mmol) and Pd(dppf)Cl₂×DCM (84 mg, 0.10 mmol) are added and the reaction mixture is stirred at 80° C. for 2 h. After the reaction mixture is cooled to RT, it is filtered through a thiol-scavenging resin and the filtrate is concentrated. The residue is purified by reversed phase chromatography (HPLC; ACN/water including TFA) to afford the product.

Analysis (method G): Rₜ: 0.38 min, [M+H]⁺: 296 (mass of corresponding boronic acid)

### Step 3: Synthesis of 1-[6-(difluoromethyl)-2-methyl-2H-pyrazolo[3, 4-b]pyridin-5-yl]-3-iodo-5-methyl-4-(propan-2-yl)-1H-pyrazole

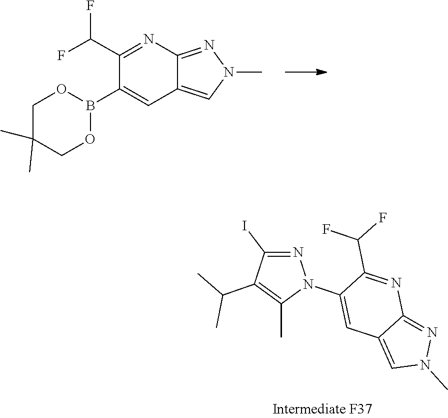

6-(Difluoromethyl)-5-(5, 5-dimethyl-1, 3, 2-dioxaborinan-2-yl)-2-methyl-2H-pyrazolo[3, 4-b]pyridine (75 mg, 0.25 mmol) and 3-iodo-5-methyl-4-(propan-2-yl)-1H-pyrazole (the synthesis is described in Intermediate A1 step 1) (60 mg, 0.24 mmol) are dissolved in DCM (2 mL) and ACN (5 mL). Boric acid (44.5 mg, 0.72 mmol), pyridine (106 μL, 1.34 mmol) and copper (II) acetate (109 mg, 0.60 mmol) are added and the reaction mixture is stirred in an open flask at RT for 2 h. The reaction mixture is quenched with aq. ammonia and is extracted 2× with DCM. The organic layer is dried (Na₂SO₄), filtered and concentrated. The residue is purified by reversed phase chromatography (HPLC; ACN/water including TFA) to afford the intermediate F37.

Analysis (method G): Rₜ: 1.08 min, [M+H]⁺: 432

### Synthesis of Intermediate F38

### 1-[8-Cyclopropyl-3-(difluoromethyl)-[1, 2, 4]triazolo[4, 3-a]pyridin-7-yl]-3-iodo-5-methyl-4-(propan-2-yl)-1H-pyrazole

### Step 1: Synthesis of 2-chloro-4-fluoro-3-iodopyridine

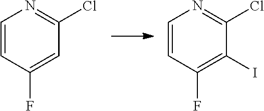 [SMILES: Fc1ccnc(Cl)c1 ; Fc1ccnc(Cl)c1I]

Under an argon atmosphere, LDA (5.44 mL, 10.9 mmol, 2 M) is cooled to −78° C. 2-chloro-4-fluoropyridine (1.00 mL, 9.9 mmol) in THE (25 mL) is added dropwise and the reaction mixture is stirred at −78° C. for 1 h. After that iodine (2.59 g, 9.9 mmol) in THE (35 mL) is added dropwise and the reaction mixture is stirred at −78° C. for 30 min. The reaction mixture is quenched with a saturated Na₂CO₃ solution and is extracted with MTBE. The organic layer is washed with Na₂S2O₃ and brine, dried (Na₂SO₄), filtered, and concentrated. The residue is purified by reversed phase chromatography (HPLC; ACN/water including TFA) to afford the product.

Analysis (method A): Rₜ: 0.51 min, [M+H]⁺: 258

### Step 2: Synthesis of 2-chloro-3-cyclopropyl-4-fluoropyridine

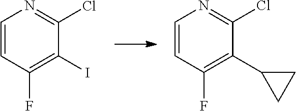 [SMILES: Fc1ccnc(Cl)c1I ; Fc1ccnc(Cl)c1C1CC1]

Under an argon atmosphere, 2-chloro-4-fluoro-3-iodopyridine (1.59 g, 6.19 mmol) and bromo(cyclopropyl)zinc (16.1 mL, 8.04 mmol, 0.5 M in THF) are mixed. Then PEPPSI(TM)-IPR (210 mg, 0.31 mmol) is added, and the reaction mixture is stirred at 50° C. for 1 h. The reaction mixture is quenched with a saturated NaHCO₃ solution and is extracted with DCM. The organic layer is washed with brine, dried (Na₂SO₄), filtered, and concentrated. The residue is purified by flash chromatography (DCM 100%) to afford the product.

Analysis (method A): Rₜ: 0.54 min, [M+H]⁺: 171/173 (Cl)

### Step 3: Synthesis of 2-chloro-3-cyclopropyl-4-[3-iodo-5-methyl-4-(propan-2-yl)-1H-pyrazol-1-yl]pyridine

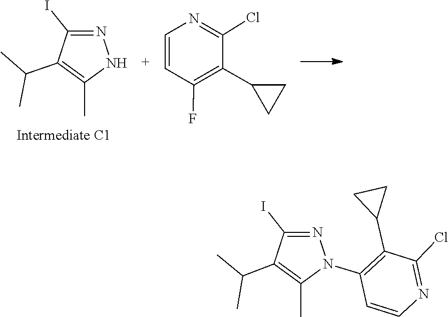

3-Iodo-5-methyl-4-(propan-2-yl)-1H-pyrazole (1.17 g, 4.66 mmol), 2-chloro-3-cyclopropyl-4-fluoropyridine (762 mg, 4.44 mmol) are dissolved in DMF (30 mL). K₂CO₃ (1.23 g, 8.9 mmol) is added, and the reaction mixture is stirred at 100° C. for 3 h and at RT over the weekend. The reaction mixture is purified by reversed phase chromatography (HPLC; ACN/water including TFA) to afford the product.

Analysis (method A): Rₜ: 0.77 min, [M+H]⁺: 402/404 (Cl)

### Step 4: Synthesis of 3-cyclopropyl-2-hydrazinyl-4-[3-iodo-5-methyl-4-(propan-2-yl)-1H-pyrazol-1-yl]pyridine

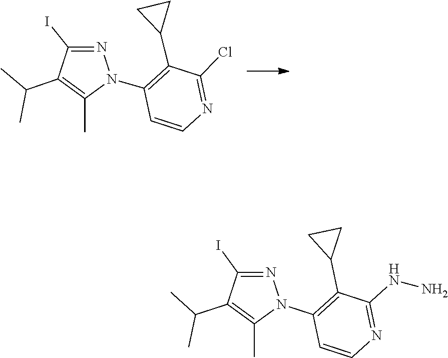 [SMILES: Cc1c(C(C)C)c(I)nn1-c1ccnc(Cl)c1C1CC1 ; Cc1c(C(C)C)c(I)nn1-c1ccnc(NN)c1C1CC1]

2-Chloro-3-cyclopropyl-4-[3-iodo-5-methyl-4-(propan-2-yl)-1H-pyrazol-1-yl]pyridine (513 mg, 1.28 mmol) is dissolved in EtOH (3 mL). Hydrazine (11.5 mL, 11.5 mmol, 1 M in THF) is added and the reaction mixture is stirred in the microwave at 150° C. for 5 h. The reaction mixture is purified by reversed phase chromatography (HPLC; ACN/water including TFA) to afford the product.

Analysis (method A): Rₜ: 0.50 min, [M+H]⁺: 398

### Step 5: Synthesis of 1-[8-cyclopropyl-3-(difluoromethyl)-[1, 2, 4]triazolo[4, 3-a]pyridin-7-yl]-3-iodo-5-methyl-4-(propan-2-yl)-1H-pyrazole

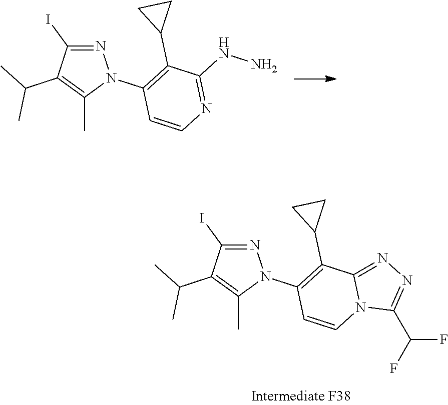 [SMILES: Cc1c(C(C)C)c(I)nn1-c1ccnc(NN)c1C1CC1 ; Cc1c(C(C)C)c(I)nn1-c1ccn2c(C(F)F)nnc2c1C1CC1]

3-Cyclopropyl-2-hydrazinyl-4-[3-iodo-5-methyl-4-(propan-2-yl)-1H-pyrazol-1-yl]pyridine (40 mg, 0.07 mmol) is dissolved in 2, 2-difluoroacetic acid (300 μL, 4.77 mmol). The reaction mixture is stirred in the microwave at 120° C. for 2 h. The reaction mixture is purified by reversed phase chromatography (HPLC; ACN/water including TFA) to afford the product F38.

Analysis (method A): Rₜ: 0.69 min, [M+H]⁺: 458

The intermediate compiled in the following table are obtained by following a procedure analogous to that described for Intermediate F38 (step 3-5).

| | | | | MS | |
|---|---|---|---|---|---|
| | | | | (ESI+): | |
| Interme- | Starting | | LC | m/z | t_{R} |
| diate | materials | Structure/Name | Method | [M + H]⁺ | [min] |
| F39 | 2-chloro-4- fluoro-3- methylpyridine (step 3) acetic acid (step 5) | 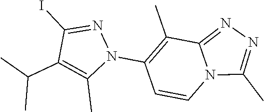 [SMILES: Cc1c(-n2nc(I)c(C(C)C)c2C)ccn2c(C)nnc12] | A | 396 | 0.52 |
| | | 1-{3,8-dimethyl-[1,2,4]triazolo[4,3- | | | |
| | | alpyridin-7-yl}-3-iodo-5-methyl-4- | | | |
| | | (propan-2-yl)-1H-pyrazole | | | |

### Synthesis of Intermediate F40: 4-bromo-5-cyclopropyl-2-{6-cyclopropyl-2-[(4-methoxyphenyl) methyl]-2H-pyrazolo[3, 4-b]pyridin-5-yl}-1-methyl-1H-imidazole

### Step 1: Synthesis of 5-cyclopropyl-2-{6-cyclopropyl-2-[(4-methoxyphenyl) methyl]-2H-pyrazolo[3, 4-b]pyridin-5-yl}-1-methyl-1H-imidazole

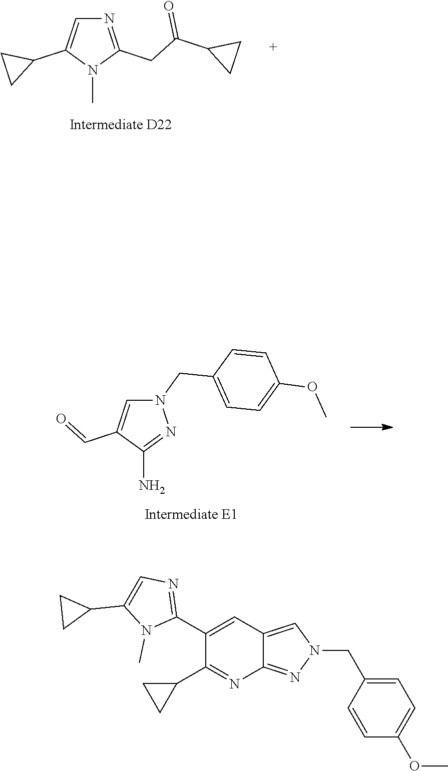 [SMILES: Cn1c(C2CC2)cnc1CC(=O)C1CC1 ; COc1ccc(Cn2cc(C=O)c(N)n2)cc1 ; COc1ccc(Cn2cc3cc(-c4ncc(C5CC5)n4C)c(C4CC4)nc3n2)cc1]

1-Cyclopropyl-2-(5-cyclopropyl-1, 2-dimethyl-1H-imidazol-4-yl) ethan-1-one (D22) (1.40 g, 5.48 mmol, 80% purity) and 3-amino-1-[(4-methoxyphenyl) methyl]-1H-pyrazole-4-carbaldehyde (E1) (1.65 g, 7.13 mmol) are dissolved in EtOH (35 mL). Piperidine (1.62 mL, 16.5 mmol) is added, and the reaction mixture is stirred at 80° C. overnight. The reaction mixture is concentrated and co-evaporated 3× with toluene. The residue is purified by flash chromatography (DCM/acetone 100/0 to DCM/Acetone 30/70) to afford the product.

Analysis (method Q): Rₜ: 1.40 min, [M+H]⁺: 400

### Step 2: Synthesis of 4-bromo-5-cyclopropyl-2-{6-cyclopropyl-2-[(4-methoxyphenyl) methyl]-2H-pyrazolo[3, 4-b]pyridin-5-yl}-1-methyl-1H-imidazole

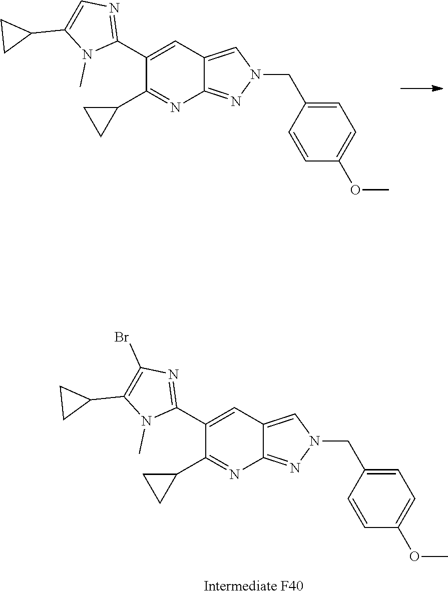 [SMILES: COc1ccc(Cn2cc3cc(-c4ncc(C5CC5)n4C)c(C4CC4)nc3n2)cc1 ; COc1ccc(Cn2cc3cc(-c4nc(Br)c(C5CC5)n4C)c(C4CC4)nc3n2)cc1]

5-Cyclopropyl-2-{6-cyclopropyl-2-[(4-methoxyphenyl) methyl]-2H-pyrazolo[3, 4-b]pyridin-5-yl}-1-methyl-1H-imidazole (0.60 g, 1.5 mmol) is dissolved in DCM (21 mL). NBS (0.29 g, 1.65 mmol) is added at 0° C. and the reaction mixture is stirred at RT for 30 min. The reaction mixture is quenched with a saturated Na₂S₂O₃ solution and extracted with DCM. The organic layer is washed with a saturated K₂CO₃ solution, dried (Na₂SO₄), filtered, and concentrated. The residue is purified by flash chromatography (DCM/Acetone 100/0 to DCM/Acetone 90/10) to afford the product F40.

Analysis (method R): Rₜ: 3.82 min, [M+H]⁺: 478/480 (Br)

### Synthesis of Intermediate F41: 4-bromo-5-cyclopropyl-1-methyl-2-[2-methyl-6-(propan-2-yl)-2H-pyrazolo[3, 4-b]pyridin-5-yl]-1H-imidazole

### Step 1

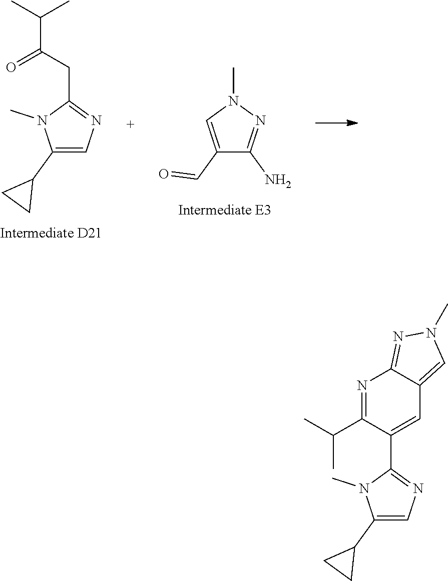

1-(5-Cyclopropyl-1-methyl-1H-imidazol-2-yl)-3-methylbutan-2-one (1.28 g, 5 mmol), 3-amino-1-methyl-1 h-pyrazole-4-carbaldehyde (684 mg, 5.5 mmol), piperidine (984 μL, 10 mmol) is dissolved in ethanol and heated at 95° C. overnight. The mixture is then purified by reversed phase preparative HPLC (Xbridge C18, ACN/water including NH3) to yield the desired product.

Analysis (method A): Rₜ: 0.32 min, [M+H]⁺: 296

### Step 2

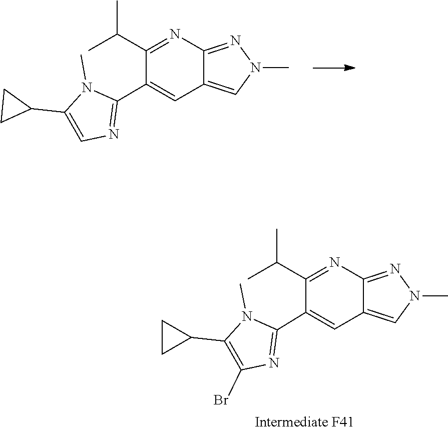 [SMILES: CC(C)c1nc2nn(C)cc2cc1-c1ncc(C2CC2)n1C ; CC(C)c1nc2nn(C)cc2cc1-c1nc(Br)c(C2CC2)n1C]

5-Cyclopropyl-1-methyl-2-[2-methyl-6-(propan-2-yl)-2H-pyrazolo[3, 4-b]pyridin-5-yl]-1H-imidazole (2.04 g, 6.9 mmol) is dissolved in DCM (21 mL). NBS (1.23 g, 6.9 mmol) is added at 0° C., and after 15 min. the reaction mixture is stirred at RT for 1 h. The reaction mixture is quenched with a saturated NaHCO₃ solution, the phases are separated, and the water phase is extracted with DCM. The combined organic phases are dried (Na₂SO₄), filtered, and concentrated. The residue is purified by flash chromatography (EtOAc/MeOH 100/0 to EtOAc/MeOH 95/5) to afford the desired product F41.

Analysis (method A): Rₜ: 0.44 min, [M+H]⁺: 374/376 (Br)

The intermediates compiled in the following table are obtained by following a procedure analogous to that described for Intermediate F41.

| | Starting | | | MS | |
|---|---|---|---|---|---|
| | materials | | | (ESI+): | |
| Interme- | for | | LC | m/z | t_{R} |
| diate | step 1 | Structure/Name | Method | [M + H]⁺ | [min] |
| F42 | E3 and D22 | 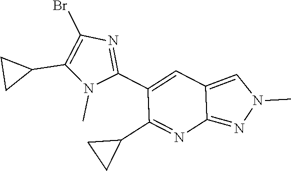 [SMILES: Cn1cc2cc(-c3nc(Br)c(C4CC4)n3C)c(C3CC3)nc2n1] | A | 372/374 (Br) | 0.44 |
| | | 4-bromo-5-cyclopropyl-2-{6-cyclopropyl-2- | | | |
| | | methyl-2H-pyrazolo[3,4-b]pyridin-5-yl}-1- | | | |
| | | methyl-1H-imidazole | | | |
| F43 | E3 + D23 | 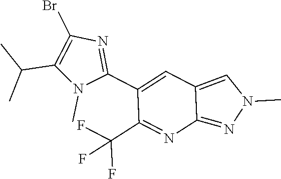 [SMILES: CC(C)c1c(Br)nc(-c2cc3cn(C)nc3nc2C(F)(F)F)n1C] | H | 402/404 (Br) | 0.96 |
| | | 4-bromo-1-methyl-2-[2-methyl-6- | | | |
| | | (trifluoromethyl)-2H-pyrazolo[3,4-b]pyridin-5- | | | |
| | | yl]-5-(propan-2-yl)-1H-imidazole | | | |
| F44 | E3 + D25 | 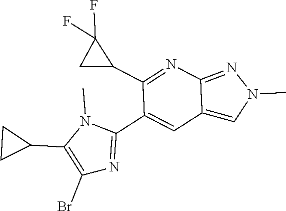 [SMILES: Cn1cc2cc(-c3nc(Br)c(C4CC4)n3C)c(C3CC3(F)F)nc2n1] | G | 408/410 | 0.88 |
| | | 4-bromo-5-cyclopropyl-2-[6-(2,2- | | | |
| | | difluorocyclopropyl)-2-methyl-2H-pyrazolo[3, | | | |
| | | 4-b]pyridin-5-yl]-1-methyl-1H-imidazole | | | |

### Synthesis of Intermediate F45: 5-(4-bromo-5-cyclopropyl-1-methyl-1H-imidazol-2-yl)-6-(difluoromethoxy)-2-methyl-2H-indazole

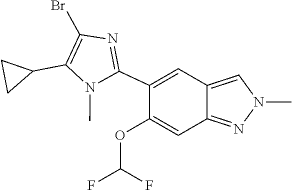 [SMILES: Cn1cc2cc(-c3nc(Br)c(C4CC4)n3C)c(OC(F)F)cc2n1]

### Intermediate F45

### Step 1: Synthesis of 4-(difluoromethoxy)-1-methyl-2-nitrobenzene

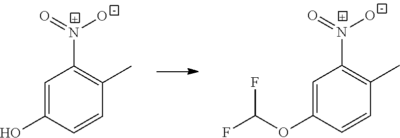

KOH (14.7 g, 262 mmol) is dissolved in ACN/water 1/1 (100 mL), 4-methyl-3-nitrophenol (2 g, 13.06 mmol) is added and the reaction mixture is frozen with a dry ice/acetone bath. Once frozen solid, bromodifluoromethyl diethylphosphonate (3.77 mL, 21.2 mmol) is added on top and the reaction mixture is left at this temperature for 10 min. Then the ice bath is removed. 1.5 h later, once the reaction has slowly thawed, resumed stirring, and warmed to 10° C., the reaction is completed. The reaction mixture is diluted with diethyl ether and water and stirred vigorously. It is extracted 2× with diethyl ether and the combined organic layers are dried (Na₂SO₄), filtered, and concentrated. The residue is passed through a small plug of silica gel (CycH/EtOAc 90/10→CycH/EtOAc 0/100) to afford the product.

Analysis (method F): Rₜ: 0.86 min

### Step 2: Synthesis of 5-(difluoromethoxy)-2-methylaniline

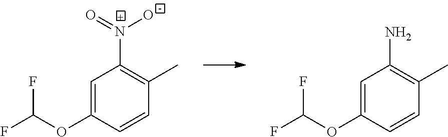

4-(Difluoromethoxy)-1-methyl-2-nitrobenzene (1.8 g, 8.86 mmol) is dissolved in EtOH (25 mL), ammonium formate (2.45 g, 17.7 mmol) and palladium on carbon (1.60 g, 10%) are added and the reaction mixture is stirred in a sealed vial at 85° C. overnight. The reaction mixture is filtered and concentrated. The residue is purified by flash chromatography (DCM/MeOH 100/0 to DCM/MeOH 90/10) to afford the product.

Analysis (method F): Rₜ: 0.52 min, [M+H]⁺: 174

### Step 3: Synthesis of 4-bromo-5-(difluoromethoxy)-2-methylaniline

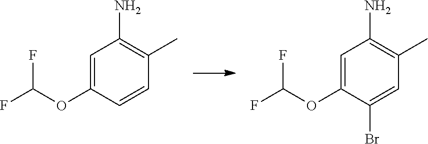

5-(Difluoromethoxy)-2-methylaniline (1.12 g, 6.47 mmol) is dissolved in chloroform (51.5 mL). At 0° C. NBS (1.15 g, 6.47 mmol) is added, and the reaction mixture is stirred at 4-6° C. overnight. The reaction mixture is concentrated, and the residue is purified by flash chromatography (DCM/MeOH 100/0 to DCM/MeOH 90/10) to afford the product.

Analysis (method F): Rₜ: 0.83 min, [M+H]⁺: 252/254 (Br)

### Step 4: Synthesis of 5-bromo-6-(difluoromethoxy)-2H-indazole

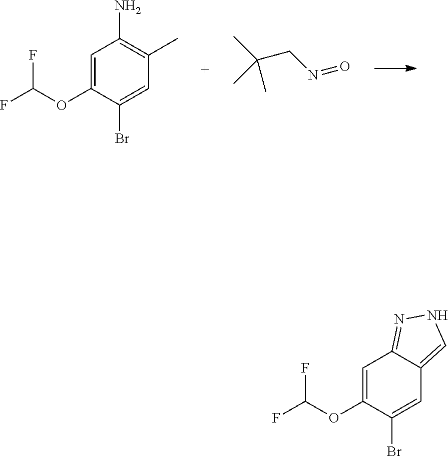

To boron trifluoride etherate (1.45 mL, 11.8 mmol) in DCM (18.8 mL) Under an argon atmosphere at −78° C. is added 4-bromo-5-(difluoromethoxy)-2-methylaniline (1.98 g, 7.84 mmol) in DCM (11 mL) followed by dropwise addition of tert-butylnitrite (1.12 mL, 9.4 mmol). The reaction mixture is allowed to warm to RT and is stirred for 22 h. To the above solution of the diazonium salt in DCM, potassium acetate (1.46 g, 14.9 mmol) and 18-crown-6 (104 mg, 0.39 mmol) is added. After stirring at RT for 1.5 h, the mixture is filtered and the solid is washed with DCM. The filtrate is concentrated and purified by flash chromatography (CycH/EtOAc 100/0 to CycH/EtOAc 0/100) to afford the product.

Analysis (method F): Rₜ: 0.78 min, [M+H]⁺: 263/265 (Br)

### Step 5: Synthesis of 5-bromo-6-(difluoromethoxy)-2-methyl-2H-indazole

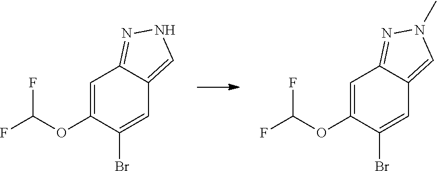

5-Bromo-6-(difluoromethoxy)-2H-indazole (960 mg, 3.07 mmol, 84% purity) is dissolved in EtOAc (19 mL). Trimethyloxonium tetrafluoroborate (589 mg, 3.99 mmol) is added and the reaction mixture is stirred at RT overnight. The reaction mixture is quenched by the dropwise addition of a 10% NaHCO₃ solution until a basic pH is reached. The reaction mixture is extracted with DCM. The organic layer is dried (Na₂SO₄), filtered, and concentrated. The residue is purified by flash chromatography (DCM/MeOH 100/0 to DCM/MeOH 90/10) to afford the product.

Analysis (method F): Rₜ: 0.79 min, [M+H]⁺: 277/279 (Br)

### Step 6: Synthesis of 6-(difluoromethoxy)-2-methyl-5-(4, 4, 5, 5-tetramethyl-1, 3, 2-dioxaborolan-2-yl)-2H-indazole

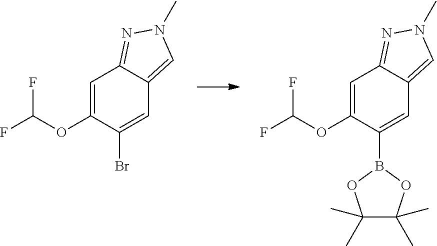 [SMILES: Cn1cc2cc(Br)c(OC(F)F)cc2n1 ; Cn1cc2cc(B3OC(C)(C)C(C)(C)O3)c(OC(F)F)cc2n1]

Under an argon atmosphere, 5-bromo-6-(difluoromethoxy)-2-methyl-2H-indazole (700 mg, 2.53 mmol) is dissolved in dioxane (18 mL), bis(pinacolato)diboron (0.74 g, 2.91 mmol), potassium acetate (744 mg, 7.58 mmol) and Pd(dppf)Cl₂×DCM (206 mg, 0.25 mmol) are added, and the reaction mixture is stirred at 90° C. for 3 h. The reaction mixture is diluted with DCM and water and filtered over celite. The layers are separated, and the organic layer is dried (Na₂SO₄), filtered, and concentrated. The residue is purified by reversed phase chromatography (HPLC; ACN/water including TFA) to afford the product.

Analysis (method F): Rₜ: 0.90 min, [M+H]⁺: 325

### Step 7: Synthesis of 5-cyclopropyl-1-methyl-1H-imidazole

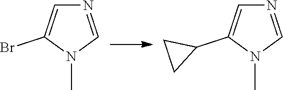

Under an argon atmosphere, 5-bromo-1-methyl-1H-imidazole (7.50 g, 46.6 mmol), cyclopropyl zinc bromide (132 mL, 66 mmol, 0.5 M in THF) and Pd(dppf)Cl₂ (2.20 g, 3 mmol) are mixed together, and the reaction mixture is stirred at 70° C. for 20 h. The reaction mixture is concentrated, and the residue is purified by flash chromatography (DCM/MeOH 100/0 to DCM/MeOH 90/10) to afford the product.

Analysis (method C): Rₜ: 0.31 min, [M+H]⁺: 123

### Step 8: Synthesis of 2, 4-dibromo-5-cyclopropyl-1-methyl-1H-imidazole

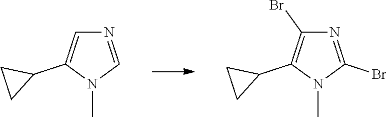

5-Cyclopropyl-1-methyl-1H-imidazole (9.20 g, 52.7 mmol,) is dissolved in ACN (200 mL). At −5° C. NBS (18.8 g, 105 mmol) is added in portions and the reaction mixture is stirred at −5° C. for 30 min and at RT for 4 h. The reaction mixture is quenched by the addition of a saturated Na₂S₂O₃ solution (40 mL, 4.4 M). The formed precipitate is filtered and washed with ACN. The filtrate is extracted with EtOAc and the organic layer is dried (Na₂SO₄), filtered and concentrated. The residue is purified by flash chromatography (CycH/EtOAc 95/5 to CycH/EtOAc 65/35) to afford the product.

Analysis (method F): Rₜ: 0.77 min, [M+H]⁺: 279/281/283 (2×Br)

### Step 9: Synthesis of 5-(4-bromo-5-cyclopropyl-1-methyl-1H-imidazol-2-yl)-6-(difluoro-methoxy)-2-methyl-2H-indazole

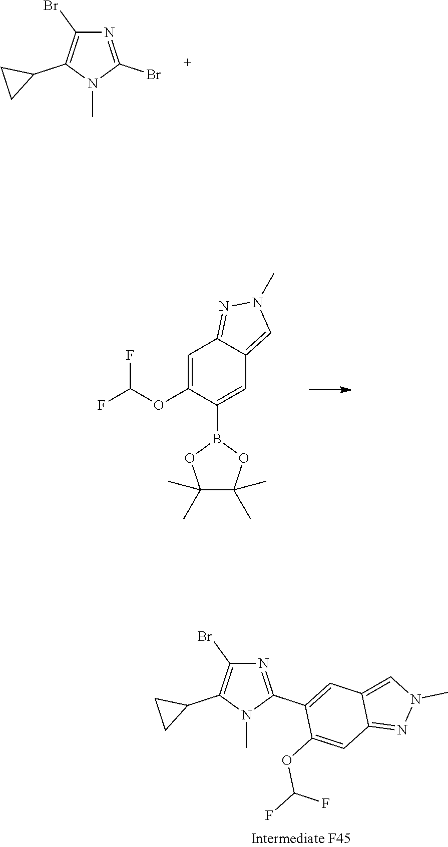 [SMILES: Cn1c(Br)nc(Br)c1C1CC1 ; Cn1cc2cc(B3OC(C)(C)C(C)(C)O3)c(OC(F)F)cc2n1 ; Cn1cc2cc(-c3nc(Br)c(C4CC4)n3C)c(OC(F)F)cc2n1]

Under an argon atmosphere, 2, 4-dibromo-5-cyclopropyl-1-methyl-1H-imidazole (108 mg, 0.39 mmol) and 6-(difluoromethoxy)-2-methyl-5-(4, 4, 5, 5-tetramethyl-1, 3, 2-dioxaborolan-2-yl)-2H-indazole (150.05 mg, 0.46 mmol) are dissolved in dioxane (3.6 mL). Cs₂CO₃ (377 mg, 1.16 mmol) and Pd(PPh₃)₄ (44.6 mg, 0.04 mmol) are added and the reaction mixture is stirred at 80° C. overnight. The reaction mixture is concentrated and purified by flash chromatography (DCM/MeOH 100/0 to DCM/MeOH 85/15). The combined fractions are concentrated, and the residue is triturated with CycH to afford the product F45.

Analysis (method F): Rₜ: 0.61 min, [M+H]⁺: 397/399 (Br)

### Synthesis of Intermediate F46: 4-bromo-5-cyclopropyl-2-{6-cyclopropyl-2-propyl-2H-pyrazolo[3, 4-b]pyridin-5-yl}-1-methyl-1H-imidazole

### Step 1: Synthesis of 4-bromo-5-cyclopropyl-2-{6-cyclopropyl-2H-pyrazolo[3, 4-b]pyridin-5-yl}-1-methyl-1H-imidazole

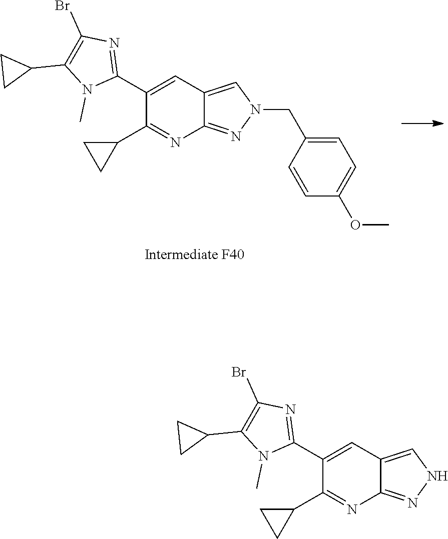 [SMILES: COc1ccc(Cn2cc3cc(-c4nc(Br)c(C5CC5)n4C)c(C4CC4)nc3n2)cc1 ; Cn1c(-c2cc3c[nH]nc3nc2C2CC2)nc(Br)c1C1CC1]

4-Bromo-5-cyclopropyl-2-{6-cyclopropyl-2-[(4-methoxyphenyl)methyl]-2H-pyrazolo[3, 4-b]pyridin-5-yl}-1-methyl-1H-imidazole (F40) (574 mg, 1.20 mmol) and anisole (264 μL, 2.40 mmol) are dissolved in DCE (5 mL) and TFA (3 mL). The reaction mixture is stirred at 60° C. over the weekend. The reaction mixture is concentrated and purified by reversed phase chromatography (HPLC; ACN/water/TFA). The fractions are combined, and the solvent is evaporated. The aqueous residue is neutralized with a 2 M K₂CO₃ solution. The formed precipitate is filtered, washed with water, and dried in an oven to afford of the product.

Analysis (method H): Rₜ: 0.96 min, [M+H]⁺: 358/360 (Br)

### Step 2: Synthesis of 4-bromo-5-cyclopropyl-2-{6-cyclopropyl-2-propyl-2H-pyrazolo[3, 4-b]pyridin-5-yl}-1-methyl-1H-imidazole

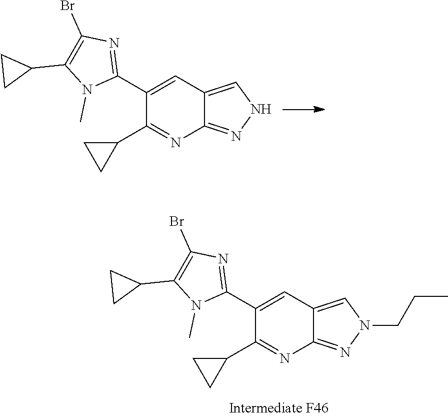 [SMILES: Cn1c(-c2cc3c[nH]nc3nc2C2CC2)nc(Br)c1C1CC1 ; CCCn1cc2cc(-c3nc(Br)c(C4CC4)n3C)c(C3CC3)nc2n1]

4-Bromo-5-cyclopropyl-2-{6-cyclopropyl-2H-pyrazolo[3, 4-b]pyridin-5-yl}-1-methyl-1H-imidazole (358 mg, 1 mmol) is dissolved in ACN (5 mL). K₂CO₃ (346 mg, 2.50 mmol) and 1-bromopropane (91 μL, 1 mmol) are added and the reaction mixture is stirred at 60° C. overnight. The reaction mixture is filtered and purified by reversed phase chromatography (HPLC; C18, ACN/water including NH₃) to afford the product F46.

Analysis (method H): Rₜ: 1.01 min, [M+H]⁺: 400/402 (Br)

### Synthesis of Intermediate F47

### 4-bromo-5-cyclopropyl-2-{6-ethyl-2-methyl-2H-pyrazolo[3, 4-b]pyridin-5-yl}-1-methyl-1H-imidazole

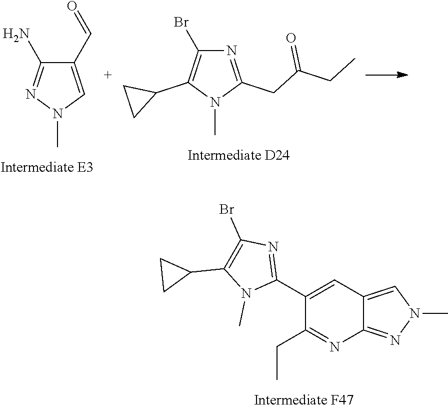

1-(4-Bromo-5-cyclopropyl-1-methyl-1H-imidazol-2-yl)butan-2-one (5.51 g, 20.3 mmol) is dissolved in EtOH (60 mL). 3-Amino-1-methyl-1-H-pyrazole-4-carbaldehyde (2.80 g, 22.4 mmol) and piperidine (4.02 mL, 40.6 mmol) are added and the reaction mixture is stirred at 90° C. overnight. The reaction mixture is filtered and purified by reversed phase chromatography (HPLC; Xbridge-C18, ACN/water including NH3) to afford the product F47.

Analysis (method F): Rₜ: 0.55 min, [M+H]⁺: 360/362 (Br)

### Synthesis of Intermediate F48: 1-{3, 8-dimethylimidazo[1, 2-a]pyridin-7-yl}-3-iodo-5-methyl-4-(propan-2-yl)-1H-pyrazole

### Step 1: Synthesis of 7-fluoro-3, 8-dimethylimidazo[1, 2-a]pyridine

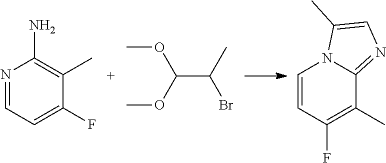

4-Fluoro-3-methylpyridin-2-amine (300 mg, 2.4 mmol) and 2-bromo-1, 1-dimethoxypropane (1.6 mL, 11.9 mmol), p-toluolsulfonic acid (82 mg, 0.48 mmol) is dissolved in 12 mL acetonitrile and heated at 80° C. for 2 days. The mixture is then diluted with DCM and purified by preparative flash column chromatography to yield 330 mg 7-fluoro-3, 8-dimethylimidazo[1, 2-a]pyridine.

Analysis (method C): Rₜ: 0.41 min, [M+H]⁺: 165

### Step 2: Synthesis of 1-{3, 8-dimethylimidazo[1, 2-a]pyridin-7-yl}-3-iodo-5-methyl-4-(propan-2-yl)-1H-pyrazole

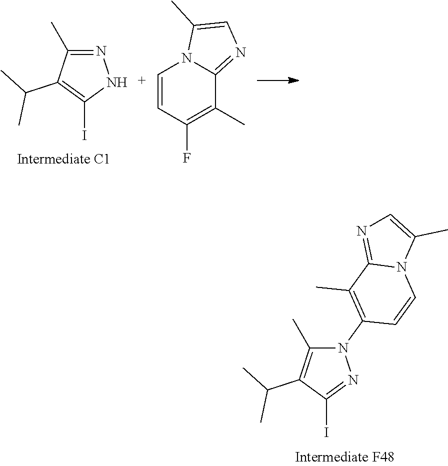

3-Iodo-4-isopropyl-5-methyl-1H-pyrazole (C1) (450 mg, 1.8 mmol) is dissolved in NMP (1 mL), then NaH (157 mg) is added and the mixture is stirred for 5 min. 7-fluoro-3, 8-dimethylimidazo[1, 2-a]pyridine (295 mg, 1.8 mmol) is added, and the mixture is placed for 16 h in the microwave at 170° C. After cooling the mixture is diluted with ACN/water, filtered, and purified by preparative HPLC to yield the intermediate F48.

Analysis (method C): Rₜ: 0.69 min, [M+H]⁺: 395

### Synthesis of Intermediate F49: 2-(1-{2, 6-dimethyl-2H-pyrazolo[3, 4-b]pyridin-5-yl}-3-iodo-5-methyl-1H-pyrazol-4-yl)propan-1-ol

### Step 1: Synthesis of ethyl 2-(1-{2, 6-dimethyl-2H-pyrazolo[3, 4-b]pyridin-5-yl}-3-iodo-5-methyl-1H-pyrazol-4-yl) propanoate

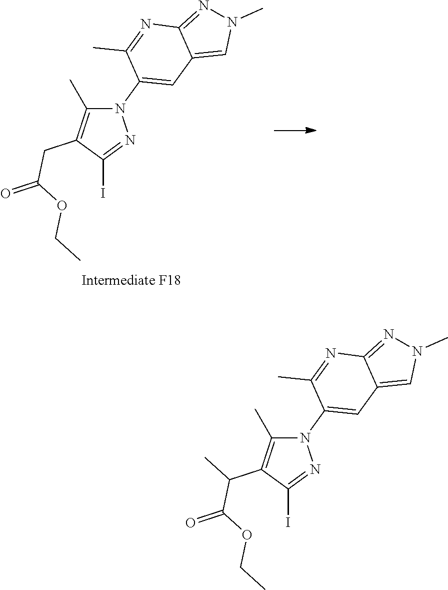 [SMILES: CCOC(=O)Cc1c(I)nn(-c2cc3cn(C)nc3nc2C)c1C ; CCOC(=O)C(C)c1c(I)nn(-c2cc3cn(C)nc3nc2C)c1C]

Ethyl-2-(1-{2, 6-dimethyl-2H-pyrazolo[3, 4-b]pyridin-5-yl}-3-iodo-5-methyl-1H-pyrazol-4-yl)acetate (138 mg, 0.31 mmol), is dissolved in THE (5 mL) and cooled to −78° C. Mel (78 μL, 1.3 mmol) and lithium bis(trimethylsilyl)amide (408 μL, 0.41 mmol) is added and the mixture warmed slowly to RT and stirring continued at RT for 1 h. NH₄Cl aqueous solution (15 mL) is added and the mixture is extracted 3× with EtOAc, the organic phase dried (Na₂SO₄) and concentrated. The product is used in the next step without further purification.

Analysis (method G): Rₜ: 0.81 min, [M+H]⁺: 454

### Step 2: Synthesis of 2-(1-{2, 6-dimethyl-2H-pyrazolo[3, 4-b]pyridin-5-yl}-3-iodo-5-methyl-1H-pyrazol-4-yl) propanoic acid

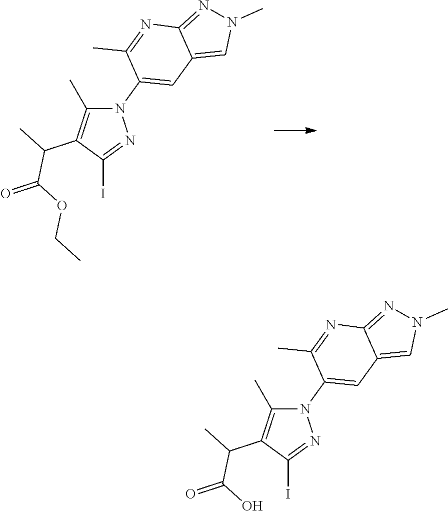 [SMILES: CCOC(=O)C(C)c1c(I)nn(-c2cc3cn(C)nc3nc2C)c1C ; Cc1nc2nn(C)cc2cc1-n1nc(I)c(C(C)C(=O)O)c1C]

Ethyl 2-(1-{2, 6-dimethyl-2H-pyrazolo[3, 4-b]pyridin-5-yl)-3-iodo-5-methyl-1H-pyrazol-4-yl) propanoate (68 mg, 0.15 mmol) is dissolved in THE (2 mL) and NaOH (94 μL, 4M, 0.375 mmol). Then 2 drops of methanol are added, and the mixture is stirred for 3 h at 50° C. The mixture is then neutralized with HCl (4M), concentrated, dissolved in water/DMF, and purified by preparative HPLC to yield the desired product.

Analysis (method G): Rₜ: 0.65 min [M+H]⁺: 426

### Step 3: Synthesis of 2-(1-{2, 6-dimethyl-2H-pyrazolo[3, 4-b]pyridin-5-yl}-3-iodo-5-methyl-1H-pyrazol-4-yl)propan-1-ol

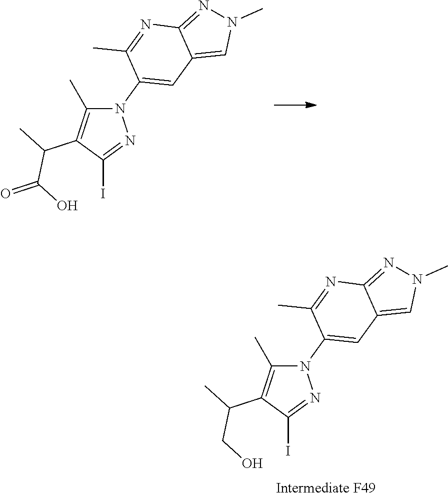 [SMILES: Cc1nc2nn(C)cc2cc1-n1nc(I)c(C(C)C(=O)O)c1C ; Cc1nc2nn(C)cc2cc1-n1nc(I)c(C(C)CO)c1C]

2-(1-{2, 6-Dimethyl-2H-pyrazolo[3, 4-b]pyridin-5-yl}-3-iodo-5-methyl-1H-pyrazol-4-yl) propanoic acid (102 mg, 0.24 mmol) is suspended in THE (2 mL) and DMF (200 μL), then triethylamine (101 μL, 0.72 mmol) and CDI (86 mg, 0.528 mmol) is added, and the mixture stirred for 45 min at RT. The mixture is then cooled with an ice bath, NaBH₄ (32 mg, 0.84 mmol) and water is added, and the mixture stirred for 2 h while warming to RT. The mixture is concentrated, then dissolved in DMF/water and purified by preparative HPLC to yield the product F49.

Analysis (method G): Rₜ: 0.66 min, [M+H]⁺: 412

### Synthesis of Intermediate F50: 5-[3-iodo-5-methyl-4-(propan-2-yl)-1H-pyrazol-1-yl]-2, 6-dimethyl-1H-1, 3-benzodiazole

### Step 1: Synthesis of 1-(5-chloro-2-methyl-4-nitrophenyl)-3-iodo-5-methyl-4-(propan-2-yl)-1H-pyrazole

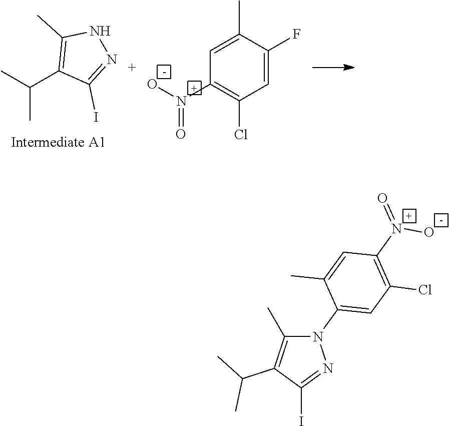

3-Iodo-4-isopropyl-5-methyl-1H-pyrazole (600 mg, 2.4 mmol) is dissolved in DMF (15 mL) and K₂CO₃ and 4-chloro-2-fluoro-5-nitrotoluene (909 mg, 4.8 mmol) is added and the mixture stirred for 2 h at 100° C. The mixture is then purified by preparative HPLC to yield the desired product.

Analysis (method G): Rₜ: 1.26 min, [M+H]⁺: 420

### Step 2: Synthesis of 5-[3-Iodo-5-methyl-4-(propan-2-yl)-1H-pyrazol-1-yl]-4-methyl-2-nitroaniline

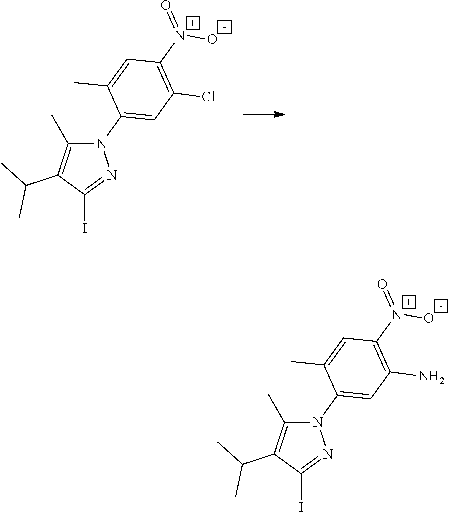

1-(5-Chloro-2-methyl-4-nitrophenyl)-3-iodo-5-methyl-4-(propan-2-yl)-1H-pyrazole (900 mg, 2.1 mmol), NMP (12 mL) and concentrated aq. ammonia (20 mL) is heated at 175° C. for 3 h and the mixture is purified by preparative HPLC to yield the desired product.

Analysis (method H): Rₜ: 1.18 min, [M+H]⁺: 401

### Step 3: Synthesis of 4-[3-iodo-5-methyl-4-(propan-2-yl)-1H-pyrazol-1-yl]-5-methylbenzene-1, 2-diamine

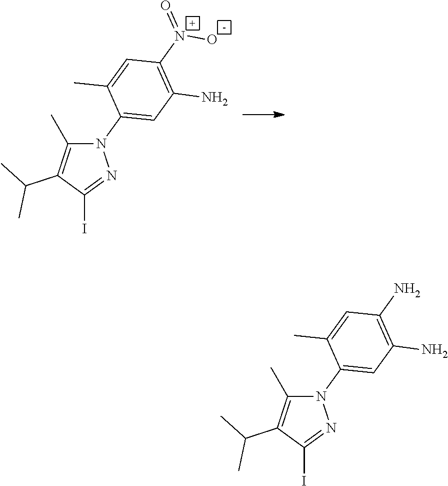

5-[3-Iodo-5-methyl-4-(propan-2-yl)-1H-pyrazol-1-yl]-4-methyl-2-nitroaniline (372 mg, 0.93 mmol) is dissolved in HOAc (10 mL) and iron powder (260 mg) is added. Then HCl (4M, 3×1.1 mL) is added, and the mixture is stirred for 3 h at 60° C. The mixture is purified by preparative HPLC to yield the desired product.

Analysis (method G): Rₜ: 0.89 min, [M+H]⁺: 371

### Step 4: Synthesis of 5-[3-iodo-5-methyl-4-(propan-2-yl)-1H-pyrazol-1-yl]-2, 6-dimethyl-1H-1, 3-benzodiazole

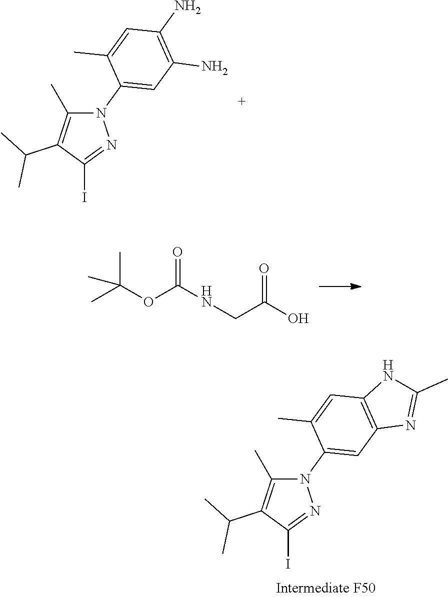 [SMILES: Cc1cc(N)c(N)cc1-n1nc(I)c(C(C)C)c1C ; CC(C)(C)OC(=O)NCC(=O)O ; Cc1nc2cc(-n3nc(I)c(C(C)C)c3C)c(C)cc2[nH]1]

4-[3-Iodo-5-methyl-4-(propan-2-yl)-1H-pyrazol-1-yl]-5-methylbenzene-1, 2-diamine (132 mg, 0.3 mmol) and {[(tert-butoxy)carbonyl]amino}acetic acid (53 mg, 0.3 mmol) is dissolved in DMF (2 mL), then HATU (114 mg, 0.3 mmol) and DIPEA (206 μL, 1.2 mmol) is added and the mixture is stirred for 2 h at RT. The mixture is filtered through basic aluminum oxide, and then washed with DMF/MeOH 9:1 and concentrated. The mixture is dissolved in HOAc (3 mL) and heated at 85° C. for 2 h. The mixture is then purified by preparative HPLC (C18, ACN/water including NH₃) to yield the desired product F50.

Analysis (method G): R: 0.89 min, [M+H]⁺: 395

### Synthesis of Intermediate F51: 5-(4-bromo-5-cyclopropyl-1-methyl-1H-imidazol-2-yl)-6-(trifluoromethoxy)-2-methyl-2H-indazole

### Step 1: Synthesis of 4-bromo-5-(difluoromethoxy)-2-methylaniline

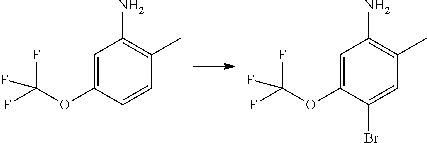

5-(Trifluoromethoxy)-2-methylaniline (656 mg) is dissolved in chloroform (25 mL). At 0° C. NBS (611 mg) is added, and the reaction mixture is stirred at 0° C. for 1 h. The reaction mixture is quenched with Na₂S₂O₃ (0.5 M solution) and diluted with DCM. The phases are separated, the organic layer is concentrated and purified by flash chromatography (DCM/MeOH 100/0 to DCM/MeOH 90/10) to afford the product.

Analysis (method F): Rₜ: 0.96 min, [M+H]⁺: 270/272 (Br)

### Step 2: Synthesis of 5-bromo-6-(trifluoromethoxy)-2H-indazole

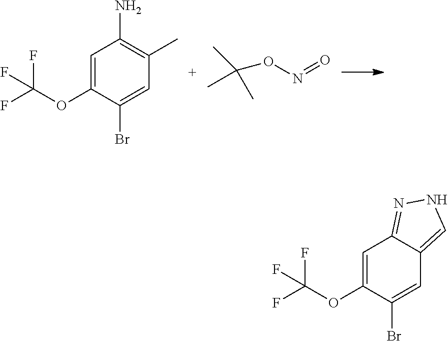

To boron trifluoride etherate (0.53 mL, 4.3 mmol) in DCM (8 mL) under an argon atmosphere at −78° C. is added 4-bromo-5-(trifluoromethoxy)-2-methylaniline (1.98 g, 7.84 mmol) in DCM (11.25 mL) followed by dropwise addition of tert-butylnitrite (0.41 mL, 3.4 mmol). The reaction mixture is allowed to warm to RT and is stirred for 22 h. To the above solution of the diazonium salt in DCM is added potassium acetate (531 mg, 5.4 mmol) and 18-crown-6 (38 mg, 0.14 mmol). After stirring at RT for 2 h, the mixture is filtered and the solid washed with DCM. The filtrate is concentrated, and the residue is purified by flash chromatography (DCM/MeOH 100/0 to DCM/MeOH 90/10) to afford the product.

Analysis (method F): Rₜ: 0.88 min, [M+H]⁺: 281/283 (Br)

### Step 3: Synthesis of 5-bromo-6-(trifluoromethoxy)-2-methyl-2H-indazole

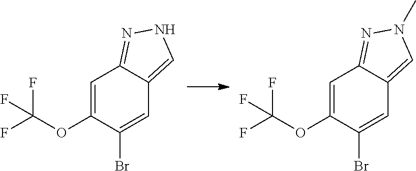 [SMILES: FC(F)(F)Oc1cc2n[nH]cc2cc1Br ; Cn1cc2cc(Br)c(OC(F)(F)F)cc2n1]

5-Bromo-6-(trifluoromethoxy)-2H-indazole (500 mg, 1.6 mmol) is dissolved in EtOAc (10 mL). Trimethyloxonium tetrafluoroborate (308 mg, 2.1 mmol) is added and the reaction mixture is stirred at RT for 1 h. The reaction mixture is quenched by the dropwise addition of a 10% NaHCO₃ solution until a basic pH is reached. The reaction mixture is diluted with DCM and passed over a phase separator. The organic layer is adsorbed on Extrelute and purified by MPLC (DCM/MeOH 100/0→DCM/MeOH 90/10) to afford the product.

Analysis (method F): Rₜ: 0.93 min, [M+H]⁺: 295/297 (Br)

### Step 4: Synthesis of 6-(difluoromethoxy)-2-methyl-5-(4, 4, 5, 5-tetramethyl-1, 3, 2-dioxaborolan-2-yl)-2H-indazole

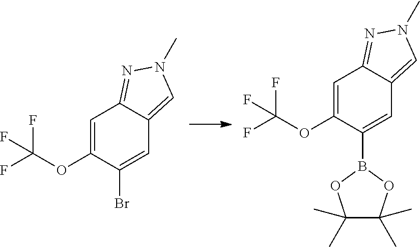 [SMILES: Cn1cc2cc(Br)c(OC(F)(F)F)cc2n1 ; Cn1cc2cc(B3OC(C)(C)C(C)(C)O3)c(OC(F)(F)F)cc2n1]

Under an argon atmosphere, 5-bromo-6-(trifluoromethoxy)-2-methyl-2H-indazole (614 mg, 2.1 mmol) is dissolved in dioxane (12 mL), bis(pinacolato)diboron (0.79 g, 3.1 mmol), potassium acetate (614 mg, 6.2 mmol) and Pd(dppf)Cl₂×DCM (170 mg, 0.21 mmol) are added, and the reaction mixture is stirred at 90° C. for 2 h. The cooled reaction mixture is then filtered through a Pd scavenger cartridge, the cartridge is rinsed with methanol and the filtrate is concentrated. The residue is purified by flash column chromatography (EtOAc/CycH 30/70 to 100/0) to afford the product.

Analysis (method A): Rₜ: 0.71 min, [M+H]⁺: 343

### Step 5: Synthesis of 5-(4-bromo-5-isopropyl-1-methyl-1H-imidazol-2-yl)-6-(triifluorometh-oxy)-2-methyl-2H-indazole

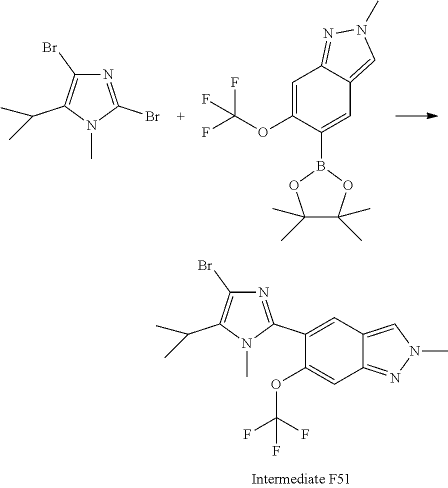 [SMILES: CC(C)c1c(Br)nc(Br)n1C ; Cn1cc2cc(B3OC(C)(C)C(C)(C)O3)c(OC(F)(F)F)cc2n1 ; CC(C)c1c(Br)nc(-c2cc3cn(C)nc3cc2OC(F)(F)F)n1C]

Under an argon atmosphere, 2, 4-dibromo-5-isopropyl-1-methyl-1H-imidazole (54 mg, 0.19 mmol, in analogy synthesized as described for intermediate F45) and 6-(trifluoromethoxy)-2-methyl-5-(4, 4, 5, 5-tetramethyl-1, 3, 2-dioxaborolan-2-yl)-2H-indazole (79 mg, 0.23 mmol) are dissolved in dioxane (1.8 mL). Cs₂CO₃ (188 mg, 0.58 mmol) and Pd(PPh₃)₄(22 mg, 0.0.02 mmol) are added at RT and the reaction mixture is stirred at 80° C. for 18 h. The reaction mixture is poured on ice and the formed solid is isolated. The solid is dissolved in dichloromethane and adsorbed on Extrelut. The mixture purified by flash column chromatography (DCM/MeOH 100/0 to DCM/MeOH 90/10 gradient. The combined fractions are concentrated to yield the desired product F51.

Analysis (method F): Rₜ: 0.72 min, [M+H]⁺: 415/417 (Br)

### Synthesis of Intermediate G1 and Examples 1, 2, 4, 6, 7, 10, 11, 12, 14, 16, 18, 21, 24, 28, 31, 32, 33, 37, 39, 43, 45, 56, 61, 62, 67, 70, 77

### Synthesis of Intermediate G1: ((1R)-2-[4-(5-methyl-1-{6-methyl-2H-pyrazolo[3, 4-b]pyridin-5-yl}-4-(propan-2-yl)-1H-pyrazol-3-yl)-2H-indazol-2-yl]-1-phenylethan-1-ol

### Step 1: Synthesis of (1R)-2-[4-(1-{2-[(4-methoxyphenyl)methyl]-6-methyl-2H-pyrazolo[3, 4-b]pyridin-5-yl}-5-methyl-4-(propan-2-yl)-1H-pyrazol-3-yl)-2H-indazol-2-yl]-1-phenylethan-1-ol

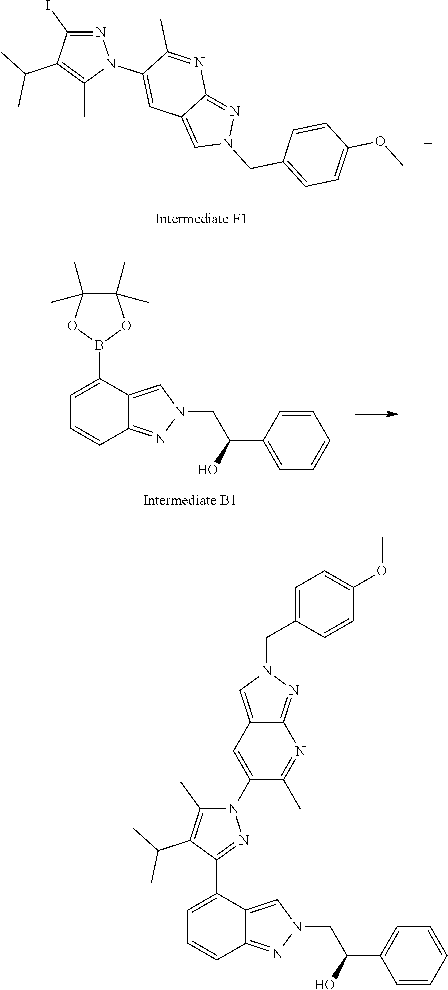

Under an argon atmosphere, 3-iodo-1-{2-[(4-methoxyphenyl)methyl]-6-methyl-2H-pyrazolo[3, 4-b]pyridin-5-yl}-5-methyl-4-(propan-2-yl)-1H-pyrazole (F1) (1.85 g, 3.7 mmol) and (1R)-1-phenyl-2-[4-(4, 4, 5, 5-tetramethyl-1, 3, 2-dioxaborolan-2-yl)-2H-indazol-2-yl]ethan-1-ol (B1) (1.64 g, 4.06 mmol, 90% purity) are dissolved in dioxane (28 mL). An aqueous K₃PO₄ solution (4.61 mL, 9.23 mmol, 2 M) and (2-dicyclohexylphosphino-2′, 4′, 6′-triisopropyl-1, 1′-biphenyl)[2-(2′-amino-1, 1′-biphenyl)]palladium(II) methanesulfonate (XPhos Pd G3, 187 mg, 0.22 mmol) are added and the reaction mixture is stirred at 90° C. overnight. Then SiliaMetS Thiol is added to the reaction mixture, and it is stirred for 15 min. Then the solids are removed by filtration. The filtrate is concentrated and triturated with MTBE; the solid is filtered off as the first batch of the title compound. The filtrate is concentrated and purified by flash chromatography (EtOAc/MeOH 100/0 to EtOAc/MeOH 90/10) to afford a second batch of the title compound.

Analysis (method A): Rₜ: 0.68 min, [M+H]⁺: 612

### Step 2: Synthesis of (1R)-2-[4-(5-methyl-1-{6-methyl-2H-pyrazolo[3, 4-b]pyridin-5-yl}-4-(propan-2-yl)-1H-pyrazol-3-yl)-2H-indazol-2-yl]-1-phenylethan-1-ol

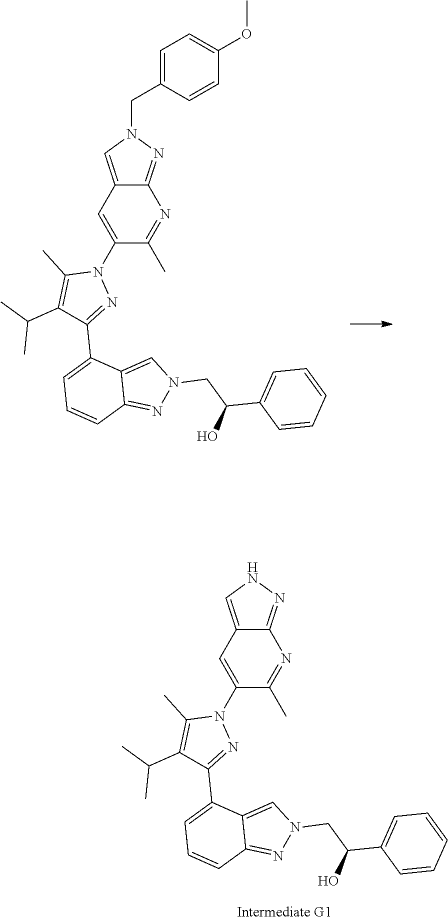 [SMILES: COc1ccc(Cn2cc3cc(-n4nc(-c5cccc6nn(C[C@H](O)c7ccccc7)cc56)c(C(C)C)c4C)c(C)nc3n2)cc1 ; Cc1nc2n[nH]cc2cc1-n1nc(-c2cccc3nn(C[C@H](O)c4ccccc4)cc23)c(C(C)C)c1C]

(1R)-2-[4-(1-{2-[(4-Methoxyphenyl) methyl]-6-methyl-2H-pyrazolo[3, 4-b]pyridin-5-yl}-5-methyl-4-(propan-2-yl)-1H-pyrazol-3-yl)-2H-indazol-2-yl]-1-phenylethan-1-ol (1.70 g, 2.50 mmol) and anisole (0.55 mL, 5 mmol) are dissolved in DCE (30 mL). TFA (20 mL, 259 mmol) is added, and the reaction mixture is stirred at 50° C. for three days. The reaction mixture is concentrated, and the residue is purified by reversed phase chromatography (HPLC; ACN/water including TFA) to afford 755 mg of the product.

Analysis (method H): Rₜ: 0.68 min, [M+H]⁺: 492

### Synthesis of Example 1: (1R)-2-[4-(5-methyl-1-{6-methyl-2-[2-(morpholin-4-yl)ethyl]-2H-pyrazolo[3, 4-b]pyridin-5-yl}-4-(propan-2-yl)-1H-pyrazol-3-yl)-2H-indazol-2-yl]-1-phenylethan-1-ol

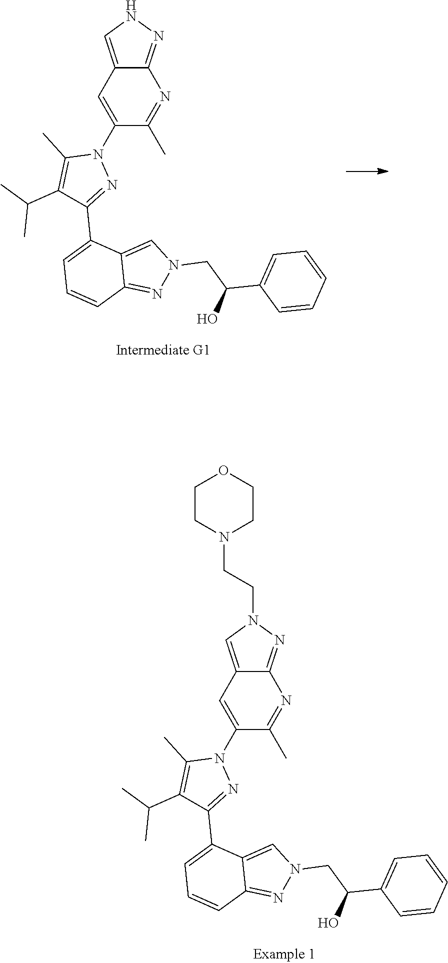 [SMILES: Cc1nc2n[nH]cc2cc1-n1nc(-c2cccc3nn(C[C@H](O)c4ccccc4)cc23)c(C(C)C)c1C ; Cc1nc2nn(CCN3CCOCC3)cc2cc1-n1nc(-c2cccc3nn(C[C@H](O)c4ccccc4)cc23)c(C(C)C)c1C]

(1R)-2-[4-(5-Methyl-1-{6-methyl-2H-pyrazolo[3, 4-b]pyridin-5-yl)-4-(propan-2-yl)-1H-pyrazol-3-yl)-2H-indazol-2-yl]-1-phenylethan-1-ol (300 mg, 0.61 mmol) is dissolved in ACN (5 mL) and DMF (3 ML), K₂CO₃ (253 mg, 1.83 mmol) and 4-(2-bromoethyl)morpholine hydrobromide (218 mg, 0.79 mmol) are added and the reaction mixture is stirred at RT overnight. The reaction mixture is purified by reversed phase chromatography (HPLC; ACN/water including NH₃) to afford the example 1.

Analysis (method H): Rₜ: 0.94 min [M+H]⁺: 605

The intermediate and the examples compiled in the following table are obtained by following a procedure analogous to that described for example 1 using intermediate G1.

| | | | | MS | |
|---|---|---|---|---|---|
| | | | | (ESI+): | |
| Ex- | Synthesis | | LC | m/z | t_{R} |
| ample | from | Structure/Name | Method | [M + H]⁺ | [min] |
| 2 | G1 + 4-bromo- butyronitrile; 60° C., overnight | 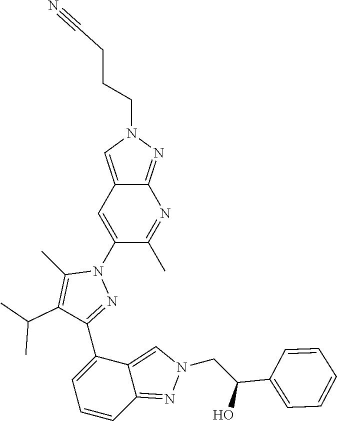 [SMILES: Cc1nc2nn(CCCC#N)cc2cc1-n1nc(-c2cccc3nn(C[C@H](O)c4ccccc4)cc23)c(C(C)C)c1C] | N | 559 | 0.84 |
| | | 4-[5-(3-{2-[(2R)-2-hydroxy-2- | | | |
| | | phenylethyl]-2H-indazol-4-yl}-5- | | | |
| | | methyl-4-(propan-2-yl)-1H-pyrazol-1- | | | |
| | | yl)-6-methyl-2H-pyrazolo[3,4- | | | |
| | | b]pyridin-2-yl]butanenitrile | | | |
| 6 | G1 + 3-(bromo- methyl) pyridine; 60° C. overnight | 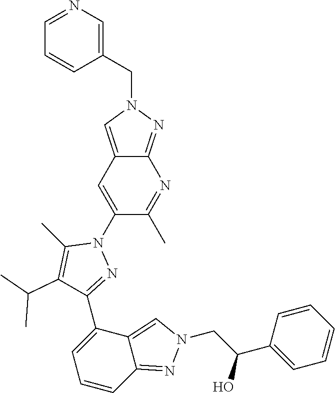 [SMILES: Cc1nc2nn(Cc3cccnc3)cc2cc1-n1nc(-c2cccc3nn(C[C@H](O)c4ccccc4)cc23)c(C(C)C)c1C] | L | 583 | 0.69 |
| | | (1R)-2-[4-(5-methyl-1-{6-methyl-2- | | | |
| | | [(pyridin-3-yl) methyl]-2H-pyrazolo[3, | | | |
| | | 4-b]pyridin-5-yl}-4-(propan-2-yl)-1H- | | | |
| | | pyrazol-3-yl)-2H-indazol-2-yl]-1- | | | |
| | | phenylethan-1-ol | | | |
| 7 | G1 + 1-(3- chloro- propyl)-4- methyl piperazine; 85° C. overnight | 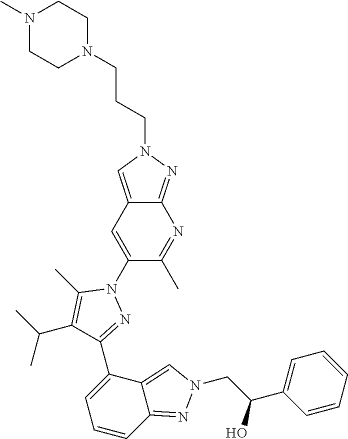 [SMILES: Cc1nc2nn(CCCN3CCN(C)CC3)cc2cc1-n1nc(-c2cccc3nn(C[C@H](O)c4ccccc4)cc23)c(C(C)C)c1C] | H | 632 | 1.03 |
| | | (1R)-2-[4-(5-methyl-1-{6-methyl-2-[3- | | | |
| | | (4-methylpiperazin-1-yl)propyl]-2H- | | | |
| | | pyrazolo[3,4-b]pyridin-5-yl}-4- | | | |
| | | (propan-2-yl)-1H-pyrazol-3-yl)-2H- | | | |
| | | indazol-2-yl]-1-phenylethan-1-ol | | | |
| 12 | Step 1: G1 + 2-(4-chloro- butoxy)- tetrahydro- 2H-pyran; 60° overnight Step 2: TFA, RT, overnight | 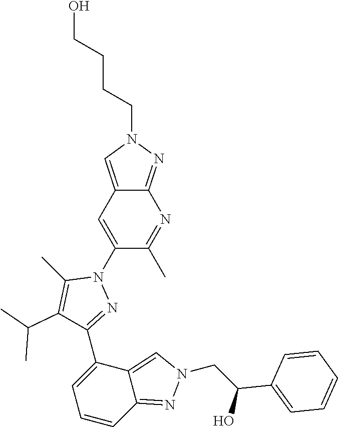 [SMILES: Cc1nc2nn(CCCCO)cc2cc1-n1nc(-c2cccc3nn(C[C@H](O)c4ccccc4)cc23)c(C(C)C)c1C] | P | 564 | 0.78 |
| | | 4-[5-(3-{2-[(2R)-2-hydroxy-2- | | | |
| | | phenylethyl]-2H-indazol-4-yl}-5- | | | |
| | | methyl-4-(propan-2-yl)-1H-pyrazol-1- | | | |
| | | yl)-6-methyl-2H-pyrazolo[3,4- | | | |
| | | b]pyridin-2-yl]butan-1-ol | | | |
| 14 | G1 + 3- bromo- tetrahydro- furan; 60° C., overnight | 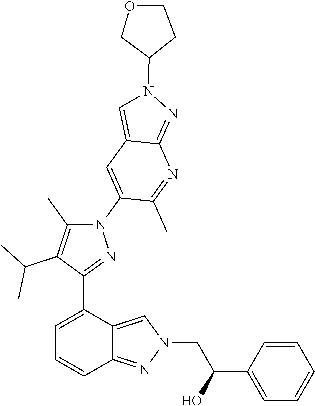 [SMILES: Cc1nc2nn(C3CCOC3)cc2cc1-n1nc(-c2cccc3nn(C[C@H](O)c4ccccc4)cc23)c(C(C)C)c1C] | H | 562 | 1.04 |
| | | (1R)-2-(4-{5-methyl-1-[6-methyl-2- | | | |
| | | (oxolan-3-yl)-2H-pyrazolo[3,4- | | | |
| | | b]pyridin-5-yl]-4-(propan-2-yl)-1H- | | | |
| | | pyrazol-3-yl}-2H-indazol-2-yl)-1- | | | |
| | | phenylethan-1-ol | | | |
| 16 | G1 + 2- bromoethyl methyl ether; 60° C. overnight | 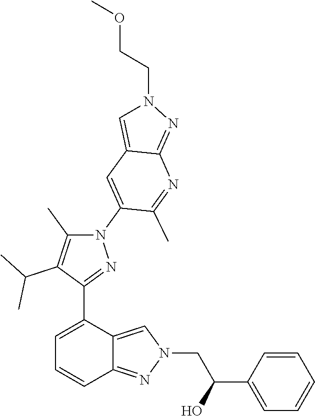 [SMILES: COCCn1cc2cc(-n3nc(-c4cccc5nn(C[C@H](O)c6ccccc6)cc45)c(C(C)C)c3C)c(C)nc2n1] | N | 550 | 0.85 |
| | | (1R)-2-(4-{1-[2-(2-methoxyethyl)-6- | | | |
| | | methyl-2H-pyrazolo[3,4-b]pyridin-5- | | | |
| | | yl]-5-methyl-4-(propan-2-yl)-1H- | | | |
| | | pyrazol-3-yl}-2H-indazol-2-yl)-1- | | | |
| | | phenylethan-1-ol | | | |
| 18 | G1 + 2- bromopropane; 60° C., overnight | 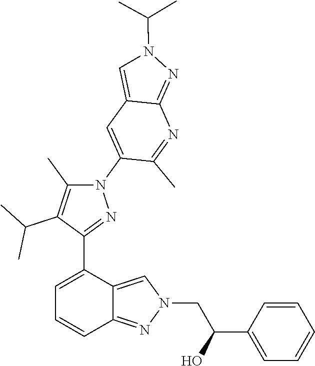 [SMILES: Cc1nc2nn(C(C)C)cc2cc1-n1nc(-c2cccc3nn(C[C@H](O)c4ccccc4)cc23)c(C(C)C)c1C] | N | 534 | 0.90 |
| | | (1R)-2-(4-{5-methyl-1-[6-methyl-2- | | | |
| | | (propan-2-yl)-2H-pyrazolo[3,4- | | | |
| | | b]pyridin-5-yl]-4-(propan-2-yl)-1H- | | | |
| | | pyrazol-3-yl}-2H-indazol-2-yl)-1- | | | |
| | | phenylethan-1-ol | | | |
| 21 | G1 + 1-(3- chloropropyl) pyrrolidin-2- one; 60° C. overnight | 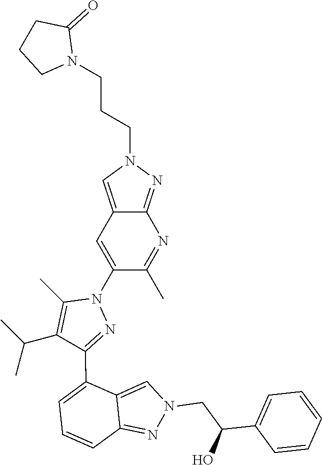 [SMILES: Cc1nc2nn(CCCN3CCCC3=O)cc2cc1-n1nc(-c2cccc3nn(C[C@H](O)c4ccccc4)cc23)c(C(C)C)c1C] | H | 617 | 1.02 |
| | | 1-{3-[5-(3-{2-[(2R)-2-hydroxy-2- | | | |
| | | phenylethyl]-2H-indazol-4-yl}-5- | | | |
| | | methyl-4-(propan-2-yl)-1H-pyrazol-1- | | | |
| | | yl)-6-methyl-2H-pyrazolo[3,4- | | | |
| | | b]pyridin-2-yl]propyl}pyrrolidin-2-one | | | |
| 24 | G1 + 4- Chloromethyl- 1-methyl- 1H-pyrazole; 60° C. overnight | 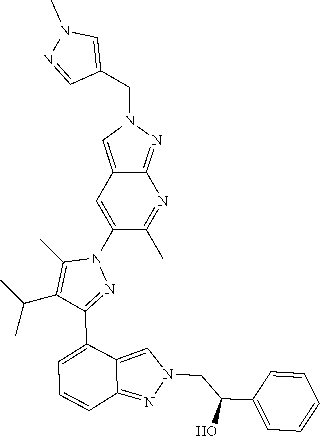 [SMILES: Cc1nc2nn(Cc3cnn(C)c3)cc2cc1-n1nc(-c2cccc3nn(C[C@H](O)c4ccccc4)cc23)c(C(C)C)c1C] | N | 586 | 0.82 |
| | | (1R)-2-[4-(5-methyl-1-{6-methyl-2-[(1- | | | |
| | | methyl-1H-pyrazol-4-yl)methyl]-2H- | | | |
| | | pyrazolo[3,4-b]pyridin-5-yl}-4- | | | |
| | | (propan-2-yl)-1H-pyrazol-3-yl)-2H- | | | |
| | | indazol-2-yl]-1-phenylethan-1-ol | | | |
| 31 | G1 + 2- (bromomethyl)- 1,4- dioxane; 60° C. overnight | 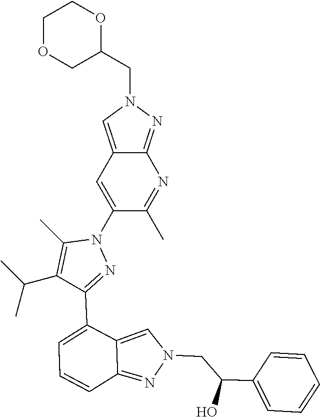 [SMILES: Cc1nc2nn(CC3COCCO3)cc2cc1-n1nc(-c2cccc3nn(C[C@H](O)c4ccccc4)cc23)c(C(C)C)c1C] | L | 592 | 0.84 |
| | | (1R)-2-[4-(1-{2-[(1,4-dioxan-2- | | | |
| | | yl)methyl]-6-methyl-2H-pyrazolo[3,4- | | | |
| | | b]pyridin-5-yl}-5-methyl-4-(propan-2- | | | |
| | | yl)-1H-pyrazol-3-yl)-2H-indazol-2-yl]-1- | | | |
| | | phenylethan-1-ol | | | |
| 32 | Step 1: G1 + 2-(3-chloro- propoxy) tetrahydro- 2H- pyran; 60° C. overnight Step 2: TFA, RT overnight | 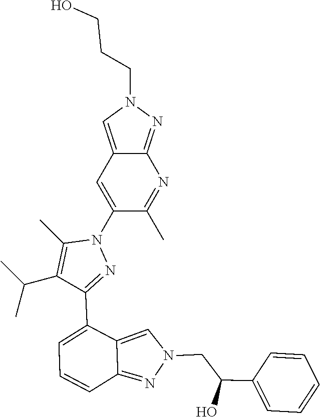 [SMILES: Cc1nc2nn(CCCO)cc2cc1-n1nc(-c2cccc3nn(C[C@H](O)c4ccccc4)cc23)c(C(C)C)c1C] | L | 550 | 0.78 |
| | | 3-[5-(3-{2-[(2R)-2-hydroxy-2- | | | |
| | | phenylethyl]-2H-indazol-4-yl}-5- | | | |
| | | methyl-4-(propan-2-yl)-1H-pyrazol-1- | | | |
| | | yl)-6-methyl-2H-pyrazolo[3,4- | | | |
| | | b]pyridin-2-yl]propan-1-ol | | | |
| 37 | Step 1: G1 + 2-(2- chloroethoxy) tetrahydro- 2H-pyran; 60° C., overnight; Step 2: TFA, RT overnight | 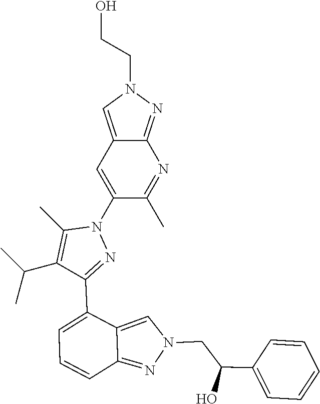 [SMILES: Cc1nc2nn(CCO)cc2cc1-n1nc(-c2cccc3nn(C[C@H](O)c4ccccc4)cc23)c(C(C)C)c1C] | P | 536 | 0.75 |
| | | (1R)-2-(4-{1-[2-(2-hydroxyethyl)-6- | | | |
| | | methyl-2H-pyrazolo[3,4-b]pyridin-5- | | | |
| | | yl]-5-methyl-4-(propan-2-yl)-1H- | | | |
| | | pyrazol-3-yl}-2H-indazol-2-yl)-1- | | | |
| | | phenylethan-1-ol | | | |
| 39 | G1 + N,N- dimethyl- aminoethyl- chlorid; 60° C., overnight | 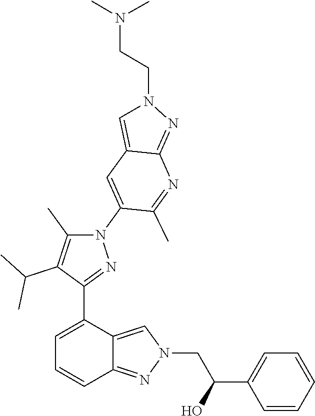 [SMILES: Cc1nc2nn(CCN(C)C)cc2cc1-n1nc(-c2cccc3nn(C[C@H](O)c4ccccc4)cc23)c(C(C)C)c1C] | N | 563 | 0.84 |
| | | (1R)-2-[4-(1-{2-[2- | | | |
| | | (dimethylamino)ethyl]-6-methyl-2H- | | | |
| | | pyrazolo[3,4-b]pyridin-5-yl}-5-methyl- | | | |
| | | 4-(propan-2-yl)-1H-pyrazol-3-yl)-2H- | | | |
| | | indazol-2-yl]-1-phenylethan-1-ol | | | |
| 43 | G1 + 3- dimethyl- aminopropyl chloride; 60° C., overnight | 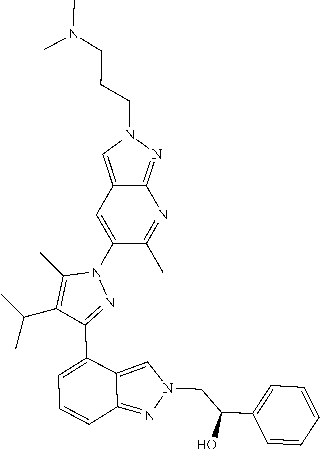 [SMILES: Cc1nc2nn(CCCN(C)C)cc2cc1-n1nc(-c2cccc3nn(C[C@H](O)c4ccccc4)cc23)c(C(C)C)c1C] | H | 577 | 1.06 |
| | | (1R)-2-[4-(1-{2-[3- | | | |
| | | (dimethylamino)propyl]-6-methyl-2H- | | | |
| | | pyrazolo[3,4-b]pyridin-5-yl}-5-methyl- | | | |
| | | 4-(propan-2-yl)-1H-pyrazol-3-yl)-2H- | | | |
| | | indazol-2-yl]-1-phenylethan-1-ol | | | |
| 45 | Step 1: G1 + 1-bromo-4- chlorobutane; 60° C. overnight; Step 2: 1- methylpipera zine, 2 h at 60° C. | 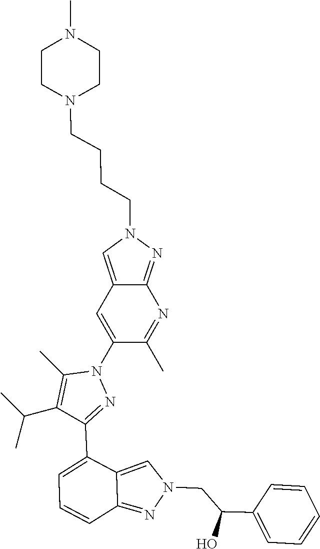 [SMILES: Cc1nc2nn(CCCCN3CCN(C)CC3)cc2cc1-n1nc(-c2cccc3nn(C[C@H](O)c4ccccc4)cc23)c(C(C)C)c1C] | H | 646 | 1.04 |
| | | (1R)-2-[4-(5-methyl-1-{6-methyl-2-[4- | | | |
| | | (4-methylpiperazin-1-yl)butyl]-2H- | | | |
| | | pyrazolo[3,4-b]pyridin-5-yl}-4- | | | |
| | | (propan-2-yl)-1H-pyrazol-3-yl)-2H- | | | |
| | | indazol-2-yl]-1-phenylethan-1-ol | | | |
| 56 | G1 + (bromo- methyl) cyclo propane; DMF, RT overnight | 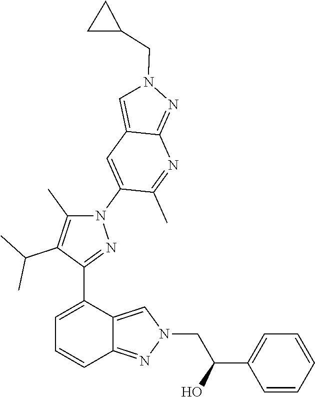 [SMILES: Cc1nc2nn(CC3CC3)cc2cc1-n1nc(-c2cccc3nn(C[C@H](O)c4ccccc4)cc23)c(C(C)C)c1C] | H | 546 | 1.08 |
| | | (1R)-2-(4-{1-[2-(cyclopropylmethyl)-6- | | | |
| | | methyl-2H-pyrazolo[3,4-b]pyridin-5- | | | |
| | | yl]-5-methyl-4-(propan-2-yl)-1H- | | | |
| | | pyrazol-3-yl}-2H-indazol-2-yl)-1- | | | |
| | | phenylethan-1-ol | | | |
| 62 | G1 + 5- (chloromethyl)- 1-methyl- 1H-1,2,4- triazole; 60° C. overnight | 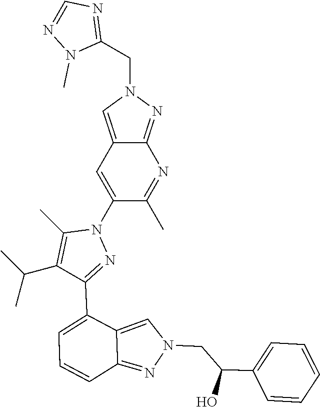 [SMILES: Cc1nc2nn(Cc3ncnn3C)cc2cc1-n1nc(-c2cccc3nn(C[C@H](O)c4ccccc4)cc23)c(C(C)C)c1C] | N | 587 | 0.79 |
| | | (1R)-2-[4-(5-methyl-1-{6-methyl-2-[(1- | | | |
| | | methyl-1H-1,2,4-triazol-5-yl) methyl]- | | | |
| | | 2H-pyrazolo[3,4-b]pyridin-5-yl}-4- | | | |
| | | (propan-2-yl)-1H-pyrazol-3-yl)-2H- | | | |
| | | indazol-2-yl]-1-phenylethan-1-ol | | | |
| 67 | G1 + 3- bromo- propane-1,2- diol; 60° C. overnight | 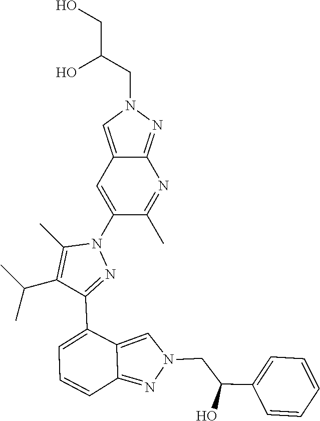 [SMILES: Cc1nc2nn(CC(O)CO)cc2cc1-n1nc(-c2cccc3nn(C[C@H](O)c4ccccc4)cc23)c(C(C)C)c1C] | N | 566 | 0.75 |
| | | 3-[5-(3-{2-[(2R)-2-hydroxy-2- | | | |
| | | phenylethyl]-2H-indazol-4-yl}-5- | | | |
| | | methyl-4-(propan-2-yl)-1H-pyrazol-1- | | | |
| | | yl)-6-methyl-2H-pyrazolo[3,4- | | | |
| | | b]pyridin-2-yl]propane-1,2-diol | | | |
| 70 | Step 1: G1 + 1,3-dibromo- 2-propanol; 60° C. overnight; Step 2: 1- methylpipera zine; 80° C. overnight | 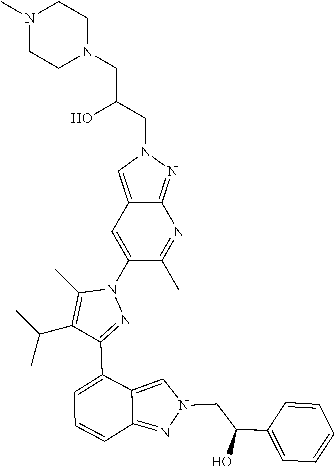 [SMILES: Cc1nc2nn(CC(O)CN3CCN(C)CC3)cc2cc1-n1nc(-c2cccc3nn(C[C@H](O)c4ccccc4)cc23)c(C(C)C)c1C] | H | 648 | 1.00 |
| | | 1-[5-(3-{2-[(2R)-2-hydroxy-2- | | | |
| | | phenylethyl]-2H-indazol-4-yl}-5- | | | |
| | | methyl-4-(propan-2-yl)-1H-pyrazol-1- | | | |
| | | yl)-6-methyl-2H-pyrazolo[3,4- | | | |
| | | b]pyridin-2-yl]-3-(4-methylpiperazin-1- | | | |
| | | yl)propan-2-ol | | | |
| 77 | G1 + chloromethyl (dimethyl)- phosphine oxide; 70° C. overnight | 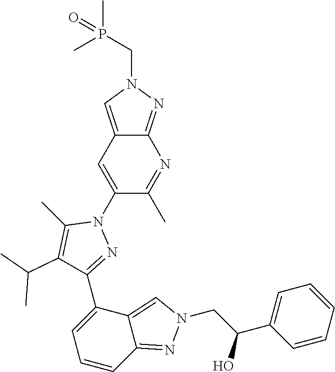 [SMILES: Cc1nc2nn(CP(C)(C)=O)cc2cc1-n1nc(-c2cccc3nn(C[C@H](O)c4ccccc4)cc23)c(C(C)C)c1C] | G | 582 | 0.96 |
| | | (1R)-2-[4-(1-{2- | | | |
| | | [(dimethylphosphoryl)methyl]-6- | | | |
| | | methyl-2H-pyrazolo[3,4-b]pyridin-5- | | | |
| | | yl}-5-methyl-4-(propan-2-yl)-1H- | | | |
| | | pyrazol-3-yl)-2H-indazol-2-yl]-1- | | | |
| | | phenylethan-1-ol | | | |
| 98 | G1 + chloro- acetonitrile; 60° C. overnight | 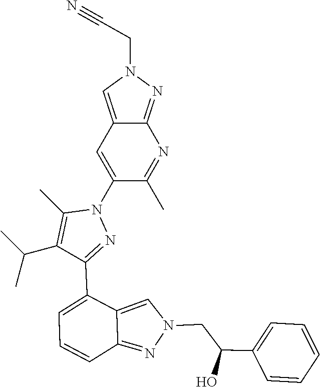 [SMILES: Cc1nc2nn(CC#N)cc2cc1-n1nc(-c2cccc3nn(C[C@H](O)c4ccccc4)cc23)c(C(C)C)c1C] | H | 531 | 1.04 |
| | | 2-[5-(3-{2-[(2R)-2-hydroxy-2- | | | |
| | | phenylethyl]-2H-indazol-4-yl}-5- | | | |
| | | methyl-4-(propan-2-yl)-1H-pyrazol-1- | | | |
| | | yl)-6-methyl-2H-pyrazolo[3,4- | | | |
| | | b]pyridin-2-yl]acetonitrile | | | |
| 99 | G1 + 3- bromo- propionitrile; 60° C. overnight | 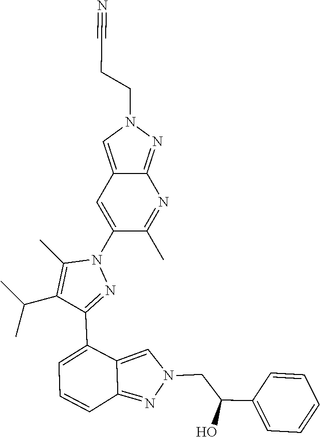 [SMILES: Cc1nc2nn(CCC#N)cc2cc1-n1nc(-c2cccc3nn(C[C@H](O)c4ccccc4)cc23)c(C(C)C)c1C] | N | 545 | 0.83 |
| | | 3-[5-(3-{2-[(2R)-2-hydroxy-2- | | | |
| | | phenylethyl]-2H-indazol-4-yl}-5- | | | |
| | | methyl-4-(propan-2-yl)-1H-pyrazol-1- | | | |
| | | yl)-6-methyl-2H-pyrazolo[3,4- | | | |
| | | b]pyridin-2-yllpropanenitrile | | | |
| 100 | G1 + iodoethane; RT overnight | 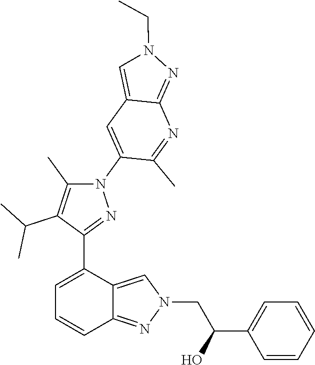 [SMILES: CCn1cc2cc(-n3nc(-c4cccc5nn(C[C@H](O)c6ccccc6)cc45)c(C(C)C)c3C)c(C)nc2n1] | H | 520 | 1.05 |
| | | (1R)-2-[4-(1-{2-ethyl-6-methyl-2H- | | | |
| | | pyrazolo[3,4-b]pyridin-5-yl}-5-methyl- | | | |
| | | 4-(propan-2-yl)-1H-pyrazol-3-yl)-2H- | | | |
| | | indazol-2-yl]-1-phenylethan-1-ol | | | |
| 103 | G1 + 2- bromo-1,1- difluoroethane; 60° C. overnight | 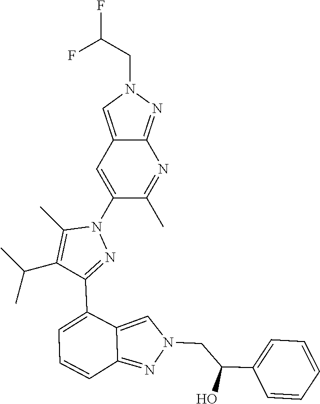 [SMILES: Cc1nc2nn(CC(F)F)cc2cc1-n1nc(-c2cccc3nn(C[C@H](O)c4ccccc4)cc23)c(C(C)C)c1C] | N | 556 | 0.87 |
| | | (1R)-2-(4-{1-[2-(2,2-difluoroethyl)-6- | | | |
| | | methyl-2H-pyrazolo[3,4-b]pyridin-5- | | | |
| | | yl]-5-methyl-4-(propan-2- | | | |
| | | yl)-1H-pyrazol-3-yl}-2H-indazol-2-yl)- | | | |
| | | 1-phenylethan-1-ol | | | |

### Synthesis of Example 3: (1R)-2-[4-(1-{2, 6-dimethyl-2H-pyrazolo[3, 4-b]pyridin-5-yl}-5-methyl-4-(propan-2-yl)-1H-pyrazol-3-yl)-2H-indazol-2-yl]-1-phenyl(1-²H)ethan-1-ol

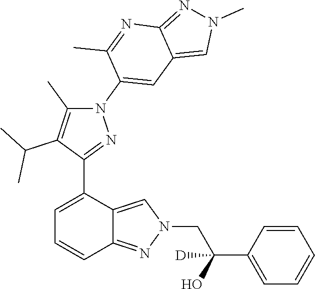 [SMILES: [2H][C@](O)(Cn1cc2c(-c3nn(-c4cc5cn(C)nc5nc4C)c(C)c3C(C)C)cccc2n1)c1ccccc1]

### Step 1: Synthesis of 2-[4-(1-{2, 6-dimethyl-2H-pyrazolo[3, 4-b]pyridin-5-yl}-5-methyl-4-(propan-2-yl)-1H-pyrazol-3-yl)-2H-indazol-2-yl]-1-phenylethan-1-one

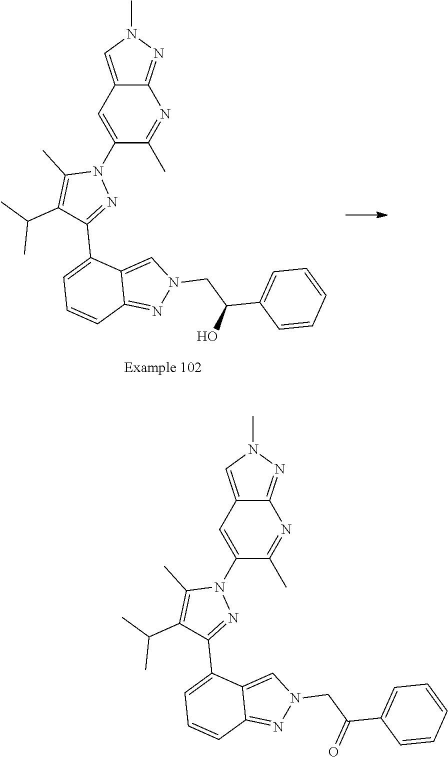 [SMILES: Cc1nc2nn(C)cc2cc1-n1nc(-c2cccc3nn(C[C@H](O)c4ccccc4)cc23)c(C(C)C)c1C ; Cc1nc2nn(C)cc2cc1-n1nc(-c2cccc3nn(CC(=O)c4ccccc4)cc23)c(C(C)C)c1C]

(1R)-2-[4-(1-{2, 6-Dimethyl-2H-pyrazolo[3, 4-b]pyridin-5-yl}-5-methyl-4-(propan-2-yl)-1H-pyrazol-3-yl)-2H-indazol-2-yl]-1-phenylethan-1-ol (Example 102) (300 mg, 0.59 mmol) is dissolved in DCM (10 mL). DMP (0.58 g, 1.31 mmol) is added at 0° C. and the reaction mixture is stirred at RT for 5 h. The reaction mixture is concentrated and purified by reversed phase chromatography (HPLC; C18, ACN/water including TFA)) to afford the product.

Analysis (method G): Rₜ: 0.96 min, [M+H]⁺: 504

### Step 2: Synthesis of 2-[4-(1-{2, 6-dimethyl-2H-pyrazolo[3, 4-b]pyridin-5-yl}-5-methyl-4-(propan-2-yl)-1H-pyrazol-3-yl)-2H-indazol-2-yl]-1-phenyl(1-²H) ethan-1-ol

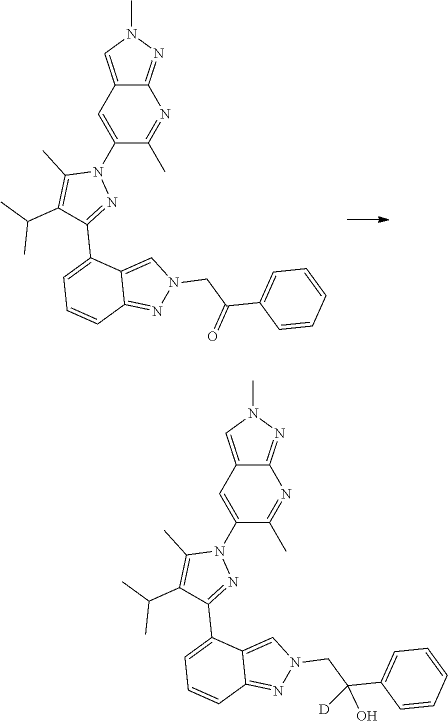 [SMILES: Cc1nc2nn(C)cc2cc1-n1nc(-c2cccc3nn(CC(=O)c4ccccc4)cc23)c(C(C)C)c1C ; [2H]C(O)(Cn1cc2c(-c3nn(-c4cc5cn(C)nc5nc4C)c(C)c3C(C)C)cccc2n1)c1ccccc1]

2-[4-(1-{2, 6-Dimethyl-2H-pyrazolo[3, 4-b]pyridin-5-yl}-5-methyl-4-(propan-2-yl)-1H-pyrazol-3-yl)-2H-indazol-2-yl]-1-phenylethan-1-one (280 mg, 0.56 mmol) is dissolved in THE (5 mL) and MeOH (5 mL). Sodium borodeuteride (52 mg, 1.11 mmol) is added and the reaction mixture is stirred at RT for 30 min. The reaction mixture is quenched with water and extracted with DCM. The organic layer is dried (Na₂SO₄), filtered and concentrated to afford the product.

Analysis (method G): Rₜ: 0.93 min, [M+H]⁺: 507

### Step 3: Synthesis of (1R)-2-[4-(1-{2, 6-dimethyl-2H-pyrazolo[3, 4-b]pyridin-5-yl}-5-methyl-4-(propan-2-yl)-1H-pyrazol-3-yl)-2H-indazol-2-yl]-1-phenyl(1-²H)ethan-1-ol

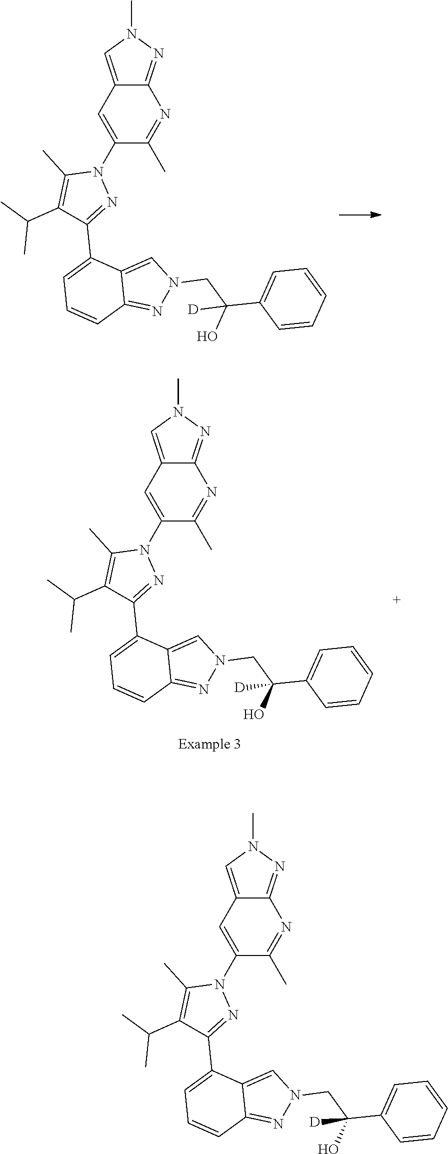 [SMILES: [2H]C(O)(Cn1cc2c(-c3nn(-c4cc5cn(C)nc5nc4C)c(C)c3C(C)C)cccc2n1)c1ccccc1 ; [2H][C@](O)(Cn1cc2c(-c3nn(-c4cc5cn(C)nc5nc4C)c(C)c3C(C)C)cccc2n1)c1ccccc1 ; [2H][C@@](O)(Cn1cc2c(-c3nn(-c4cc5cn(C)nc5nc4C)c(C)c3C(C)C)cccc2n1)c1ccccc1]

2-[4-(1-{2, 6-Dimethyl-2H-pyrazolo[3, 4-b]pyridin-5-yl}-5-methyl-4-(propan-2-yl)-1H-pyrazol-3-yl)-2H-indazol-2-yl]-1-phenyl(1-²H)ethan-1-ol (149 mg, 0.29 mmol) is separated by chiral purification method W to afford the compounds (1R)-2-[4-(1-{2, 6-dimethyl-2H-pyrazolo[3, 4-b]pyridin-5-yl}-5-methyl-4-(propan-2-yl)-1H-pyrazol-3-yl)-2H-indazol-2-yl]-1-phenyl(1-²H)ethan-1-ol (example 3) (Analysis (method W): Rₜ: 4.89 min) and (1S)-2-[4-(1-{2, 6-dimethyl-2H-pyrazolo[3, 4-b]pyridin-5-yl}-5-methyl-4-(propan-2-yl)-1H-pyrazol-3-yl)-2H-indazol-2-yl]-1-phenyl(1-²H)ethan-1-ol Analysis (method W): Rₜ: 3.78 min).

### Synthesis of Examples 4, 5, 9, 13, 15, 17, 19, 20, 22, 23, 26, 29, 34, 35, 38, 42, 46, 47, 48, 49, 50, 51, 53, 54, 57, 58, 59, 60, 64, 65, 71, 72, 73, 75, 76, 80, 83, 84, 86, 87, 89, 91, 97, 102

### Synthesis of Example 5: (1R)-2-[4-(1-{6-ethoxy-2-methyl-2H-pyrazolo[3, 4-b]pyridin-5-yl}-5-methyl-4-(propan-2-yl)-1H-pyrazol-3-yl)-2H-indazol-2-yl]-1-phenylethan-1-ol

### Step 1: Synthesis of (1R)-2-[4-(1-{2-[(4-methoxyphenyl) methyl]-6-methyl-2H-pyrazolo[3, 4-b]pyridin-5-yl}-5-methyl-4-(propan-2-yl)-1H-pyrazol-3-yl)-2H-indazol-2-yl]-1-phenylethan-1-ol

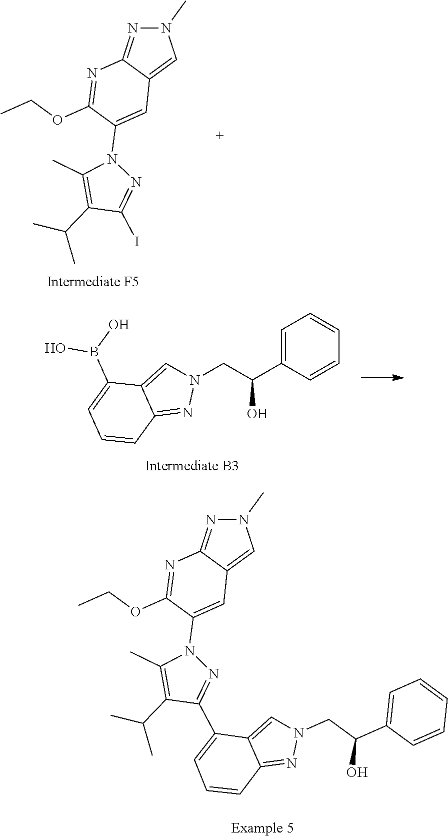 [SMILES: CCOc1nc2nn(C)cc2cc1-n1nc(I)c(C(C)C)c1C ; OB(O)c1cccc2nn(C[C@H](O)c3ccccc3)cc12 ; CCOc1nc2nn(C)cc2cc1-n1nc(-c2cccc3nn(C[C@H](O)c4ccccc4)cc23)c(C(C)C)c1C]

1-{6-Ethoxy-2-methyl-2H-pyrazolo[3, 4-b]pyridin-5-yl}-3-iodo-5-methyl-4-(propan-2-yl)-1H-pyrazole (F5) (75 mg, 0.12 mmol) and {2-[(2R)-2-hydroxy-2-phenylethyl]-2H-indazol-4-yl}boronic acid (B3) (52 mg, 0.15 mmol) are dissolved in dioxane (1 mL). Cs₂CO₃ (112 mg) and water (100 μL) and Pd(dppf)Cl₂×DCM (10 mg) are added, and the reaction mixture is stirred at 85° C. overnight. The mixture is then filtered, diluted with ACN and HOAc and purified by reversed phase HPLC to yield the desired product example 5.

Analysis (method G): Rₜ: 0.98 min, [M+H]⁺: 536

The Intermediate and Examples compiled in the following table are obtained by following a procedure analogous to that described for Intermediate G1 step 1.

| | | | | MS | |
|---|---|---|---|---|---|
| | | | | (ESI+): | |
| Exam- | | | LC | m/z | t_{R} |
| ple | Synthesis | Structure/Name | Method | [M + H]⁺ | [min] |
| 4 | F42 + B7 XPhos Pd G3 Cs₂CO₃ 90° C., 3 h | 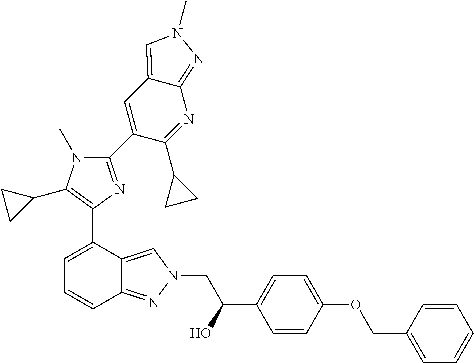 [SMILES: Cn1cc2cc(-c3nc(-c4cccc5nn(C[C@H](O)c6ccc(OCc7ccccc7)cc6)cc45)c(C4CC4)n3C)c(C3CC3)nc2n1] | A | 636 | 0.59 |
| | | (1R)-1-[4-(benzyloxy)phenyl]-2-[4-(5- | | | |
| | | cyclopropyl-2-{6-cyclopropyl-2- | | | |
| | | methyl-2H-pyrazolo[3,4-b]pyridin-5- | | | |
| | | yl}-1-methyl-1H-imidazol-4-yl)-2H- | | | |
| | | indazol-2-yl]ethan-1-ol | | | |
| 9 | F1 + B1 XPhos Pd G3 K₃PO₄ 90° C. 10 h | 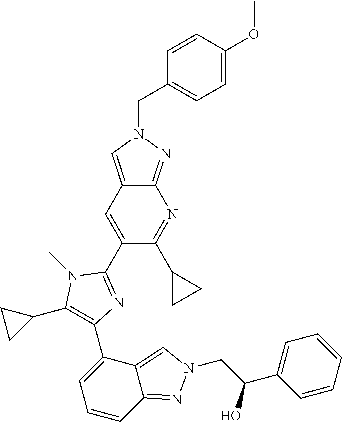 [SMILES: COc1ccc(Cn2cc3cc(-c4nc(-c5cccc6nn(C[C@H](O)c7ccccc7)cc56)c(C5CC5)n4C)c(C4CC4)nc3n2)cc1] | H | 636 | 1.10 |
| | | (1R)-2-[4-(5-cyclopropyl-2-{6- | | | |
| | | cyclopropyl-2-[(4-methoxyphenyl) | | | |
| | | methyl]-2H-pyrazolo[3, 4-b]pyridin-5- | | | |
| | | yl}-1-methyl-1H-imidazol-4-yl)-2H- | | | |
| | | indazol-2-yl]-1-phenylethan-1-ol | | | |
| 13 | F3 + B1 XPhos Pd G3 K₃PO₄ 90° C. 2 h | 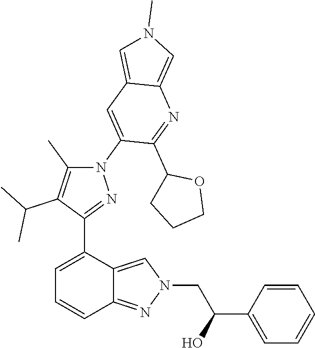 [SMILES: Cc1c(C(C)C)c(-c2cccc3nn(C[C@H](O)c4ccccc4)cc23)nn1-c1cc2cn(C)cc2nc1C1CCCO1] | H | 562 | 1.05 |
| | | (1R)-2-(4-{5-methyl-1-[2-methyl-6- | | | |
| | | (oxolan-2-yl)-2H-pyrazolo[3,4- | | | |
| | | b]pyridin-5-yl]-4-(propan-2-yl)-1H- | | | |
| | | pyrazol-3-yl}-2H-indazol-2-yl)-1- | | | |
| | | phenylethan-1-ol | | | |
| 15 | F4 + B6 XPhos Pd G3 K₃PO₄ 100° C. 1 h | 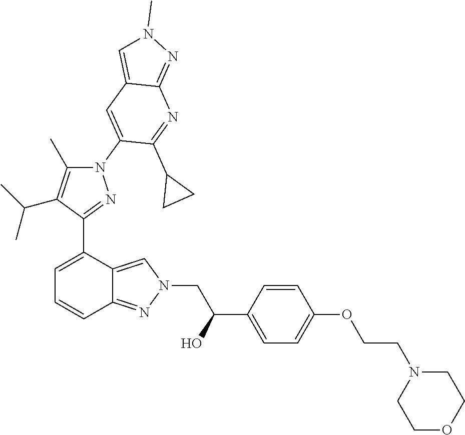 [SMILES: Cc1c(C(C)C)c(-c2cccc3nn(C[C@H](O)c4ccc(OCCN5CCOCC5)cc4)cc23)nn1-c1cc2cn(C)nc2nc1C1CC1] | J | 661 | 0.69 |
| | | (1R)-2-[4-(1-{6-cyclopropyl-2-methyl- | | | |
| | | 2H-pyrazolo[3,4-b]pyridin-5-yl}-5- | | | |
| | | methyl-4-(propan-2-yl)-1H-pyrazol-3- | | | |
| | | yl)-2H-indazol-2-yl]-1-{4-[2- | | | |
| | | (morpholin-4-yl)ethoxy]phenyl}ethan- | | | |
| | | 1-ol | | | |
| 17 | F5 + B3 XPhos Pd G3 Cs₂CO₃ 90° C. 4 h | 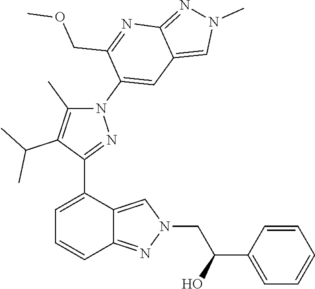 [SMILES: COCc1nc2nn(C)cc2cc1-n1nc(-c2cccc3nn(C[C@H](O)c4ccccc4)cc23)c(C(C)C)c1C] | G | 536 | 1.01 |
| | | (1R)-2-(4-{1-[6-(methoxymethyl)-2- | | | |
| | | methyl-2H-pyrazolo[3,4-b]pyridin-5- | | | |
| | | yl]-5-methyl-4-(propan-2-yl)-1H- | | | |
| | | pyrazol-3-yl}-2H-indazol-2-yl)-1- | | | |
| | | phenylethan-1-ol | | | |
| 19 | F29 + B3 xphos crotyl-Pd—Cl Cs₂CO₃ 90° C. 16 h | 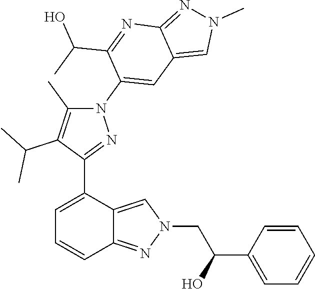 [SMILES: Cc1c(C(C)C)c(-c2cccc3nn(C[C@H](O)c4ccccc4)cc23)nn1-c1cc2cn(C)nc2nc1C(C)O] | N | 536 | 0.80 |
| | | (1R)-2-(4-{1-[6-(1-hydroxyethyl)-2- | | | |
| | | methyl-2H-pyrazolo[3,4-b]pyridin-5- | | | |
| | | yl]-5-methyl-4-(propan-2-yl)-1H- | | | |
| | | pyrazol-3-yl}-2H-indazol-2-yl)-1- | | | |
| | | phenylethan-1-ol | | | |
| 20 | F30 + B3 Pd(dppf)Cl₂ × DCM Cs₂CO₃ 80° C. 5 h | 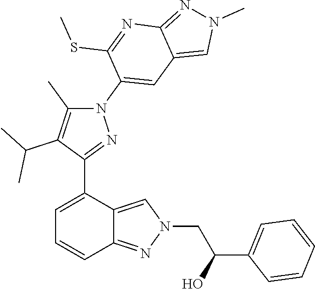 [SMILES: CSc1nc2nn(C)cc2cc1-n1nc(-c2cccc3nn(C[C@H](O)c4ccccc4)cc23)c(C(C)C)c1C] | G | 538 | 1.08 |
| | | (1R)-2-(4-{5-methyl-1-[2-methyl-6- | | | |
| | | (methylsulfanyl)-2H-pyrazolo[3,4- | | | |
| | | b]pyridin-5-yl]-4-(propan-2-yl)-1H- | | | |
| | | pyrazol-3-yl}-2H-indazol-2-yl)-1- | | | |
| | | phenylethan-1-ol | | | |
| 22 | Step 1: F41 + B8 PdCl₂(dtbpf) Cs₂CO₃ 80° C. 3 h Step 2: (chiral separation X) | 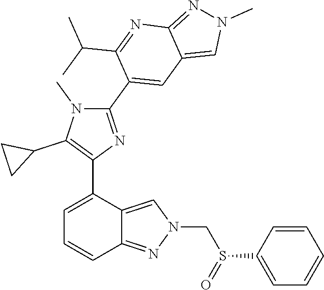 [SMILES: CC(C)c1nc2nn(C)cc2cc1-c1nc(-c2cccc3nn(C[S@@](=O)c4ccccc4)cc23)c(C2CC2)n1C] | G (X) | 550 | 0.74 (1.06) |
| | | 2-{[(S)-benzenesulfinyl]methyl}-4-{5- | | | |
| | | cyclopropyl-1-methyl-2-[2-methyl-6- | | | |
| | | (propan-2-yl)-2H-pyrazolo[3,4- | | | |
| | | b]pyridin-5-yl]-1H-imidazol-4-yl}-2H- | | | |
| | | indazole | | | |
| 23 | F23 + B3 Pd(dppf)Cl₂ × DCM Cs₂CO₃ 85° C., 16 h | 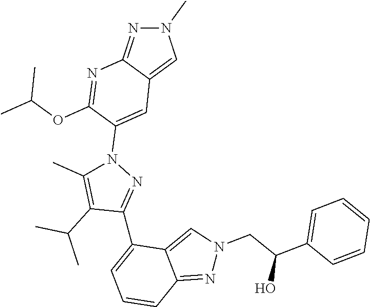 [SMILES: Cc1c(C(C)C)c(-c2cccc3nn(C[C@H](O)c4ccccc4)cc23)nn1-c1cc2cn(C)nc2nc1OC(C)C] | L | 550 | 1.00 |
| | | (1R)-2-(4-{5-methyl-1-[2-methyl-6- | | | |
| | | (propan-2-yloxy)-2H-pyrazolo[3, 4- | | | |
| | | b]pyridin-5-yl]-4-(propan-2-yl)-1H- | | | |
| | | pyrazol-3-yl}-2H-indazol-2-yl)-1- | | | |
| | | phenylethan-1-ol | | | |
| 25 | Step 1: F41 + B9 PdCl2(dtbpf) Cs₂CO₃ 80° C., 3 h Step 2: chiral separation method Y | 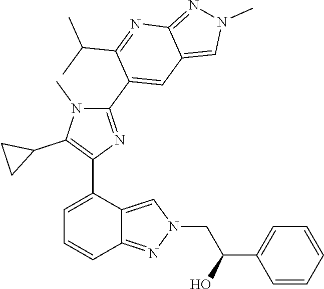 [SMILES: CC(C)c1nc2nn(C)cc2cc1-c1nc(-c2cccc3nn(C[C@H](O)c4ccccc4)cc23)c(C2CC2)n1C] | G (Y) | 546 | 0.77 (1.28) |
| | | (1R)-2-(4-{5-cyclopropyl-1-methyl-2- | | | |
| | | [2-methyl-6-(propan-2-yl)-2H- | | | |
| | | pyrazolo[3,4-b]pyridin-5-yl]-1H- | | | |
| | | imidazol-4-yl}-2H-indazol-2-yl)-1-(4- | | | |
| | | methylphenyl)ethan-1-ol | | | |
| 26 | F32 + B3 XPhos Pd G3 Cs₂CO₃ 90° C. 16 h | 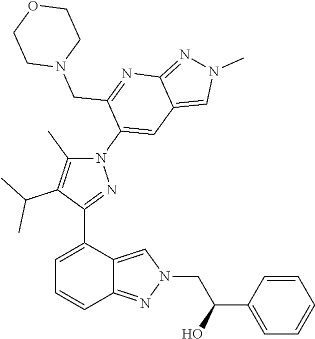 [SMILES: Cc1c(C(C)C)c(-c2cccc3nn(C[C@H](O)c4ccccc4)cc23)nn1-c1cc2cn(C)nc2nc1CN1CCOCC1] | C | 591 | 0.65 |
| | | (1R)-2-[4-(5-methyl-1-{2-methyl-6- | | | |
| | | [(morpholin-4-yl)methyl]-2H- | | | |
| | | pyrazolo[3,4-b]pyridin-5-yl}-4- | | | |
| | | (propan-2-yl)-1H-pyrazol-3-yl)-2H- | | | |
| | | indazol-2-yl]-1-phenylethan-1-ol | | | |
| 29 | F6 + B3 Pd(dppf)Cl₂ × DCM Cs₂CO₃ 100° C. 16 h | 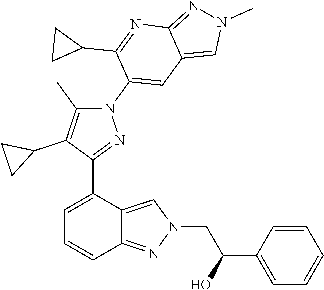 [SMILES: Cc1c(C2CC2)c(-c2cccc3nn(C[C@H](O)c4ccccc4)cc23)nn1-c1cc2cn(C)nc2nc1C1CC1] | P | 530 | 0.88 |
| | | (1R)-2-[4-(4-cyclopropyl-1-{6- | | | |
| | | cyclopropyl-2-methyl-2H-pyrazolo[3,4- | | | |
| | | b]pyridin-5-yl}-5-methyl-1H- | | | |
| | | pyrazol-3-yl)-2H-indazol-2-yl]-1- | | | |
| | | phenylethan-1-ol | | | |
| 34 | F45 + B3 PdCl₂(dtbpf) Na₂CO₃ 90° C. 19 h | 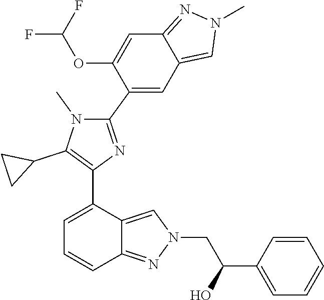 [SMILES: Cn1cc2cc(-c3nc(-c4cccc5nn(C[C@H](O)c6ccccc6)cc45)c(C4CC4)n3C)c(OC(F)F)cc2n1] | A | 555 | 0.49 |
| | | (1R)-2-(4-{5-cyclopropyl-2-[6- | | | |
| | | (difluoromethoxy)-2-methyl-2H- | | | |
| | | indazol-5-yl]-1-methyl-1H-imidazol-4- | | | |
| | | yl}-2H-indazol-2-yl)-1-phenylethan-1- | | | |
| | | ol | | | |
| 35 | F6 + B12 Pd(dppf)Cl₂ × DCM Cs₂CO₃ 80° C. 3 h | 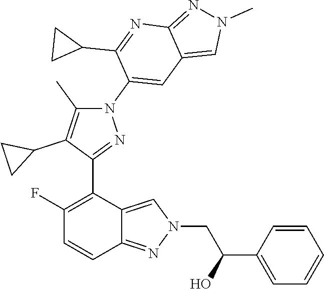 [SMILES: Cc1c(C2CC2)c(-c2c(F)ccc3nn(C[C@H](O)c4ccccc4)cc23)nn1-c1cc2cn(C)nc2nc1C1CC1] | A | 548 | 0.65 |
| | | (1R)-2-[4-(4-cyclopropyl-1-{6- | | | |
| | | cyclopropyl-2-methyl-2H-pyrazolo[3, | | | |
| | | 4-b]pyridin-5-yl}-5-methyl-1H- | | | |
| | | pyrazol-3-yl)-5-fluoro-2H-indazol-2- | | | |
| | | yl]-1-phenylethan-1-ol | | | |
| 38 | F7 + B3 PdCl₂(dtbpf) Na₂CO₃ 100° C. 16 h | 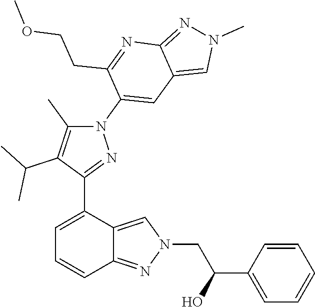 [SMILES: COCCc1nc2nn(C)cc2cc1-n1nc(-c2cccc3nn(C[C@H](O)c4ccccc4)cc23)c(C(C)C)c1C] | O | 550 | 0.83 |
| | | (1R)-2-(4-{1-[6-(2-methoxyethyl)-2- | | | |
| | | methyl-2H-pyrazolo[3,4-b]pyridin-5- | | | |
| | | yl]-5-methyl-4-(propan-2-yl)-1H- | | | |
| | | pyrazol-3-yl}-2H-indazol-2-yl)-1- | | | |
| | | phenylethan-1-ol | | | |
| 42 | F33 + B3 XPhos Pd G3 Cs₂CO₃ 90° C. 4 h | 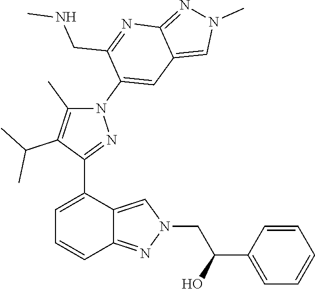 [SMILES: CNCc1nc2nn(C)cc2cc1-n1nc(-c2cccc3nn(C[C@H](O)c4ccccc4)cc23)c(C(C)C)c1C] | H | 535 | 1.02 |
| | | (1R)-2-[4-(5-methyl-1-{2-methyl-6- | | | |
| | | [(methylamino)methyl]-2H- | | | |
| | | pyrazolo[3,4-b]pyridin-5-yl}-4- | | | |
| | | (propan-2-yl)-1H-pyrazol-3-yl)-2H- | | | |
| | | indazol-2-yl]-1-phenylethan-1-ol | | | |
| 44 | Step 1: F2 + B15 Pd(dppf)Cl₂ × DCM Cs₂CO₃ 80 °C 16 h Step 2: Chiral separation: method Z | 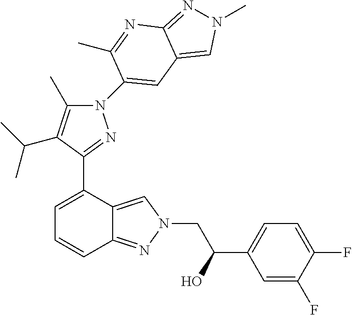 [SMILES: Cc1nc2nn(C)cc2cc1-n1nc(-c2cccc3nn(C[C@H](O)c4ccc(F)c(F)c4)cc23)c(C(C)C)c1C] | G (Z) | 542 | 1.02 (4.96) |
| | | (1R)-1-(3, 4-difluorophenyl)-2-[4-(1- | | | |
| | | {2, 6-dimethyl-2H-pyrazolo[3, 4- | | | |
| | | b]pyridin-5-yl}-5-methyl-4-(propan-2- | | | |
| | | yl)-1H-pyrazol-3-yl)-2H-indazol-2- | | | |
| | | yl]ethan-1-ol | | | |
| 46 | F2 + B21 Pd(dppf)Cl₂ × DCM Cs₂CO₃ 80° C. 3 h | 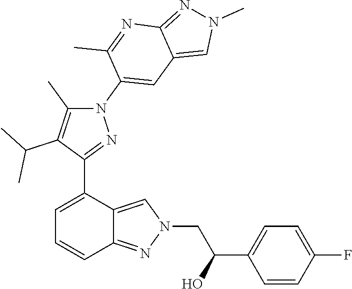 [SMILES: Cc1nc2nn(C)cc2cc1-n1nc(-c2cccc3nn(C[C@H](O)c4ccc(F)cc4)cc23)c(C(C)C)c1C] | P | 524 | 0.81 |
| | | (1R)-2-[4-(1-{2, 6-dimethyl-2H- | | | |
| | | pyrazolo[3,4-b]pyridin-5-yl}-5- | | | |
| | | methyl-4-(propan-2-yl)-1H-pyrazol-3- | | | |
| | | yl)-2H-indazol-2-yl]-1-(4- | | | |
| | | fluorophenyl)ethan-1-ol | | | |
| 47 | F8 + B1 XPhos Pd G3 K₃PO₄ 90° C. 2 h | 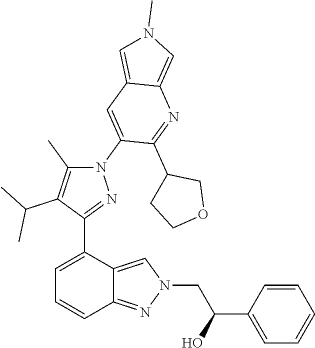 [SMILES: Cc1c(C(C)C)c(-c2cccc3nn(C[C@H](O)c4ccccc4)cc23)nn1-c1cc2cn(C)cc2nc1C1CCOC1] | H | 562 | 1.04 |
| | | (1R)-2-(4-{5-methyl-1-[2-methyl-6- | | | |
| | | (oxolan-3-yl)-2H-pyrazolo[3, 4- | | | |
| | | b]pyridin-5-yl]-4-(propan-2-yl)-1H- | | | |
| | | pyrazol-3-yl}-2H-indazol-2-yl)-1- | | | |
| | | phenylethan-1-ol | | | |
| 48 | F46 + B16 XPhos Pd G3 K₃PO₄ 80° C. 5 h | 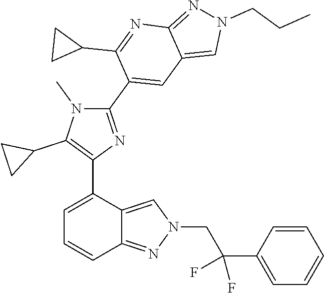 [SMILES: CCCn1cc2cc(-c3nc(-c4cccc5nn(CC(F)(F)c6ccccc6)cc45)c(C4CC4)n3C)c(C3CC3)nc2n1] | N | 578 | 0.98 |
| | | 4-(5-cyclopropyl-2-{6-cyclopropyl-2- | | | |
| | | propyl-2H-pyrazolo[3,4-b]pyridin-5- | | | |
| | | yl}-1-methyl-1H-imidazol-4-yl)-2-(2, | | | |
| | | 2-difluoro-2-phenylethyl)-2H- | | | |
| | | indazole | | | |
| 49 | F35 + B17 Pd(dppf)Cl₂ × DCM Cs₂CO₃ 80° C. 3 h | 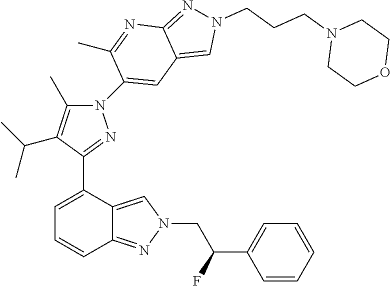 [SMILES: Cc1nc2nn(CCCN3CCOCC3)cc2cc1-n1nc(-c2cccc3nn(C[C@H](F)c4ccccc4)cc23)c(C(C)C)c1C] | N | 621 | 0.93 |
| | | 2-[(2R)-2-fluoro-2-phenylethyl]-4-(5- | | | |
| | | methyl-1-{6-methyl-2-[3-(morpholin- | | | |
| | | 4-yl)propyl]-2H-pyrazolo[3,4- | | | |
| | | b]pyridin-5-yl}-4-(propan-2-yl)-1H- | | | |
| | | pyrazol-3-yl)-2H-indazole | | | |
| 50 | F36 + B1 XPhos Pd G3 Cs₂CO₃ 90° C. 2 h | 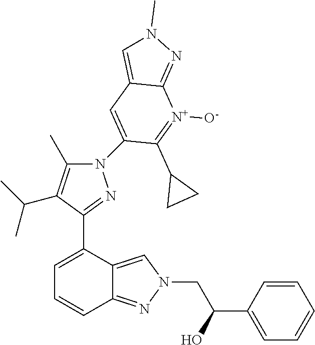 [SMILES: Cc1c(C(C)C)c(-c2cccc3nn(C[C@H](O)c4ccccc4)cc23)nn1-c1cc2cn(C)nc2[n+]([O-])c1C1CC1] | 0 | 548 | 0.76 |
| | | 6-cyclopropyl-5-(3-{2-[(2R)-2- | | | |
| | | hydroxy-2-phenylethyl]-2H-indazol-4- | | | |
| | | yl}-5-methyl-4-(propan-2-yl)-1H- | | | |
| | | pyrazol-1-yl)-2-methyl-2H- | | | |
| | | pyrazolo[3,4-b]pyridin-7-ium-7-olate | | | |
| 51 | F9 + B1 XPhos Pd G3 K₃PO₄ 90° C. 2 h | 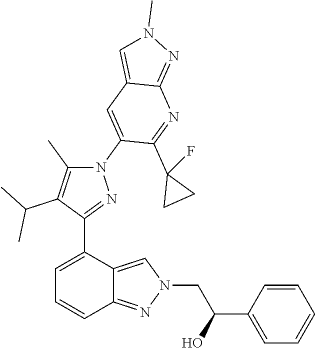 [SMILES: Cc1c(C(C)C)c(-c2cccc3nn(C[C@H](O)c4ccccc4)cc23)nn1-c1cc2cn(C)nc2nc1C1(F)CC1] | H | 550 | 1.08 |
| | | (1R)-2-(4-{1-[6-(1-fluorocyclopropyl)- | | | |
| | | 2-methyl-2H-pyrazolo[3,4-b]pyridin- | | | |
| | | 5-yl]-5-methyl-4-(propan-2-yl)-1H- | | | |
| | | pyrazol-3-yl}-2H-indazol-2-yl)-1- | | | |
| | | phenylethan-1-ol | | | |
| 53 | F10 + B3 Pd(dppf)Cl₂ × DCM Cs₂CO₃ 80° C. 3 h | 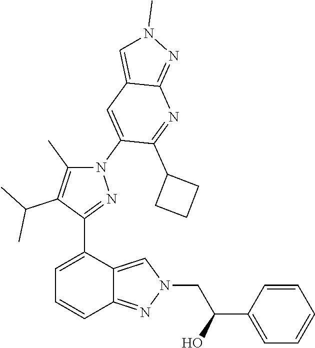 [SMILES: Cc1c(C(C)C)c(-c2cccc3nn(C[C@H](O)c4ccccc4)cc23)nn1-c1cc2cn(C)nc2nc1C1CCC1] | H | 546 | 1.15 |
| | | (1R)-2-[4-(1-{6-cyclobutyl-2-methyl- | | | |
| | | 2H-pyrazolo[3,4-b]pyridin-5-yl}-5- | | | |
| | | methyl-4-(propan-2-yl)-1H-pyrazol-3- | | | |
| | | yl)-2H-indazol-2-yl]-1-phenylethan-1- | | | |
| | | ol | | | |
| 54 | F11 + B1 XPhos Pd G3 K₃PO₄ 90° C. 2 h | 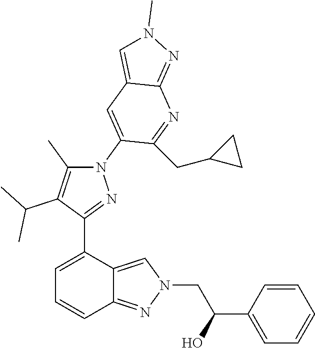 [SMILES: Cc1c(C(C)C)c(-c2cccc3nn(C[C@H](O)c4ccccc4)cc23)nn1-c1cc2cn(C)nc2nc1CC1CC1] | H | 546 | 1.09 |
| | | (1R)-2-(4-{1-[6-(cyclopropylmethyl)- | | | |
| | | 2-methyl-2H-pyrazolo[3,4-b]pyridin- | | | |
| | | 5-yl]-5-methyl-4-(propan-2-yl)-1H- | | | |
| | | pyrazol-3-yl}-2H-indazol-2-yl)-1- | | | |
| | | phenylethan-1-ol | | | |
| 55 | F42 + B14 XPhos Pd G3 Cs₂CO₃ 90° C. 2 h | 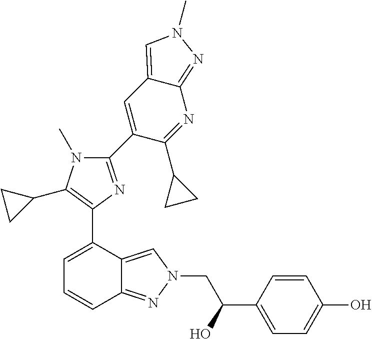 [SMILES: Cn1cc2cc(-c3nc(-c4cccc5nn(C[C@H](O)c6ccc(O)cc6)cc45)c(C4CC4)n3C)c(C3CC3)nc2n1] | A | 546 | 0.41 |
| | | 4-[(1R)-2-[4-(5-cyclopropyl-2-{6- | | | |
| | | cyclopropyl-2-methyl-2H-pyrazolo[3,4- | | | |
| | | b]pyridin-5-yl}-1-methyl-1H- | | | |
| | | imidazol-4-yl)-2H-indazol-2-yl]-1- | | | |
| | | hydroxyethyl]phenol | | | |
| 57 | F43 + B3 XPhos Pd G3 Cs₂CO₃ 90° C. 1 h | 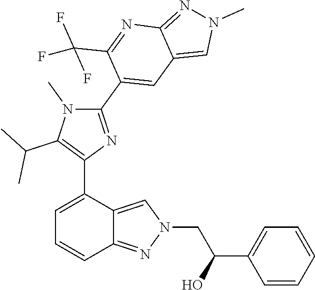 [SMILES: CC(C)c1c(-c2cccc3nn(C[C@H](O)c4ccccc4)cc23)nc(-c2cc3cn(C)nc3nc2C(F)(F)F)n1C] | G | 560 | 0.85 |
| | | (1R)-2-(4-{1-methyl-2-[2-methyl-6- | | | |
| | | (trifluoromethyl)-2H-pyrazolo[3, 4- | | | |
| | | b]pyridin-5-yl]-5-(propan-2-yl)-1H- | | | |
| | | imidazol-4-yl}-2H-indazol-2-yl)-1- | | | |
| | | phenylethan-1-ol | | | |
| 58 | F12 + B1 XPhos Pd G3 K₃PO₄ 90° C. 2 h | 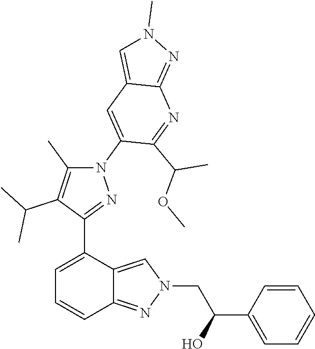 [SMILES: COC(C)c1nc2nn(C)cc2cc1-n1nc(-c2cccc3nn(C[C@H](O)c4ccccc4)cc23)c(C(C)C)c1C] | H | 550 | 1.04 |
| | | (1R)-2-(4-{1-[6-(1-methoxyethyl)-2- | | | |
| | | methyl-2H-pyrazolo[3,4-b]pyridin-5- | | | |
| | | yl]-5-methyl-4-(propan-2-yl)-1H- | | | |
| | | pyrazol-3-yl}-2H-indazol-2-yl)-1- | | | |
| | | phenylethan-1-ol | | | |
| 59 | F24 + B3 Pd(dppf)Cl₂ × DCM Cs₂CO₃ 80° C. 16 h | 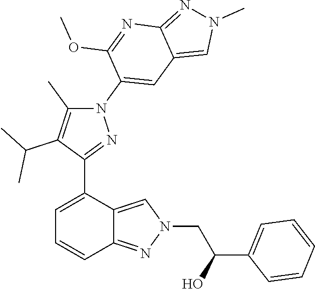 [SMILES: COc1nc2nn(C)cc2cc1-n1nc(-c2cccc3nn(C[C@H](O)c4ccccc4)cc23)c(C(C)C)c1C] | H | 522 | 1.08 |
| | | (1R)-2-[4-(1-{6-methoxy-2-methyl- | | | |
| | | 2H-pyrazolo[3,4-b]pyridin-5-yl}-5- | | | |
| | | methyl-4-(propan-2-yl)-1H-pyrazol-3- | | | |
| | | yl)-2H-indazol-2-yl]-1-phenylethan-1- | | | |
| | | ol | | | |
| 60 | F13 + B1 XPhos Pd G3 K₃PO₄ 100° C. 16 h | 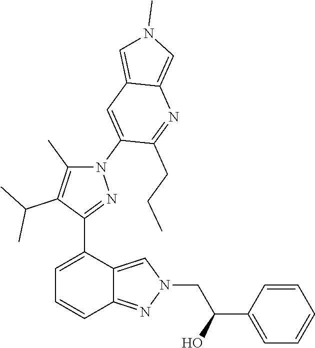 [SMILES: CCCc1nc2cn(C)cc2cc1-n1nc(-c2cccc3nn(C[C@H](O)c4ccccc4)cc23)c(C(C)C)c1C] | H | 534 | 1.09 |
| | | (1R)-2-[4-(5-methyl-1-{2-methyl-6- | | | |
| | | propyl-2H-pyrazolo[3,4-b]pyridin-5- | | | |
| | | yl}-4-(propan-2-yl)-1H-pyrazol-3-yl)- | | | |
| | | 2H-indazol-2-yl]-1-phenylethan-1-ol | | | |
| 64 | F37 + B3 Pd(dppf)Cl₂ × DCM Cs₂CO₃ 80° C. 3 h | 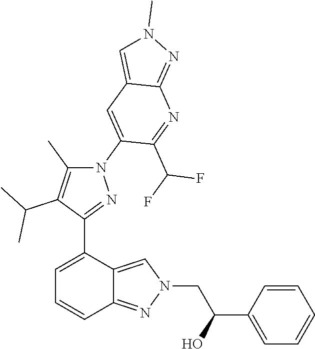 [SMILES: Cc1c(C(C)C)c(-c2cccc3nn(C[C@H](O)c4ccccc4)cc23)nn1-c1cc2cn(C)nc2nc1C(F)F] | K | 542 | 2.28 |
| | | (1R)-2-(4-{1-[6-(difluoromethyl)-2- | | | |
| | | methyl-2H-pyrazolo[3,4-b]pyridin-5- | | | |
| | | yl]-5-methyl-4-(propan-2-yl)-1H- | | | |
| | | pyrazol-3-yl}-2H-indazol-2-yl)-1- | | | |
| | | phenylethan-1-ol | | | |
| 65 | F2 + B18 Pd Xphos Gen3 Cs₂CO₃ 90° C. 4 h | 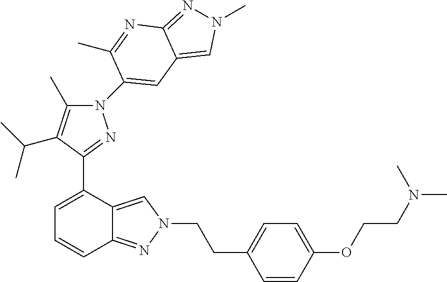 [SMILES: Cc1nc2nn(C)cc2cc1-n1nc(-c2cccc3nn(CCc4ccc(OCCN(C)C)cc4)cc23)c(C(C)C)c1C] | J | 577 | 0.64 |
| | | [2-(4-{2-[4-(1-{2, 6-dimethyl-2H- | | | |
| | | pyrazolo[3,4-b]pyridin-5-yl}-5- | | | |
| | | methyl-4-(propan-2-yl)-1H-pyrazol-3- | | | |
| | | yl)-2H-indazol-2- | | | |
| | | yl]ethyl}phenoxy)ethyl]dimethylamine | | | |
| 71 | F25 + B3 Xphos Crotyl-Pd—Cl Cs₂CO₃ 90° C. 16 h | 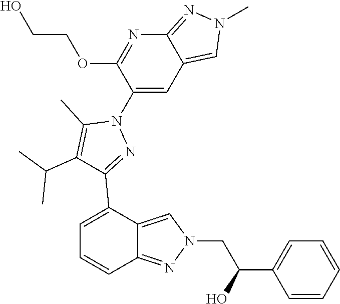 [SMILES: Cc1c(C(C)C)c(-c2cccc3nn(C[C@H](O)c4ccccc4)cc23)nn1-c1cc2cn(C)nc2nc1OCCO] | H | 552 | 0.99 |
| | | 2-{[5-(3-{2-[(2R)-2-hydroxy-2- | | | |
| | | phenylethyl]-2H-indazol-4-yl}-5- | | | |
| | | methyl-4-(propan-2-yl)-1H-pyrazol-1- | | | |
| | | yl)-2-methyl-2H-pyrazolo[3, 4- | | | |
| | | b]pyridin-6-yl]oxy}ethan-1-ol | | | |
| 72 | F47 + B19 PdCl₂(dtbpf) Na₂CO₃ 100° C. 5 h | 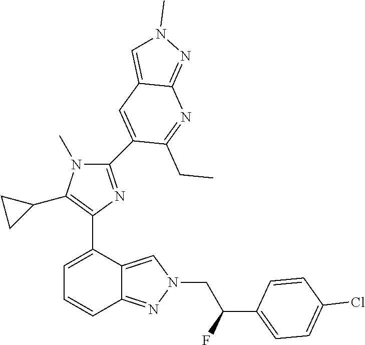 [SMILES: CCc1nc2nn(C)cc2cc1-c1nc(-c2cccc3nn(C[C@H](F)c4ccc(Cl)cc4)cc23)c(C2CC2)n1C] | A | 554/556 (Cl) | 0.54 |
| | | 2-[(2R)-2-(4-chlorophenyl)-2- | | | |
| | | fluoroethyl]-4-(5-cyclopropyl-2-{6- | | | |
| | | ethyl-2-methyl-2H-pyrazolo[3, 4- | | | |
| | | b]pyridin-5-yl}-1-methyl-1H-imidazol- | | | |
| | | 4-yl)-2H-indazole | | | |
| 73 | F14 + B3 Pd(dppf)Cl₂ × DCM Cs₂CO₃ 80° C. 3 h | 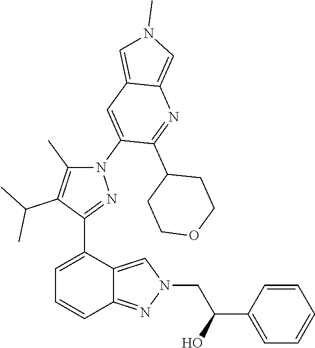 [SMILES: Cc1c(C(C)C)c(-c2cccc3nn(C[C@H](O)c4ccccc4)cc23)nn1-c1cc2cn(C)cc2nc1C1CCOCC1] | H | 576 | 1.07 |
| | | (1R)-2-(4-{5-methyl-1-[2-methyl-6- | | | |
| | | (oxan-4-yl)-2H-pyrazolo[3, 4- | | | |
| | | b]pyridin-5-yl]-4-(propan-2-yl)-1H- | | | |
| | | pyrazol-3-yl}-2H-indazol-2-yl)-1- | | | |
| | | phenylethan-1-ol | | | |
| 75 | F12 + B1 XPhos Pd G3 K₃PO₄ 90° C. 2 h | 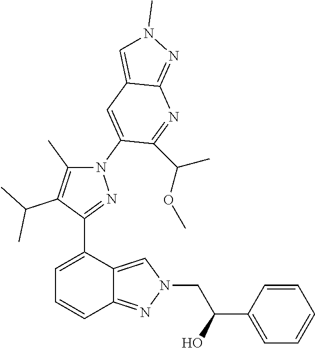 [SMILES: COC(C)c1nc2nn(C)cc2cc1-n1nc(-c2cccc3nn(C[C@H](O)c4ccccc4)cc23)c(C(C)C)c1C] | G | 550 | 1.05 |
| | | (1R)-2-[4-(1-{6-[(1-methoxyethyl]-2- | | | |
| | | methyl-2H-pyrazolo[3,4-b]pyridin-5- | | | |
| | | yl}-5-methyl-4-(propan-2-yl)-1H- | | | |
| | | pyrazol-3-yl)-2H-indazol-2-yl]-1- | | | |
| | | phenylethan-1-ol | | | |
| 76 | F38 + B3 Pd(dppf)Cl₂ × DCM Cs₂CO₃ 80° C. 5 h | 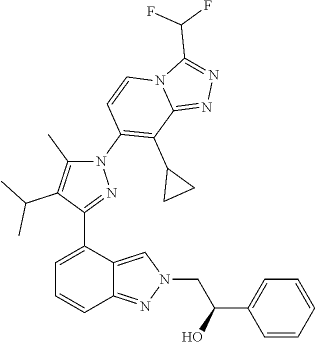 [SMILES: Cc1c(C(C)C)c(-c2cccc3nn(C[C@H](O)c4ccccc4)cc23)nn1-c1ccn2c(C(F)F)nnc2c1C1CC1] | A | 568 | 0.68 |
| | | (1R)-2-(4-{1-[8-cyclopropyl-3- | | | |
| | | (difluoromethyl)-[1,2,4]triazolo[4, 3- | | | |
| | | alpyridin-7-yl]-5-methyl-4-(propan-2- | | | |
| | | yl)-1H-pyrazol-3-yl}-2H-indazol-2-yl)- | | | |
| | | 1-phenylethan-1-ol | | | |
| 80 | F15 + B1 XPhos Pd G3 K₃PO₄ 90° C. 2 h | 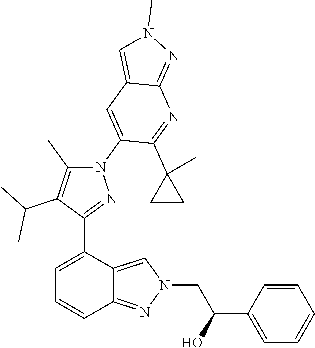 [SMILES: Cc1c(C(C)C)c(-c2cccc3nn(C[C@H](O)c4ccccc4)cc23)nn1-c1cc2cn(C)nc2nc1C1(C)CC1] | H | 546 | 1.08 |
| | | (1R)-2-(4-{5-methyl-1-[2-methyl-6-(1- | | | |
| | | methylcyclopropyl)-2H-pyrazolo[3, 4- | | | |
| | | b]pyridin-5-yl]-4-(propan-2-yl)-1H- | | | |
| | | pyrazol-3-yl}-2H-indazol-2-yl)-1- | | | |
| | | phenylethan-1-ol | | | |
| 82 | F2 + B10 Pd(dppf)Cl₂ × DCM Cs₂CO₃ 80° C. 16 h Step 2: chiral separation ZA): | 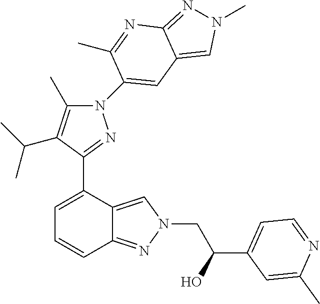 [SMILES: Cc1cc([C@@H](O)Cn2cc3c(-c4nn(-c5cc6cn(C)nc6nc5C)c(C)c4C(C)C)cccc3n2)ccn1] | Achiral: method G Chiral: Method ZA): | 521 | 0.81 (6.01) |
| | | (1R)-2-[4-(1-{2, 6-dimethyl-2H- | | | |
| | | pyrazolo[3,4-b]pyridin-5-yl}-5- | | | |
| | | methyl-4-(propan-2-yl)-1H-pyrazol-3- | | | |
| | | yl)-2H-indazol-2-yl]-1-(2- | | | |
| | | methylpyridin-4-yl)ethan-1-ol | | | |
| 83 | F2 + B20 Pd(dppf)Cl₂ × DCM Cs₂CO₃ 90° C. 3 h | 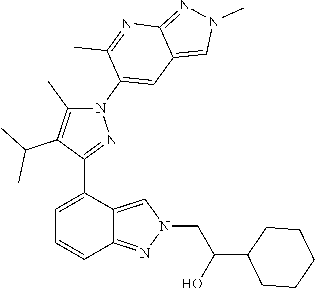 [SMILES: Cc1nc2nn(C)cc2cc1-n1nc(-c2cccc3nn(CC(O)C4CCCCC4)cc23)c(C(C)C)c1C] | J | 512 | 0.91 |
| | | 1-cyclohexyl-2-[4-(1-{2, 6-dimethyl- | | | |
| | | 2H-pyrazolo[3, 4-b]pyridin-5-yl}-5- | | | |
| | | methyl-4-(propan-2-yl)-1H-pyrazol-3- | | | |
| | | yl)-2H-indazol-2-yl]ethan-1-ol | | | |
| 84 | F26 + B3 Pd(dppf)Cl₂ × DCM Cs₂CO₃ 80° C. 2 d | 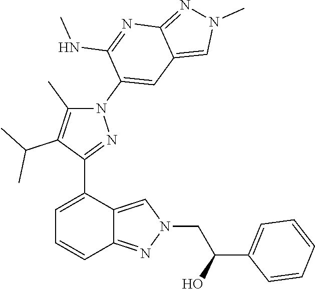 [SMILES: CNc1nc2nn(C)cc2cc1-n1nc(-c2cccc3nn(C[C@H](O)c4ccccc4)cc23)c(C(C)C)c1C] | G | 521 | 1.07 |
| | | (1R)-2-(4-{5-methyl-1-[2-methyl-6- | | | |
| | | (methylamino)-2H-pyrazolo[3, 4- | | | |
| | | b]pyridin-5-yl]-4-(propan-2-yl)-1H- | | | |
| | | pyrazol-3-yl}-2H-indazol-2-yl)-1- | | | |
| | | phenylethan-1-ol | | | |
| 86 | F39 + B3 Pd(dppf)Cl₂ × DCM Cs₂CO₃ 80° C. 4 h | 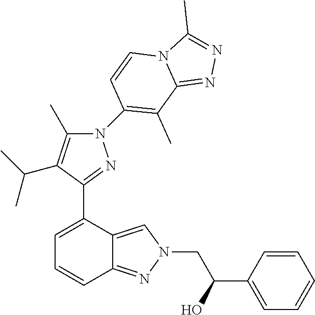 [SMILES: Cc1c(-n2nc(-c3cccc4nn(C[C@H](O)c5ccccc5)cc34)c(C(C)C)c2C)ccn2c(C)nnc12] | A | 506 | 0.57 |
| | | (1R)-2-[4-(1-{3, 8-dimethyl-[1,2,4] | | | |
| | | triazolo[4,3-a]pyridin-7-yl}-5- | | | |
| | | methyl-4-(propan-2-yl)-1H-pyrazol-3- | | | |
| | | yl)-2H-indazol-2-yl]-1-phenylethan-1- | | | |
| | | ol | | | |
| 87 | F27 + B3 Pd(dppf)Cl₂ × DCM Cs₂CO₃ 80° C. 16 h | 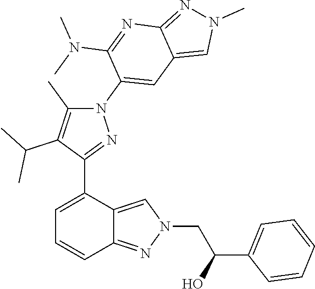 [SMILES: Cc1c(C(C)C)c(-c2cccc3nn(C[C@H](O)c4ccccc4)cc23)nn1-c1cc2cn(C)nc2nc1N(C)C] | N | 535 | 0.89 |
| | | (1R)-2-(4-{1-[6-(dimethylamino)-2- | | | |
| | | methyl-2H-pyrazolo[3,4-b]pyridin-5- | | | |
| | | yl]-5-methyl-4-(propan-2-yl)-1H- | | | |
| | | pyrazol-3-yl}-2H-indazol-2-yl)-1- | | | |
| | | phenylethan-1-ol | | | |
| 89 | F16 + B3 Pd(dppf)Cl₂ × DCM Na₂CO₃ 80° C. 16 h | 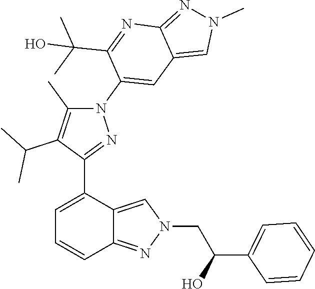 [SMILES: Cc1c(C(C)C)c(-c2cccc3nn(C[C@H](O)c4ccccc4)cc23)nn1-c1cc2cn(C)nc2nc1C(C)(C)O] | M | 550 | 2.10 |
| | | 2-[5-(3-{2-[(2R)-2-hydroxy-2- | | | |
| | | phenylethyl]-2H-indazol-4-yl}-5- | | | |
| | | methyl-4-(propan-2-yl)-1H-pyrazol-1- | | | |
| | | yl)-2-methyl-2H-pyrazolo[3, 4- | | | |
| | | b]pyridin-6-yl]propan-2-ol | | | |
| 91 | F42 + B11 XPhos Pd G3 K₃PO₄ 100° C. 16 h Step 2: chiral separation: W | 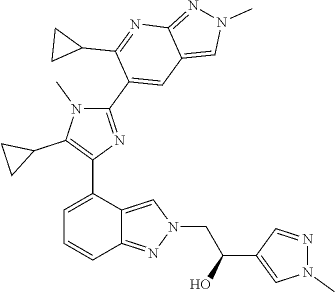 [SMILES: Cn1cc([C@@H](O)Cn2cc3c(-c4nc(-c5cc6cn(C)nc6nc5C5CC5)n(C)c4C4CC4)cccc3n2)cn1] | G (W) | 534 | 0.76 (5.22) |
| | | (1R)-2-[4-(5-cyclopropyl-2-{6- | | | |
| | | cyclopropyl-2-methyl-2H-pyrazolo[3,4- | | | |
| | | b]pyridin-5-yl}-1-methyl-1H- | | | |
| | | imidazol-4-yl)-2H-indazol-2-yl]-1-(1- | | | |
| | | methyl-1H-pyrazol-4-yl)ethan-1-ol | | | |
| 93 | F17 + B1 Pd(dppf)Cl₂ × DCM Na₂CO₃ 85° C. 3 h | 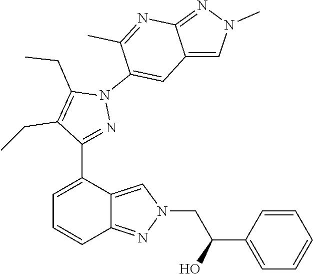 [SMILES: CCc1c(-c2cccc3nn(C[C@H](O)c4ccccc4)cc23)nn(-c2cc3cn(C)nc3nc2C)c1CC] | G | 506 | 1.00 |
| | | (1R)-2-[4-(1-{2, 6-dimethyl-2H- | | | |
| | | pyrazolo[3,4-b]pyridin-5-yl}-4, 5- | | | |
| | | diethyl-1H-pyrazol-3-yl)-2H-indazol- | | | |
| | | 2-yl]-1-phenylethan-1-ol | | | |
| 94 | F48 + B3 Pd(dppf)Cl₂ × DCM Cs₂CO₃ 80° C. 4 h | 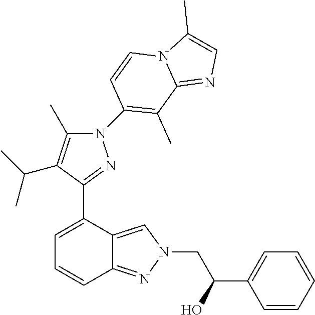 [SMILES: Cc1c(-n2nc(-c3cccc4nn(C[C@H](O)c5ccccc5)cc34)c(C(C)C)c2C)ccn2c(C)cnc12] | A | 505 | 0.65 |
| | | (1R)-2-[4-(1-{3, 8-dimethylimidazo[1,2- | | | |
| | | a]pyridin-7-yl}-5-methyl-4-(propan- | | | |
| | | 2-yl)-1H-pyrazol-3-yl)-2H-indazol-2- | | | |
| | | yl]-1-phenylethan-1-ol | | | |
| 95 | F49 + B3 XPhos Pd G3 Cs₂CO₃ 87° C. 4 h | 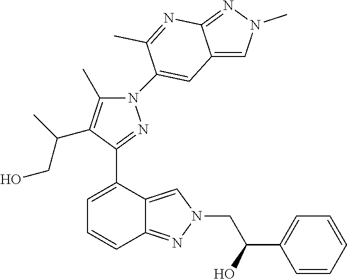 [SMILES: Cc1nc2nn(C)cc2cc1-n1nc(-c2cccc3nn(C[C@H](O)c4ccccc4)cc23)c(C(C)CO)c1C] | H | 522 | 0.73 |
| | | 2-(1-{2, 6-dimethyl-2H-pyrazolo[3, 4- | | | |
| | | b]pyridin-5-yl}-3-{2-[(2R)-2-hydroxy- | | | |
| | | 2-phenylethyl]-2H-indazol-4-yl}-5- | | | |
| | | methyl-1H-pyrazol-4-yl)propan-1-ol | | | |
| 101 | F4 + B22 Pd dppf Cs₂CO₃ 80° C., 3h & RT 15 h | 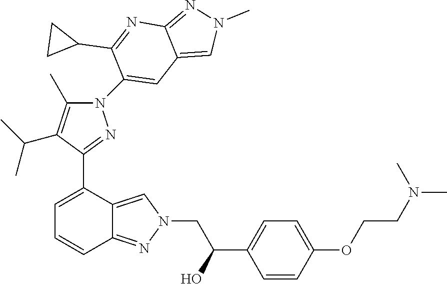 [SMILES: Cc1c(C(C)C)c(-c2cccc3nn(C[C@H](O)c4ccc(OCCN(C)C)cc4)cc23)nn1-c1cc2cn(C)nc2nc1C1CC1] | J | 619 | 0.68 |
| 102 | F2 + B2 XPhos Crotyl-Pd—Cl Cs₂CO₃ 90° C. 4 h | 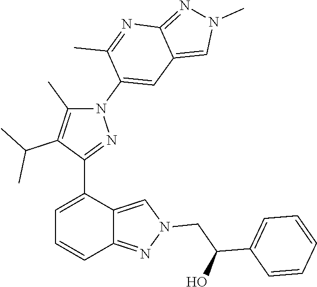 [SMILES: Cc1nc2nn(C)cc2cc1-n1nc(-c2cccc3nn(C[C@H](O)c4ccccc4)cc23)c(C(C)C)c1C] | H | 506 | 1.01 |
| | | (1R)-2-[4-(1-{2,6-dimethyl-2H- | | | |
| | | pyrazolo[3,4-b]pyridin-5-yl}-5- | | | |
| | | methyl-4-(propan-2-yl)-1H-pyrazol-3- | | | |
| | | yl)-2H-indazol-2-yl]-1-phenylethan-1- | | | |
| | | ol | | | |
| 105 | F50 + B1 | 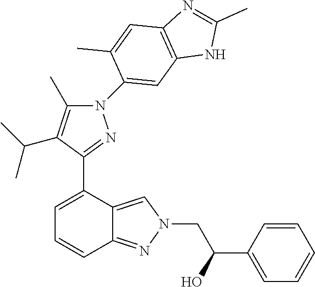 [SMILES: Cc1nc2cc(C)c(-n3nc(-c4cccc5nn(C[C@H](O)c6ccccc6)cc45)c(C(C)C)c3C)cc2[nH]1] | G | 505 | 0.92 |
| | | (1R)-2-{4-[1-(2,5-dimethyl-1H-1, 3- | | | |
| | | benzodiazol-6-yl)-5-methyl-4- | | | |
| | | (propan-2-yl)-1H-pyrazol-3-yl]-2H- | | | |
| | | indazol-2-yl}-1-phenylethan-1-ol | | | |
| 106 | F28 + B3 | 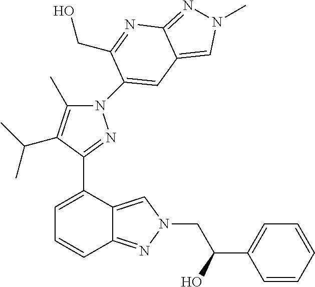 [SMILES: Cc1c(C(C)C)c(-c2cccc3nn(C[C@H](O)c4ccccc4)cc23)nn1-c1cc2cn(C)nc2nc1CO] | L | 522 | 0.79 |
| | | (1R)-2-(4-{1-[6-(hydroxymethyl)-2- | | | |
| | | methyl-2H-pyrazolo[3,4-b]pyridin-5- | | | |
| | | yl]-5-methyl-4-(propan-2-yl)-1H- | | | |
| | | pyrazol-3-yl}-2H-indazol-2-yl)-1- | | | |
| | | phenylethan-1-ol | | | |
| 107 | F47 + B17 | 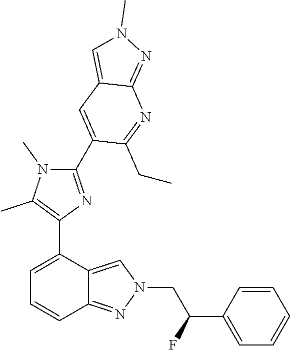 [SMILES: CCc1nc2nn(C)cc2cc1-c1nc(-c2cccc3nn(C[C@H](F)c4ccccc4)cc23)c(C)n1C] | L | 520 | 0.65 |
| | | 4-(5-cyclopropyl-2-{6-ethyl-2-methyl- | | | |
| | | 2H-pyrazolo[3,4-b]pyridin-5-yl}-1- | | | |
| | | methyl-1H-imidazol-4-yl)-2-[(2R)-2- | | | |
| | | fluoro-2-phenylethyl]-2H-indazole | | | |
| 109 | F51 + B2 | 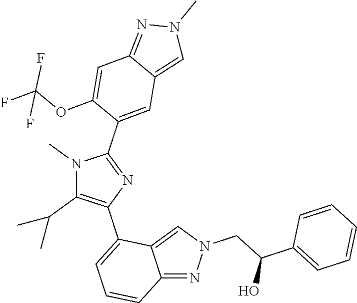 [SMILES: CC(C)c1c(-c2cccc3nn(C[C@H](O)c4ccccc4)cc23)nc(-c2cc3cn(C)nc3cc2OC(F)(F)F)n1C] | I | 575 | 0.84 |
| | | (1R)-2-(4-{1-methyl-2-[2-methyl-6- | | | |
| | | (trifluoromethoxy)-2H-indazol-5-yl]-5- | | | |
| | | (propan-2-yl)-1H-imidazol-4-yl}-2H- | | | |
| | | indazol-2-yl)-1-phenylethan-1-ol | | | |
| 110 | F44 + B1 | 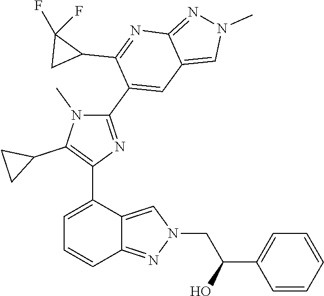 [SMILES: Cn1cc2cc(-c3nc(-c4cccc5nn(C[C@H](O)c6ccccc6)cc45)c(C4CC4)n3C)c(C3CC3(F)F)nc2n1] | G | 566 | 0.83 |
| | | (1R)-2-(4-{5-cyclopropyl-2-[6-(2, 2- | | | |
| | | difluorocyclopropyl)-2-methyl-2H- | | | |
| | | pyrazolo[3,4-b]pyridin-5-yl]-1- | | | |
| | | methyl-1H-imidazol-4-yl}-2H-indazol- | | | |
| | | 2-yl)-1-phenylethan-1-ol | | | |
| 112 | F34 + B3 | 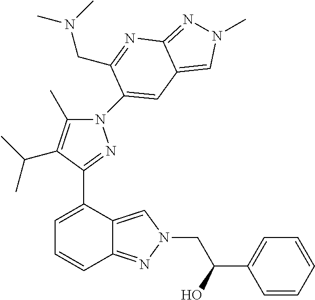 [SMILES: Cc1c(C(C)C)c(-c2cccc3nn(C[C@H](O)c4ccccc4)cc23)nn1-c1cc2cn(C)nc2nc1CN(C)C] | J | 549 | 0.65 |
| | | (1R)-2-[4-(1-{6- | | | |
| | | [(dimethylamino)methyl]-2-methyl- | | | |
| | | 2H-pyrazolo[3,4-b]pyridin-5-yl}-5- | | | |
| | | methyl-4-(propan-2-phenylethan-1-ol | | | |
| 113 | F20 + B3 | 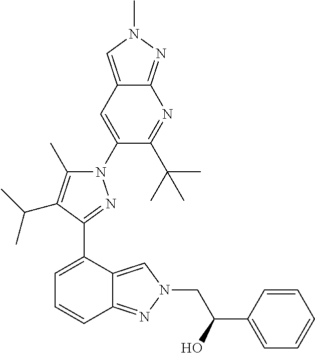 [SMILES: Cc1c(C(C)C)c(-c2cccc3nn(C[C@H](O)c4ccccc4)cc23)nn1-c1cc2cn(C)nc2nc1C(C)(C)C] | H | 548 | 1.18 |
| | | (1R)-2-[4-(1-{6-tert-butyl-2-methyl- | | | |
| | | 2H-pyrazolo[3,4-b]pyridin-5-yl}-5- | | | |
| | | methyl-4-(propan-2-yl)-1 H-pyrazol-3- | | | |
| | | yl)-2H-indazol-2-yl]-1-phenylethan-1- | | | |
| | | ol | | | |

### Synthesis of Intermediate G2, example 10, 11, 28, 33, 61 and 104

### Synthesis of Intermediate G2: (1R)-2-[4-(5-cyclopropyl-2-{6-cyclopropyl-2H-pyrazolo[3, 4-b]pyridin-5-yl)-1-methyl-1H-imidazol-4-yl)-21H-indazol-2-yl]-1-phenylethan-1-ol

### Step 1: (1R)-2-[4-(5-cyolopropyl-2-{6-cyolopropyl-2-[(4-methoxyphenyl)methyl]-2H-pyrazolo[3, 4-b]pyridin-5-yl)-1-methyl-1H-imidazol-4-yl)-2H-indazol-2-yl]-1-phenylethan-1-ol

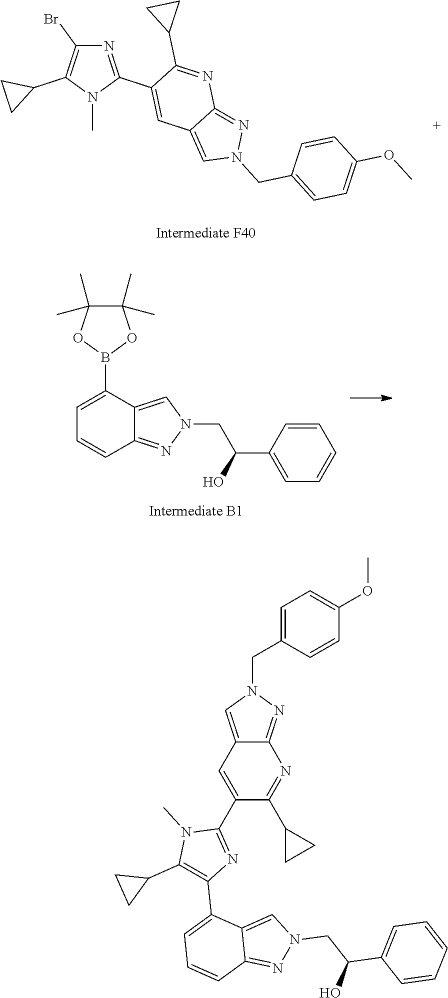

Under an argon atmosphere, 5-(4-bromo-5-cyclopropyl-1-methyl-imidazol-2-yl)-6-cyclopropyl-2-[(4-methoxyphenyl) methyl]pyrazolo[3, 4-b]pyridine (F40) (500 mg, 1.05 mmol) and (1R)-1-phenyl-2-[4-(4, 4, 5, 5-tetramethyl-1, 3, 2-dioxaborolan-2-yl)-2H-indazol-2-yl]ethan-1-ol (B1) (508 mg, 1.25 mmol) are dissolved in dioxane (10 mL). An aqueous K₃PO₄ solution (1 mL, 2 M) and (2-dicyclohexylphosphino-2′, 4′, 6′-triisopropyl-1, 1′-biphenyl) [2-(2′-amino-1, 1′-biphenyl)]palladium(II) methanesulfonate (XPhos Pd G3, 44.2 mg, 0.05 mmol) are added and the reaction mixture is stirred at 90° C. for 1 h. Then the solids are removed by filtration. The filtrate is concentrated to provide the product, which is used in the next step without further purification.

Analysis (method H): Rₜ: 1.1 min, [M+H]⁺: 636

### Step 2: Synthesis of (1R)-2-[4-(5-cyclopropyl-2-{6-cyclopropyl-2H-pyrazolo[3, 4-b]pyridin-5-yl}-1-methyl-1H-imidazol-4-yl)-2H-indazol-2-yl]-1-phenylethan-1-ol

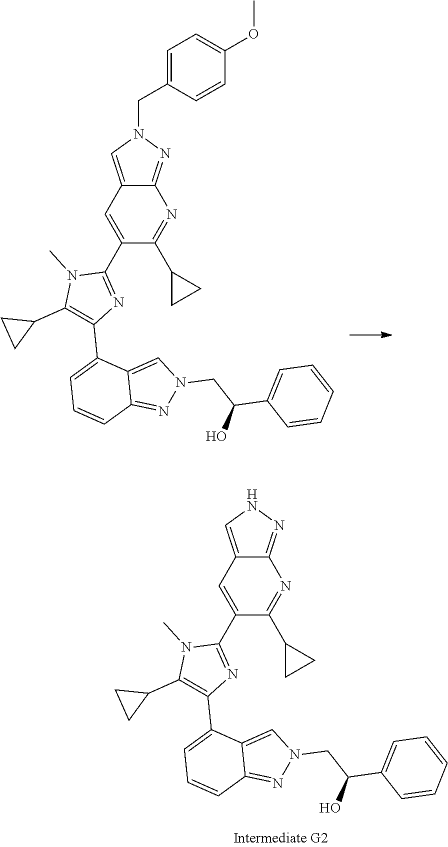 [SMILES: COc1ccc(Cn2cc3cc(-c4nc(-c5cccc6nn(C[C@H](O)c7ccccc7)cc56)c(C5CC5)n4C)c(C4CC4)nc3n2)cc1 ; Cn1c(-c2cc3c[nH]nc3nc2C2CC2)nc(-c2cccc3nn(C[C@H](O)c4ccccc4)cc23)c1C1CC1]

(1R)-2-[4-(5-cyclopropyl-2-{6-cyclopropyl-2-[(4-methoxyphenyl)methyl]-2H-pyrazolo[3, 4-b]pyridin-5-yl}-1-methyl-1H-imidazol-4-yl)-2H-indazol-2-yl]-1-phenylethan-1-ol (600 mg, 0.94 mmol) and anisole (207 μL, 1.88 mmol) are dissolved in DCE (20 mL). TFA (5 mL) is added, and the reaction mixture is stirred at 70° C. over the weekend. The reaction mixture is concentrated, and the residue is purified by reversed phase chromatography (HPLC; ACN/water including TFA) to afford intermediate G2.

Analysis (method H): Rₜ: 1, 03 min, [M+H]⁺: 516

### Synthesis of Example 10 and Example 61: (1R)-2-[4-(5-cyclopropyl-2-{6-cyclopropyl-2-[2-(morpholin-4-yl)ethyl]-2H-pyrazolo[3, 4-b]pyridin-5-yl}-1-methyl-1H-imidazol-4-yl)-2H-indazol-2-yl]-1-phenylethan-1-ol (10) and (1R)-2-[4-(5-cyclopropyl-2-{6-cyclopropyl-2-ethenyl-2H-pyrazolo[3, 4-b]pyridin-5-yl}-1-methyl-1H-imidazol-4-yl)-2H-indazol-2-yl]-1-phenylethan-1-ol (61)

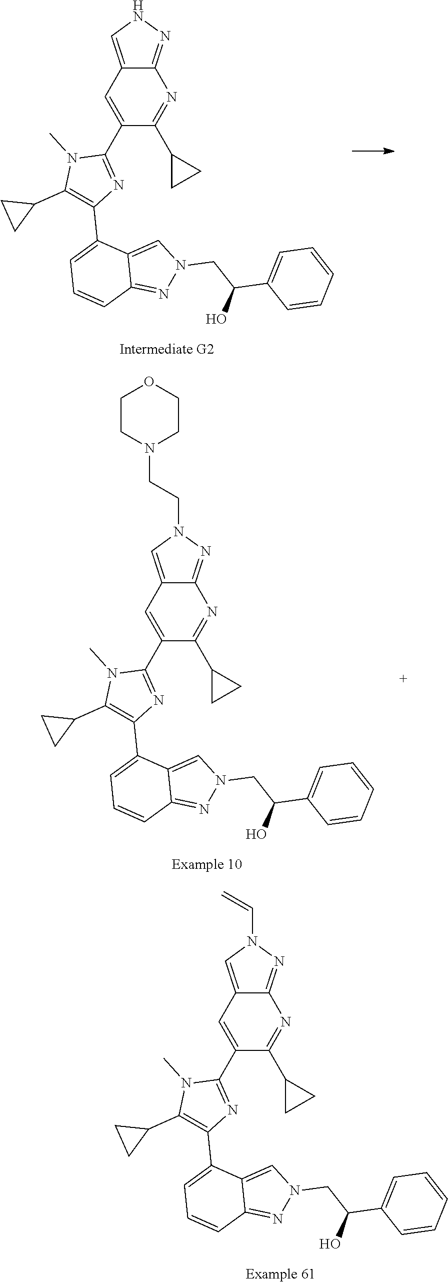 [SMILES: Cn1c(-c2cc3c[nH]nc3nc2C2CC2)nc(-c2cccc3nn(C[C@H](O)c4ccccc4)cc23)c1C1CC1 ; Cn1c(-c2cc3cn(CCN4CCOCC4)nc3nc2C2CC2)nc(-c2cccc3nn(C[C@H](O)c4ccccc4)cc23)c1C1CC1 ; C=Cn1cc2cc(-c3nc(-c4cccc5nn(C[C@H](O)c6ccccc6)cc45)c(C4CC4)n3C)c(C3CC3)nc2n1]

((1R)-2-[4-(5-Cyclopropyl-2-{6-cyclopropyl-2H-pyrazolo[3, 4-b]pyridin-5-yl}-1-methyl-1H-imidazol-4-yl)-2H-indazol-2-yl]-1-phenylethan-1-ol (40 mg, 0.08 mmol), 1-bromo-2-chloroethane (16 μL, 0.19 mmol) is dissolved in ACN (2 mL) and K₂CO₃ (53 mg) is added and the mixture stirred at 40° C. overnight. Then morpholine (34 μL, 0.39 mmol) is added, the mixture is stirred for 4 h at 80° C., then additional morpholine (50 μL) is added and the mixture is stirred overnight at 80° C. The mixture is then diluted with water, filtered, and purified by reversed phase chromatography (HPLC; ACN/water including NH₃) to afford the title compounds.

Analysis (method H): Rₜ: 1.01 min, [M+H]⁺: 629 (example 10)

Analysis (method H): Rₜ: 1.02 min, [M+H]⁺: 542 (example 61)

### Synthesis of Example 11: (1R)-2-[4-(5-cyclopropyl-2-{6-cyclopropyl-2-[3-(morpholin-4-yl)propyl]-2H-pyrazolo[3, 4-b]pyridin-5-yl}-1-methyl-1H-imidazol-4-yl)-2H-indazol-2-yl]-1-phenylethan-1-ol

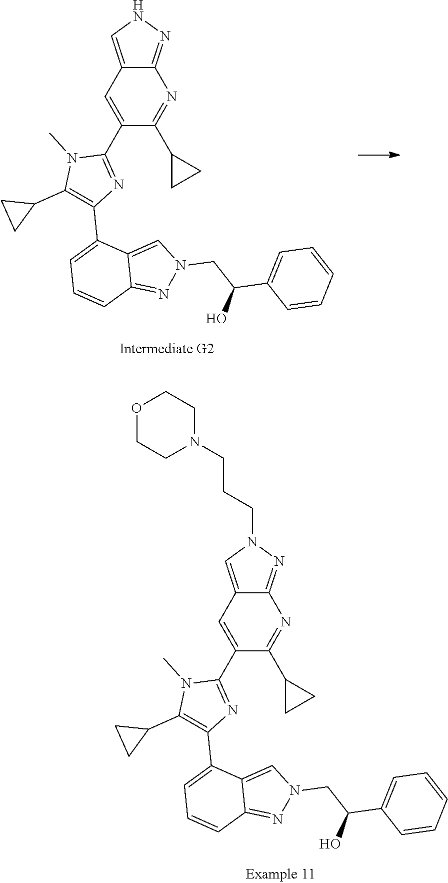 [SMILES: Cn1c(-c2cc3c[nH]nc3nc2C2CC2)nc(-c2cccc3nn(C[C@H](O)c4ccccc4)cc23)c1C1CC1 ; Cn1c(-c2cc3cn(CCCN4CCOCC4)nc3nc2C2CC2)nc(-c2cccc3nn(C[C@H](O)c4ccccc4)cc23)c1C1CC1]

((1R)-2-[4-(5-Cyclopropyl-2-{6-cyclopropyl-2H-pyrazolo[3, 4-b]pyridin-5-yl}-1-methyl-1H-imidazol-4-yl)-2H-indazol-2-yl]-1-phenylethan-1-ol (40 mg, 0.08 mmol) and 4-(3-bromopropyl)morpholine 1-bromo-2-chloroethane (25.5 mg, 0.12 mmol) is dissolved in ACN (1.9 mL) and K₂CO₃ (32 mg) is added and the mixture is stirred at 40° C. overnight. The mixture is then diluted with water/ACN (2 mL), filtered, and purified by reversed phase chromatography (HPLC; C18, ACN/water including NH3) to afford example 11.

Analysis (method H): Rₜ: 1.01 min, [M+H]⁺: 643

The intermediate and the examples compiled in the following table are obtained by following a procedure analogous to that described for example 11 using intermediate G2.

| | | | | MS | |
|---|---|---|---|---|---|
| | | | | (ESI+): | |
| | | | LC | m/z | t_{R} |
| Example | Synthesis from | Structure/Name | Method | [M + H]⁺ | [min] |
| 28 | G2 + 1-(3- chloropropyl) pyrrolidin-2-one; 40° C. overnight | 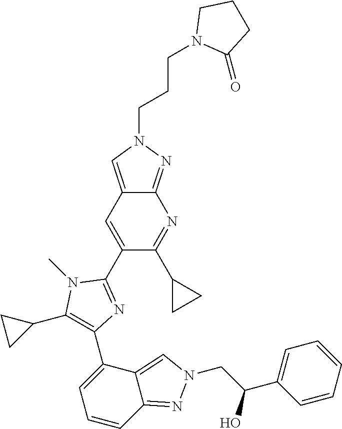 [SMILES: Cn1c(-c2cc3cn(CCCN4CCCC4=O)nc3nc2C2CC2)nc(-c2cccc3nn(C[C@H](O)c4ccccc4)cc23)c1C1CC1] | H | 641 | 1.00 |
| | | 1-{3-[6-cyclopropyl-5-(5-cyclopropyl-4-{2- | | | |
| | | [(2R)-2-hydroxy-2-phenylethyl]-2H-indazol-4-yl}- | | | |
| | | 1-methyl-1H-imidazol-2-yl)-2H-pyrazolo[3, 4-b] | | | |
| | | pyridin-2-yl]propyl}pyrrolidin-2-one | | | |
| 33 | G2 + 2- bromopropane; 60° C. overnight | 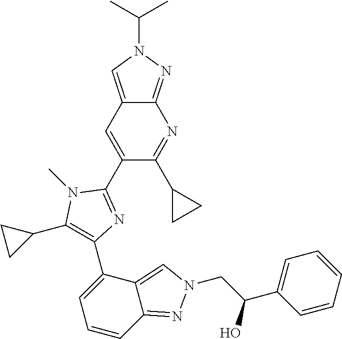 [SMILES: CC(C)n1cc2cc(-c3nc(-c4cccc5nn(C[C@H](O)c6ccccc6)cc45)c(C4CC4)n3C)c(C3CC3)nc2n1] | H | 558 | 1.07 |
| | | (1R)-2-(4-{5-cyclopropyl-2-[6- | | | |
| | | cyclopropyl-2-(propan-2-yl)-2H- | | | |
| | | pyrazolo[3, 4-b]pyridin-5-yl]-1-methyl-1H- | | | |
| | | imidazol-4-yl}-2H-indazol-2-yl)-1-phenylethan-1-ol | | | |
| 104 | G2 + 3- bromopropionitrile; 40° C. overnight | 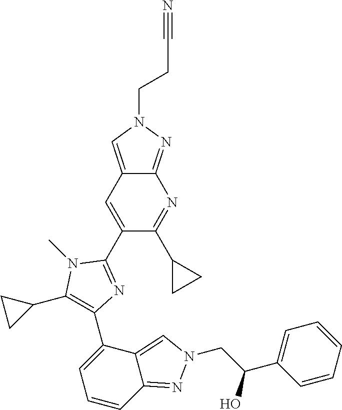 [SMILES: Cn1c(-c2cc3cn(CCC#N)nc3nc2C2CC2)nc(-c2cccc3nn(C[C@H](O)c4ccccc4)cc23)c1C1CC1] | H | 569 | 1.00 |
| | | 3-[6-cyclopropyl-5-(5-cyclopropyl-4-{2-[(2R)-2- | | | |
| | | hydroxy-2-phenylethyl]-2H-indazol-4-yl}- | | | |
| | | 1-methyl-1H-imidazol-2-yl)-2H- | | | |
| | | pyrazolo[3, 4-b]pyridin-2-yl]propanenitrile | | | |

### Synthesis of Examples 8, 27, 30, 52, 63, 66, 74, 78, 79, 81, 85

### Synthesis of Example 8: 2-[(1R)-2-[4-(1-{2, 6-dimethyl-2H-pyrazolo[3, 4-b]pyridin-5-yl}-5-methyl-4-(propan-2-yl)-1H-pyrazol-3-yl)-2H-indazol-2-yl]-1-phenylethoxy]ethan-1-ol

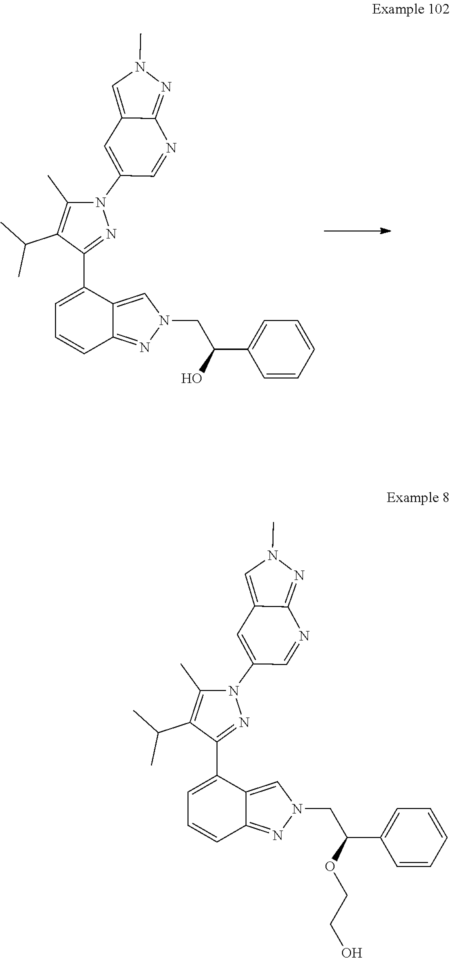 [SMILES: Cc1c(C(C)C)c(-c2cccc3nn(C[C@H](O)c4ccccc4)cc23)nn1-c1cnc2nn(C)cc2c1 ; Cc1c(C(C)C)c(-c2cccc3nn(C[C@H](OCCO)c4ccccc4)cc23)nn1-c1cnc2nn(C)cc2c1]

(1R)-2-[4-(1-{2, 6-Dimethyl-2H-pyrazolo[3, 4-b]pyridin-5-yl}-5-methyl-4-(propan-2-yl)-1H-pyrazol-3-yl)-2H-indazol-2-yl]-1-phenylethan-1-ol (102) (25.3 mg, 0.05 mmol) is dissolved in DMF (1 mL). NaH (6.6 mg, 0.15 mmol, 55% purity) is added and the reaction mixture is stirred at RT for 10 min. Then 2-(2-chloroethoxy) tetrahydro-2H-pyran (32.9 mg, 0.20 mmol) is added and the reaction mixture is stirred at RT overnight. The reaction mixture is purified by reversed phase chromatography (HPLC; ACN/water/TFA). To remove the THF protecting group, the residue is dissolved in TFA (1 mL) and stirred at 50° C. for 30 min. The reaction mixture is concentrated and purified by reversed phase chromatography (HPLC; ACN/water including TEA) to afford the title compound.

Analysis (method G): Rₜ: 1.01 min [M+H]⁺: 550

The examples compiled in the following table are obtained by following a procedure analogous to that described for example 8.

| | | | | MS | |
|---|---|---|---|---|---|
| | | | | (ESI+): | |
| | | | LC | m/z | t_{R} |
| Example | Synthesis from | Structure/Name | Method | [M + H]⁺ | [min] |
| 27 | 102 + fluoroiodomethane | 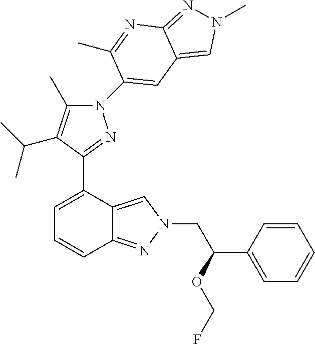 [SMILES: Cc1nc2nn(C)cc2cc1-n1nc(-c2cccc3nn(C[C@H](OCF)c4ccccc4)cc23)c(C(C)C)c1C] | G | 538 | 1.08 |
| | | 4-(1-{2, 6-dimethyl-2H-pyrazolo[3, 4-b] | | | |
| | | pyridin-5-yl}-5-methyl-4-(propan-2-yl)-1H- | | | |
| | | pyrazol-3-yl)-2-[(2R)-2-(fluoromethoxy)-2- | | | |
| | | phenylethyl]-2H-indazole | | | |
| 30 | 102 + iodoethane | 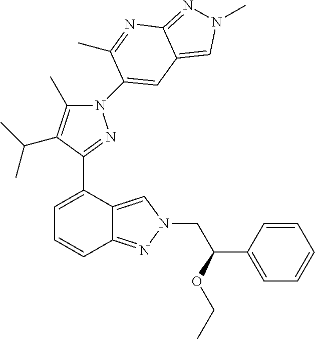 [SMILES: CCO[C@@H](Cn1cc2c(-c3nn(-c4cc5cn(C)nc5nc4C)c(C)c3C(C)C)cccc2n1)c1ccccc1] | G | 534 | 1.12 |
| | | 4-(1-{2, 6-dimethyl-2H-pyrazolo[3, 4-b]pyridin- | | | |
| | | 5-yl}-5-methyl-4-(propan-2-yl)-1H-pyrazol-3- | | | |
| | | yl)-2-[(2R)-2-ethoxy-2-phenylethyl]-2H- | | | |
| | | indazole | | | |
| 52 | 102 + methyliodide | 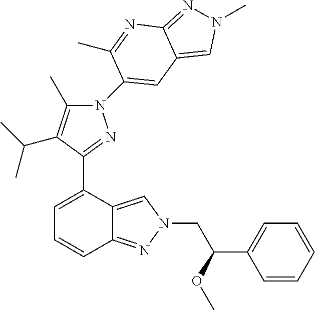 [SMILES: CO[C@@H](Cn1cc2c(-c3nn(-c4cc5cn(C)nc5nc4C)c(C)c3C(C)C)cccc2n1)c1ccccc1] | E | 520 | 0.78 |
| | | 4-(1-{2, 6-dimethyl-2H-pyrazolo[3, 4- | | | |
| | | b]pyridin-5-yl}-5-methyl-4-(propan-2-yl)-1H- | | | |
| | | pyrazol-3-yl)-2-[(2R)-2-methoxy-2- | | | |
| | | phenylethyl]-2H-indazole | | | |
| 63 | 102 + (bromomethyl) cyclopropane | 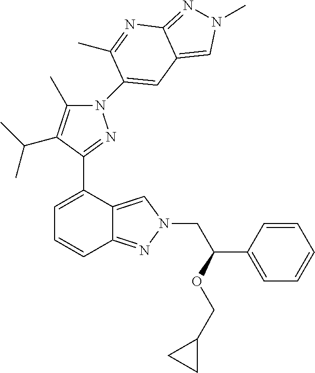 [SMILES: Cc1nc2nn(C)cc2cc1-n1nc(-c2cccc3nn(C[C@H](OCC4CC4)c4ccccc4)cc23)c(C(C)C)c1C] | G | 560 | 1.15 |
| | | 2-[(2R)-2-(cyclopropylmethoxy)-2- | | | |
| | | phenylethyl]-4-(1-{2, 6-dimethyl-2H- | | | |
| | | pyrazolo[3, 4-b]pyridin-5-yl}-5-methyl-4- | | | |
| | | (propan-2-yl)-1H-pyrazol-3-yl)-2H-indazole | | | |
| 66 | Step 1: 102 + 2-(4-chlorobutoxy) tetrahydro-2H- pyran; Step 2: TFA, 30 min, 50° C. | 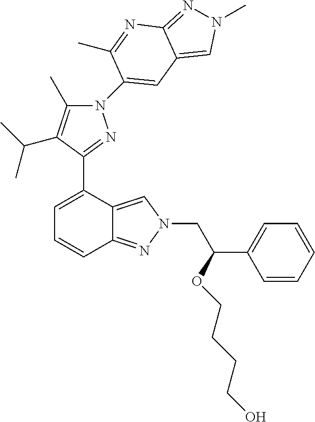 [SMILES: Cc1nc2nn(C)cc2cc1-n1nc(-c2cccc3nn(C[C@H](OCCCCO)c4ccccc4)cc23)c(C(C)C)c1C] | G | 578 | 1.04 |
| | | 4-[(1R)-2-[4-(1-{2, 6-dimethyl-2H-pyrazolo[3, | | | |
| | | 4-b]pyridin-5-yl}-5-methyl-4-(propan-2-yl)-1H- | | | |
| | | pyrazol-3-yl)-2H-indazol-2-yl]-1- | | | |
| | | phenylethoxy]butan-1-ol | | | |
| 74 | 102 + 4-bromo- 2-methylbutan-2-ol | 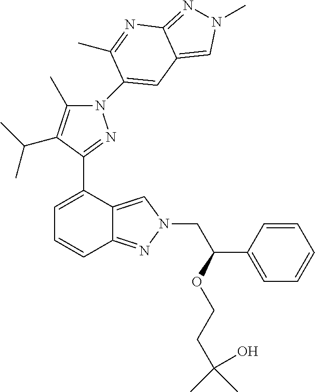 [SMILES: Cc1nc2nn(C)cc2cc1-n1nc(-c2cccc3nn(C[C@H](OCCC(C)(C)O)c4ccccc4)cc23)c(C(C)C)c1C] | G | 592 | 1.08 |
| | | 4-[(1R)-2-[4-(1-{2, 6-dimethyl-2H-pyrazolo[3, | | | |
| | | 4-b]pyridin-5-yl}-5-methyl-4-(propan-2-yl)-1H- | | | |
| | | pyrazol-3-yl)-2H-indazol-2-yl]-1- | | | |
| | | phenylethoxy]-2-methylbutan-2-ol | | | |
| 78 | 102 + 1-fluoro- 2-iodo-ethane | 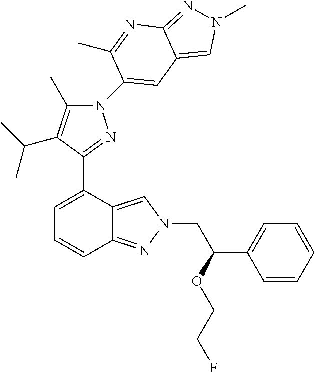 [SMILES: Cc1nc2nn(C)cc2cc1-n1nc(-c2cccc3nn(C[C@H](OCCF)c4ccccc4)cc23)c(C(C)C)c1C] | G | 552 | 1.08 |
| | | 4-(1-{2, 6-dimethyl-2H-pyrazolo[3, 4- | | | |
| | | b]pyridin-5-yl}-5-methyl-4-(propan-2-yl)-1H- | | | |
| | | pyrazol-3-yl)-2-[(2R)-2-(2-fluoroethoxy)-2- | | | |
| | | phenylethyl]-2H-indazole | | | |
| 79 | 102 + allyl bromide | 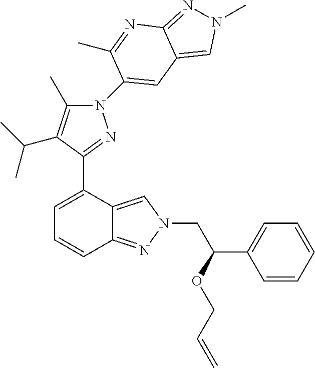 [SMILES: C=CCO[C@@H](Cn1cc2c(-c3nn(-c4cc5cn(C)nc5nc4C)c(C)c3C(C)C)cccc2n1)c1ccccc1] | G | 546 | 1.13 |
| | | 4-(1-{2, 6-dimethyl-2H-pyrazolo[3, 4- | | | |
| | | b]pyridin-5-yl}-5-methyl-4-(propan-2-yl)-1H- | | | |
| | | pyrazol-3-yl)-2-[(2R)-2-phenyl-2-(prop-2-en-1- | | | |
| | | yloxy)ethyl]-2H-indazole | | | |
| 81 | 102 + 1- bromopropane | 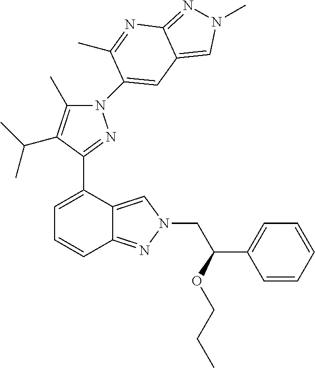 [SMILES: CCCO[C@@H](Cn1cc2c(-c3nn(-c4cc5cn(C)nc5nc4C)c(C)c3C(C)C)cccc2n1)c1ccccc1] | G | 548 | 1.15 |
| | | 4-(1-{2, 6-dimethyl-2H-pyrazolo[3, 4- | | | |
| | | b]pyridin-5-yl}-5-methyl-4-(propan-2-yl)-1H- | | | |
| | | pyrazol-3-yl)-2-[(2R)-2-phenyl-2- | | | |
| | | propoxyethyl]-2H-indazole | | | |
| 85 | 102 + 2- bromoethyl methyl ether | 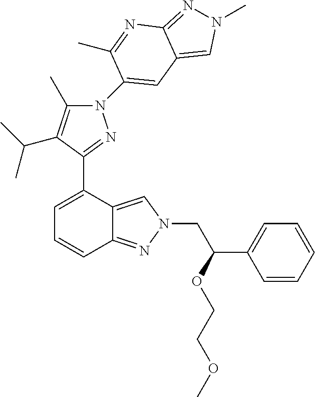 [SMILES: COCCO[C@@H](Cn1cc2c(-c3nn(-c4cc5cn(C)nc5nc4C)c(C)c3C(C)C)cccc2n1)c1ccccc1] | G | 564 | 1.08 |
| | | 4-(1-{2, 6-dimethyl-2H-pyrazolo[3, 4- | | | |
| | | b]pyridin-5-yl}-5-methyl-4-(propan-2-yl)-1H- | | | |
| | | pyrazol-3-yl)-2-[(2R)-2-(2-methoxyethoxy)-2- | | | |
| | | phenylethyl]-2H-indazole | | | |

### Synthesis of Example 36: 2-[(1R)-2-[4-(1-{2, 6-dimethyl-2H-pyrazolo[3, 4-b]pyridin-5-yl)-5-methyl-4-(propan-2-yl)-1H-pyrazol-3-yl)-2H-indazol-2-yl]-1-phenylethoxy]-N-methylacetamide

### Step 1: Synthesis of ethyl 2-[(1R)-2-[4-(1-{2, 6-dimethyl-2H-pyrazolo[3, 4-b]pyridin-5-yl)-5-methyl-4-(propan-2-yl)-1H-pyrazol-3-yl)-2H-indazol-2-yl]-1-phenylethoxy]acetate

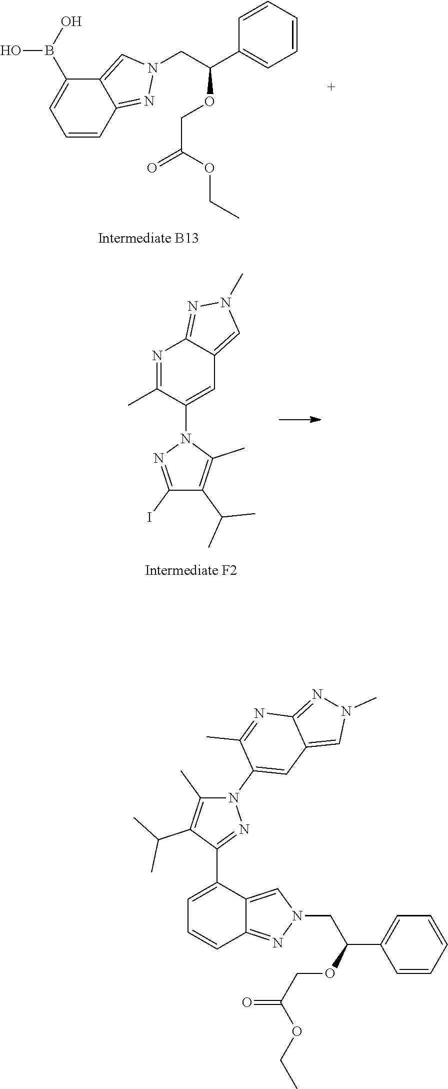 [SMILES: CCOC(=O)CO[C@@H](Cn1cc2c(B(O)O)cccc2n1)c1ccccc1 ; Cc1nc2nn(C)cc2cc1-n1nc(I)c(C(C)C)c1C ; CCOC(=O)CO[C@@H](Cn1cc2c(-c3nn(-c4cc5cn(C)nc5nc4C)c(C)c3C(C)C)cccc2n1)c1ccccc1]

{2-[(2R)-2-(2-Ethoxy-2-oxoethoxy)-2-phenylethyl]-2H-indazol-4-yl}boronic acid (300 mg, 0.82 mmol), 1-{2, 6-dimethyl-2H-pyrazolo[3, 4-b]pyridin-5-yl}-3-iodo-5-methyl-4-(propan-2-yl)-1H-pyrazole (300 mg), Cs₂CO₃ (0.74 g) and [1, 1′-bis(diphenylphosphino)ferrocene]dichloropalladium(ii), complex with dichloromethane (1:1) (100 mg) are suspended in dioxane (8 mL) and water (2 mL) and the mixture is stirred under an argon atmosphere at 80° C. for 3 h. The mixture is concentrated and HOAc, ACN and water is added. The mixture is filtered and the filtrate is purified by reversed phase preparative HPLC to yield the product.

Analysis (method F): Rₜ: 0.95 min, [M+H]⁺: 592

### Step 2: Synthesis of 2-[(1R)-2-[4-(1-{2, 6-dimethyl-2H-pyrazolo[3, 4-b]pyridin-5-yl}-5-methyl-4-(propan-2-yl)-1H-pyrazol-3-yl)-2H-indazol-2-yl]-1-phenylethoxy]acetic acid

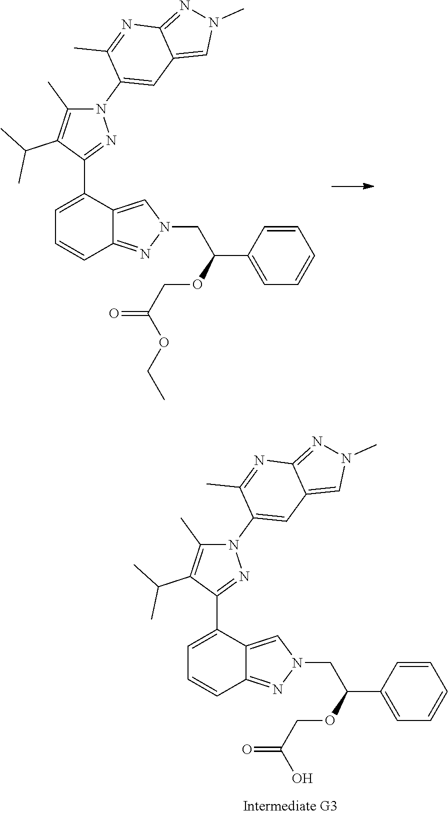 [SMILES: CCOC(=O)CO[C@@H](Cn1cc2c(-c3nn(-c4cc5cn(C)nc5nc4C)c(C)c3C(C)C)cccc2n1)c1ccccc1 ; Cc1nc2nn(C)cc2cc1-n1nc(-c2cccc3nn(C[C@H](OCC(=O)O)c4ccccc4)cc23)c(C(C)C)c1C]

Ethyl 2-[(1R)-2-[4-(1-{2, 6-dimethyl-2H-pyrazolo[3, 4-b]pyridin-5-yl}-5-methyl-4-(propan-2-yl)-1H-pyrazol-3-yl)-2H-indazol-2-yl]-1-phenylethoxy]acetate (375 mg, 0.53 mmol) is dissolved in EtOH (3 mL). NaOH (2.65 mL, 2.65 mmol, 1 M) is added and the reaction mixture is stirred at RT for 1.25 h. The reaction mixture is diluted with water and TFA (0.5 mL) is added. The reaction mixture is concentrated and the residue is diluted with water (5 mL) and brine (5 mL) and is extracted with DCM (10 mL). The organic layer is dried (Na₂SO₄), filtered and concentrated to afford intermediate G3.

Analysis (method F): Rₜ: 0.83 min, [M+H]⁺: 564

### Step 3: Synthesis of 2-[(1R)-2-[4-(1-{2, 6-dimethyl-2H-pyrazolo[3, 4-b]pyridin-5-yl}-5-methyl-4-(propan-2-yl)-1H-pyrazol-3-yl)-2H-indazol-2-yl]-1-phenylethoxy]-N-methylacetamide

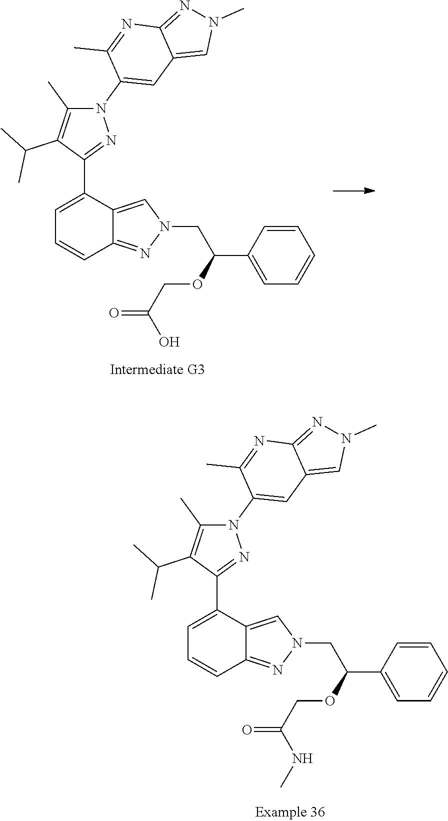 [SMILES: Cc1nc2nn(C)cc2cc1-n1nc(-c2cccc3nn(C[C@H](OCC(=O)O)c4ccccc4)cc23)c(C(C)C)c1C ; CNC(=O)CO[C@@H](Cn1cc2c(-c3nn(-c4cc5cn(C)nc5nc4C)c(C)c3C(C)C)cccc2n1)c1ccccc1]

2-[(1R)-2-[4-(1-{2, 6-Dimethyl-2H-pyrazolo[3, 4-b]pyridin-5-yl}-5-methyl-4-(propan-2-yl)-1H-pyrazol-3-yl)-2H-indazol-2-yl]-1-phenylethoxy]acetic acid (50 mg, 0.074 mmol) is dissolved in DMF (1 mL). DIPEA (26.8 μL, 0.15 mmol) and HATU (32.3 mg, 0.085 mmol) are added, and the reaction mixture is stirred at RT for 10 min. Then methylamine (74 μL, 0.15 mmol, 2 M in THF) is added and the reaction mixture is stirred at RT overnight. The reaction mixture is purified by reversed phase chromatography (HPLC; ACN/water including NH3) to afford the example 36.

Analysis (method C): Rₜ: 0.65 min, [M+H]⁺: 577

The Examples compiled in the following table are obtained by following a procedure analogous to that described for example 36.

| | | | | MS | |
|---|---|---|---|---|---|
| | | | | (ESI+): | |
| | Synthesis | | LC | m/z | t_{R} |
| Example | from | Structure/Name | Method | [M + H]⁺ | [min] |
| 108 | G3 + NH3 (7M in MeOH) | 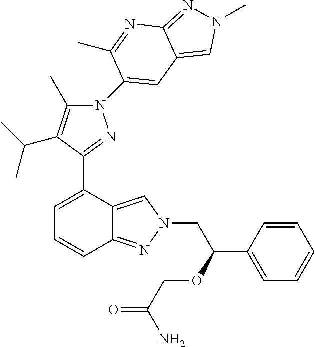 [SMILES: Cc1nc2nn(C)cc2cc1-n1nc(-c2cccc3nn(C[C@H](OCC(N)=O)c4ccccc4)cc23)c(C(C)C)c1C] | A | 563 | 0.56 |
| | | 2-[(1R) -2-[4-(1-{2, 6-dimethyl-2H- | | | |
| | | pyrazolo[3, 4-b] pyridin-5-yl}-5- | | | |
| | | methyl-4-(propan-2-yl)-1H-pyrazol-3- | | | |
| | | yl)-2H-indazol-2-yl]-1-phenylethoxy]acetamide | | | |

### Synthesis of Example 40: (1R)-2-[4-(1-{2-[3-(3, 3-difluoropyrrolidin-1-yl) propyl]-6-methyl-2H-pyrazolo[3, 4-b]pyridin-5-yl}-5-methyl-4-(propan-2-yl)-1H-pyrazol-3-yl)-2H-indazol-2-yl]-1-phenylethan-1-ol

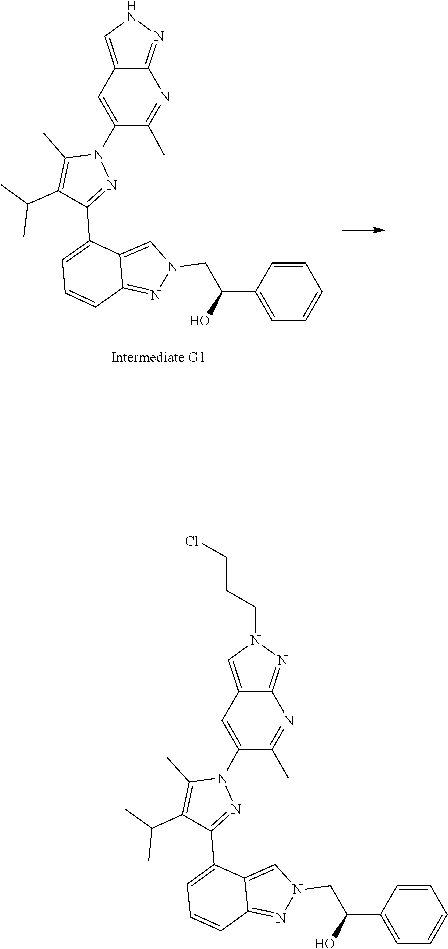 [SMILES: Cc1nc2n[nH]cc2cc1-n1nc(-c2cccc3nn(C[C@H](O)c4ccccc4)cc23)c(C(C)C)c1C ; Cc1nc2nn(CCCCl)cc2cc1-n1nc(-c2cccc3nn(C[C@H](O)c4ccccc4)cc23)c(C(C)C)c1C]

### Step 1: Synthesis of (1R)-2-(4-{1-[2-(3-Chloropropyl)-6-methyl-2H-pyrazolo[3, 4-b]pyridin-5-yl]-5-methyl-4-(propan-2-yl)-1H-pyrazol-3-yl}-2H-indazol-2-yl)-1-phenylethan-1-ol

(1R)-2-[4-(5-Methyl-1-{6-methyl-2H-pyrazolo[3, 4-b]pyridin-5-yl}-4-(propan-2-yl)-1H-pyrazol-3-yl)-2H-indazol-2-yl]-1-phenylethan-1-ol (200 mg, 0.41 mmol), 1-bromo-3-chloropropane (80.2 μM, 0.814 mmol) and K₂CO₃ (281 mg) are suspended in ACN (5 mL) and stirred overnight at 60° C. The mixture is filtered and purified by preparative reversed phase chromatography to yield of the desired product.

Analysis: (method G): Rₜ: 1.10 min [M+H]⁺: 568/570 (Cl)

### Step 2: Synthesis of (1R)-2-[4-(1-{2-[3-(3, 3-difluoropyrrolidin-1-yl) propyl]-6-methyl-2H-pyrazolo[3, 4-b]pyridin-5-yl}-5-methyl-4-(propan-2-yl)-1H-pyrazol-3-yl)-2H-indazol-2-yl]-1-phenylethan-1-ol

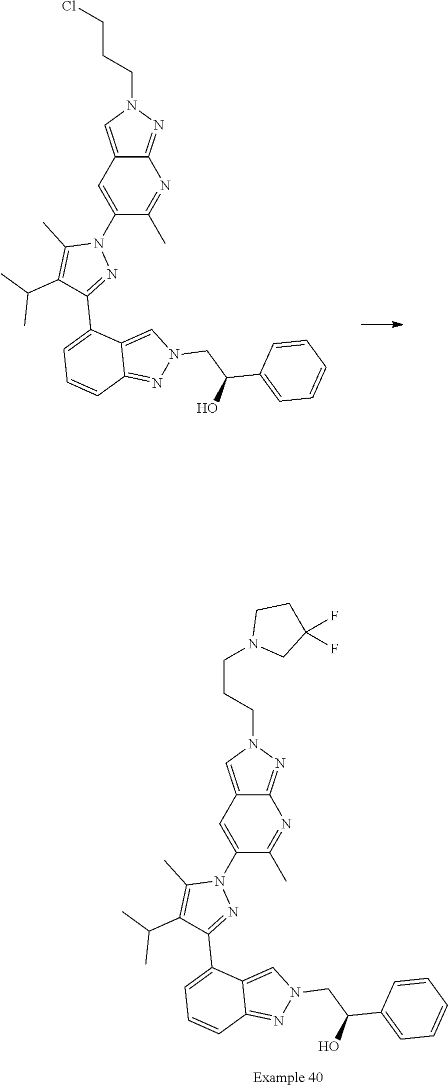 [SMILES: Cc1nc2nn(CCCCl)cc2cc1-n1nc(-c2cccc3nn(C[C@H](O)c4ccccc4)cc23)c(C(C)C)c1C ; Cc1nc2nn(CCCN3CCC(F)(F)C3)cc2cc1-n1nc(-c2cccc3nn(C[C@H](O)c4ccccc4)cc23)c(C(C)C)c1C]

(1R)-2-(4-{1-[2-(3-Chloropropyl)-6-methyl-2H-pyrazolo[3, 4-b]pyridin-5-yl]-5-methyl-4-(propan-2-yl)-1H-pyrazol-3-yl}-2H-indazol-2-yl)-1-phenylethan-1-ol (15 mg, 0.03 mmol) is dissolved in ACN (2 mL). K₂CO₃ (14.6 mg, 0.11 mmol) and 3, 3-difluoropyrrolidine×HCl (13.6 mg, 0.096 mmol) are added, and the reaction mixture is stirred at 90° C. overnight. The reaction mixture is purified by reversed phase chromatography (HPLC; ACN/water including NH3) to afford example 40.

Analysis: (method H): Rₜ: 1.10 min [M+H]⁺: 639

### Synthesis of Example 41: 4-{4-[(1R)-2-[4-(5-cyclopropyl-2-{6-cyclopropyl-2-methyl-2H-pyrazolo[3, 4-b]pyridin-5-yl}-1-methyl-1H-imidazol-4-yl)-2H-indazol-2-yl]-1-hydroxyethyl]phenoxy}-2-methylbutan-2-ol

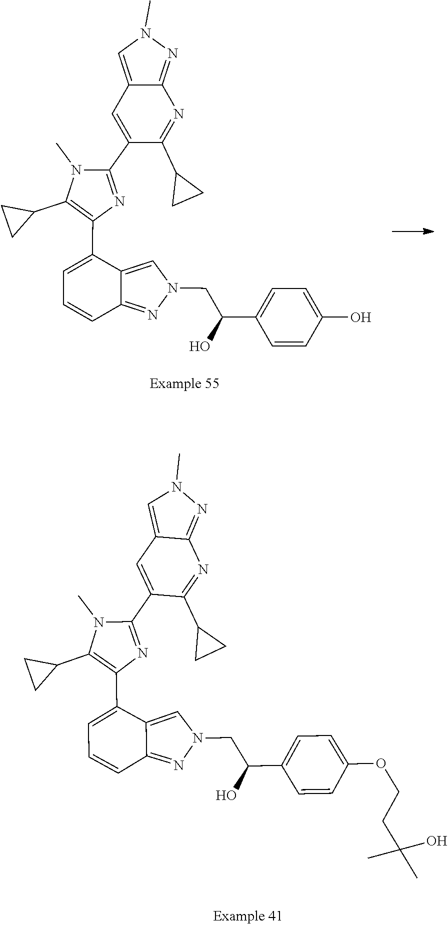 [SMILES: Cn1cc2cc(-c3nc(-c4cccc5nn(C[C@H](O)c6ccc(O)cc6)cc45)c(C4CC4)n3C)c(C3CC3)nc2n1 ; Cn1cc2cc(-c3nc(-c4cccc5nn(C[C@H](O)c6ccc(OCCC(C)(C)O)cc6)cc45)c(C4CC4)n3C)c(C3CC3)nc2n1]

4-[(1R)-2-[4-(5-Cyclopropyl-2-{6-cyclopropyl-2-methyl-2H-pyrazolo[3, 4-b]pyridin-5-yl}-1-methyl-1H-imidazol-4-yl)-2H-indazol-2-yl]-1-hydroxyethyl]phenol (30 mg, 0.06 mmol) is dissolved in DMF (1 mL). K₂CO₃ (15.2 mg, 0.11 mmol) and 4-bromo-2-methylbutan-2-ol (45.9 mg, 0.28 mmol) are added and the reaction mixture is stirred at 60° C. for 4 h. The reaction mixture is purified by reversed phase chromatography (HPLC; ACN/water including NH3) to afford example 41.

Analysis (method H): Rₜ: 0.99 min, [M+H]⁺: 632

### Synthesis of Example 68 and 69 and 90

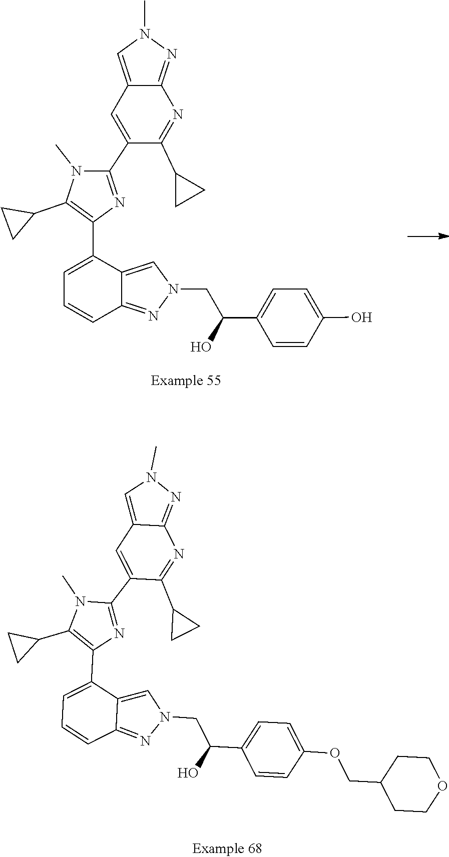 [SMILES: Cn1cc2cc(-c3nc(-c4cccc5nn(C[C@H](O)c6ccc(O)cc6)cc45)c(C4CC4)n3C)c(C3CC3)nc2n1 ; Cn1cc2cc(-c3nc(-c4cccc5nn(C[C@H](O)c6ccc(OCC7CCOCC7)cc6)cc45)c(C4CC4)n3C)c(C3CC3)nc2n1]

4-[(1R)-2-[4-(5-Cyclopropyl-2-{6-cyclopropyl-2-methyl-2H-pyrazolo[3, 4-b]pyridin-5-yl}-1-methyl-1H-imidazol-4-yl)-2H-indazol-2-yl]-1-hydroxyethyl]phenol (example 55) (30 mg, 0.06 mmol) is dissolved in DMF (1 mL). K₂CO₃ (69.1 mg, 0.50 mmol) and 4-bromomethyltetrahydropyran (39.4 mg, 0.22 mmol) are added and the reaction mixture is stirred at 60° C. overnight. The reaction mixture is purified by reversed phase chromatography (HPLC; ACN/water including TFA) to afford the example 68.

Analysis (method G): Rₜ: 0.87 min, [M+H]⁺: 644

The examples compiled in the following table are obtained by following a procedure analogous to that described for example 68.

| | | | | MS | |
|---|---|---|---|---|---|
| | | | | (ESI+): | |
| | | | LC | m/z | t_{R} |
| Example | Synthesis from | Structure/Name | Method | [M + H]⁺ | [min] |
| 69 | example 55 + 3-bromooxetane | 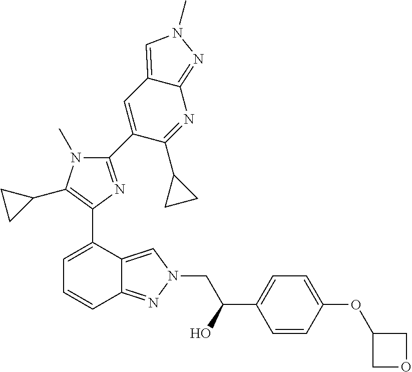 [SMILES: Cn1cc2cc(-c3nc(-c4cccc5nn(C[C@H](O)c6ccc(OC7COC7)cc6)cc45)c(C4CC4)n3C)c(C3CC3)nc2n1] | G | 602 | 0.82 |
| | | (1R)-2-[4-(5-cyclopropyl-2-{6-cyclopropyl-2-methyl- | | | |
| | | 2H-pyrazolo[3, 4-b] pyridin-5-yl}-1-methyl-1H-imidazol-4- | | | |
| | | yl)-2H-indazol-2-yl]-1-[4-(oxetan-3- | | | |
| | | yloxy)phenyl]ethan-1-ol | | | |
| 90 | example 55 + 2- chloroacetonitrile | 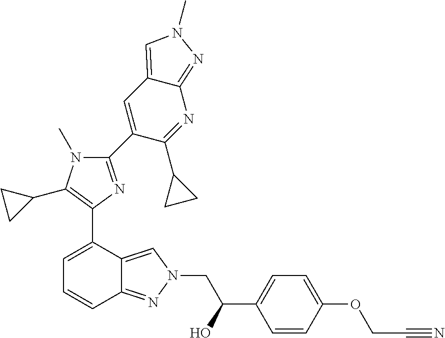 [SMILES: Cn1cc2cc(-c3nc(-c4cccc5nn(C[C@H](O)c6ccc(OCC#N)cc6)cc45)c(C4CC4)n3C)c(C3CC3)nc2n1] | G | 585 | 0.83 |
| | | 2-{4-[(1R)-2-[4-(5-cyclopropyl-2-{6- | | | |
| | | cyclopropyl-2-methyl-2H-pyrazolo[3, 4- | | | |
| | | b]pyridin-5-yl}-1-methyl-1H-imidazol-4- | | | |
| | | yl)-2H-indazol-2-yl]-1- | | | |
| | | hydroxyethyl]phenoxy} acetonitrile | | | |

### Synthesis of Example 88: 1-(4-Bromophenyl)-2-[4-(1-{2, 6-dimethyl-2H-pyrazolo[3, 4-b]pyridin-5-yl}-5-methyl-4-(propan-2-yl)-1H-pyrazol-3-yl)-2H-indazol-2-yl]ethan-1-ol

### Step 1: Synthesis of (4-(1-{2, 6-dimethyl-2H-pyrazolo[3, 4-b]pyridin-5-yl}-5-methyl-4-(propan-2-yl)-1H-pyrazol-3-yl)-2-[(trimethylsilyl)methyl]-2H-indazole)

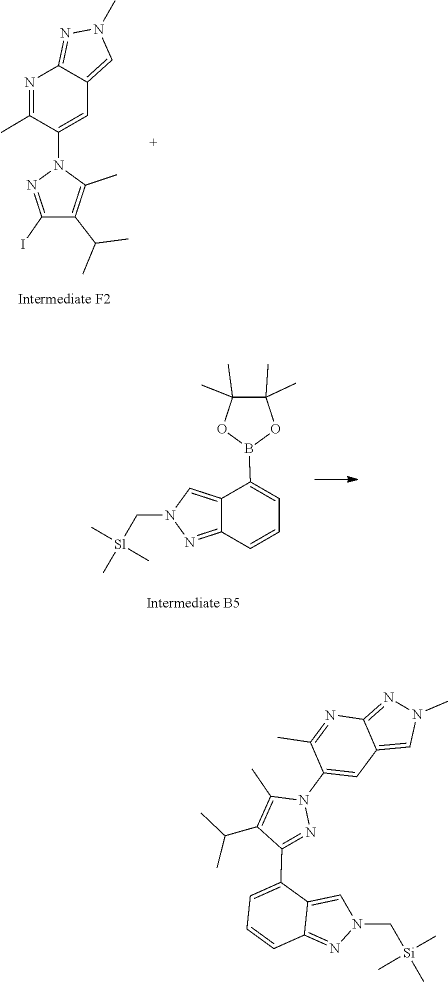

5-(3-Iodo-4-isopropyl-5-methyl-pyrazol-1-yl)-2, 6-dimethyl-pyrazolo[3, 4-b]pyridine (140 mg, 0.35 mmol) and 4-(4, 4, 5, 5-tetramethyl-1, 3, 2-dioxaborolan-2-yl)-2-[(trimethylsilyl)methyl]-2H-indazol (71 mg, 0.21 mmol) is suspended in dioxane (1 mL). Cs₂CO₃ (346 mg) and Pd(dppf)Cl₂ (28 mg complex with DCM 1:1) and water (150 μL) is added, and the mixture is stirred overnight at 80° C. The mixture is filtered, diluted with MeOH and HOAc and purified by preparative HPLC (ACN/water including TFA) to yield the desired product.

Analysis (method G): Rₜ: 1.09 min, [M+H]⁺: 472

### Step 2: Synthesis of 1-(4-bromophenyl)-2-[4-(1-{2, 6-dimethyl-2H-pyrazolo[3, 4-b]pyridin-5-yl}-5-methyl-4-(propan-2-yl)-1H-pyrazol-3-yl)-2H-indazol-2-yl]ethan-1-ol

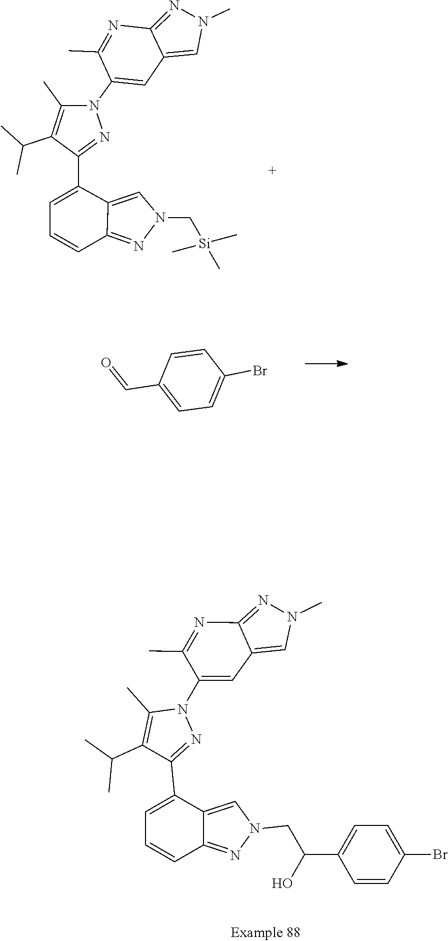 [SMILES: Cc1nc2nn(C)cc2cc1-n1nc(-c2cccc3c2=CN(C[Si](C)(C)C)CN=3)c(C(C)C)c1C ; O=Cc1ccc(Br)cc1 ; Cc1nc2nn(C)cc2cc1-n1nc(-c2cccc3nn(CC(O)c4ccc(Br)cc4)cc23)c(C(C)C)c1C]

(4-(1-{2, 6-Dimethyl-2H-pyrazolo[3, 4-b]pyridin-5-yl}-5-methyl-4-(propan-2-yl)-1H-pyrazol-3-yl)-2-[(trimethylsilyl)methyl]-2H-indazole) (84 mg, 0.18 mmol) and 4-bromobenzaldehyde (90 mg, 0.49 mmol) are dissolved in DMF (1 mL). CsF (30 mg, 0.20 mmol) is added and the reaction mixture is stirred at RT for 4 h. The reaction mixture is purified by reversed phase chromatography (HPLC; ACN/water including TFA) to afford the example 88.

Analysis (method G): Rₜ: 1.05 min, [M+H]⁺: 584/586 (Br)

### Synthesis of Example 92: (1R)-2-[4-(1-{2, 6-Dimethyl-2H-pyrazolo[3, 4-b]pyridin-5-yl}-5-methyl-4-(1-methylcyclopropyl)-1H-pyrazol-3-yl)-2H-indazol-2-yl]-1-phenylethan-1-ol

### Step 1: Synthesis of (1R)-2-[4-(5-methyl-1H-pyrazol-3-yl)-2H-indazol-2-yl]-1-phenylethan-1-ol

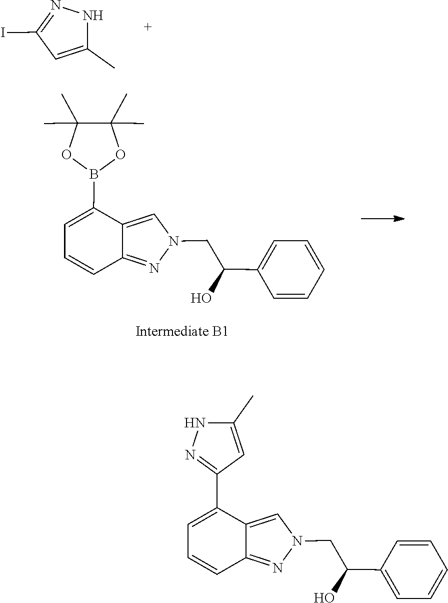

(1R)-1-Phenyl-2-[4-(4, 4, 5, 5-tetramethyl-1, 3, 2-dioxaborolan-2-yl)-2H-indazol-2-yl]ethan-1-ol (700 mg, 1.9 mmol), 3-iodo-5-methyl-1H-pyrazole (400 mg, 1.9 mmol), [1,1′-bis(diphenylphosphino)ferrocene]dichloropalladium(ii), complex with dichloromethane (1:1) (157 mg) are suspended in dioxane (7 mL) and Na₂CO₃ (2M, 2.9 mL) and the mixture is stirred under an argon atmosphere for 4 h at 80° C. and overnight at room temperature. The mixture is then diluted with ACN and purified by preparative HPLC to yield the desired compound.

Analysis (method A): Rₜ: 0.45 min, [M+H]⁺: 319

### Step 2: Synthesis of (1R)-2-[4-(4-bromo-5-methyl-1H-pyrazol-3-yl)-2H-indazol-2-yl]-1-phenylethan-1-ol

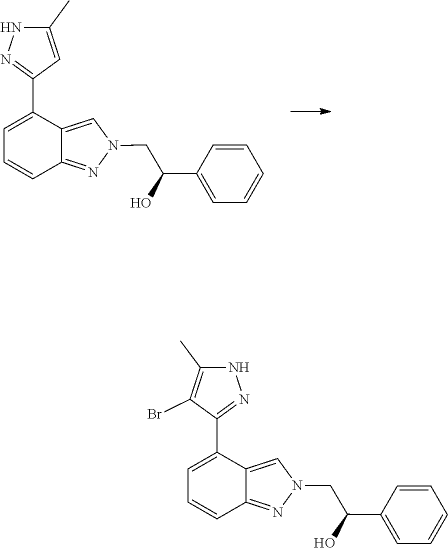 [SMILES: Cc1cc(-c2cccc3nn(C[C@H](O)c4ccccc4)cc23)n[nH]1 ; Cc1[nH]nc(-c2cccc3nn(C[C@H](O)c4ccccc4)cc23)c1Br]

(1R)-2-[4-(5-Methyl-1H-pyrazol-3-yl)-2H-indazol-2-yl]-1-phenylethan-1-ol (598 mg, 1.87 mmol) is suspended in ACN (40 mL), NBS (300 mg, 1.69 mmol) is added over 30 min and the mixture stirred 1 h at RT. The mixture is extracted with Na₂S₂O₃ (0.5 M). The aqueous phase is extracted with n-butanol, and the organic phases are combined and concentrated. The residue is dissolved in ACN/MeOH and purified by preparative HPLC. ACN is evaporated from the desired HPLC fractions, and the precipitated product collected to yield the product.

Analysis (method A): Rₜ: 0.56 min, [M+H]⁺: 397/399

### Step 3: Synthesis of (1R)-2-{4-[5-methyl-4-(prop-1-en-2-yl)-1H-pyrazol-3-yl]-2H-indazol-2-yl}-1-phenylethan-1-ol

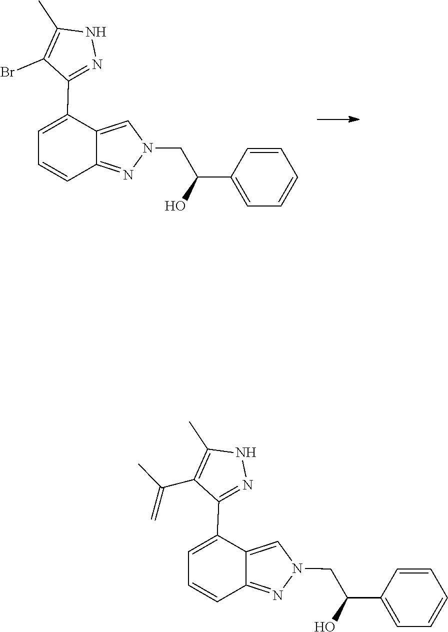 [SMILES: Cc1[nH]nc(-c2cccc3nn(C[C@H](O)c4ccccc4)cc23)c1Br ; C=C(C)c1c(-c2cccc3nn(C[C@H](O)c4ccccc4)cc23)n[nH]c1C]

(1R)-2-[4-(4-Bromo-5-methyl-1H-pyrazol-3-yl)-2H-indazol-2-yl]-1-phenylethan-1-ol (305 mg, 0.77 mmol), potassium trifluoro(prop-1-en-2-yl)boranuide (193 mg, 1.3 mmol), Cs₂CO₃ (750 mg), palladium acetate(12 mg), butyldi-1-adamantylphosphine (62 mg), toluol (10 mL) and water (1 mL) is mixed and stirred under an argon atmosphere at 120° C. overnight. The mixture is then filtered through a thiol scavenger resin cartridge and purified by preparative HPLC to yield the product.

Analysis (method A): Rₜ: 0.55 min, [M+H]⁺: 359

### Step 4: Synthesis of (1R)-2-{4-[5-methyl-4-(1-methylcyclopropyl)-1H-pyrazol-3-yl]-2H-indazol-2-yl}-1-phenylethan-1-ol

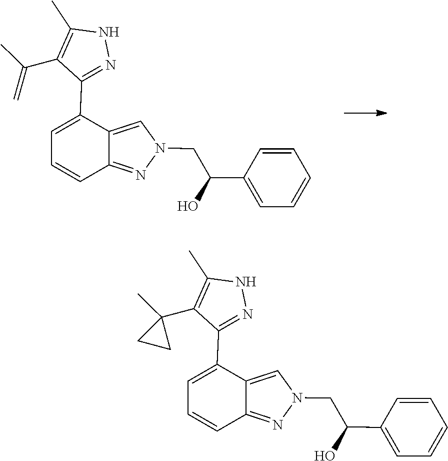

(1R)-2-{4-[5-Methyl-4-(prop-1-en-2-yl)-1H-pyrazol-3-yl]-2H-indazol-2-yl}-1-phenylethan-1-ol (215 mg) is dissolved in DCM (15 mL). Diiodomethane (219 μL) and then diethylzinc (2.7 mL, 1 M in hexane) is added at 0° C. and the mixture is stirred for 1 h at 0° C. Then aq. NH₄Cl solution (60 mL) is added, and the mixture is extracted with DCM. The organic phase is dried (Na₂SO₄), concentrated and purified by preparative HPLC to yield the desired product.

Analysis (method A): Rₜ: 0.64 min, [M+H]⁺: 373

### Step 5: Synthesis of 1-(3-{2-[(2R)-2-hydroxy-2-phenylethyl]-2H-indazol-4-yl}-5-methyl-4-(1-methylcyclopropyl)-1H-pyrazol-1-yl) propan-2-one

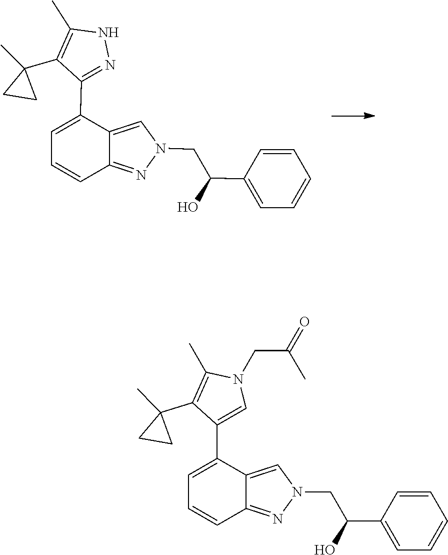 [SMILES: Cc1[nH]nc(-c2cccc3nn(C[C@H](O)c4ccccc4)cc23)c1C1(C)CC1 ; CC(=O)Cn1cc(-c2cccc3nn(C[C@H](O)c4ccccc4)cc23)c(C2(C)CC2)c1C]

(1R)-2-{4-[5-Methyl-4-(1-methylcyclopropyl)-1H-pyrazol-3-yl]-2H-indazol-2-yl}-1-phenylethan-1-ol (28 mg, 0.075 mmol) is dissolved in ACN (0.6 mL) then K₂CO₃ (26 mg) and chloroacetone (12 μL) added, and the mixture is stirred for 7 days at 50° C. During this time, additional chloroacetone (2-4 eq) is added every day to the mixture. The mixture is then concentrated and purified by preparative HPLC to yield the product.

Analysis (method A): Rₜ: 0.62 min, [M+H]⁺: 429.

### Step 6: Synthesis of (1R)-2-[4-(1-{2, 6-dimethyl-2H-pyrazolo[3, 4-b]pyridin-5-yl}-5-methyl-4-(1-methylcyclopropyl)-1H-pyrazol-3-yl)-2H-indazol-2-yl]-1-phenylethan-1-ol

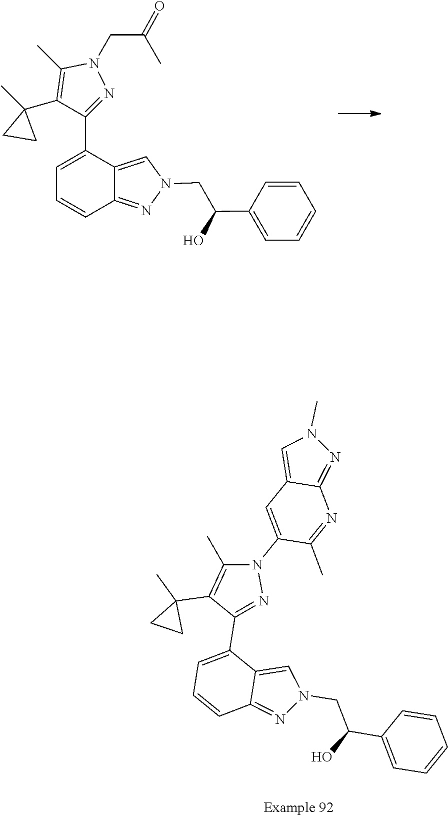 [SMILES: CC(=O)Cn1nc(-c2cccc3nn(C[C@H](O)c4ccccc4)cc23)c(C2(C)CC2)c1C ; Cc1nc2nn(C)cc2cc1-n1nc(-c2cccc3nn(C[C@H](O)c4ccccc4)cc23)c(C2(C)CC2)c1C]

1-(3-{2-[(2R)-2-Hydroxy-2-phenylethyl]-2H-indazol-4-yl}-5-methyl-4-(1-methylcyclopropyl)-1H-pyrazol-1-yl) propan-2-one (12 mg), 3-amino-1-methyl-1 h-pyrazole-4-carbaldehyde (intermediate E3) (3.5 mg), and piperidine (7 μL) are suspended in ethanol (500 μL) and the mixture is stirred at 80° C. for 3 h. The mixture is concentrated, dissolved in MeOH and purified by preparative HPLC and subsequently by preparative TLC (DCM/MeOH 93/7) to yield example 92.

Analysis (method A): Rₜ: 0.62 min, [M+H]⁺: 518.

### Synthesis of Example 96: 2-[(Benzenesulfonyl)methyl]-4-{5-cyclopropyl-1-methyl-2-[2-methyl-6-(propan-2-yl)-2H-pyrazolo[3, 4-b]pyridin-5-yl]-1H-imidazol-4-yl}-2H-indazole

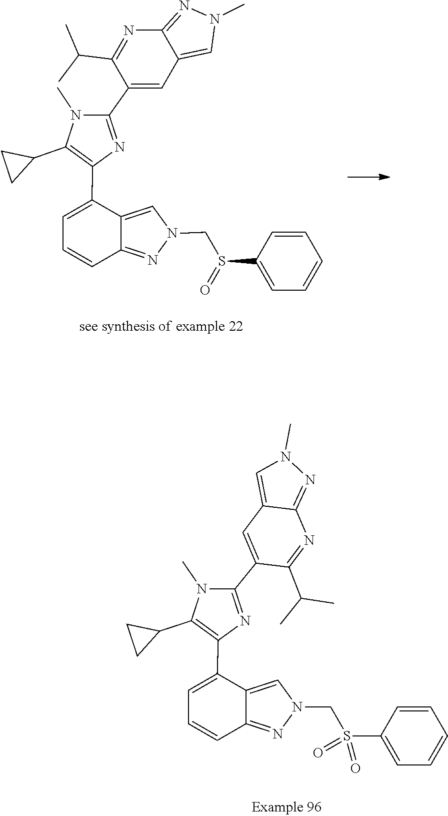 [SMILES: CC(C)c1nc2nn(C)cc2cc1-c1nc(-c2cccc3nn(C[S@](=O)c4ccccc4)cc23)c(C2CC2)n1C ; CC(C)c1nc2nn(C)cc2cc1-c1nc(-c2cccc3nn(CS(=O)(=O)c4ccccc4)cc23)c(C2CC2)n1C]

2-{[(R)-Benzenesulfinyl]methyl}-4-{5-cyclopropyl-1-methyl-2-[2-methyl-6-(propan-2-yl)-2H-pyrazolo[3, 4-b]pyridin-5-yl]-1H-imidazol-4-yl}-2H-indazole (15.9 mg, enantiomer eluting at 0.81 min) is suspended in DCM (1 mL) and 3-chloroperbenzoic acid (10 mg) is added at 5° C. and the mixture is stirred for 1 h. The mixture is diluted with DCM, extracted with Na₂CO₃ and the organic phase is concentrated. The mixture is purified by preparative HPLC (Xbridge-C18, ACN/water including TFA) to yield example 96.

Analysis (method A): Rₜ: 0.47 min, [M+H]⁺: 566.

### Example 97: (1R)-2-(4-{5-Cyclopropyl-1-methyl-2-[2-methyl-6-(propan-2-yl)-2H-pyrazolo[3, 4-b]pyridin-5-yl]-1H-imidazol-4-yl}-2H-indazol-2-yl)-1-phenylethan-1-ol

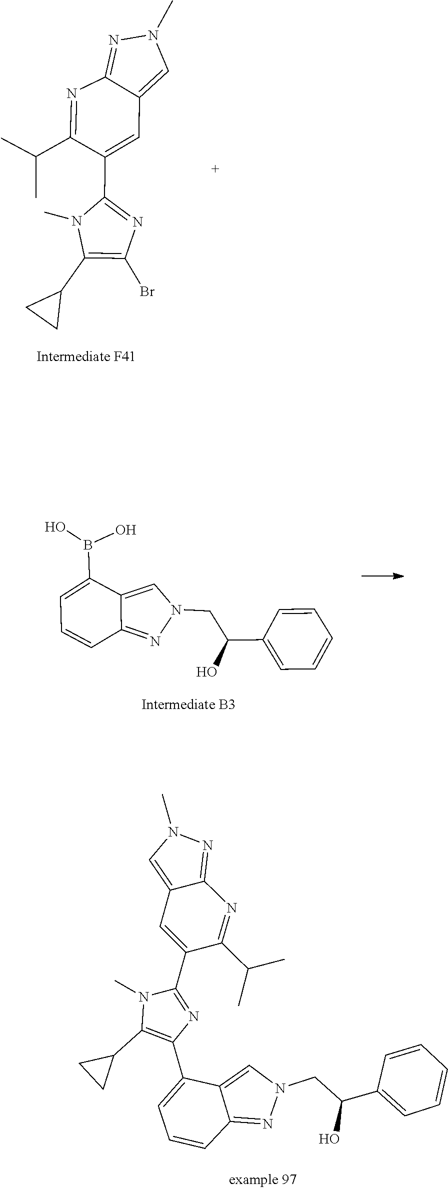 [SMILES: CC(C)c1nc2nn(C)cc2cc1-c1nc(Br)c(C2CC2)n1C ; OB(O)c1cccc2nn(C[C@H](O)c3ccccc3)cc12 ; CC(C)c1nc2nn(C)cc2cc1-c1nc(-c2cccc3nn(C[C@H](O)c4ccccc4)cc23)c(C2CC2)n1C]

4-Bromo-5-cyclopropyl-1-methyl-2-[2-methyl-6-(propan-2-yl)-2H-pyrazolo[3, 4-b]pyridin-5-yl]-1H-imidazole (150 mg, 0.32 mmol), {2-[(2R)-2-hydroxy-2-phenylethyl]-2H-indazol-4-yl}boronic acid (108 mg, 0.39 mmol) and 1, 1′-bis(di-tert-butylphosphino)ferrocene palladium dichloride (21 mg) are suspended in dioxane (2.5 mL). Na₂CO₃ (400 μL, 2M aqueous solution) is added, the mixture is degassed in vacuo and flushed with argon. The reaction mixture is heated to 80° C. for 3 h under an Argon atmosphere. The mixture is diluted with ACN/MeOH, filtered through a thiol scavenger resin cartridge and purified by preparative HPLC (Xbridge-C18, ACN/water including TFA, gradient 10 to 100% ACN) and a second HPLC purification (Xbridge-C18, ACN/water including NH₃, 10 to 80% ACN) to yield the example 97.

Analysis (method A): Rₜ: 0.46 min, [M+H]⁺: 532.

### Synthesis of Example 111: (1R)-2-(4-{5-Methyl-1-[2-methyl-6-(pyrrolidin-2-yl)-2H-pyrazolo[3, 4-b]pyridin-5-yl]-4-(propan-2-yl)-1H-pyrazol-3-yl}-2H-indazol-2-yl)-1-phenylethan-1-ol

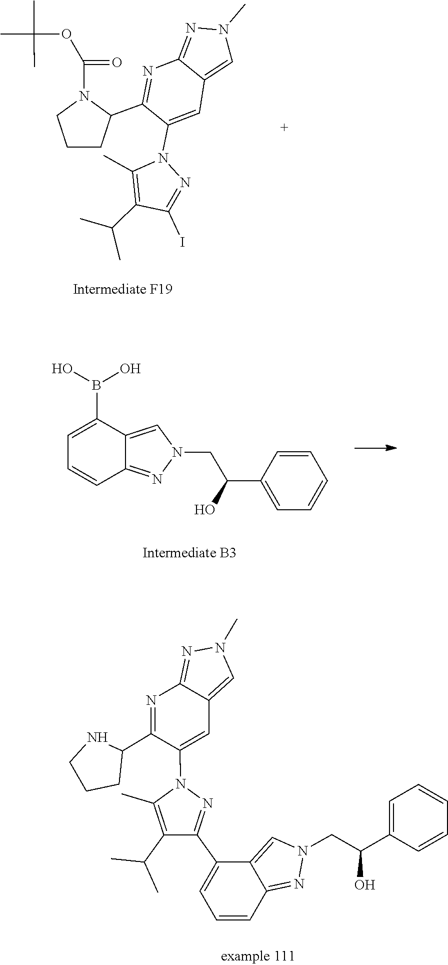 [SMILES: Cc1c(C(C)C)c(I)nn1-c1cc2cn(C)nc2nc1C1CCCN1C(=O)OC(C)(C)C ; OB(O)c1cccc2nn(C[C@H](O)c3ccccc3)cc12 ; Cc1c(C(C)C)c(-c2cccc3nn(C[C@H](O)c4ccccc4)cc23)nn1-c1cc2cn(C)nc2nc1C1CCCN1]

Under an argon atmosphere, tert-butyl 2-{5-[3-iodo-5-methyl-4-(propan-2-yl)-1H-pyrazol-1-yl]-2-methyl-2H-pyrazolo[3, 4-b]pyridin-6-yl}pyrrolidine-1-carboxylate (96 mg, 0.17 mmol) and {2-[(2R)-2-hydroxy-2-phenylethyl]-2H-indazol-4-yl}boronic acid (59 mg, 0.21 mmol) are suspended in dioxane (2 mL), then Cs₂CO₃ (0.43 mL, 1M aq) is added. Then XPhos Pd G3 (8.3 mg) is added, and the mixture is stirred at 90° C. for 4 h. The mixture is then diluted with EtOAc and extracted with water. The organic phase is concentrated and suspended in DCM (2 mL) and TFA (1 mL) for 1 h at RT. Then the mixture is concentrated and purified by preparative HPLC to yield the example 111.

Analysis (method G): Rₜ: 0.74 min, [M+H]⁺: 561

### LIST OF ABBREVIATIONS

Ac acetyl
ACN acetonitrile
AIBN 2, 2′-azobis(isobutyronitrile)
Boc tert-butyloxycarbonyl
Cbz benzyloxycarbonyl
CycH cyclohexane
d day(s)
DAST diethylamino sulfur trifluoride
DCE 1, 2-dichloroethane
DCM dichloromethane
DEAD diethyl azodicarboxylate
DIAD diisopropyl azodicarboxylate
DIPEA N, N-diisopropylethylamine
DMF N, N-dimethylformamide
DMP Dess-Martin Periodinane
DMSO dimethyl sulfoxide
EtOAc ethyl acetate
EtOH ethanol
h hour(s)
HATU O-(7-azabenzotriazol-1-yl)-N, N, N′, N′-tetramethyluronium-hexafluorophosphate
HPLC high performance liquid chromatography
HPLC-MS coupled high performance liquid chromatography-mass spectrometry
IPA isopropyl alcohol
LC liquid chromatography
LC-MS coupled liquid chromatography-mass spectrometry
LiHMDS Lithium-bis(trimethylsilyl)amide
M molar (mol/L)
Mel methyl iodide
MeTHF 2-methyltetrahydrofuran
MeOH methanol
min minute(s)
MS mass spectrometry
MTBE methyl-tertbutyl-ether
n-BuLi n-Buthyllithium
NBS N-Bromosuccinimide
NIS N-Iodosuccinimide
NMP N-methyl-2-pyrrolidone
NMR nuclear magnetic resonance
PEPPSI(TM)-IPR (1, 3-Bis(2, 6-diisopropylphenyl)imidazolidene) (3-chloropyridyl) palladium(II) dichloride
PdCl2(dtbpf) 1, 1′-Bis-(di-tert-butylphosphino-)ferrocene-palladiumdichloride
Pd(dppf)Cl₂ 1, 1′-bis(diphenylphosphino)ferrocenedichloropalladium(II)
Pd(PPh₃)₄ palladium (0) tetrakis(triphenylphosphine)
XPhos Pd G3 2-Dicyclohexylphosphino-2′, 4′, 6′-triisopropyl-1, 1′-biphenyl)[2-(2′-amino-1, 1′-biphenyl)]palladium(II) methanesulfonat
pet. petroleum
Rₜ retention factor
RP reverse phase
rt room temperature
t_{R} retention time (in HPLC/LC)
SFC supercritical fluid chromatography
TBAF tetrabutylammonium fluoride
TBTU O-(benzotriazol-1-yl)-N, N, N′, N′-tetramethyluronium tetrafluoroborate
TEA triethylamine
TFA trifluoroacetic acid
THE tetrahydrofuran
THP tetrahydro-2 h-pyran
TLC thin-layer chromatography
TMAD N, N, N′N′-Tetramethylazodicarboxamide
UV ultraviolet
V volume

### HPLC Methods:

Method A

| | Vol % water | | |
|---|---|---|---|
| time (min) | (incl. 0.1% TFA) | Vol % ACN | Flow [mL/min] |
| 0.00 | 99 | 1 | 1.6 |
| 0.02 | 99 | 1 | 1.6 |
| 1.00 | 0 | 100 | 1.6 |
| 1.10 | 0 | 100 | 1.6 |

column: XBridge BEH C18_2.1×30 mm, 1.7 μm; column temperature: 60° C.

Method B

| | Vol % water | | |
|---|---|---|---|
| time (min) | (incl. 0.1% NH₃) | Vol % ACN | Flow [mL/min] |
| 0.00 | 95 | 5 | 1.3 |
| 0.02 | 95 | 5 | 1.3 |
| 1.00 | 0 | 100 | 1.3 |
| 1.10 | 0 | 100 | 1.3 |

column: XBridge BEH C18_2.1×30 mm, 1.7 μm; column temperature: 60° C.

Method C

| | Vol % water | | |
|---|---|---|---|
| time (min) | (incl. 0.1% NH₃) | Vol % ACN | Flow [mL/min] |
| 0.00 | 95 | 5 | 1.3 |
| 0.02 | 95 | 5 | 1.3 |
| 1.00 | 0 | 100 | 1.3 |
| 1.10 | 0 | 100 | 1.3 |

column: XBridge BEH C18_2.1×30 mm, 2.5 μm; column temperature: 60° C.

Method D

| | Vol % water | | |
|---|---|---|---|
| time (min) | (incl. 0.1% NH₃) | Vol % ACN | Flow [mL/min] |
| 0.00 | 95 | 5 | 1.3 |
| 0.02 | 95 | 5 | 1.3 |
| 1.00 | 0 | 100 | 1.3 |
| 1.10 | 0 | 100 | 1.3 |

column: XBridge BEH Phenyl-2.1×30 mm, 1.7 μm; column temperature: 60° C.

Method E

| | Vol % water | | |
|---|---|---|---|
| time (min) | (incl. 0.1% TFA) | Vol % ACN | Flow [mL/min] |
| 0.00 | 99 | 1 | 1.5 |
| 0.02 | 99 | 1 | 1.5 |
| 1.00 | 0 | 100 | 1.5 |
| 1.10 | 0 | 100 | 1.5 |

column: Sunfire C18_2.1×30 mm, 2.5 μm; column temperature: 60° C.

Method F

| | Vol. % water | | |
|---|---|---|---|
| time (min) | (incl. 0.1% TFA) | Vol. % ACN | Flow [mL/min] |
| 0.00 | 95 | 5 | 1.5 |
| 1.30 | 0 | 100 | 1.5 |
| 1.50 | 0 | 100 | 1.5 |

column: Sunfire C18 (Waters) 2.5 μm; 3.0×30 mm; column temperature: 60° C.

Method G

| | Vol % water | | |
|---|---|---|---|
| time (min) | (incl. 0.1% TFA) | Vol % ACN | Flow [mL/min] |
| 0.00 | 97 | 3 | 2.2 |
| 0.20 | 97 | 3 | 2.2 |
| 1.20 | 0 | 100 | 2.2 |
| 1.25 | 0 | 100 | 3.0 |
| 1.40 | 0 | 100 | 3.0 |

column: Sunfire (Waters) 2.5 μm; 3.0×30 mm; column temperature: 60° C.

Method H

| | Vol. % water | | |
|---|---|---|---|
| time (min) | (incl. 0.1% NH₃) | Vol % ACN | Flow [mL/min] |
| 0.00 | 97 | 3 | 2.2 |
| 0.20 | 97 | 3 | 2.2 |
| 1.20 | 0 | 100 | 2.2 |
| 1.25 | 0 | 100 | 3.0 |
| 1.40 | 0 | 100 | 3.0 |

column: Xbridge (Waters); C18_3.0×30 mm_2.5 μm; column temperature: 60° C.

Method I

| | Vol. % water | | |
|---|---|---|---|
| time (min) | (incl. 0.1% formic acid) | Vol % ACN | Flow [mL/min] |
| 0.00 | 97 | 3 | 2.2 |
| 0.20 | 97 | 3 | 2.2 |
| 1.20 | 0 | 100 | 2.2 |
| 1.25 | 0 | 100 | 3.0 |
| 1.40 | 0 | 100 | 3.0 |

column: Xbridge (Waters); C18_3.0×30 mm_2.5 μm; column temperature: 60° C.

Method J

| | Vol. % water | | |
|---|---|---|---|
| time (min) | (incl. 0.1% TFA) | Vol. % ACN | Flow [mL/min] |
| 0.00 | 95 | 5 | 1.5 |
| 1.30 | 0 | 100 | 1.5 |
| 1.50 | 0 | 100 | 1.5 |
| 1.60 | 95 | 5 | 1.5 |

column: Sunfire C18 (Waters) 2.5 μm; 3.0×30 mm; CT: 60° C.

Method K

| time (min) | % Sol Carbon dioxide | % Sol methanol | Flow [mL/min] |
|---|---|---|---|
| 0.00 | 97 | 3 | 1.3 |
| 2.5 | 55 | 45 | 1.3 |
| 3.5 | 55 | 45 | 1.3 |
| 3.51 | 97 | 3 | 1.3 |
| 4 | 97 | 3 | 1.3 |

column: Acquity UPC2 Torus 2-PIC_3.0×100 mm_1.7 μm; column temperature: 30° C.

Method L

| | Vol. % water | | |
|---|---|---|---|
| time (min) | (incl. 0.1% TFA) | Vol. % ACN | Flow [mL/min] |
| 0.00 | 95 | 5 | 1.5 |
| 1.30 | 0 | 100 | 1.5 |
| 1.50 | 0 | 100 | 1.5 |
| 1.60 | 95 | 5 | 1.5 |

column: Sunfire C18 (Waters) 2.5 μm; 3.0×30 mm; CT: 60° C.

Method M

| time (min) | % Sol Carbon dioxide | % Sol methanol | Flow [mL/min] |
|---|---|---|---|
| 0.00 | 97 | 3 | 1.3 |
| 2.5 | 55 | 45 | 1.3 |
| 3.5 | 55 | 45 | 1.3 |
| 3.51 | 97 | 3 | 1.3 |
| 4 | 97 | 3 | 1.3 |

column: Acquity UPC2 Torus 2-PIC_3.0×100 mm_1.7 μm; column temperature: 30° C.

Method N

| | Vol. % water | | |
|---|---|---|---|
| time (min) | (incl. 0.1% NH₃) | Vol. % ACN | Flow [mL/min] |
| 0.00 | 95 | 5 | 1.5 |
| 1.30 | 0 | 100 | 1.5 |
| 1.50 | 0 | 100 | 1.5 |
| 1.60 | 95 | 5 | 1.5 |

column: XBridge C18_3.0×30 mm_2.5 μm (Waters); column temperature: 60° C.

Method O

| | Vol. % water | | |
|---|---|---|---|
| time (min) | (incl. 0.1% NH₃) | Vol. % ACN | Flow [mL/min] |
| 0.00 | 95 | 5 | 1.5 |
| 1.30 | 0 | 100 | 1.5 |
| 1.50 | 0 | 100 | 1.5 |
| 1.60 | 95 | 5 | 1.5 |

column: XBridge C18_3.0×30 mm_2.5 μm (Waters); column temperature: 60° C.

Method P

| | Vol. % water | | |
|---|---|---|---|
| time (min) | (incl. 0.1% NH₃) | Vol. % ACN | Flow [mL/min] |
| 0.00 | 95 | 5 | 1.5 |
| 1.30 | 0 | 100 | 1.5 |
| 1.50 | 0 | 100 | 1.5 |
| 1.60 | 95 | 5 | 1.5 |

column: XBridge C18_3.0×30 mm_2.5 μm (Waters); column temperature: 60° C.

Method Q

| | Vol % water | Vol. % ACN | |
|---|---|---|---|
| time | (incl. 0.1% | (including 0.1% | Flow |
| (min) | formic acid) | formic acid) | [mL/min] |
| 0.00 | 99 | 1 | 0.5 |
| 2.00 | 0 | 100 | 0.5 |
| 3.00 | 0 | 100 | 0.5 |

column: Acquity UPLC BEH C18 1.7 μm (2.1×100 mm); column temperature: 40° C.

Method R

| | Vol % water | Vol. % ACN | |
|---|---|---|---|
| time | (incl. 0.1% | (including 0.1% | Flow |
| (min) | formic acid) | formic acid) | [mL/min] |
| 0.00 | 90 | 10 | 1.0 |
| 3.35 | 30 | 70 | 1.0 |
| 3.75 | 30 | 70 | 1.0 |
| 3.90 | 5 | 95 | 1.0 |
| 4.75 | 5 | 95 | 1.0 |
| 5.00 | 90 | 10 | 1.0 |
| 6.00 | 90 | 10 | 1.0 |

column: Kinetex XB-C18 2.6 μm (4.6×50 mm), 100 Å; column temperature: 25° C.

Method S

| | Vol % water | Vol. % ACN | |
|---|---|---|---|
| time | (incl. 0.1% | (including 0.1% | Flow |
| (min) | formic acid) | formic acid) | [mL/min] |
| 0.00 | 80 | 20 | 0.5 |
| 0.1 | 80 | 20 | 0.5 |
| 1.1 | 0 | 100 | 0.5 |
| 2.5 | 80 | 20 | 0.5 |
| 3 | 80 | 20 | 0.5 |

column: Acquity UPLC BEH C18 1.7 μm (2.1×100 mm); column temperature: 40° C.

Method T

| | | % Sol | |
|---|---|---|---|
| | | (MeOH | |
| time | % Sol | 20 mM | Flow |
| (min) | (scCO₂) | NH3) | [mL/min] |
| 0.00 | 80 | 20 | 4 |
| 10 | 80 | 20 | 4 |

column: CHIRAL ART® Cellulose SB_4.6×250 mm_5 μm; column temperature: 40° C.

Method U

| | | % Sol | |
|---|---|---|---|
| | | (IPA | |
| time | % Sol | 20 mM | Flow |
| (min) | (scCO₂) | NH₃) | [mL/min] |
| 0.00 | 80 | 20 | 4 |
| 10 | 80 | 20 | 4 |

column: CHIRAL ART® Cellulose SB_4.6×250 mm_5 μm; column temperature: 40° C.

Method V

| | | % Sol | |
|---|---|---|---|
| | | (IPA | |
| time | % Sol | 20 mM | Flow |
| (min) | (scCO₂) | NH₃) | [mL/min] |
| 0.00 | 70 | 30 | 4 |
| 10 | 70 | 30 | 4 |

column: CHIRAL ART® Cellulose SB_4.6×250 mm_5 μm; column temperature: 40° C.

Method W

| | | % Sol | |
|---|---|---|---|
| | | (IPA | |
| time | % Sol | 20 mM | Flow |
| (min) | (scCO₂) | NH₃) | [mL/min] |
| 0.00 | 60 | 40 | 4 |
| 10 | 60 | 40 | 4 |

column: CHIRAL ART® Cellulose SB_4.6×250 mm_5 μm; column temperature: 40° C.

Method X

| | | % Sol | |
|---|---|---|---|
| | | (MeOH | |
| time | % Sol | 20 mM | Flow |
| (min) | (scCO₂) | NH₃) | [mL/min] |
| 0.00 | 65 | 35 | 2 |
| 10 | 65 | 35 | 2 |

column: CHIRAL ART® Cellulose SB_4.6×250 mm_5 μm; column temperature: 40° C.

Method Y

| | | % Sol | |
|---|---|---|---|
| | | (EtOH | |
| time | % Sol | 20 mM | Flow |
| (min) | (scCO₂) | NH₃) | [mL/min] |
| 0.00 | 65 | 35 | 2 |
| 4 | 65 | 35 | 2 |

column: CHIRAL ART® Cellulose SB_4.6×250 mm_5 μm; column temperature: 40° C.

Method Z

| | | % Sol | |
|---|---|---|---|
| | | (MeOH | |
| time | % Sol | 20 mM | Flow |
| (min) | (scCO₂) | NH₃) | [mL/min] |
| 0.00 | 75 | 25 | 4 |
| 10 | 75 | 25 | 4 |

column: CHIRAL ART® Cellulose SB_4.6×250 mm_5 μm; column temperature: 40° C.

Method ZA

| | | % Sol | |
|---|---|---|---|
| | | (IPA | |
| time | % Sol | 20 mM | Flow |
| (min) | (scCO₂) | NH₃) | [mL/min] |
| 0.00 | 60 | 40 | 2 |
| 10 | 60 | 40 | 2 |

column: CHIRAL ART® Amylose-SA_3×100 mm_3 μm; column temperature: 40° C.

Method ZB

| | | % Sol | |
|---|---|---|---|
| | | (IPA | |
| time | % Sol | 20 mM | Flow |
| (min) | (scCO₂) | NH₃) | [mL/min] |
| 0.00 | 85 | 15 | 2 |
| 4 | 85 | 15 | 2 |

column: Lux(R) Cellulose-2_3×100 mm_3 μm; column temperature: 40° C.

Method ZC

| | | % Sol | |
|---|---|---|---|
| | | (MeOH | |
| time | % Sol | 20 mM | Flow |
| (min) | (scCO₂) | NH₃) | [mL/min] |
| 0.00 | 80 | 20 | 4 |
| 10 | 80 | 20 | 4 |

column: Chiralpak® IG_4.6×250 mm_5 μm; column temperature: 40° C.

Method ZD

| | | % Sol | |
|---|---|---|---|
| | | (IPA | |
| time | % Sol | 20 mM | Flow |
| (min) | (scCO₂) | NH₃) | [mL/min] |
| 0.00 | 80 | 35 | 4 |
| 10 | 80 | 35 | 4 |

column: CHIRAL ART® Amylose SA_4.6×250 mm_5 μm; column temperature: 40° C.

### Biological Assay

The activity of the compounds of the invention may be demonstrated using the following in vitro STING biochemical and cell assays.

### Human Sting HTRF Binding Assay

Binders to human STING WT (R232) were identified using a competitive HTRF assay format (Cisbio 64BDSTGPEG), which uses d2-labeled STING ligand, a 6His (SEQ ID NO: 2) tagged human STING protein, and an anti 6His (SEQ ID NO: 2) Cryptate-labeled antibody. Compounds compete with the STING ligand-d2 and thereby prevents FRET from occurring, which can be measured by an EnVision™ reader (PerkinElmer).

Assay method: Compounds were delivered as 10 mM DMSO solution, serially diluted by an Agilent Bravo Workstation and transferred to the 384 well assay plate (Perkin Elmer #6005359) using a Cybiwell dispenser. Typically, 8 concentrations were used with the highest concentration at 10 μM or 1 μM in the final assay volume followed by ~1:5 dilution steps. DMSO concentration was set to 1% in the final assay volume. The 384 well assay plate contained 20 test compounds and DMSO in column 23 and 24. A cGAMP standard dilution row was prepared according to the manufacturer and transferred to each assay plate. After transfer of compound solution or dilution buffer for negative (high) and positive (low) controls, 5 μl of the human STING protein (cyclic binding domain (residues 138-379) of the WT R232 human version, fused to a 6 His tag (SEQ ID NO: 2) at the Nter part; 1:50 dilution in detection buffer) were dispensed to all wells except of the positive control, which received detection buffer only. Plates were the centrifuged for 20 sec at 1000 rpm. After that, 10 μl of Anti-6His-Cryptate antibody/Sting ligand-d2 mix was added to all wells using a Multidrop combi dispenser, followed by another sec/1000 rpm centrifugation step. After an incubation of the plates for 180 min at room temperature, excitation at 665/620 nM (HTRF ratio) was measured using an Envison Reader (PerkinElmer).

Data evaluation and calculation: For data evaluation and calculation, HTRF ratios were calibrated using the cGAMP standard curve. After that, the measurement of the low control was set as 0% control and the measurement of the high control was set as 100% control. The IC50 values were calculated using the standard 4 parameter logistic regression formula. Calculation: [y=(a−d)/(1+(x/c)\hat{ }b)+d], a=low value, d=high value; x=conc M; c=IC50 M; b=slope;

The results of this assay are shown in the table below.

### Determination of the Increase of Stability of Sting Protein Against Thermal Denaturation, Differential Scanning Fluorimetry (DSF)

The binding affinity of the compounds of the invention may be demonstrated using a thermal shift assay that measures the stability of a suitable protein material of human STING against thermal denaturation in the presence of compounds. In this assay, the unfolding temperature of a protein is monitored in the presence of a fluorescent dye which exhibits affinity for the hydrophobic amino acids of the protein that are buried in its folded state and are gradually exposed during unfolding. Dye fluorescence is quenched in aqueous environment and increases upon association of the dye with the hydrophobic parts of the unfolding protein. A plot of the fluorescence intensity as a function of temperature typically displays a sigmoidal curve that is interpreted by a two-state model of protein unfolding (Differential Scanning Fluorimetry). The inflection point of the curve represents the “melting” temperature of the protein (Tm) which is calculated numerically using the Boltzmann equation.

Method: The thermal stability of the STING protein was measured using a specific expression construct of the cGAMP binding domain of wild-type (GRR) human STING comprising residues 155-341 and a N-terminal 8×His-tag (SEQ ID NO: 3) in assay buffer containing 20 mM Tris, 150 mM NaCl at pH7.5.

The assay uses Hard-Shell® PCR Plates 384-Well CLR/WHT (Catalog #HSP3805, BIO-RAD), Microseal® ‘B’ Adhesive Seals for PCR Plates (Catalog #MSB-1001, BIO-RAD) and was run on a CFX384 Real-Time System (Bio-Rad).

A DMSO stock solution of SYPRO orange (SIGMA S5692-500UL) was prepared.

Compound stock solutions (10 mM in DMSO) were diluted 1:2 in DMSO to an intermediate compound concentration of 5 mM and then further diluted 1:40 in assay buffer resulting in a compound concentration of 125 μM and 2.5% DMSO.

Fluorescent dye stock solution (5000×SYPRO Orange) was then mixed with target protein and buffer to a concentration of 15 uM Protein and 25×SYPRO Orange. 2 ul of this protein-dye-mixture was added to 8 ul compound solution. Final volume was 10 uL. 3-6 well positions were used as negative control (protein with 2% DMSO). The plates were prepared for duplicate measurement and centrifuged for 2 min at 1000 g. In the measurement, 160 cycles of 0.5° C. were used (temperature ramp 15 s/cycle, 15° C. to 95° C.).

Final Assay concentrations for compound characterization were as follows: 100 uM compound, 3 uM target protein, 5×SYPRO Orange, 2% DMSO in 10 ul. All dispensing steps were performed using a HamiltonStar pipetting robot (Hamilton).

Dissociation curves were processed in Bio-Rad CFX Manager. Peak type was set to “negative”. Compound codes for screen were assigned in the plate layout.

Two replicates of TM measurements were averaged, and the standard deviation was calculated. In cases of SD>1.5° C. the measurement was repeated.

The melting point (Tm) obtained for STING protein alone was subtracted from T obtained for protein incubated with ligand to generate ΔTm values.

Protein production and purification: The protein used for the biophysical experiments was a recombinant human STING protein comprising its cytosolic ectodomain. A codon optimized DNA sequence (for expression in Escherichia coli) encoding amino acid residues 155 to 341 (Swiss Prot Q86WV6) of human STING (WT) was synthesized by GeneArt (Regensburg, Germany) and inserted into a pET17b E. coli expression vector. The protein construct encodes an N-terminal 8×His-tag (SEQ ID NO: 3) followed by tobacco etch virus protease (TEV) cleavage site and the above STING gene sequence. The resulting protein sequence for the used STING variant is listed below:

His-TEV-hSTING (WT)
(SEQ ID NO: 1)
MHHHHHHHHENLYFQSGVAHGLAWSYYIGYLRLILPELQARIRTYNQH
YNNLLRGAVSQRLYILLPLDCGVPDNLSMADPNIRFLDKLPQQTGDRA
GIKDRVYSNSIYELLENGQRAGTCVLEYATPLQTLFAMSQYSQAGFSR
EDRLEQAKLFCRTLEDILADAPESQNNCRLIAYQEPADDSSFSLSQEV
LRHLRQEEKEEV

For expression of recombinant human STING above construct was transformed into E. coli BL21 DE3 strain and grown in shake flasks in LB-medium at 37° C. Expression was induced by addition of isopropyl β-D-1-thiogalactopyranoside to a final concentration of 1 mM and cultures shaken overnight. Cell pellets were centrifuged and stored at −70° C. until further use. Protein was purified by cell thawing in lysis buffer (20 mM TRIS-HCl, pH 8, 300 mM NaCl, 2 mM mercaptoethanol, 20 mM imidazole, Complete Protease Inhibitor (Roche) and DNase (Roche)), followed by metal affinity purification using Ni-NTA resins and elution buffer consisting of 20 mM TRIS-HCl, pH 8, 300 mM NaCl, 2 mM mercaptoethanol, 300 mM imidazole and size exclusion chromatography in running buffer (20 mM TRIS-HCl, pH 8, 100 mM NaCl, 2 mM DTT). The peak fraction was collected and concentrated to 2.5 mg/mL.

The results of this assay are shown in the table below.

### Human Whole Blood Assay (HWBA)

For the detection of STING inhibition in physiological environment human whole blood was stimulated by the cyclic dinucleotide cGAMP. Pathway activity was monitored by measuring the IFNα2α production.

Assay method: Compounds were delivered as 10 mM DMSO solution and serial diluted and transferred to the 96-well Cell culture Plate (Corning #3595), prefilled with 20 μl OptiMEM (Gibco #11058-021) in each well, using an Echo acoustic dispenser. Typically, 8 concentrations were used with the highest concentration at 10 μM in the final assay volume followed by ~1:5 dilution steps. DMSO concentration was set to 0.1% in the final assay volume. The 96 well assay plate contained 9 test compounds, a reference compound and DMSO in control wells.

Collection of human whole blood from 3 or more healthy donors (male or female, no medication for 7 days, exception contraceptive and thyroxine) as Na-citrate blood (e.g. 3.8% in Monovettes from Sarstedt) is conducted in parallel. Whole blood was kept at room temperature for a maximum of 3 hours after collection until use in the assay.

160 μl of the whole blood samples were transferred to each well of the 96-well assay plates filled with compound/OptiMEM. All assay plates are prepared as duplicates with blood from different donors. Blood plates were kept at room temperature for 60 minutes and continuous shaking with 450 rpm, covered with the lid, but not sealed.

A 10× cGAMP assay solution was diluted from a 2 mM stock solution in 1×HBSS immediately before use at room temperature. 20 μl of the 10× cGAMP/HBSS were added to all compound and all high control wells, whereas HBSS only was added to all low control wells.

After covering assay plates with aera seals and the lid, blood plates were kept at room temperature for 30 minutes and continuous shaking with 450 rpm, followed by an overnight incubation of 22 h at 37° C. in the incubator, without shaking.

For the detection of IFNα-2α in human plasma, the biotinylated capture antibody (Antibody set IFNA2, Meso Scale Diagnostics #B21VH-3, including coating and capture antibody) was diluted 1:17.5 in Diluent 100 (Meso Scale Diagnostics #R50AA-4, according to the manufacturer. U-Plex MSD GOLD 96-well Small Spot Streptavidin SECTOR Plates (Meso Scale Diagnostics #L45SA-5) were coated with 25 μl diluted capture antibody. Coated plates were incubated for 60 min at room temperature under continuous shaking at 700 rpm. MSD IFNα-2α plates were washed three times with 150 μl wash buffer (1×HBSS, 0.05% Tween).

After blocking the plates with 100 μl block solution/well (1×HBSS with 0.2% Tween, 2% BSA) for 60 min at room temperature and continuous shaking at 700 rpm, plates were emptied as dry as possible by dumping just before continuing with the human plasma. Whole Blood assay plates were centrifuged at 1600 rpm for 10 minutes. 25 μl of supernatant was transferred with a pipetting robotics from each whole blood plate to the corresponding IFNα-2α plate. Plates were sealed with microplate seals and kept at room temperature again under continuous shaking at 700 rpm for two hours. Next MSD IFNα-2α plates were washed three times with 150 μl wash buffer (1×HBSS, 0.05% Tween), before adding 25 μl MSD SULFO-TAG IFNα-2α Antibody solution (1:100 diluted in Diluent 3 (Meso Scale Diagnostics #R50AP-2) to each well of the plates. Afterwards plates were sealed with microplate seals and kept at room temperature again under continuous shaking at 700 rpm for two hours. Finally MSD IFNα-2α plates were washed three times with 150 μl wash buffer (1×HBSS, 0.05% Tween). 150 μl 2×Read buffer was added to each well and plates were immediately measured with the MSD Sector S600 Reader using the vendor barcode.

Data evaluation and calculation: For data evaluation and calculation, % control calculation of each well was based on the mean of high (cGAMP stimulated control) and mean of low (unstimulated control) controls by using the following formula:

[counts(sample)−counts(low))/(counts(high)−counts(low))]*100

The IC50 values were calculated using the standard 4 parameter logistic regression formula.

Calculation: [y=(a−d)/(1+(x/c)\hat{ }b)+d], a=low value, d=high value; x=conc M; c=IC50 M; b=slope;

The results of this assay are shown in the table below.

### Human Sting Reportergene Assay

A THP1-BluelSG reporter cell line expressing wildtype STING and IRF dependent alkaline phosphatase reporter was used for the potency measurement of activators of human wildtype STING.

Assay Method: Compounds were delivered 10 mM DMSO solution and serially diluted in assay medium (RPMI 1640 (Life Technologies #A10491-01), 10% FCS (Life Technologies #10500-064), 1×Pen/Strep solution (Life Technologies #15140-122). Typically, 8 concentrations were used with the highest concentration at 10 or 100 μM in the final assay volume followed by ~1:5 dilution steps. DMSO concentration was set to 1% in the final assay volume. The 384 well assay plate contained 21 test compounds (column 1-21), a reference compound (column 22) and DMSO in column 23 and 24;

Cells, cultivated according to manufacturer's conditions (culture medium: RPMI 1640 (Life Technologies #A10491-01), 10% FCS (Life Technologies #10500-064), 1×Pen/Strep solution (Life Technologies #15140-122), 100 μg/mL Normocin (Life Technologies #ant-nr-1), 100 μg/mL Zeocin (Life Technologies #R25001) were harvested, resuspended and diluted in fresh assay medium. The cells were then seeded in 15 μl assay media to the assay plates (10000 cells/well), followed by addition of 5 μl prediluted compound solution to wells of the assay plates. Afterwards 5 ul per well of assay medium was added to the wells containing compounds, followed by a 30 min incubation at RT and a 24 h incubation at 37° C. Then 5 ul per well of assay medium with DMSO (1% f.c.) was added to the wells for the controls, plus 5 μl of assay medium alone for negative controls (low values) or 5 μl of prediluted 2′3′-cGAMP (20 μM f.c.; BIOLOG Life Science Institute #C 161 or Invivogen #tlrl-nacga23) for positive controls (high values). Finally 75 μl of Quanti Blue reagent was added to the plates using a MultiDrop Combi, followed by 30 min incubation at 37° C. The absorbance was measured on the EnVision™ reader (PerkinElmer).

Data evaluation and calculation: For data evaluation and calculation, the measurement of the low control was set as 100% control and the measurement of the high control was set as 200% control. The EC50 values were calculated using the standard 4 parameter logistic regression formula. Calculation: [y=(a−d)/(1+(x/c)\hat{ }b)+d], a=low value, d=high value; x=conc M; c=IC50 M; b=slope;

The results of this assay are negative for agonism, wherein the threshold was set larger than 30 μM.

### Characterising Data

| | STING | | |
|---|---|---|---|
| | HTRF | | HWBA |
| | IC50 | DSF | IC50 |
| Example | (nM) | [K] | (μM) |
| 1 | 12.1 | 26 | 0.073 |
| 2 | 15.8 | 28 | 0.054 |
| 3 | 5.1 | 28 | 0.058 |
| 4 | | 27 | 0.8 |
| 5 | | | 0.061 |
| 6 | 14.2 | 29 | 0.061 |
| 7 | 24.6 | 25 | 0.061 |
| 8 | 14.6 | 24 | 0.062 |
| 9 | 14.1 | 33 | 0.063 |
| 10 | 9.3 | 30 | 0.069 |
| 11 | | 30 | 0.07 |
| 12 | 4.4 | 29 | 0.071 |
| 13 | 17.5 | 30 | 0.073 |
| 14 | 9.1 | 28 | 0.076 |
| 15 | 19.1 | 30 | 0.077 |
| 16 | 6.6 | 28 | 0.079 |
| 17 | 1.4 | 27 | 0.081 |
| 18 | 14.6 | 28 | 0.081 |
| 19 | 16.1 | 29 | 0.082 |
| 20 | 17.4 | 27 | 0.084 |
| 21 | 4.9 | 30 | 0.085 |
| 22 | 20.0 | 33 | 0.086 |
| 23 | 18.8 | 25 | 0.089 |
| 24 | 4.9 | 30 | 0.092 |
| 25 | 18.1 | 30 | 0.097 |
| 26 | 42.8 | 26 | 0.11 |
| 27 | 14.5 | 25 | 0.11 |
| 28 | 8.4 | 31 | 0.11 |
| 29 | 2.8 | 29 | 0.11 |
| 30 | 18.2 | 25 | 0.11 |
| 31 | | 27 | 0.11 |
| 32 | 8.6 | 28 | 0.11 |
| 33 | 6.1 | 31 | 0.11 |
| 34 | 7.1 | 29 | 0.12 |
| 35 | 19.3 | 29 | 0.13 |
| 36 | 14.6 | 24 | 0.13 |
| 37 | 5.5 | 27 | 0.13 |
| 38 | | 26 | 0.13 |
| 39 | 15.4 | 25 | 0.13 |
| 40 | 28.4 | 27 | 0.14 |
| 41 | 6.4 | 30 | 0.14 |
| 42 | 16.7 | 26 | 0.14 |
| 43 | | | 0.14 |
| 44 | 16.9 | 26 | 0.14 |
| 45 | 18.1 | 27 | 0.14 |
| 46 | 15.2 | 27 | 0.14 |
| 47 | 11.9 | 30 | 0.15 |
| 48 | 22.7 | 31 | 0.15 |
| 49 | 22.2 | 26 | 0.16 |
| 50 | 16.3 | 26 | 0.16 |
| 51 | 16.8 | 26 | 0.17 |
| 52 | 19.9 | 25 | 0.18 |
| 53 | 28.6 | 30 | 0.19 |
| 54 | 86.9 | 28 | 0.2 |
| 55 | 3.7 | 29 | 0.2 |
| 56 | 18.1 | 25 | 0.21 |
| 57 | 24.9 | 27 | 0.23 |
| 58 | 15.8 | 28 | 0.23 |
| 59 | | 27 | 0.24 |
| 60 | 23.9 | 28 | 0.24 |
| 61 | | 30 | 0.27 |
| 62 | 19.1 | 25 | 0.3 |
| 63 | 39.8 | 22 | 0.3 |
| 64 | 7.6 | 26 | 0.3 |
| 65 | 16.9 | 26 | 0.32 |
| 66 | 8.0 | 23 | 0.32 |
| 67 | 10.8 | 26 | 0.33 |
| 68 | 17.7 | 28 | 0.35 |
| 69 | 7.5 | 29 | 0.36 |
| 70 | 37.7 | 24 | 0.37 |
| 71 | | 26 | 0.37 |
| 72 | 17.6 | 30 | 0.4 |
| 73 | 11.4 | 28 | 0.44 |
| 74 | 16.9 | 25 | 0.45 |
| 75 | 9.8 | 28 | 0.45 |
| 76 | 17.4 | 26 | 0.52 |
| 77 | | 27 | 0.55 |
| 78 | 16.7 | 24 | 0.56 |
| 79 | 20.1 | 23 | 0.58 |
| 80 | 17.5 | 26 | 0.58 |
| 81 | 31.1 | 22 | 0.61 |
| 82 | 3.2 | 27 | 0.61 |
| 83 | 23.7 | 24 | 0.63 |
| 84 | 21.0 | 27 | 0.64 |
| 85 | 15.9 | 23 | 0.66 |
| 86 | 6.9 | 28 | 0.69 |
| 87 | 20.2 | 24 | 0.85 |
| 88 | 97.9 | 25 | 0.93 |
| 89 | 17.2 | 24 | 1 |
| 90 | 19.9 | 29 | 1.1 |
| 91 | 7.6 | 29 | 1.6 |
| 92 | 4.3 | 24 | 2 |
| 93 | 17.4 | 26 | 2.4 |
| 94 | 17.1 | 27 | 2.4 |
| 95 | 11.4 | 24 | 2.5 |
| 96 | 34.0 | 29 | 2.9 |
| 97 | 3.4 | 31 | 0.052 |
| 98 | 3.1 | 29 | 0.031 |
| 99 | | 29 | 0.035 |
| 100 | 6.9 | 28 | 0.037 |
| 101 | 10.6 | 30 | 0.039 |
| 102 | 3.6 | 28 | 0.04 |
| 103 | 9.3 | 29 | 0.044 |
| 104 | 2.0 | 31 | 0.049 |
| 105 | 78.1 | 24 | 1.6 |
| 106 | 3.2 | 28 | 0.028 |
| 107 | 3.8 | 31 | 0.031 |
| 108 | 5.8 | 27 | 0.038 |
| 109 | 18.8 | 27 | 0.078 |
| 110 | 8.3 | 29 | 0.085 |
| 111 | 25.2 | 24 | 0.75 |
| 112 | 31.7 | 23 | 0.17 |
| 113 | 62.8 | 23 | 1.2 |

### Indications

As has been found, the compounds of formula (I) or of formula (I′) are characterized by their range of applications in the therapeutic field.

Particular mention should be made of those applications for which the compounds of the invention are used on the basis of their pharmaceutical activity as STING inhibitors. While the cGAS/STING pathway is important for host defense against invading pathogens, such as viral infection and invasion by some intracellular bacteria, cellular stress and genetic factors may also cause production of aberrant cellular dsDNA, e.g. by nuclear or mitochondrial leakage, and thereby trigger autoinflammatory responses. Consequently, STING inhibitors have a strong therapeutic potential to be used in the treatment of diverse autoinflammatory and autoimmune diseases.

A STING inhibitor will block inflammation and aberrant tissue remodeling in a cluster of autoimmune and inflammatory diseases including systemic lupus erythematosus (SLE), systemic sclerosis, inflammatory bowel disease, sepsis, Sjogren's syndrome, dermatomyositis, rheumatoid arthritis, as well as a cluster fibrosis diseases including NASH, IPF, chronic kidney fibrosis.

A STING inhibitor also has applications to additional diseases such as cancer, heart failure AMD, retinopathy, glaucoma, diabetes, obesity, aging, muscle disorders, osteoarthritis, ALS, Parkinson's disease, COVID-19.
An et al., Arthritis Rheumatol. 2017 April; 69(4):800-807, disclosed that cGAS expression in peripheral blood mononuclear cells (PBMCs) was significantly higher in patients with the autoimmune disease systemic lupus erythematosus (SLE) than in normal controls. Targeted measurement of cGAMP by tandem mass spectrometry detected cGAMP in 15% of the tested SLE patients, but none of the normal or rheumatoid arthritis controls. Disease activity was higher in SLE patients with cGAMP versus those without cGAMP.
Thim-Uam et al (iScience. 2020 Sep. 4; 23(9):101530) demonstrated that STING deficiency ameliorated lupus development in Fcgr2b-deficient mice. Prabakaran et al (EBioMedicine. 2021 April; 66:103314) shows that a STING pathway inhibitor ISD017 blocks STING activity in vivo and ameliorates disease development in a mouse model for lupus. ISD017 treatment also blocks pathological cytokine responses in PBMCs from lupus patients with elevated IFN-I levels.
Ryu et al (Arthritis Rheumatol. 2020 November; 72(11):1905-1915) showed that plasma mtDNA concentrations were increased in the 2 Systemic sclerosis-associated interstitial lung disease (SSc-ILD) cohorts, reflective of ventilatory decline, and were positively associated with both TLR-9 and cGAS/STING activation as well as type I IFN and IL-6 expression. Liu et al (Rheumatology (Oxford) 2022 Jun. 10; keac324.) showed increased DNA leakage, STING expression and vascular inflammation in skins of SSc patients, and STING deficiency or H151 administration ameliorated fibrosis and vasculopathy both in vitro and in BLM-induced SSc mice.
Li et al show that plasma-derived DNA containing-extracellular vesicles induce STING-mediated proinflammatory responses in dermatomyositis (Theranostics. 2021; 11(15): 7144-7158). Zhou et al (J Clin Lab Anal. 2022 October; 36(10): e24631) describes a correlation between activation of cGAS-STING pathway and myofiber atrophy/necrosis in dermatomyositis.
Haag et al (Nature. 2018 July; 559(7713):269-273) demonstrated that a covalent STING inhibitor attenuated pathological features of autoinflammatory disease in TREX1_KO mice. Loss of function mutation of TREX1 leads rare monogenic interferonopathies such as Aicardi-Goutieres syndrome (AGS).
Hu et al (EBioMedicine. 2019 March; 41:497-508) showed that in human abdominal sepsis, STING expression was elevated in peripheral blood mononuclear cells and intestinal biopsies compared with healthy controls. In human abdominal sepsis, STING expression was elevated in peripheral blood mononuclear cells and intestinal biopsies compared with healthy controls. STING knockout mice attenuated alleviated inflammatory response, gut permeability, and decreased bacterial translocation in a sepsis model. Zeng et al (ci Transl Med. 2017 Oct. 18; 9(412):eaan5689) also showed that STING deficiency in mice protected two sepsos modeled (LPS model and cecal ligation and puncture model) and the degree of STING expression in the human intestinal lamina propria correlated with the intestinal inflammation in septic patients. Inhibition of the ALK-STING pathway protects mice against CLP-induced polymicrobial sepsis.
In Schuliga et al., Clin. Sci. (Lond). 2020 Apr. 17; 134(7):889-905, it is described that self-DNA perpetuates IPF lung fibroblast senescence in a cGAS-dependent manner. Benmerzoug et al (Nat. Commun. 9, 1-19 (2018)) shows that STING-dependent sensing of self-DNA drives silica-induced lung inflammation, which can lead to lung fibrosis.
Additional scientific hints linking the cause for metabolic diseases such as non-alcoholic fatty liver disease (NAFLD) other fibrosing diseases such as non-alcoholic steatohepatitis (NASH) with the cGAS/STING pathway have been described in Yu et al., J. Clin. Invest. 2019 Feb. 1; 129(2):546-555, and in Cho et al., Hepatology. 2018 October; 68(4): 1331-1346, and in Qiao et al., Metabolism 2018 April; 81:13-24 doi: 10.1016/j.metabol.2017.09.010. Epub 2017 Oct. 26
Nascimento et al., Sci. Rep. 2019 Oct. 16; 9(1):14848, discloses that self-DNA release and STING-dependent sensing drives inflammation due to cigarette smoke in mice hinting at a link between the cGAS-STING pathway and chronic obstructive pulmonary disease (COPD).
Ahn et al (Cell Rep 2017 21:3873-3884) describes that STING-deficient mice protects in an Inflammatory Colitis model. Martin et al (Sci Rep 2019 Oct. 3; 9:14281) describes that STING deletion protects while or STING stimulation, exacerbates intestinal inflammation in the dextran sodium sulphate (DSS) model of colitis. These publications support STING as a potential therapeutic target for prevention of inflammatory bowel disease (IBD).
Kerur et al., Nat. Med. 2018 January; 24(1):50-61, describes that cGAS plays a significant role in noncanonical-inflammasome activation in age-related macular degeneration (AMD).
Further, the STING inhibitors also have a therapeutic potential in the treatment of cancer (see Hoong et al., Oncotarget. 2020 Jul. 28; 11(30):2930-2955, and Chen et al., Sci. Adv. 2020 Oct. 14; 6(42):eabb8941). Furthermore shown in Bakhoum et el., Nature. 2018 Jan. 25; 553(7689):467-472: “Chromosomal instability drives metastasis through a cytosolic DNA response”, and in Liu et al., Nature. 2018 November; 563(7729):131-136: “Nuclear cGAS suppresses DNA repair and promotes tumorigenesis”.
STING inhibitors have also the potential in the treatment of obesity and diabetes as shown in Mao et al., Arterioscler Thromb Vasc Biol (2017) 37(5):920-9. doi: 10.1161/ATVBAHA.117.309017
Additionally, the STING inhibitors have also a therapeutic potential in the treatment of heart failure (King et al, Nat Med 2017 December; 23(12):1481-1487; Hu et al., Am. J. Physiol. Heart Circ. Physiol. 2020 Jun. 1; 318(6):H1525-H1537).
Further scientific hints at a correlation between Parkinson's disease and the cGAS/STING pathway (Sliter et al., Nature. 2018 September; 561(7722):258-262), between amyotrophic lateral sclerosis (ALS) and STING (Yu et al, Cell 2020; 183:636-649) and between Sjogren's syndrome and the cGAS/STING pathway (Papinska et al., J. Dent. Res. 2018 July; 97(8):893-900) exist.
Furthermore, STING inhibitors have also a therapeutic potential in the treatment of COVID-19/SARS-CoV-2 infections as shown in Di Domizio et al., Nature. 2022 Jan. 19. doi: 10.1038/s41586-022-04421-w: “The cGAS-STING pathway drives type I IFN immunopathology in COVID-19”, and in Neufeldt et al., Commun Biol. 2022 Jan. 12; 5(1):45. doi: 10.1038/s42003-021-02983-5: “SARS-CoV-2 infection induces a pro-inflammatory cytokine response through cGAS-STING and NF-kappaB”.
Additionally, STING inhibitors have a therapeutic potential in the treatment of renal inflammation and renal fibrosis as shown in Chung et al., Cell Metab. 2019 30:784-799: “Mitochondrial Damage and Activation of the STING Pathway Lead to Renal Inflammation and Fibrosis”, and in Maekawa et al., Cell Rep. 2019 29:1261-1273: “Mitochondrial Damage Causes Inflammation via cGAS-STING Signaling in Acute Kidney Injury”.
Further, it has been shown that STING promotes senescence, apoptosis, and extracellular matrix degradation in osteoarthritis (Guo et al, Cell Death Dis. 2021 Jan. 4; 12(1):13. doi: 10.1038). cGAS/STING null-mice have reduced tissue inflammation, improved heart/muscle function and have an extended lifespan (Dou et al, Nature. 2017 550: 402-406). Furthermore, in humans a variation within the STING gene is associated with healthy aging, most likely due to a decreased inflammaging (Hamann et al, Gerontology 2019; 65:145-154). Taken together, a STING inhibitor will reduce senescence associated inflammation and senescent cell accumulation and will leads improvement in senescence associated diseases such as aging/muscle disorders and osteoarthritis.

### Combinations

The compounds of formula 1 may be administered to the patient alone or in combination with one or more other pharmacologically active agents.

In a preferred embodiment of the invention the compounds may be combined with one or more pharmacologically active agents selected from the group of PDE 4 inhibitors (preferably 1-[[(5R)-2-[4-(5-chloropyrimidin-2-yl)-1-piperidyl]-5-oxo-6,7-dihydrothieno[3,2-d]pyrimidin-4-yl]amino]cyclobutyl]methanol and [1-[[(5R)-2-[4-(5-chlorophenyl-2-yl)-1-piperidyl]-5-oxo-6,7-dihydrothieno[3,2-d]pyrimidin-4-yl]amino]cyclobutyl]methanol as disclosed in WO 2013/026797), anti-inflammatory agents, anti-fibrotic agents, anti-allergic agents/anti-histamines, bronchodilators, beta 2 agonists/betamimetics, adrenergic agonists, anticholinergic agents, methotrexate, mycophenolate mofetil, leukotriene modulators, JAK inhibitors, anti-interleukin antibodies, non-specific immunotherapeutics such as interferons or other cytokines/chemokines, cytokine/chemokine receptor modulators (i.e. cytokine receptor agonists or antagonists), Toll-like receptor agonists (=TLR agonists), immune checkpoint regulators, anti-TNF antibodies and anti-BAFF agents.

### Formulations

The compounds of the invention may be administered by any suitable route of administration, including both systemic administration and topical administration. Systemic administration includes oral administration, parenteral administration, transdermal administration, rectal administration, and administration by inhalation. Parenteral administration refers to routes of administration other than enteral, transdermal, or by inhalation, and is typically by injection or infusion. Parenteral administration includes intravenous, intramuscular, intrasternal, and subcutaneous injection or infusion. Inhalation refers to administration into the patient's lungs whether inhaled through the mouth or through the nasal passages. Topical administration includes application to the skin. The compounds of the invention may be administered via eye drops to treat Sjogren's syndrome.

Suitable forms for administration are for example tablets, capsules, solutions, syrups, emulsions or inhalable powders or aerosols. The content of the pharmaceutically effective compound(s) in each case should be in the range from 0.1 to 90 wt. %, preferably 0.5 to 50 wt. % of the total composition, i.e. in amounts which are sufficient to achieve the dosage range specified hereinafter.

The preparations may be administered orally in the form of a tablet, as a powder, as a powder in a capsule (e.g. a hard gelatin capsule), as a solution or suspension. When administered by inhalation the active substance combination may be given as a powder, as an aqueous or aqueous-ethanolic solution or using a propellant gas formulation.

Preferably, therefore, pharmaceutical formulations are characterized by the content of one or more compounds of formula (I) or of formula (I′) according to the preferred embodiments above. It is particularly preferable if the compounds of formula (I) or of formula (I′) are administered orally, and it is also particularly preferable if they are administered once or twice a day. Suitable tablets may be obtained, for example, by mixing the active substance(s) with known excipients, for example inert diluents such as calcium carbonate, calcium phosphate or lactose, disintegrants such as corn starch or alginic acid, binders such as starch or gelatine, lubricants such as magnesium stearate or talc and/or agents for delaying release, such as carboxymethyl cellulose, cellulose acetate phthalate, or polyvinyl acetate. The tablets may also comprise several layers.

Coated tablets may be prepared accordingly by coating cores produced analogously to the tablets with substances normally used for tablet coatings, for example kollidone or shellac, gum arabic, talc, titanium dioxide or sugar. To achieve delayed release or prevent incompatibilities the core may also consist of a number of layers. Similarly, the tablet coating may consist of a number of layers to achieve delayed release, possibly using the excipients mentioned above for the tablets.

Syrups containing the active substances or combinations thereof according to the invention may additionally contain a sweetener such as saccharine, cyclamate, glycerol or sugar and a flavor enhancer, e.g. a flavoring such as vanillin or orange extract. They may also contain suspension adjuvants or thickeners such as sodium carboxymethyl cellulose, wetting agents such as, for example, condensation products of fatty alcohols with ethylene oxide, or preservatives such as p-hydroxybenzoates.

Capsules containing one or more active substances or combinations of active substances may for example be prepared by mixing the active substances with inert carriers such as lactose or sorbitol and packing them into gelatin capsules. Suitable suppositories may be made for example by mixing with carriers provided for this purpose, such as neutral fats or polyethylene glycol or the derivatives thereof.

Excipients which may be used include, for example, water, pharmaceutically acceptable organic solvents such as paraffins (e.g. petroleum fractions), vegetable oils (e.g. groundnut or sesame oil), mono- or polyfunctional alcohols (e.g. ethanol or glycerol), carriers such as e.g. natural mineral powders (e.g. kaolins, clays, talc, chalk), synthetic mineral powders (e.g. highly dispersed silicic acid and silicates), sugars (e.g. cane sugar, lactose and glucose), emulsifiers (e.g. lignin, spent sulphite liquors, methylcellulose, starch and polyvinylpyrrolidone) and lubricants (e.g. magnesium stearate, talc, stearic acid and sodium lauryl sulphate).

For oral administration the tablets may, of course, contain, apart from the abovementioned carriers, additives such as sodium citrate, calcium carbonate and dicalcium phosphate together with various additives such as starch, preferably potato starch, gelatin and the like. Moreover, lubricants such as magnesium stearate, sodium lauryl sulphate and talc may be used at the same time for the tableting process. In the case of aqueous suspensions, the active substances may be combined with various flavor enhancers or colorings in addition to the excipients mentioned above.

1. A compound of formula 1: 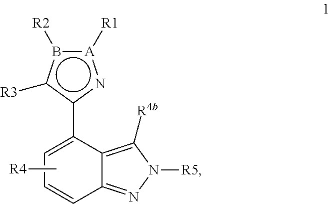
wherein
B-A is ═C—N— or —N—C═;
R1 is selected from 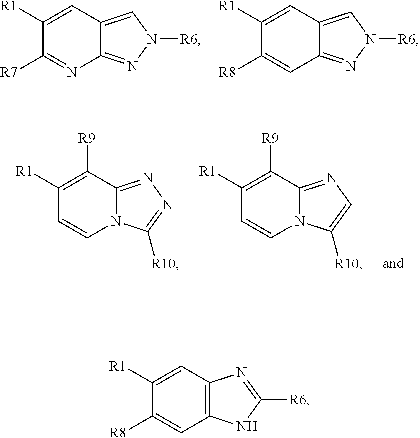
wherein R1 is the attaching point to the structure of formula 1;
R2, when B-A is —N—C═, is C₁₋₆-alkyl-, C₃₋₆-cycloalkyl-, or C₁₋₆-haloalkyl-;
R2, when B-A is ═C—N—, is C₁₋₆-alkyl-, C₃₋₆-cycloalkyl-, C₁₋₆-haloalkyl-, C₁₋₆-alkyl-O—, HO—, H₂N—, C₁₋₆-alkyl-HN—, or (C₁₋₆-alkyl)₂N—;
R3 is H—
or
C₁₋₆-alkyl-, C₃₋₇-cycloalkyl-, C₂₋₆alkenyl-, or C₃₋₇-heterocycloalkyl-, each optionally substituted with a group selected from F—, HO—, Me-, EtO-, and NH₂(O)C—;
R4 is H—, F—, or HO—;
R4^{b} is H—, F—, Cl—, Br—, NC—, or HO—;
R5- is 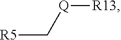
wherein R5 is the attaching point to the structure of formula 1;
Q is —C(R11)(R12)-, —S(O)—, or —S(O)₂—,
R6 is C₂₋₆-alkenyl-,
or
C₁₋₆-alkyl-, optionally substituted independently of one another by one or two substituents selected from the group consisting of C₃₋₆-cycloalkyl-, halogen-, HO—, C₁₋₆-alkyl-O—, C₁₋₆-alkyl-HN—, (C₁₋₆-alkyl)₂N—, NC—, (C₁₋₆-alkyl)₂(O)P—, and (4-Methoxyphenyl)methyl-,
or
a heterocyclyl selected from tetrahydrofuran-, 1,-4-dioxane-, pyrrolidine-, piperazine-, morpholine-, pyridine-, pyrazole-, and triazole-, each optionally substituted independently of one another by one or two substituents selected from the group consisting of C₁₋₆-alkyl-, halogen-, and O═;
R7, R8, and R9 are C₃₋₆-cycloalkyl-, optionally substituted with C₁₋₆-alkyl- or one or two halogen atoms,
or
cyclopropylmethyl-, C₁₋₆-haloalkyl-, C₁₋₆-alkyl-O—, C₁₋₆-alkyl-HN—, (C₁₋₆ alkyl)₂N—, or C₁₋₆-alkyl-S—,
or
C₁₋₆-alkyl-, straight or branched, optionally substituted with HO—, C₁₋₆-alkyl-O—, C₁₋₆-alkyl-HN—, (C₁₋₆-alkyl)₂N—, or morpholine-,
or
a heterocyclyl selected from pyrrolidine-, tetrahydrofuran-, and tetrahydropyran,
or
C₁₋₆-haloalkyl-O—;
R10 is C₁₋₆-alkyl- or C₁₋₆-haloalkyl-;
R11 is H—, HO—, halogen-, R14-O—, or R14-NH—;
R12 is H— or F—;
R13 is carbocyclyl-, heterocyclyl-, aryl-, or heteroaryl, each optionally substituted with one or two substituents selected from the group consisting of C₁₋₆alkyl-, C₁₋₆-haloalkyl-, HO—, NC—, halogen-, R15-(CH₂)ₙ—O—, R15-(CH₂)ₙ—NH—, (R15-(CH₂)ₙ)₂—N—, R15-(CH₂), —S(O)—, and R15-(CH₂)ₙ—S(O)₂—;
R14 is C₁₋₆-haloalkyl-,
or
C₁₋₆-alkyl-, optionally substituted with C₃₋₆-cycloalkyl-, C₂₋₆-alkenyl-, HO—, C₁₋₆-alkyl-O—, H₂N—C(O)—, C₁₋₆-alkyl-HN—C(O)—, or (C₁₋₆-alkyl)₂N—C(O)—;
R15 is C₁₋₄-alkyl-, C₁₋₆-haloalkyl-, NC—, C₁₋₆-alkyl-HN—, (C₁₋₆-alkyl)₂N—, (C₁₋₆-alkyl)₂(HO)C—, aryl- or heterocyclyl-; and
n is 0, 1, 2, or 3.

2. A compound according to claim 1, wherein
R1 is selected from

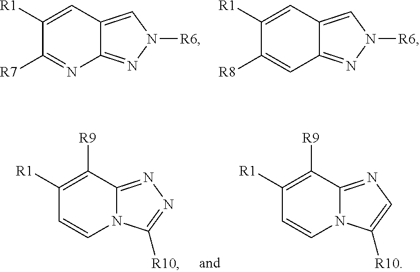

3. A compound according to claim 1, wherein
R3 is H—
or
C₁₋₆-alkyl-, C₃₋₇-cycloalkyl-, C₂₋₅-alkenyl-, or C₃₋₇-heterocycloalkyl-, each optionally substituted with a group selected from F—, HO—, Me-, EtO-, and NH₂(O)C—;
R4 is H—, F—, or HO—;
R4^{b} is H—, F—, Cl—, Br—, NC—, or HO—;
R5 is 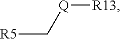
wherein R5 is the attaching point to the structure of formula 1;
Q is —C(R11)(R12)-, —S(O)—, or —S(O)₂—;
R6 is C₂₋₄-alkenyl-,
or
C₁₋₄-alkyl-, optionally substituted independently of one another by one or two substituents selected from the group consisting of C₃₋₄-cycloalkyl-, halogen-, HO—, C₁₋₄-alkyl-O—, C₁₋₄-alkyl-HN—, (C₁₋₄-alkyl)₂N—, NC—, (C₁₋₄-alkyl)₂(O)P—, and (4-Methoxyphenyl)methyl-,
or
a heterocyclyl selected from tetrahydrofuran-, 1,-4-dioxane-, pyrrolidine-, piperazine-, morpholine-, pyridine-, pyrazole-, and triazole-, each optionally substituted independently of one another by one or two substituents selected from the group consisting of C₁₋₄-alkyl-, halogen-, and O═;
R7, R8, and R9 are C₃₋₄-cycloalkyl-, optionally substituted with C₁₋₄-alkyl- or one or two halogen atoms,
or
cyclopropylmethyl-, C₁₋₄-haloalkyl-, C₁₋₄-alkyl-O—, C₁₋₄-alkyl-HN—, (C₁₋₄-alkyl)₂N—, or C₁₋₄-alkyl-S—,
or
C₁₋₄-alkyl-, straight or branched, optionally substituted with HO—, C₁₋₄-alkyl-O—, C₁₋₄-alkyl-HN—, (C₁₋₄-alkyl)₂N—, or morpholine-,
or
a heterocyclyl selected from pyrrolidine-, tetrahydrofuran-, and tetrahydropyran-,
or
C₁₋₄-haloalkyl-O—;
R10 is C₁₋₄-alkyl- or C₁₋₄-haloalkyl-;
R11 is H—, HO—, halogen-, R14-O—, or R14-NH—;
R12 is H— or F—;
R13 is carbocyclyl-, heterocyclyl-, aryl-, or heteroaryl, each optionally substituted with one or two substituents selected from the group consisting of C₁₋₄-alkyl-, C₁₋₄-haloalkyl-, HO—, NC—, halogen-, R15-(CH₂)ₙ—O—, R15-(CH₂)ₙ—NH—, (R15-(CH₂)ₙ)₂—N—, R15-(CH₂), —S(O)—, and R15-(CH₂)ₙ—S(O)₂—;
R14 is C₁₋₄-haloalkyl-,
or
C₁₋₅-alkyl, optionally substituted with C₃₋₄-cycloalkyl-, C₂₋₄-alkenyl-, HO—, C₁₋₄-alkyl-O—, H₂N—C(O)—, C₁₋₄-alkyl-HN—C(O)—, or (C₁₋₄-alkyl)₂N—C(O)—;
R15 is C₁₋₄-alkyl-, C₁₋₄-haloalkyl-, NC—, C₁₋₆-alkyl-HN—, (C₁₋₄-alkyl)₂N—, (C₁₋₄-alkyl)₂(HO)C—, aryl, or heterocyclyl; and
n is 0, 1, 2, or 3.

4. A compound according to claim 1, wherein
R2 is C₁₋₄-alkyl-;
R3 is C₁₋₄-alkyl-, optionally substituted with HO—,
or
C₃₋₄-cycloalkyl-, optionally substituted with methyl-;
R4 is H— or F—;
R5 is selected from 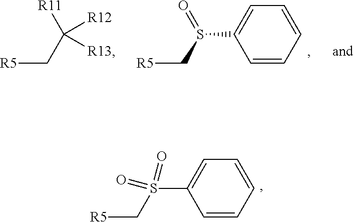
wherein R5 is the attaching point to the structure of formula 1;
R6 is C₂₋₄-alkenyl-,
or
C₁₋₄-alkyl-, optionally substituted independently of one another by one or two substituents selected from the group consisting of C₃₋₄-cycloalkyl-, halogen-, HO—, C₁₋₄-alkyl-O—, (C₁₋₄-alkyl)₂N—, NC—, (C₁₋₄-alkyl)₂(O)P—, and (4-Methoxyphenyl)methyl-,
or
a heterocyclyl selected from tetrahydrofuran-, 1,-4-dioxane-, pyrrolidine-, piperazine-, morpholine-, pyridine-, pyrazole-, and triazole-, each optionally substituted independently of one another by one or two substituents selected from the group consisting of C₁₋₄-alkyl-, halogen-, and O═;
R7 is C₃₋₄-cycloalkyl-, optionally substituted with C₁₋₄-alkyl- or one or two halogen atoms,
or
cyclopropylmethyl-, C₁₋₄-haloalkyl-, C₁₋₄-alkyl-O—, C₁₋₄-alkyl-HN—, (C₁₋₄-alkyl)₂N—, or C₁₋₄-alkyl-S—,
or
C₁₋₄-alkyl-, straight or branched, optionally substituted with HO—, C₁₋₄-alkyl-O—, C₁₋₄-alkyl-HN—, (C₁₋₄-alkyl)₂N—, or morpholine-,
or
a heterocyclyl selected from pyrrolidine-, tetrahydrofuran-, and tetrahydropyran-;
R8 is C₁₋₄-haloalkyl-O—;
R9 is C₁₋₄-alkyl- or C₃₋₄-cycloalkyl-;
R10 is C₁₋₄-alkyl- or C₁₋₄-haloalkyl-;
R11 is H—, HO—, halogen-, or R14-O—;
R12 is H— or F—;
R13 is cyclohexyl-, 3,-4-difluorphenyl-, 3-methyl-4N-pyridinyl-, or phenyl-, each optionally substituted at the 4-position with methyl-, HO—, F—, Cl—, Br—, or
R15-(CH₂)ₙ—O—;
R14 is C₁₋₄-haloalkyl-,
or
C₁₋₅-alkyl-, optionally substituted with C₃₋₄-cycloalkyl-, C₂₋₄-alkenyl-, HO—, C₁₋₄-alkyl-O—, H₂N—C(O)—, (C₁₋₄-alkyl)NH—C(O)—, or (C₁₋₄-alkyl)₂N—C(O)—;
R15 is NC—, (C₁₋₄-alkyl)₂N—, (C₁₋₄-alkyl)₂(HO)C—, phenyl-, or a heterocyclyl selected from oxetane-, tetrahydropyran-, and morpholine-; and
n is 0, 1, or 2.

5. A compound according to claim 1, wherein
R2 is methyl- or ethyl-;
R3 is ethyl-, iso-propyl-, cyclopropyl-, 1-methyl-cyclopropyl-, or 1-hydroxy-iso-propyl-;
R4 is H— or F—:
R5 is selected from 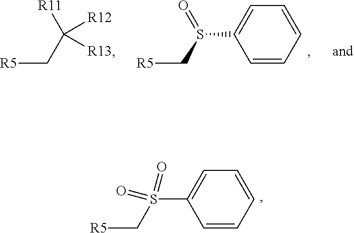
wherein R5 is the attaching point to the structure of formula 1;
R6 is C₂₋₄-alkenyl-,
or
C₁₋₄-alkyl-, optionally substituted independently of one another by one or two substituents selected from the group consisting of cyclopropyl-, F—, HO—, H₃C—O—, (H₃C)₂N—, NC—, (H₃C)₂(O)P—, and (4-methoxyphenyl)methyl-,
or
a heterocyclyl selected from tetrahydrofuran-, 1,-4-dioxane-, pyrrolidine-, piperazine-, morpholine-, pyridine-, pyrazole-, and triazole-, each optionally substituted independently of one another by one or two substituents selected from the group consisting of H₃C—, F—, and O═;
R7 is cyclopropyl-, 1-flourocyclopropyl-, 2,-2-diflourocyclopropyl-, 1-methyl-cyclopropyl-, cyclobutanyl-, cyclopropylmethyl-, F₂HC—, F₃C—, (iPr)—O—, (H₃C)NH—, (H₃C)₂N—, or H₃C—S—,
or
C₁₋₄-alkyl-, straight or branched, optionally substituted with HO—, H₃C—O—, H₃C—CH₂—O—, (H₃C)NH—, (H₃C)₂N—, or morpholine-,
or
a heterocyclyl selected from pyrrolidine-, tetrahydrofuran-, and tetrahydropyran-;
R8 is F₂HC—O or F₃C—O—;
R9 is methyl- or cyclopropyl-;
R10 is methyl- or F₂C—;
R11 is H—, HO—, F—, or R14-O—;
R12 is H— or F—;
R13 is cyclohexyl-, 3,-4-difluorphenyl-, 3-methyl-4N-pyridinyl-, or phenyl-, each optionally substituted at the 4-position with methyl-, HO—, F—, Cl—, Br—, or R15-(CH₂)ₙ—O—;
R14 is FH₂C or FH₂C—CH₂—,
or
C₁₋₅-alkyl-, optionally substituted with cyclopropyl-, H₂C═CH—, HO—, H₃C—O—, H₂N—C(O)—, or (H₃C)NH—C(O)—;
R15 is NC—, (H₃C)₂N—, (H₃C)₂(HO)C—, phenyl-, or a heterocyclyl selected from oxetane-, tetrahydropyran-, and morpholine-; and
n is 0, or 2.

6. A compound according to claim 1, wherein
B-A is ═C—N— or —N—C═;
R1 is 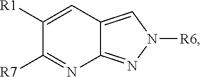
wherein R1 is the attaching point to the structure of formula 1;
R2 is methyl-;
R3 is ethyl-, iso-propyl-, cyclopropyl-, 1-methyl-cyclopropyl-, or 1-hydroxy-iso-propyl-;
R4 is H— or F—;
R5 is selected from 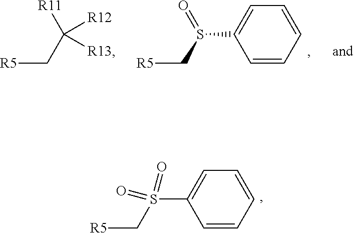
wherein R5 is the attaching point to the structure of formula 1;
R6 is methyl-, ethyl-, iso-propyl-, or cyclopropyl-;
R7 is cyclopropyl-, 1-fluorocyclopropyl-, 2,-2-difluorocyclopropyl-, 1-methyl-cyclopropyl-, cyclobutanyl-, cyclopropylmethyl-, F₂HC—, F₃C—, (iPr)—O—, (H₃C)NH—, (H₃C)₂N—, or H₃C—S—,
or
C₁₋₄-alkyl-, straight or branched, optionally substituted with HO—, H₃C—O—, H₃C—CH₂—O—, (H₃C)NH—, (H₃C)₂N—, or morpholine-,
or
a heterocyclyl selected from pyrrolidine-, tetrahydrofuran-, and tetrahydropyran-;
R11 is H—, HO—, F—, or R14-O—;
R12 is H—;
R13 is cyclohexyl-, 3,-4-difluorphenyl-, 3-methyl-4N-pyridinyl-, or phenyl-, each optionally substituted at the 4-position with methyl-, HO—, F—, Cl—, Br—, or R15-(CH₂)ₙ—O—;
R14 is FH₂C or FH₂C—CH₂—,
or
C₁₋₅-alkyl-, optionally substituted with cyclopropyl-, H₂C═CH—, HO—, H₃C—O—, H₂N—C(O)—, or (H₃C)NH—C(O)—;
R15 is NC—, (H₃C)₂N—, (H₃C)₂(HO)C—, phenyl-, or a heterocyclyl selected from oxetane-, tetrahydropyran-, and morpholine-; and
n is 0, 1, or 2.

7. A compound according to claim 1, wherein
B-A is ═C—N— or —N—C═;
R1 is selected from 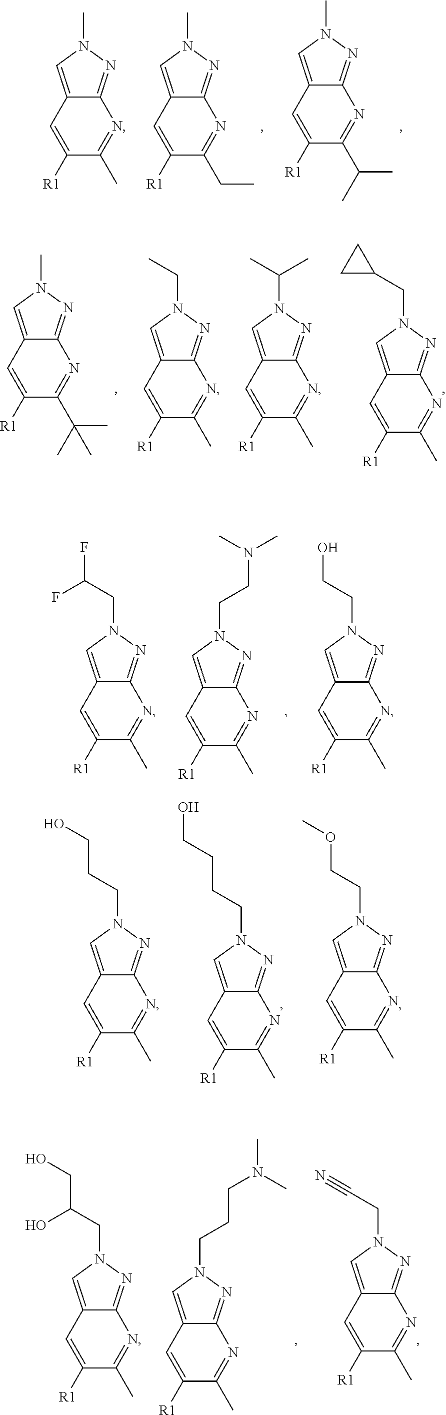 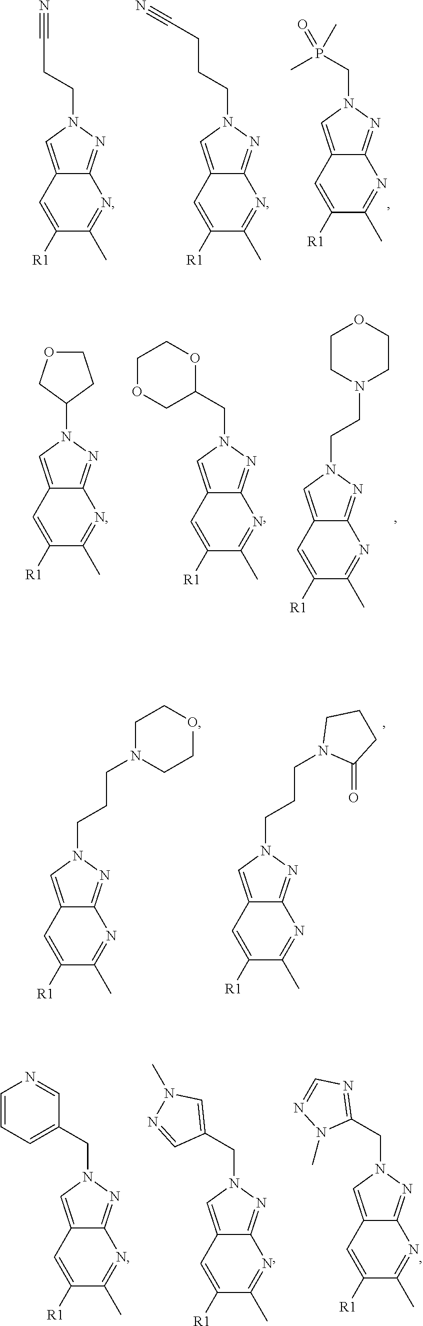 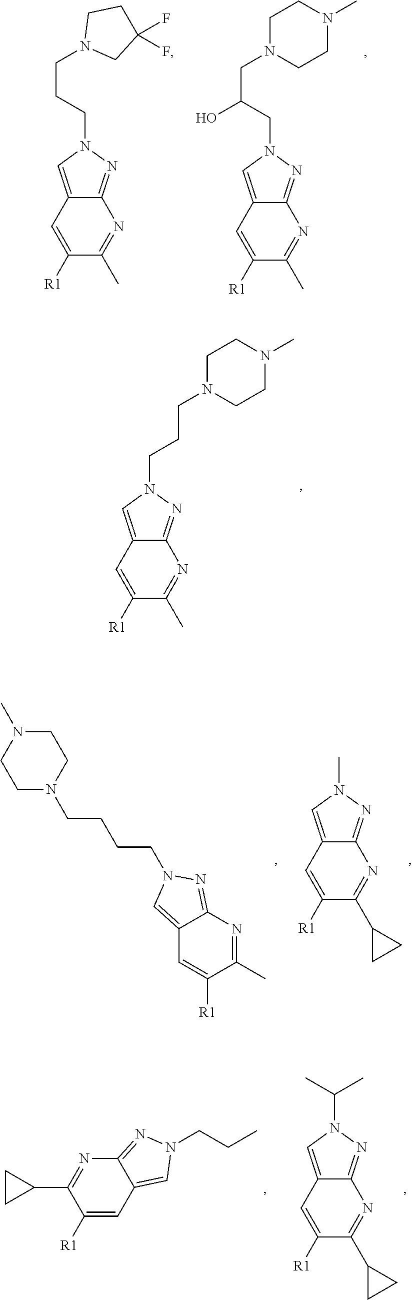 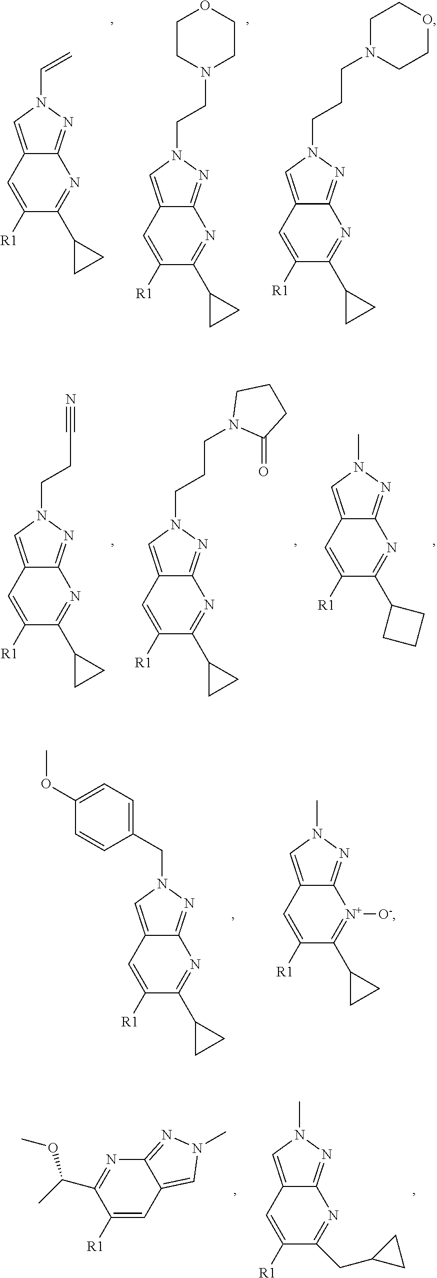 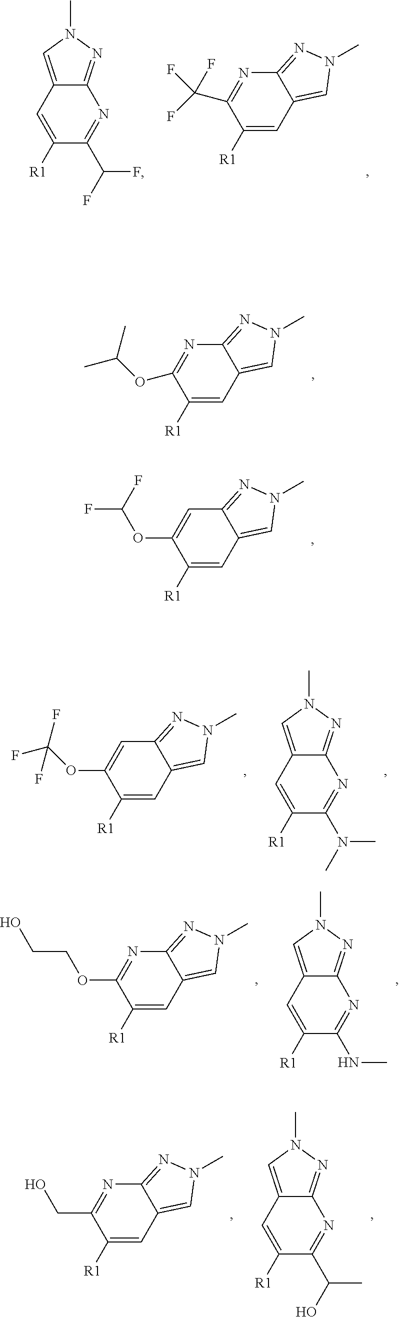 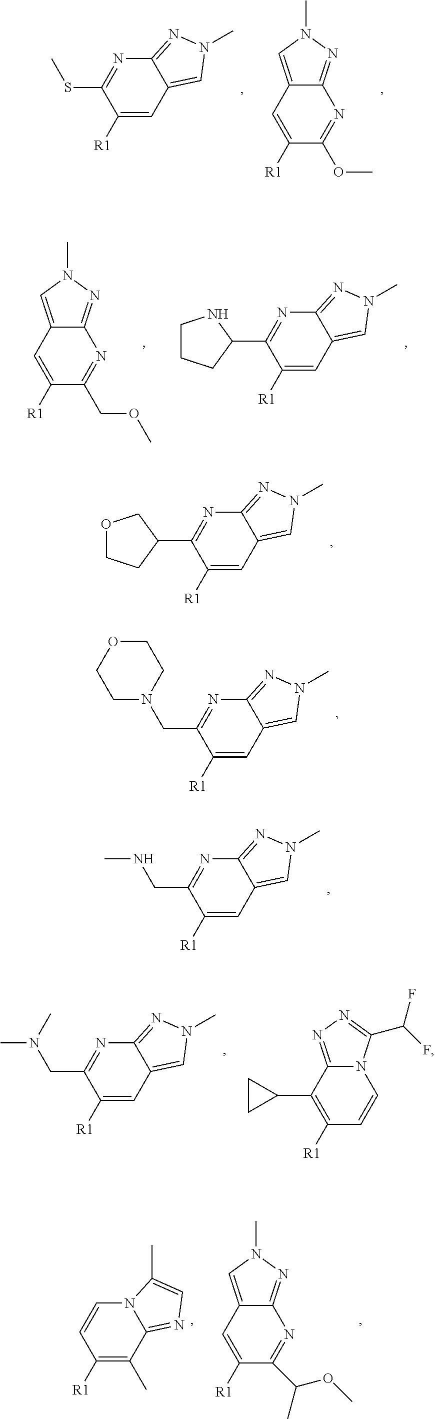 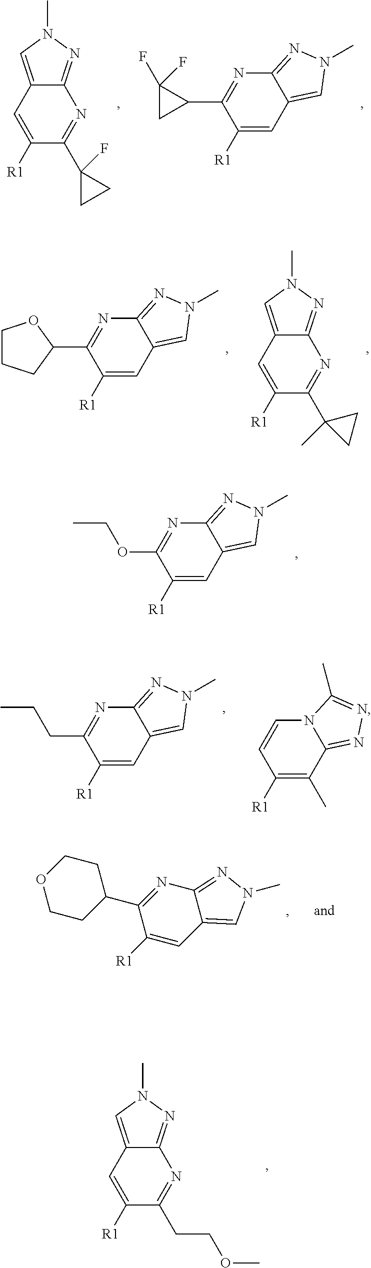
wherein R1 is the attaching point to the structure of formula 1;
R2 is methyl- or ethyl-;
R3 is ethyl-, iso-propyl-, cyclopropyl-, 1-methyl-cyclopropyl-, or ((HO)H₂C)(H₃C)HC—;
R4 is H— or F—;
R5 is selected from 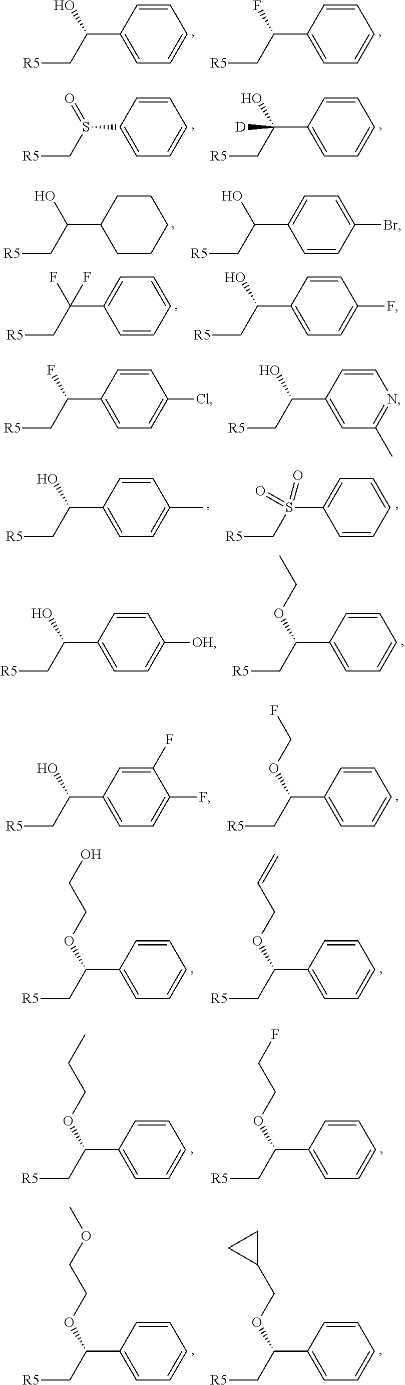 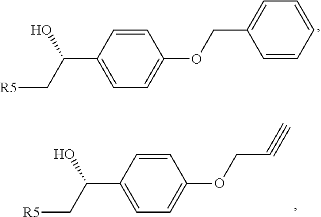 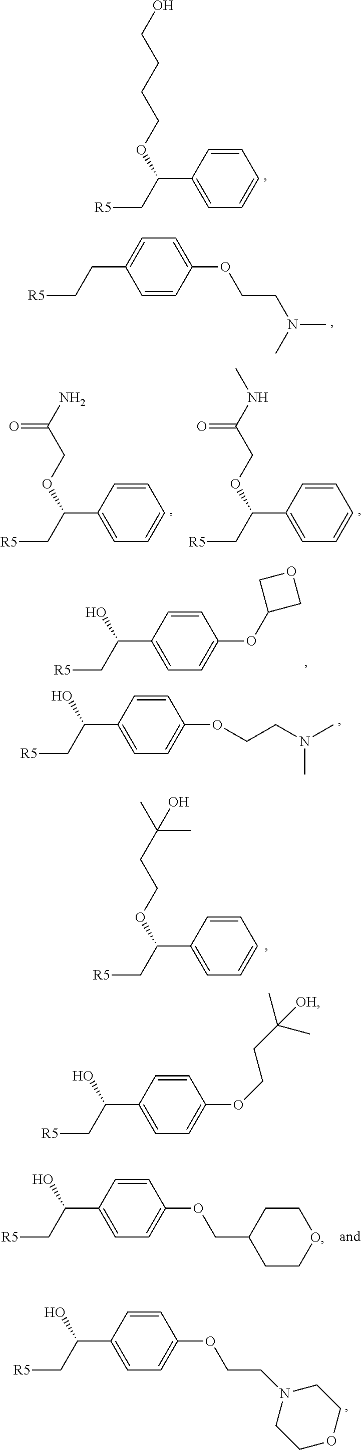
wherein R5 is the attaching point to the structure of formula 1.

8. A compound according to claim 1, wherein the compound of formula 1 is a compound of formula 1a:

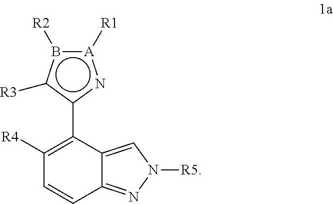

9. A compound according to claim 1, wherein the compound of formula 1 is a compound of formula 1b:

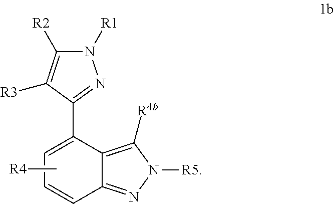

10. A compound according to claim 1, wherein the compound of formula 1 is a compound of formula 1c:

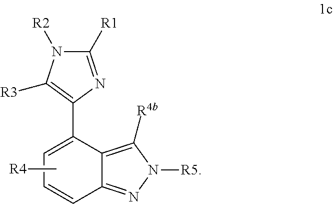

11. A compound according to claim 1, selected from the group consisting of

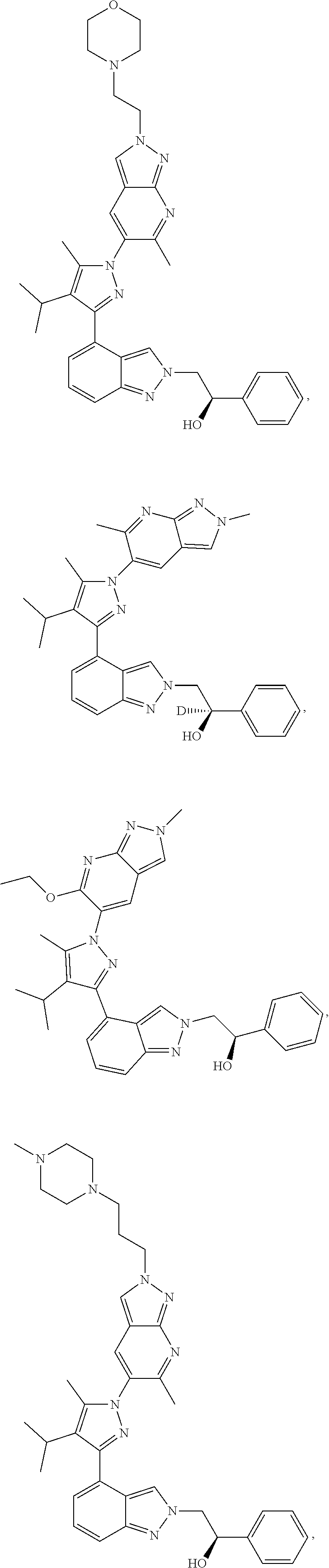 [SMILES: Cc1nc2nn(CCN3CCOCC3)cc2cc1-n1nc(-c2cccc3nn(C[C@H](O)c4ccccc4)cc23)c(C(C)C)c1C ; [2H][C@](O)(Cn1cc2c(-c3nn(-c4cc5cn(C)nc5nc4C)c(C)c3C(C)C)cccc2n1)c1ccccc1 ; CCOc1nc2nn(C)cc2cc1-n1nc(-c2cccc3nn(C[C@H](O)c4ccccc4)cc23)c(C(C)C)c1C ; Cc1nc2nn(CCCN3CCN(C)CC3)cc2cc1-n1nc(-c2cccc3nn(C[C@H](O)c4ccccc4)cc23)c(C(C)C)c1C]

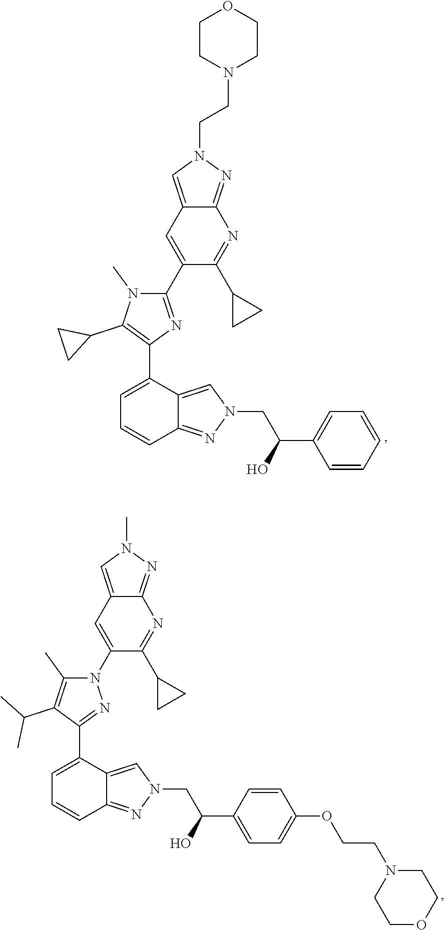 [SMILES: Cn1c(-c2cc3cn(CCN4CCOCC4)nc3nc2C2CC2)nc(-c2cccc3nn(C[C@H](O)c4ccccc4)cc23)c1C1CC1 ; Cc1c(C(C)C)c(-c2cccc3nn(C[C@H](O)c4ccc(OCCN5CCOCC5)cc4)cc23)nn1-c1cc2cn(C)nc2nc1C1CC1]

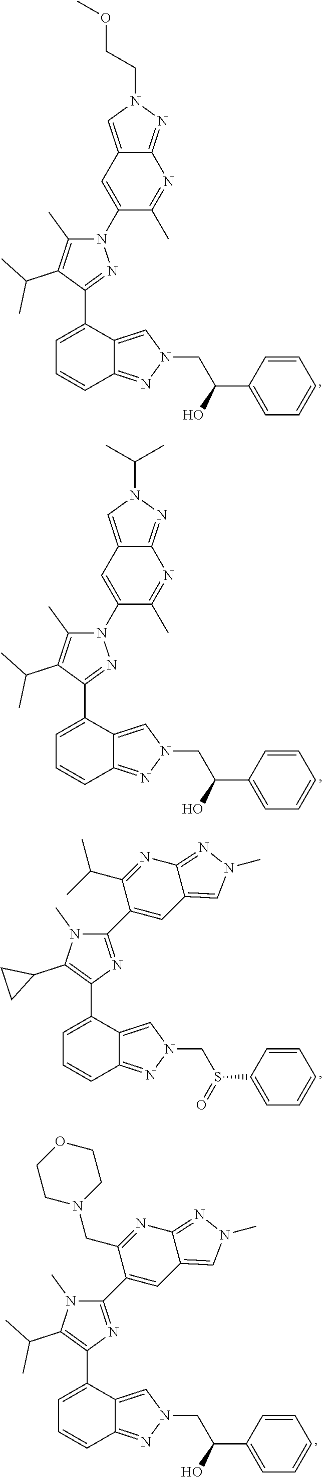

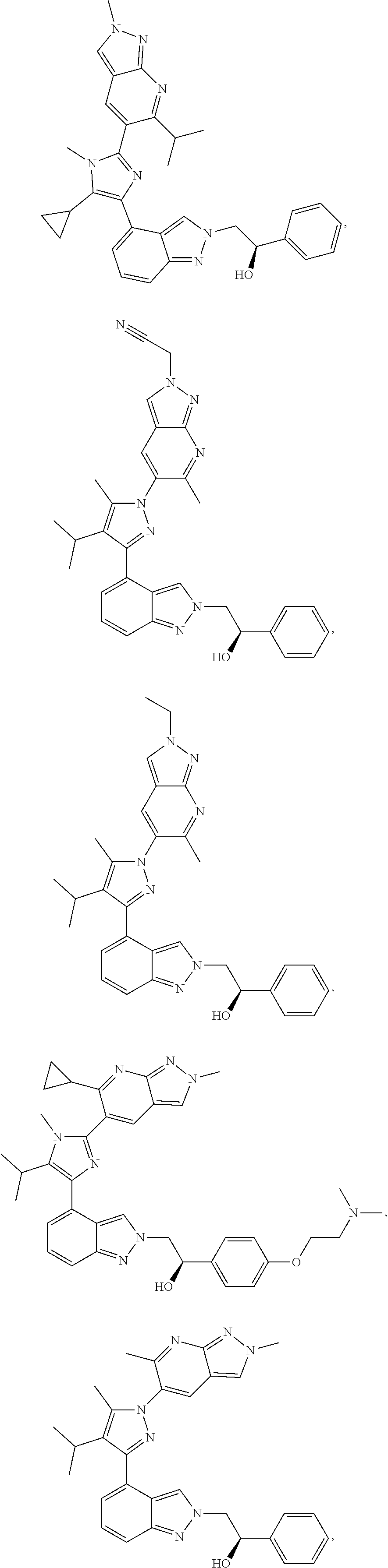

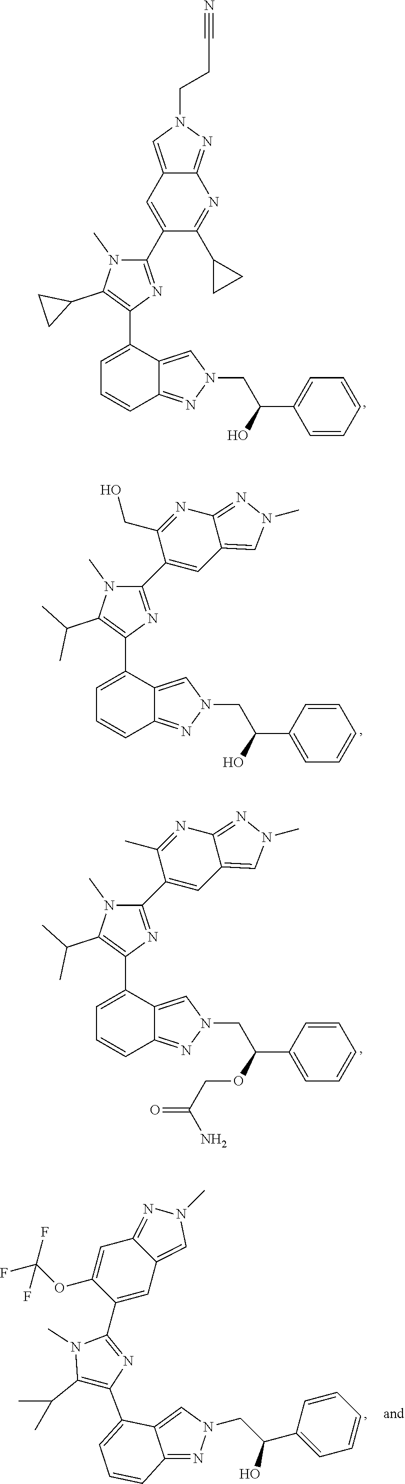 [SMILES: Cn1c(-c2cc3cn(CCC#N)nc3nc2C2CC2)nc(-c2cccc3nn(C[C@H](O)c4ccccc4)cc23)c1C1CC1 ; CC(C)c1c(-c2cccc3nn(C[C@H](O)c4ccccc4)cc23)nc(-c2cc3cn(C)nc3nc2CO)n1C ; Cc1nc2nn(C)cc2cc1-c1nc(-c2cccc3nn(C[C@H](OCC(N)=O)c4ccccc4)cc23)c(C(C)C)n1C ; CC(C)c1c(-c2cccc3nn(C[C@H](O)c4ccccc4)cc23)nc(-c2cc3cn(C)nc3cc2OC(F)(F)F)n1C]

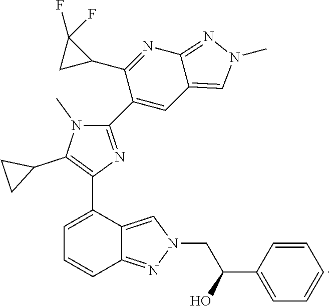 [SMILES: Cn1cc2cc(-c3nc(-c4cccc5nn(C[C@H](O)c6ccccc6)cc45)c(C4CC4)n3C)c(C3CC3(F)F)nc2n1]

12. A salt of a compound of formula 1 according to claim 1.

13. A pharmaceutically acceptable salt of a compound of formula 1 according to claim 1.

14. A method of treating a disease in a subject, said method comprising administering to the subject a compound of formula 1 according to claim 1, wherein the disease is a disease that can be treated by the inhibition of STING.

15. A method of treating a disease in a subject, said method comprising administering to the subject a compound of formula 1 according to claim 1, wherein the disease is:
(i) selected from the group consisting of systemic lupus erythematosus (SLE), monogenic and digenic interferonopathies, type 1 interferonopathies with mutations in DNASE2 or ATAD3A genes, age-related macular degeneration (AMD), retinopathy, glaucoma, amyotrophic lateral sclerosis (ALS), diabetes, obesity, inflammatory bowel disease (IBD), chronic obstructive pulmonary disease (COPD), Bloom's syndrome, Sjogren's syndrome, Parkinson's disease, heart failure, cancer, systemic sclerosis (SSc), dermatomyositis, non-alcoholic fatty liver disease (NAFLD), non-alcoholic steatotic hepatitis (NASH), acute on chronic liver failure (ACLF), interstitial lung disease (ILD), idiopathic pulmonary fibrosis (IPF), aging/muscle disorders, sepsis, rheumatoid arthritis, and osteoarthritis;
(ii) a fibrosing disease selected from the group consisting of systemic sclerosis (SSc), non-alcoholic steatohepatitis (NASH), acute on chronic liver failure (ACLF), monogenic and digenic interferonopathies, type 1 interferonopathies with mutations in DNASE2 or ATAD3A genes, and interstitial lung disease (ILD);
(iii) an interferonopathy selected from the group consisting of STING-associated vasculopathy with onset in infancy (SAVI), Aicardi-Goutieres syndrome (AGS), COPA syndrome, and familial chilblain lupus;
(iv) selected from the group consisting of age-related macular degeneration (AMD), retinopathy, glaucoma, aging, muscle disorders, heart failure, COVID-19/SARS-CoV-2 infection, renal inflammation, renal fibrosis, dysmetabolism, vascular diseases, cardiovascular diseases, diabetes, obesity, and cancer.

16-17. (canceled)

18. A pharmaceutical composition comprising a compound of formula 1 according to claim 1, or a pharmaceutically acceptable salt thereof, and optionally one or more pharmaceutically acceptable carriers and/or excipients.

19. A pharmaceutical composition comprising a compound of formula 1 according to claim 1, or a pharmaceutically acceptable salt thereof, in combination with one or more active agents selected from the group consisting of PDE 4 inhibitors, anti-inflammatory agents, anti-fibrotic agents, anti-allergic agents/anti-histamines, bronchodilators, beta 2 agonists/betamimetics, adrenergic agonists, anticholinergic agents, methotrexate, mycophenolate mofetil, leukotriene modulators, JAK inhibitors, anti-interleukin antibodies, non-specific immunotherapeutics, interferons or other cytokines/chemokines, cytokine/chemokine receptor modulators, toll-like receptor agonists, immune checkpoint regulators, an anti-TNF antibody, and an anti-BAFF antibody.

20. A pharmaceutical composition according to claim 18, wherein the compound of formula 1 is a compound of formula 1a:

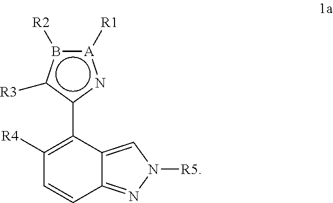

21. A pharmaceutical composition according to claim 18, wherein the compound of formula 1 is a compound of formula 1b:

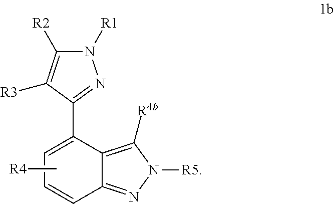

22. A pharmaceutical composition according to claim 18, wherein the compound of formula 1 is a compound of formula 1c:

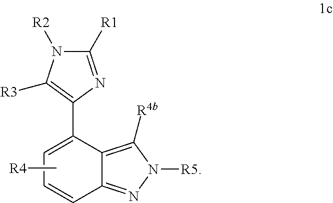

23. A pharmaceutical composition according to claim 18, wherein the compound is selected from the group consisting of 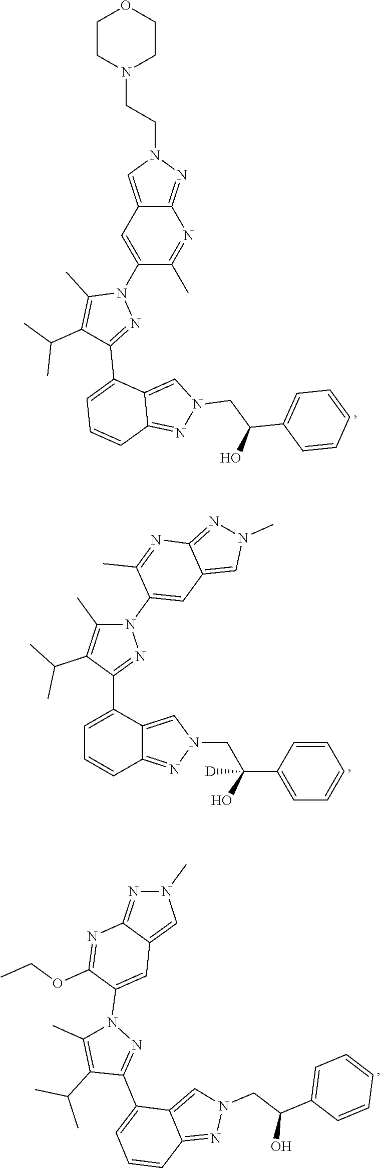 [SMILES: Cc1nc2nn(CCN3CCOCC3)cc2cc1-n1nc(-c2cccc3nn(C[C@H](O)c4ccccc4)cc23)c(C(C)C)c1C ; [2H][C@](O)(Cn1cc2c(-c3nn(-c4cc5cn(C)nc5nc4C)c(C)c3C(C)C)cccc2n1)c1ccccc1 ; CCOc1nc2nn(C)cc2cc1-n1nc(-c2cccc3nn(C[C@H](O)c4ccccc4)cc23)c(C(C)C)c1C] 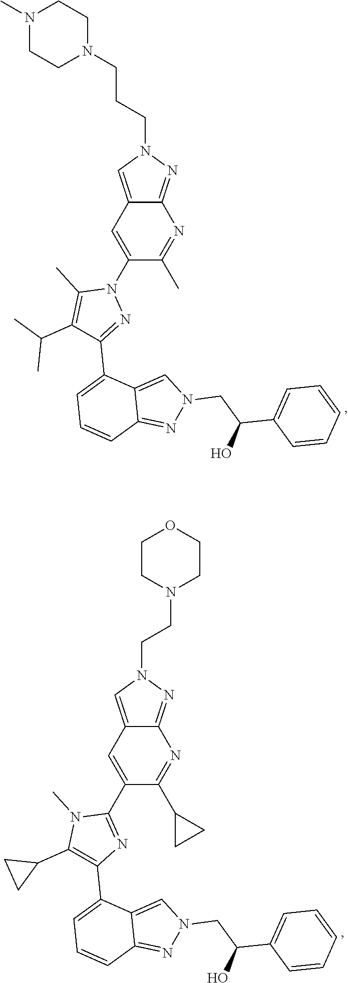 [SMILES: Cc1nc2nn(CCCN3CCN(C)CC3)cc2cc1-n1nc(-c2cccc3nn(C[C@H](O)c4ccccc4)cc23)c(C(C)C)c1C ; Cn1c(-c2cc3cn(CCN4CCOCC4)nc3nc2C2CC2)nc(-c2cccc3nn(C[C@H](O)c4ccccc4)cc23)c1C1CC1] 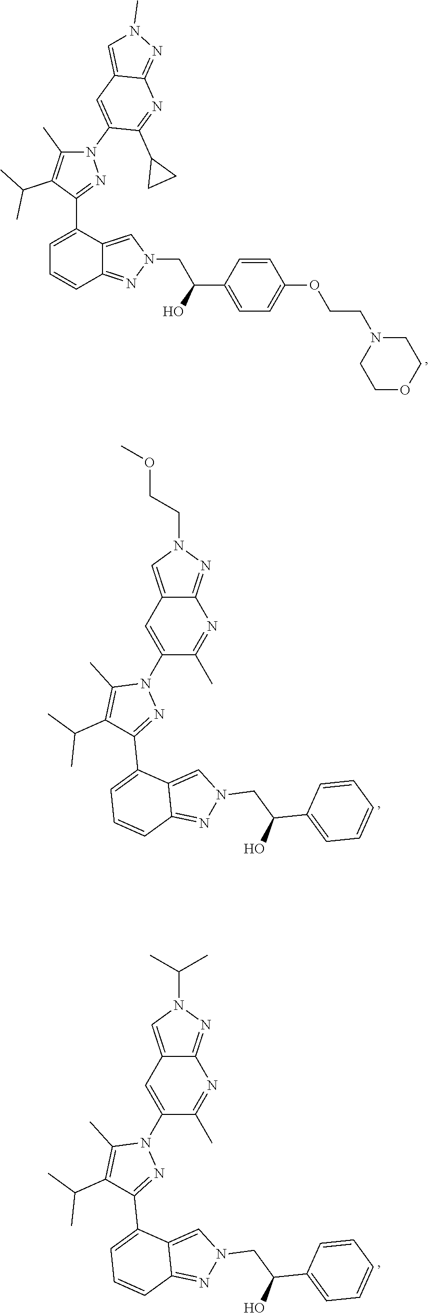 [SMILES: Cc1c(C(C)C)c(-c2cccc3nn(C[C@H](O)c4ccc(OCCN5CCOCC5)cc4)cc23)nn1-c1cc2cn(C)nc2nc1C1CC1 ; COCCn1cc2cc(-n3nc(-c4cccc5nn(C[C@H](O)c6ccccc6)cc45)c(C(C)C)c3C)c(C)nc2n1 ; Cc1nc2nn(C(C)C)cc2cc1-n1nc(-c2cccc3nn(C[C@H](O)c4ccccc4)cc23)c(C(C)C)c1C] 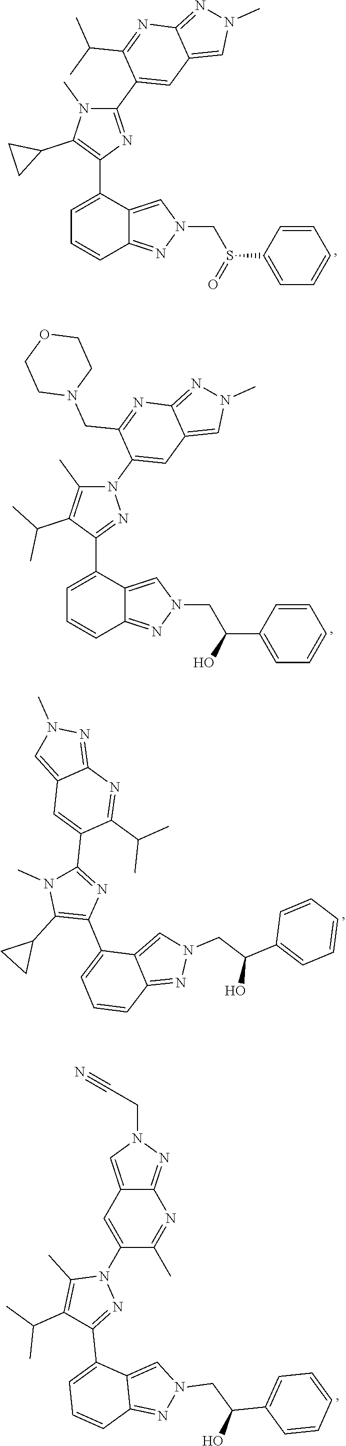 [SMILES: CC(C)c1nc2nn(C)cc2cc1-c1nc(-c2cccc3nn(C[S@@](=O)c4ccccc4)cc23)c(C2CC2)n1C ; Cc1c(C(C)C)c(-c2cccc3nn(C[C@H](O)c4ccccc4)cc23)nn1-c1cc2cn(C)nc2nc1CN1CCOCC1 ; CC(C)c1nc2nn(C)cc2cc1-c1nc(-c2cccc3nn(C[C@H](O)c4ccccc4)cc23)c(C2CC2)n1C ; Cc1nc2nn(CC#N)cc2cc1-n1nc(-c2cccc3nn(C[C@H](O)c4ccccc4)cc23)c(C(C)C)c1C] 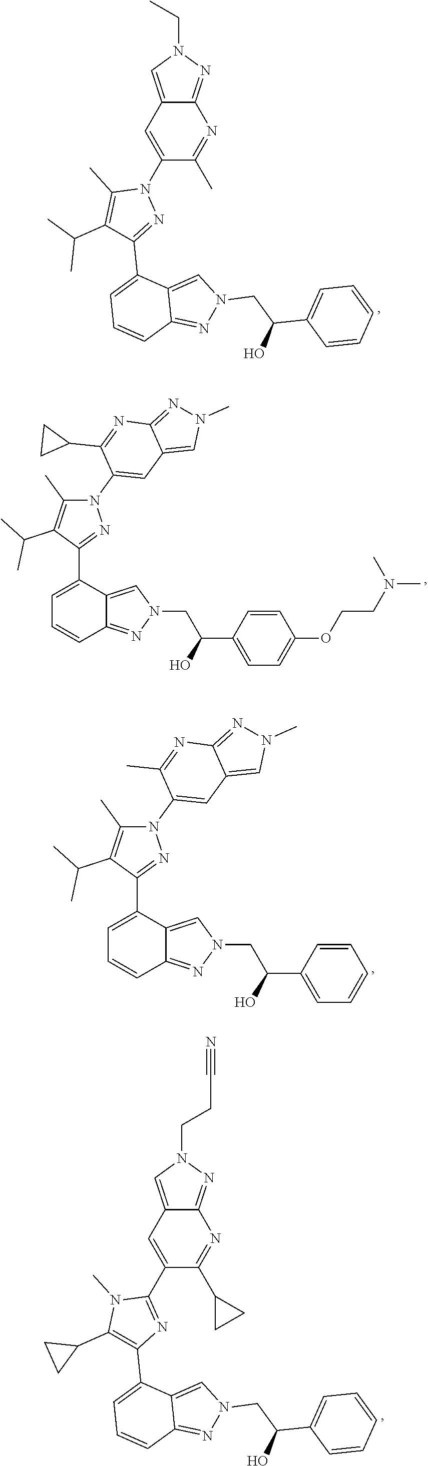 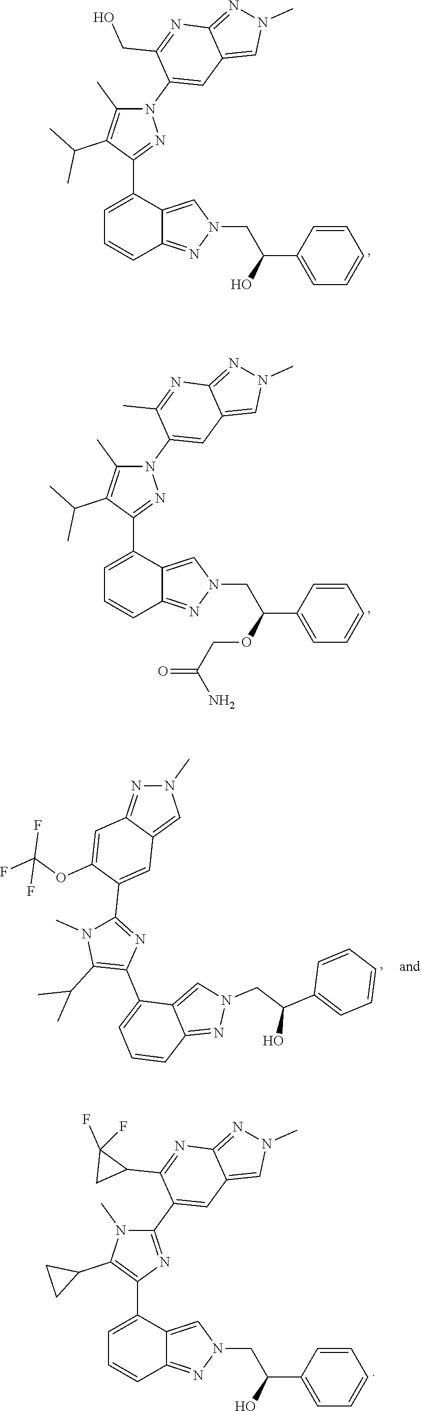 [SMILES: Cc1c(C(C)C)c(-c2cccc3nn(C[C@H](O)c4ccccc4)cc23)nn1-c1cc2cn(C)nc2nc1CO ; Cc1nc2nn(C)cc2cc1-n1nc(-c2cccc3nn(C[C@H](OCC(N)=O)c4ccccc4)cc23)c(C(C)C)c1C ; CC(C)c1c(-c2cccc3nn(C[C@H](O)c4ccccc4)cc23)nc(-c2cc3cn(C)nc3cc2OC(F)(F)F)n1C ; Cn1cc2cc(-c3nc(-c4cccc5nn(C[C@H](O)c6ccccc6)cc45)c(C4CC4)n3C)c(C3CC3(F)F)nc2n1]


SEQUENCE LISTING (3 sequences)

SEQ ID NO: 1 - protein, 204 aa, synthetic construct
  source 1..204: note = His-TEV-hSTING (WT)
MHHHHHHHHENLYFQSGVAHGLAWSYYIGYLRLILPELQARIRTYNQHYNNLLRGAVSQRLYILLPLDCGVPDNLSMADPNIRFLDKLPQQTGDRAGIKDRVYSNSIYELLENGQRAGTCVLEYATPLQTLFAMSQYSQAGFSREDRLEQAKLFCRTLEDILADAPESQNNCRLIAYQEPADDSSFSLSQEVLRHLRQEEKEEV

SEQ ID NO: 2 - protein, 6 aa, synthetic construct
HHHHHH

SEQ ID NO: 3 - protein, 8 aa, synthetic construct
HHHHHHHH